(19) United States
(12) Patent Application Publication
(10) Pub. No.: US 2020/0399428 A1
(43) Pub. Date: Dec. 24, 2020

(54) SYSTEMS, METHODS AND HYDROGELS FOR CELL CULTURE AND ANALYSIS

(71) Applicant: EVORION BIOTECHNOLOGIES GMBH, Münster (DE)

(72) Inventors: Hans KLEINE-BRÜGGENEY, Münster (DE); Robert WEINGARTEN, Münster (DE); Sebastian BÜHREN, Nottuln (DE)

(21) Appl. No.: 16/645,091

(22) PCT Filed: Sep. 11, 2018
(86) PCT No.: PCT/EP2018/074527

§ 371 Date: Mar. 6, 2020

Related U.S. Application Data
(60) Provisional application No. 62/623,772, filed on Jan. 30, 2018.

(30) Foreign Application Priority Data
Sep. 11, 2017 (EP) 17 190 298.4
Sep. 11, 2017 (EP) 17 190 299.2

(51) Int. Cl. C08J 3/075; C12M 1/12; C08G 83/00; C08G 73/02; C07K 14/00; C08L 79/02; C08J 11/10; C08G 81/02

(52) U.S. Cl. CPC C08J 3/075; C12M 25/14; C08G 83/002; C08G 73/0233; C07K 14/003; C08L 79/02; F16K 99/0015; C08G 81/028; C08G 2210/00; C08J 2379/02; C08L 2207/53; C08L 2203/02; C08J 11/105

(57) ABSTRACT

The present invention relates to hydrogels and polymers suitable as building blocks for hydrogels as well as advantageous methods for encapsulating cells and/or particles. Also provided are kits and methods for producing the hydrogels.

### FIELD OF THE INVENTION

The present invention pertains to a novel microfabricated array of hydrogel matrices that includes novel microstructures for flow control within said array as well as novel methods for producing said array including novel methods for the formation of emulsions as well as for the encapsulation of single or multiple cells of the same type or of different types into novel spherical hydrogel matrices with defined characteristics (including mechanical, physical and biological characteristics) and defined sizes, subsequent controlled positioning/immobilization of said spherical hydrogel matrices within said microfabricated array for long-term imaging, perfusion culture, stimulation and on-chip characterization as well as recovery of hydrogel beads that are of interest at any time point from any location for further downstream analysis (e.g. RT-PCR, NGS).

In addition, the present invention is directed to novel chemical compounds and reactions for the formation of defined hydrogel structures located in said array that are composed of heterocyclic chemical compounds such as 2-oxazoline and unsaturated imides such as 3-(maleimido)-propionic acid N-hydroxysuccinimide ester that can be used among others for the immobilization of biological compounds as well as for the encapsulation of cells and their cultivation. Finally, the present invention is directed to methods for the analysis of cells and cellular compounds located within said array such as the repeated on-demand stimulation of cells located at defined position, the generation of time-lapse cytokine profiles of cultivated cells as well as for the analysis of cellular characteristics such as mRNAs/miRNA or surface proteins.

### BACKGROUND

To date, most cell-based assays use traditional two-dimensional (2D) monolayer cells cultured on flat and rigid substrates or animal derived three-dimensional cell culture systems as models to investigate complex cell behavior, especially in the field of cancer, stem cell and immunooncology research. Because nearly every cell in the human body is surrounded by an extracellular matrix (ECM), 2D cell culture does not adequately consider the natural 3D environment of cells. Thus, cells cultivated within two-dimensional cell culture systems differ morphologically and physiologically from cells surrounded by a natural environment. Altered cellular responses to external stimulus e.g. drug(s) in 2D cell culture tests sometimes provide misleading and nonpredictive data for in vivo responses. Consequently, only about 10% of the compounds tested with 2D monolayer culture systems progress successfully through clinical development. A better solution for the investigation of cells within their natural environment are 3D cell culture systems. But to date, naturally derived 3D cell culture systems have poorly defined compositions and show batch to batch variability regarding mechanical and/or biochemical properties. Because of the natural origin, the risk of contamination with several toxins is very high. These drawbacks make it impossible to investigate responses from precise alterations of mechanical and biochemical properties in systematic ways to independently control key parameters responsible for cell behavior and cell characteristics.

The current understanding of cellular responses to external stimuli is generally based on using bulk assays on populations of cells though cell cultures are not of a homogeneous nature. Nearly all cell populations can be divided into subpopulations and even within the subpopulations of the same cell type each cell is different from the other. In addition, the transcriptional response to stimuli is heterogenous and a digital process at the single cell level. Analyzing a collection of cells does not give an accurate assessment of the behavior of a particular cell in that culture or tissue. Accordingly, the average response of the cells is interpreted as the response of all cells in that sample. Specialized cells which exist in nearly all cell populations (e.g. cancer stem cells) are ignored in such bulk assays and valuable information about these cells is lost.

Accordingly, it is an object of the present disclosure to overcome the drawbacks of the prior art, in particular to provide hydrogel matrices as cell carriers as well as microfabricated systems and methods that enable the rapid and precise positioning and recovery of encapsulated single cells and small cell population within these hydrogel matrices. The hydrogel matrices controlling cell behavior and cell characteristics enable together with novel microfabricated systems and methods performing dynamic studies of living single cells and small populations of cells which can increase the understanding of the interconnecting molecular events coupling phenotypic events to the underlying genotype of particular cells.

### SUMMARY OF THE INVENTION

In a first aspect, the present disclosure pertains to a microfabricated valve (10), comprising a first channel (11);

a second channel (12);

a connection channel (13) connecting the first channel (11) and the second channel (12);

a valve portion (14) arranged within the connection channel (13),

wherein the valve portion (14) is adapted to selectively open and close the connection channel (13).

In a second aspect, the present disclosure pertains to a test device (30), in particular for biological applications, comprising a plurality of observation chambers (32), wherein the observation chamber (32) is adapted to accommodate at least one droplet (31), the droplet in particular comprising a hydrogel particle, provided within a fluid.

In a third aspect, the present disclosure pertains to methods of creating droplets, in particular encapsulations, within a first fluid, comprising the following steps:

a) providing a microfabricated valve (10) according to the present disclosure,

wherein the first channel (11) is filled with a first fluid,

wherein the second channel (12) is filled with a second fluid,

wherein the second fluid is insoluble in the first second fluid,

b) applying a pressure difference (p2-p1) to the fluids, wherein the second fluid is pressurized by a second pressure (p2) and the first fluid is pressurized by a first pressure (p1), wherein the second pressure (p2) is larger than the first pressure (p1),

c) selectively opening the valve portion (14),

d) subsequently closing the valve portion (14) as soon as a defined quantity of the second fluid has passed the valve portion (14) in direction from the second channel (12) to the first channel (11).

In a further aspect, the present disclosure pertains to methods for performing a biological test cycle, in particular using a test device (10) according to the present disclosure, comprising the steps providing a plurality of droplets, in particular comprising particles (20), within a stream of fluid; selectively trapping one individual droplet (31) or a preset number of droplets within an observation chamber (32), in particular within a trap (33) of the observation chamber (32).

In a further aspect, the present disclosure pertains to a method for demulsification of droplet comprised within a first fluid, comprising the following steps:

a) providing a microfabricated valve (10) according to the present disclosure or a test device according to the present disclosure,

wherein the first channel (11) is filled with a first fluid,

wherein the second channel (12) is filled with a second fluid,

wherein in the first channel (11) a droplet (31) of a second fluid is comprised,

wherein the second fluid is insoluble in the first second fluid,

In a further aspect, the present disclosure pertains to a pump (50), comprising at least two, in particular at least three, valves (10) according to any of claims 1 to 6, arranged in series, wherein the pump (50) is adapted to pump a fluid upon, in particular a sequential, activation of the valves (10A, 10C; 10C), in particular wherein, considered in a direction (F) of fluid, an outlet channel (12A) of a first valve (10A) is connected to an inlet channel (12B) of a second valve (10B), and/or in particular wherein, considered in a direction (F) of fluid, an outlet channel (11B) of a second valve (10B) is connected to an inlet channel (11A) of a third valve (10C).

In a further aspect, the present disclosure pertains to organic monomers comprising a covalently functionalized D-substituted alkylamine.

Furthermore, the present disclosure pertains to hydrogel matrices composed of a mixture of at least two different organic polymers according to the present disclosure. In particular, the hydrogel matrices according to the present disclosure are useful for single cell assays and/or microfluidic arrays. They are described in detail below.

In a further aspect, the present disclosure pertains to a microfluidic array having microfabricated structures for the generation and/or immobilization and/or recovery of a hydrogel matrix according to any one of the proceeding claims containing at least one particle and/or cell located for analysis of cell characteristics and/or behavior and methods for producing said array.

The present disclosure relates also to methods of assigning secretome phenotypes of cells to the underlying genotypes of the cells by sequential reverse flow cherry picking comprising:
forming individual matrices of at least two different types:
one type of matrices “A” wherein each individual matrix comprises a single cell and/or at least two cells of different cell types.
one type of matrices “B” wherein each individual matrix comprises a first binding agent specific for a first binding region on the target analyte secreted from the single and/or multiple cells within matrix “A” wherein the first binding agent comprises a functionalized portion for immobilization to a solid support.
immobilizing the present disclosure;
binding target analyte expressed by single and/or multiple cells encapsulated in matrix “A” in Matrix “B” with the polypeptide capture molecules (second binding agent) to generate bound target analyte—polypeptide capture molecule (second binding agent) complexes;
labeling the bound target analyte in Matrix “B” by perfusion or diffusion with a second binding agent specific for a second binding region on the target analyte and comprising a target identifier (e.g. barcoded oligonucleotides) for identifying the target molecule; isolating Matrix “B” from the array by reverse flow cherry picking into a well plate or similar format. This step links the expressed target to the immobilized single cell and/or cell populations within Matrix “A”;
detecting and quantifying the target identifier (e.g. by using qRT-PCR, sequencing (Illumina, Pacific Bioscience, Oxford Nanopores);
immobilizing an analyte free Matrix “B” for further binding of target analytes expressed by single and/or multiple cells encapsulated in matrix “A”.

The present disclosure relates also to organic building blocks comprising a substituted tertiary amide group represented by the formula:

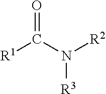

The present disclosure relates also to methods for manufacturing an organic building block comprising a substituted tertiary amide group represented by the formula:

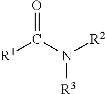

wherein the tertiary amide group results from a copolymerization of at least two components,

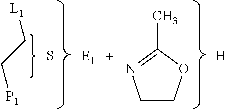

wherein the first component (E1) comprises at least one of three different parts:
   a first functional group (P1) for the copolymerization with the second component,
   a second functional group (L1) for crosslinking to a biologically active compound, and
   an optional spacer (S) between the two functional groups, and
wherein the second component for polymerization with the first functional group is a heterocyclic chemical compound (H).

A preferred polymer, especially for hydrogel formation, is a polymer comprising at least one unit (m is at least 1) having the structure of the following formula

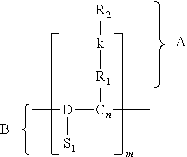

wherein
R₂ is independently a residue R⁴, comprising at least one functional group
   for crosslinking and/or
   for binding biologically active compounds,
S₁ is independently defined according to R¹ of claim 1,
fragment D-Cn is part of the polymer backbone,

wherein said structure results from polymerization of a heterocyclic molecule B in presence of a first component A (vide infra).

For the formation of a hydrogel according to this invention, it is mandatory, that a polymer used as a building-block for said hydrogel comprises at least one residue comprising a functional group, independently selected from a functional group
for crosslinking and/or
for binding biologically active compounds.

Said at least one residue preferably comprises in addition to said functional group a spacer moiety connecting said functional group with the binding site for said respective residue to the polymer backbone. In order to provide an on demand degradation of said hydrogel it is preferred, that said spacer moiety is degradable.

Preferred polymers, especially as building-block for hydrogel formation, comprise at least one moiety of formula (I) and one moiety of formula (II)

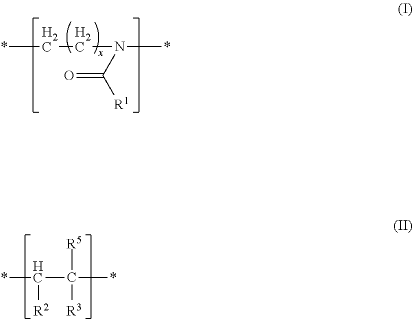

wherein
R¹ is a hydrogen atom, a hydrocarbon with 1-18 carbon atoms, a C₁-C₂₅-hydrocarbon with at least one hydroxyl group, a C₁-C₂₅-hydrocarbon with at least one carboxy group, (C₂-C₆)alkylthiol, (C₂-C₆)alkylamine, protected (C₂-C₆)alkylamine (preferably-(CH₂)₂₋₆—NH—CO—R (with R=benzylhydryloxy, 9-fluorenylmethoxy)), (C₂-C₆)alkylazide, polyethylene glycol, a crosslink to R¹ of another moiety of formula (I), polylactic acid, polyglycolic acid or polyoxazoline, or wherein R¹ is a residue R⁴,
R² and R³ R² and R³ are linked to form a cyclic moiety of formula (II) comprising at least one residue R⁴
   or R² and R³ are independently selected from hydrogen, —COOH, methyl or a residue R⁴, wherein optionally, at least one of R² and R³ is a residue R⁴,
R⁴ is a moiety, comprising at least one functional group, independently selected from a functional group
   for crosslinking and/or
   for binding biologically active compounds, and optionally comprising a (preferably degradable) spacer moiety connecting said functional group with the binding site of the respective moiety of formula (I) or formula (II), and
R⁵ denotes a hydrogen atom, a carboxymethyl group or a methyl group,
x is 1, 2 or 3, and
* denotes a chemical bond of the polymer backbone or to a terminating moiety,

with the proviso, that at least one moiety of formula (I) or formula (II) comprises a residue R⁴, wherein preferably only the moieties of formula (I) or only the moieties of formula (II) comprise at least one moiety R⁴.

Another preferred polymer, especially for hydrogel formation, is a polymer comprising at least one unit having the structure of formula

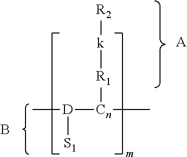

wherein
R₂ is independently a residue R⁴, comprising at least one functional group
   for crosslinking and/or
   for binding biologically active compounds,
S₁ is independently selected from a hydrogen atom, a hydrocarbon with 1-18 carbonatoms, a C₁-C₂₅-hydrocarbon with at least one hydroxy group, a C₁-C₂₅-hydrocarbon with at least one carboxy group, (C₂-C₆)alkylthiol, (C₂-C₆)alkylamine, protected (C₂-C₆)alkylamine, (C₂-C₆)alkylazide, polyethylene glycol, a crosslink to R¹ of another moiety of formula (I), polylactic acid, polyglycolic acid or polyoxazoline, or wherein R¹ is a residue R⁴,
fragment D-Cn is part of the polymer backbone,

wherein said structure results from polymerization of a heterocyclic molecule B in presence of a first component A (vide infra).

A preferable useful polymer, especially for hydrogel formation, is a polymer of formula (P1)

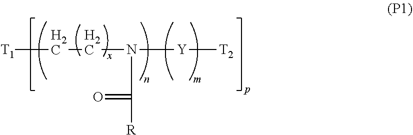

wherein
R is independently selected from a hydrogen atom, a hydrocarbon with 1-18 carbonatoms, a C₁-C₂₅-hydrocarbon with at least one hydroxy group, a C₁-C₂₅-hydrocarbon with at least one carboxy group, (C₂-C₆)alkylthiol, (C₂-C₆)alkylamine, protected (C₂-C₆)alkylamine (preferably-(CH₂)₂₋₆—NH—CO—R (with R=tert-Butyl, perfluoroalkyl)), (C₂-C₆)alkylazide, polyethylene glycol, polylactic acid, polyglycolic acid, polyoxazoline, or wherein R is a residue R⁴
   Y is a moiety containing at least one graft, comprising at least one residue R⁴,
   T₁ is a terminating moiety, which may contain a residue R⁴,
   T₂ is a terminating moiety, which contains a residue R⁴,
   p is an integer from 1 to 10,
   n is an integer greater than 1 and preferably, below 500,
   m is zero or an integer of at least, preferably greater than 1, and preferably, below 500, the sum n+m is greater than 10,
   x is independently 1, 2 or 3, preferably x is independently 1 or 2, most preferably x is 1,
   R⁴ independently comprise at least one functional group
      for crosslinking and/or
      for binding biologically active compounds, and
   optionally comprising a (preferably degradable) spacer moiety connecting said functional group with the binding site to the respective moiety of the structure of formula (P1),
   wherein the entirety of all m-fold and n-fold repeating units are distributed in any order within the polymer chain and wherein optionally, the polymer is a random copolymer or a block copolymer.

The present disclosure relates also to the use of an organic monomer and/or organic building block according to the present disclosure for polymerization resulting in a hydrophilic polymer comprising at least two organic monomers of any one of the preceding claims.

The present disclosure relates also to organic polymers comprising at least two organic monomers and/or organic building blocks according to the present disclosure.

The present disclosure relates also to hydrogels and biomaterials for cell applications composed of a mixture of at least two different organic polymers according to.

Particularly preferred hydrogels are described herein that allow the encapsulation of cells and/or particles.

The present disclosure relates also to methods for the production of a biomaterial for cell-based applications, which method has the following consecutive steps:

a) providing of one or more organic polymer and/or organic building blocks according to any one of the preceding claims

b) functionalization of the polymer from step a) with at least one biologically active molecule

c) addition of a crosslinking agent for crosslinking the polymer functionalized in step b) to generate the biomaterial.

d) degradation of the crosslinking agent to release ingredients from the biomaterial The present disclosure relates also to organic building blocks manufactured with a method according to the present disclosure.

The present disclosure relates also to droplets comprising a hydrogel/hydrogel matrix composed of an organic monomer, organic building block and/or an organic polymer according to the present disclosure.

The present disclosure relates also to the use of an organic monomer and/or an organic building block according to the present disclosure for the polymerization of a hydrophilic polymer comprising at least two organic monomers and/or organic building blocks according to the present disclosure.

The present disclosure relates also to a hydrogel matrix composed of a mixture of least two different organic polymers and/or organic building blocks according to the present disclosure, and/or composed of an organic polymer according to the present disclosure.

The present disclosure relates further to methods of bioactive modifications of hydrogel vehicles (carrier) with components for forward and backward genetic analysis (genome editing), comprising:
forming individual vehicles of at least two different types:
vehicle “A” comprises a single cell and/or at least two cells of different cell types.
vehicle “B” comprises immobilized cell-penetrating peptide coupled to Cas9 protein and cell-penetrating peptide complexed with guide RNA
immobilizing Matrix “A” and Matrix “B” next to each other onto the array according to the present disclosure;
releasing immobilized cell-penetrating peptide coupled to Cas9 protein and cell-penetrating peptide complexed with guide RNA either on demand by the user or pulsed by a cell cycle dependent signal via enzymatically degradation or photo-cleavage of the linker;
Formation of Cas9-guide RNA complexes and targeting of the complex into the nucleus of the cell incorporated in vehicle “A” by an incorporated nuclear localization sequence; Removing empty vehicle “B” from the array by reverse flow cherry picking and immobilizing a fresh vehicle “B” for further treatments.

Before the disclosure is described in detail, it is to be understood that the terminology used herein is for purposes of describing particular embodiments only, and is not intended to be limiting. It must be noted that, as used in the specification and the appended claims, the singular forms “a,” “an” and “the” include singular and/or plural reference unless the context clearly dictates otherwise. It is moreover to be understood that, in case parameter ranges are given which are delimited by numeric values, the ranges are deemed to include these limitation values.

### BRIEF DESCRIPTION OF THE DRAWINGS

FIG. 1 shows an overall workflow.

FIG. 2 shows a microfluidic array 30 as an inventive test device 30, having microfabricated structures. The array 30 comprises a plurality of observation chambers 32m1n1 to 32 m4n4, arranged in columns m1 to m4 and lines n1 to n4. The array 30 could have any number y of columns and numbers z of lines, resulting in a number x=y*z chambers. All observation chambers 32 are connected in series by a feeding channel 41, connecting an inlet for loading 42 with the series of chambers 32 and subsequently with a feeding exit 43, when viewed in first fluid direction. In the feeding channel a pump 50 described later can be provided to pump the fluid in channels.

FIG. 3 shows a microfluidic array 30 having a plurality of observation chambers 32, such a chamber 32m2n2 at position m2 n2, each loaded with (single) cell(20)-laden spherical hydrogels 31 under perfusion culture. Depicted are the rows n and columns m of the array as well as corresponding observation chambers. Lines representing rows and columns are illustrating pressure lines for providing common group commands as will be described. Circles illustrate individual observation chambers. Each observation chamber may contain at least one particle/droplet with defined characteristics. In particular, each observation chamber may contain hydrogel particle/matrices with defined characteristics (e.g. elasticity, immobilized ECM proteins and/or peptides, in particular RGD sites, fibronectin, YIGSR peptides, collagen, LDV peptides, laminin). Said hydrogel particles/matrices may contain at least one biological cell (e.g. an immune cell, a cancer cell, a stem cell).

FIG. 4 shows a layer description of microfabricated elastomer valve 10. In a particular embodiment, a bottom microfabricated layer 21 contains an oily fluid. The space above the microfabricated layer 21 is connected with the top microfabricated layer 23 by first recess 19a within microfabricated layer. The space above microfabricated layer 21 may be an open reservoir. The first recess 19a is separated from a second recess 19b within intermediate microfabricated layer 22 by a thin membrane 15 with defined thickness, which is part of a valve portion 14. Applying an actuation force on this membrane 15 results in membrane bending and closing the connection between recesses 19a, 19b thus in a separation of the space above microfabricated layer 21 and the recess of microfabricated layer 23.

The term “insoluble means” in particular that max. 0.1 g of the first fluid is soluble in 100 ml the second fluid.

The term “Microfabricated” indicates that the dimensions of the structures within the claimed devices are in the area of micrometers, in particular between 0.1 micrometers and 1000 micrometers. For manufacturing the devices in particular lithographical methods are used.

FIG. 60 shows an exemplary method to arrange the first 21, second 22 and third layer 23 after providing, e.g. inserting the first channel 11 into the first layer 21, the second channel 12 into the third layer 23 and the connection channel 13 into the second layer 22. The second layer 22 is arranged between the first 21 and third layer 23.

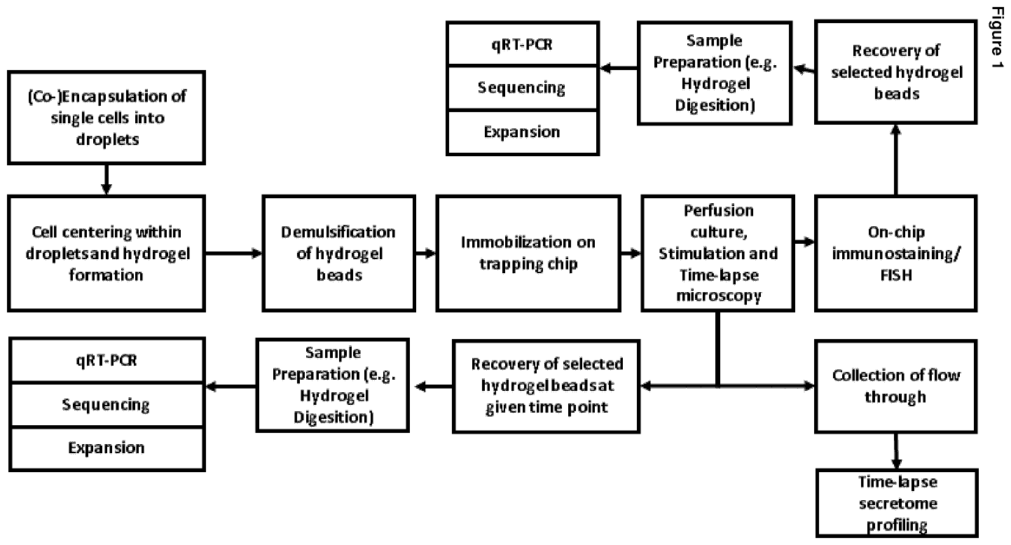

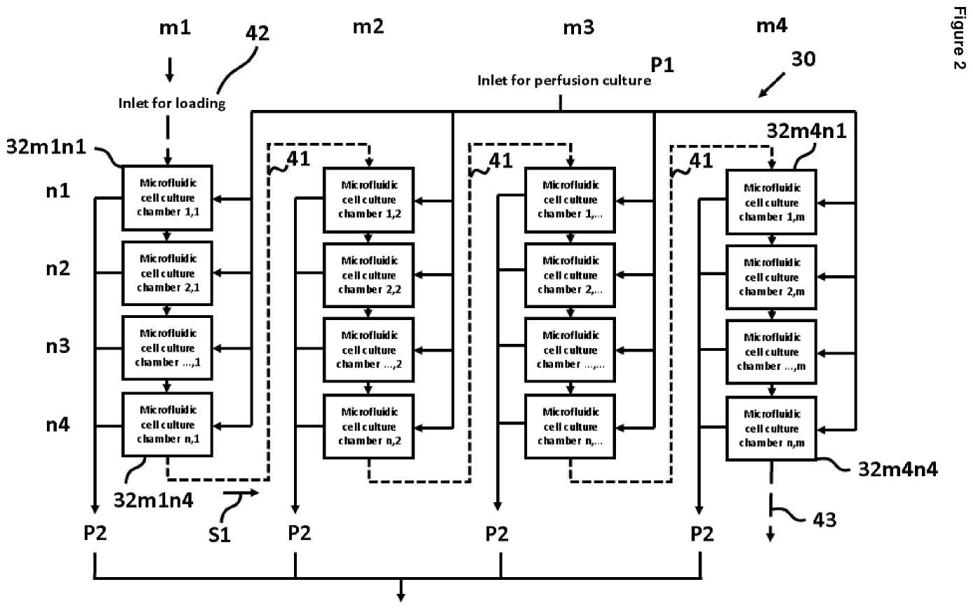

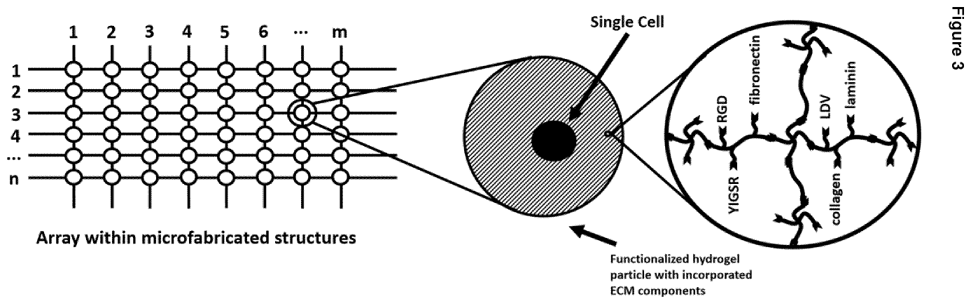

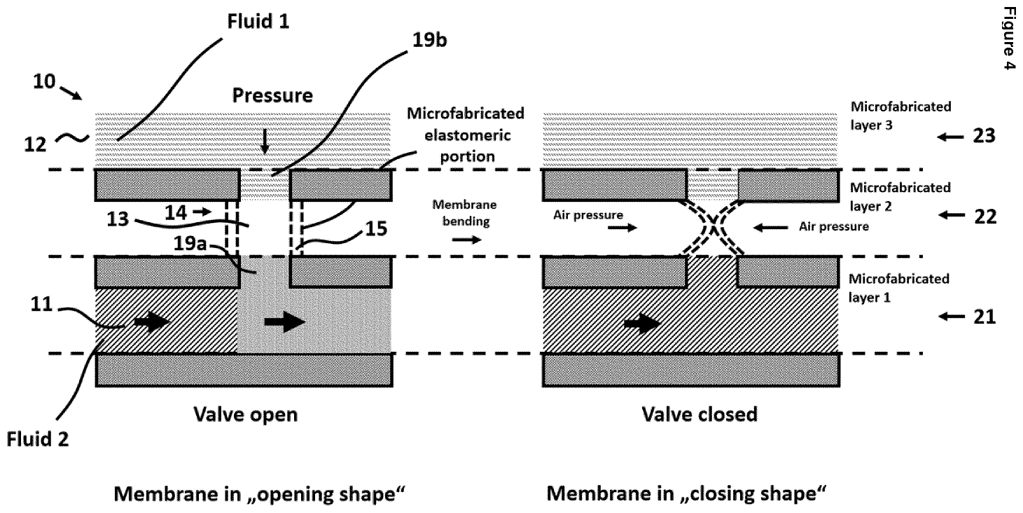

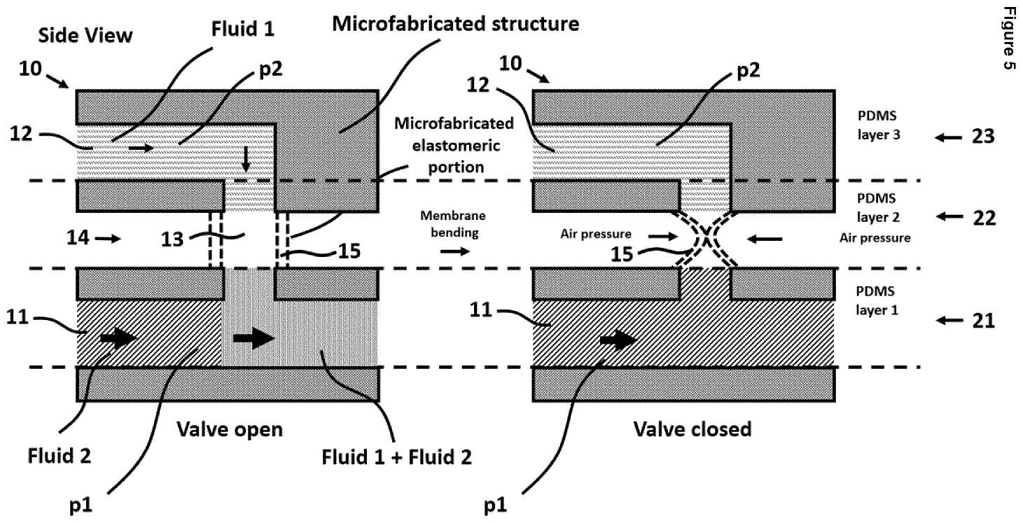

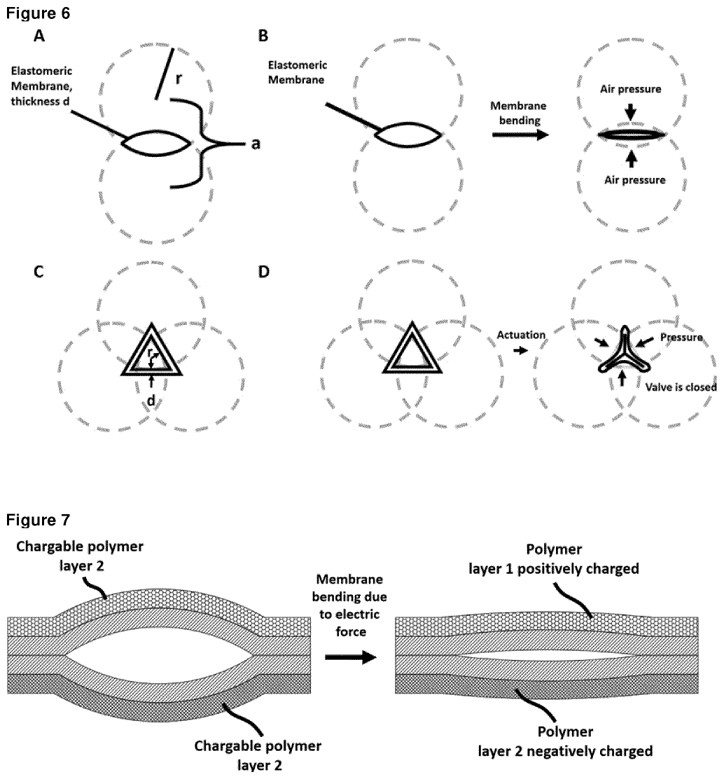

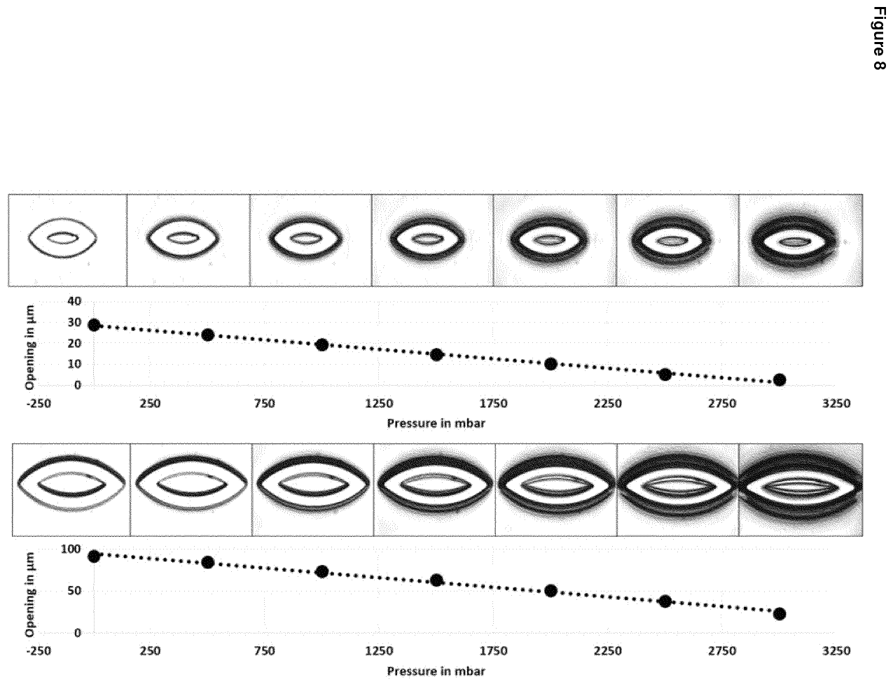

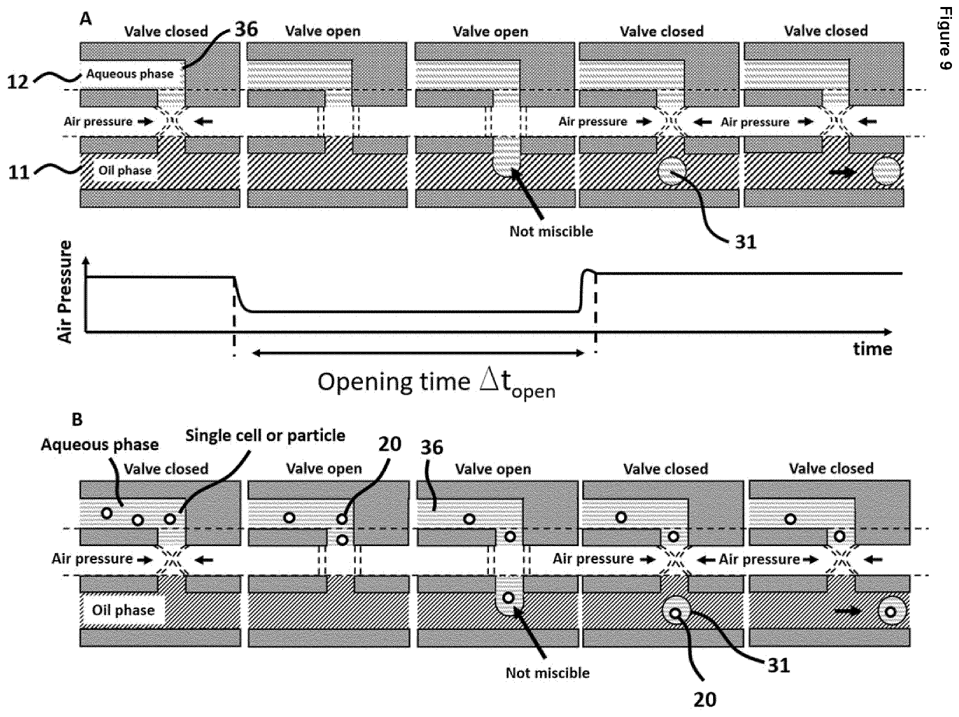

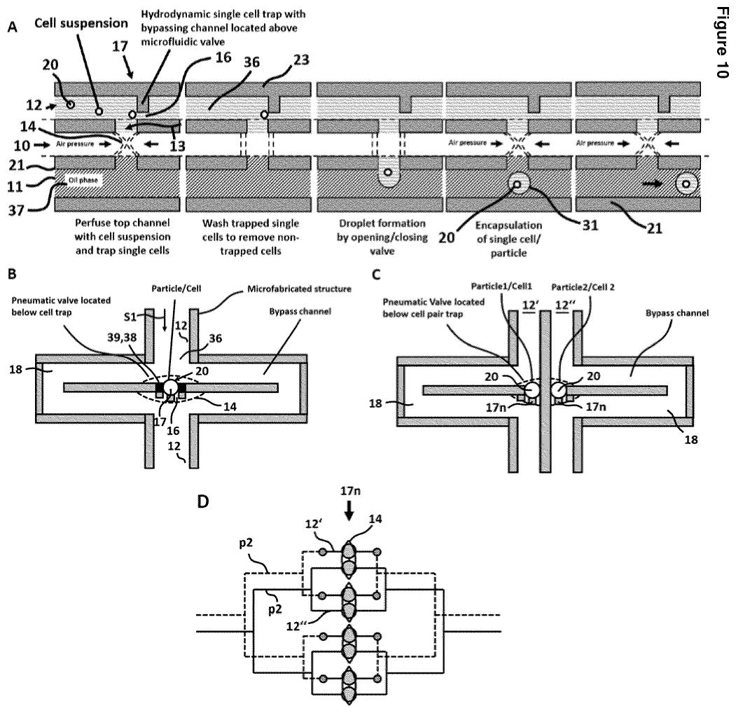

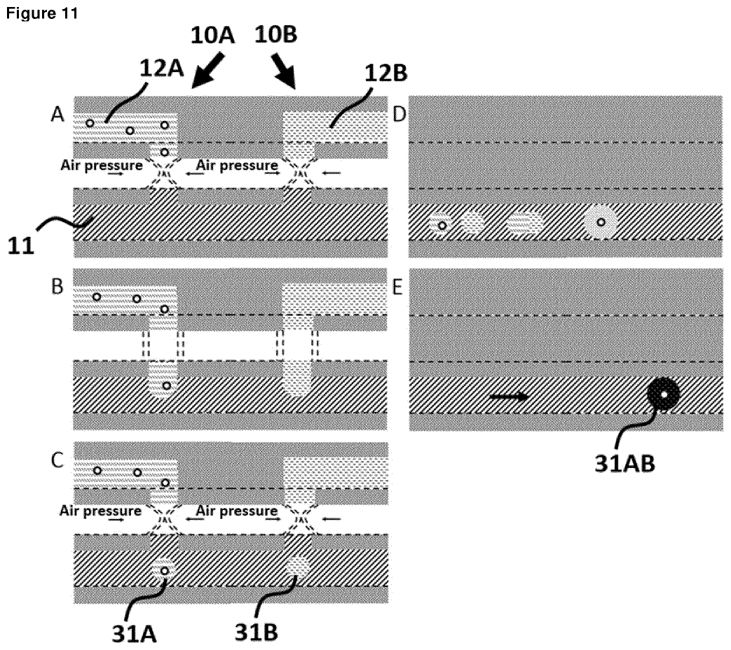

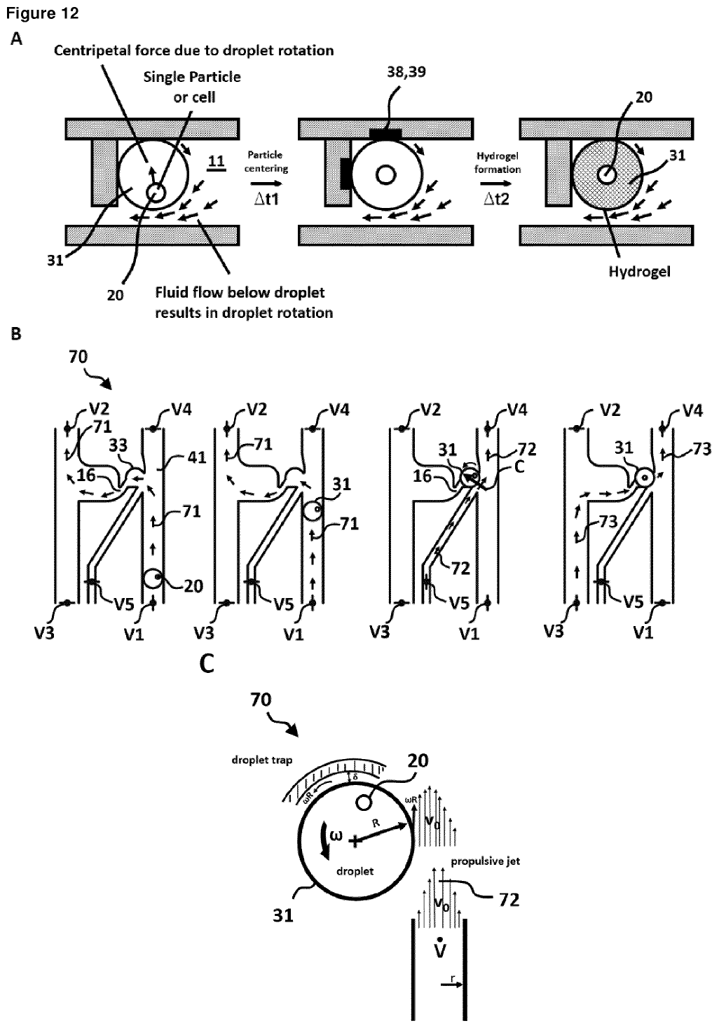

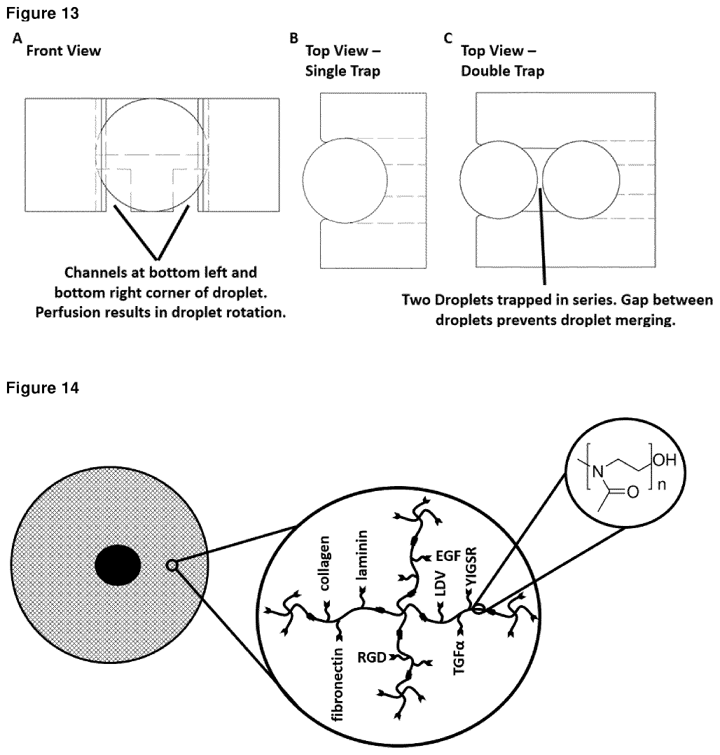

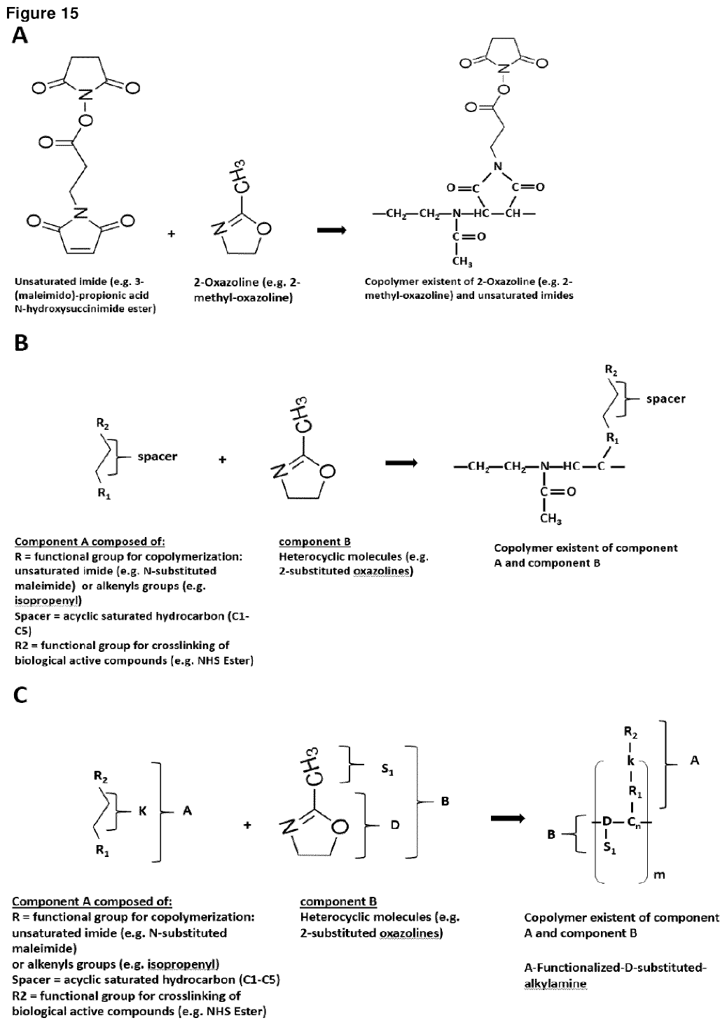

In this example, the first layer 21 is first connected to a glass plate 120 and then the second layer is laid on top of the first 21 layer and connected to it. The binding of the different layers as well as the glass plate may be done using established binding methods for microfluidic devices such as surface functionalization using an oxygen plasma. Then the third layer 23 is placed on the second layer 22 and connected to it. Alternatively, it is possible to first connect the three layers to each other and then to place the composite of the three layers on the glass plate 120 and connect it to the glass plate. Various other sequences of these steps are possible and known to the skilled person. In this example, the glass plate 120 forms a wall of the first channel (11). As can be seen in the figure, in this example the second opening (1) is in the second layer 22, while the first opening (2) is in the first layer 21.

FIG. 5 is an illustration of a microfabricated elastomer valve 10 with a microfabricated channel located above microfabricated layer 21.

FIG. 61 illustrates the consequences of the thickness d_{N} of the actuation chamber 3 being too large above or below the first 11/second channel 12. FIG. 61a shows a cross-section through the microfabricated valve with the flexible membrane (15) in an unloaded state (pressure P0). If high pressure P1 is applied to the actuation chamber 3 to close the flexible membrane 15 (see FIG. 61b), not only is the membrane wall displaced laterally, but there is also an upward deflection of the part of the second channel 12 above the actuation chamber 3. The same applies to the part of the first channel 11 below the actuation chamber 3. If the actuation chamber 3 is thin in this area, this undesirable side effect can be reduced or even completely eliminated.

FIG. 6 is an illustration of different valve geometries (top view) and corresponding naming. A) Biconvex microfabricated valve structure. B) Valve actuation results in membrane bending and closing of the inner opening. C) Triangular shape valve. D) Illustration of triangular valve actuation.

FIG. 62 shows an example of a portion of the microfabricated valve. The valve portion (14) is arranged within the connection channel (13) which means that it is at least part of the connection channel (13). The valve portion (14) comprises a flexible membrane (15), wherein the flexible membrane forms at least part of the outer wall of the connection channel 13. The flexible membrane (15) can have a homogeneous or inhomogeneous thickness, wherein the flexible inhomogeneous membrane has a thinned section which has a reduced thickness compared to at least one other section of the flexible membrane, this section being the one adjacent to the first layer, and a projection of the first channel along the longitudinal axis of the connecting channel meets this thinned section 121 and/or wherein the flexible membrane has a thinned section which has a reduced thickness compared to at least one other section of the flexible membrane. In one embodiment, the thinnest section 121 of the membrane is the section which undergoes the highest deflection distance required for fully closing/operating the valve portion (14). The thinnest section might be for example 20 μm. The largest membrane thickness might for example be 44 μm. Furthermore, the flexible membrane comprises an inner boundary (4) forming the outer wall of the connection channel (13) or encompassing at least one section of the connection channel (13) and an outer boundary (5) forming the outer wall of the flexible membrane. The valve portion (14) is adapted to be selectively opened and closed, and in particular transferred into an intermediate shape, upon modification of a pressure difference between the actuation chamber (3) and the connection channel (13) by modification of the pressure inside the actuation chamber (3), wherein the pressure inside the chamber is adjusted, in particular by an actuation fluid which can flow into the actuation chamber to increase the pressure inside the chamber or to flow out of the chamber to decrease the pressure inside the chamber, in particular to generate a vacuum inside the actuation chamber (3). The thickness of the membrane and also the homogeneity/inhomogeneity can change throughout the adaption of the valve portion. The actuation chamber (3) surrounds the valve portion (14) and the boundary of the actuation channel (6) is not in direct contact with the flexible membrane.

FIG. 63 shows an example of a portion of the microfabricated valve, which is similar to FIG. 62. In this example, the inner boundary and the outer boundary of the flexible membrane have a round shape, which is advantageous as it has a smallest footprint allowing a very high density of valves per area. The flexible membrane has a homogeneous thickness. Depicted is further the cross-section (7) of the connection channel (13), wherein the cross-section and the connection channel have a round shape. The actuation chamber (3) comprises the boundary section of the actuation chamber (6) and both comprise a round shape. Arrows indicate direction of membrane deflection if a sufficient actuation pressure is applied.

FIG. 64 shows an example of a portion of the microfabricated valve, which is similar to FIG. 62. In this example, the inner boundary and the outer boundary of the flexible membrane have a triangular shape. The flexible membrane has a homogeneous thickness. Depicted is further the cross-section (7) of the connection channel (13), wherein the cross-section and the connection channel have a triangular shape. The actuation chamber (3) comprises the boundary section of the actuation chamber (6) and both comprise a triangular shape. Arrows indicate direction of membrane deflection if a sufficient actuation pressure is applied.

FIG. 65 shows an example of a portion of the microfabricated valve, which is similar to FIG. 62. In this example, the inner boundary and the outer boundary of the flexible membrane have a rectangular shape. The flexible membrane has a homogeneous thickness. Depicted is further the cross-section (7) of the connection channel (13), wherein the cross-section and the connection channel have a rectangular shape. The actuation chamber (3) comprises the boundary section of the actuation chamber (3) and both comprise a rectangular shape. Arrows indicate direction of membrane deflection when actuation pressure is increased.

FIG. 66 shows an example of a portion of the microfabricated valve, which is similar to FIG. 62. In this example, the inner boundary and the outer boundary of the flexible membrane have a pentagonal shape. The flexible membrane has a homogeneous thickness. Depicted is further the cross-section (7) of the connection channel (13), wherein the cross-section and the connection channel have a pentagonal shape. The actuation chamber (3) comprises the boundary section of the actuation chamber (6) and both comprise a pentagonal shape. Arrows indicate direction of membrane deflection if a sufficient actuation pressure is applied.

FIG. 67 shows an example of a portion of the microfabricated valve, which is similar to FIG. 62. In this example, the inner boundary and the outer boundary of the flexible membrane have a biconvex shape. At least two membrane sections deflect towards each other. Depicted is further the cross-section (7) of the connection channel (13), wherein the cross-section and the connection channel have a biconvex shape. The actuation chamber (3) comprises the boundary section of the actuation chamber (6) and both comprise a biconvex shape. Arrows indicate direction of membrane deflection if a sufficient actuation pressure is applied.

FIG. 68 shows an example of a portion of the microfabricated valve, which is similar to FIG. 62. In this example, the inner boundary and the outer boundary of the flexible membrane have a concave shape at two sections and a not concave shape two sections, wherein concave and not concave sections are adjacent to each other. At least two membrane sections deflect towards each other. The flexible membrane has a homogeneous thickness. Depicted is further the cross-section (7) of the connection channel (13), wherein the cross-section and the connection channel mostly have a biconcave shape. The actuation chamber (3) comprises the boundary section of the actuation chamber (6) and both comprise a mostly biconvex shape. Arrows indicate direction of membrane deflection if a sufficient actuation pressure is applied.

FIG. 69A shows an example of a portion of the microfabricated valve, which is similar to FIG. 68. In this example, the inner boundary and the outer boundary of the flexible membrane have a concave shape at four sections, resulting in a biconcave-biconcave shape At least four membrane sections deflect towards each other, which is advantageous as all edges of the inner boundary are curved (8) towards the connection channel. Depicted are further the openings (1, 2), wherein the openings are substantially coaxial.

FIG. 69B shows an example of a portion of the microfabricated valve, which is similar to FIG. 69A. In contrast to FIG. 69A, the curved inner boundary is straight and comprises another edge, which is adjacent to the openings (1, 2), wherein the inside turned edges are lamellas (9). The lamellas are advantageous, as they enable a smaller dead volume of the connection channel (13), decrease the deflection distance required for fully closing/operating the valve portion (14), which results in a faster valve operation and additionally, in a smaller actuation pressure. Furthermore, this example is advantageous as the valve portion (14) has a small footprint allowing a very high density of valves per area. Arrows indicate direction of membrane deflection if a sufficient actuation pressure is applied.

FIG. 70 shows an example of a portion of the microfabricated valve, which is similar to FIG. 62. In this example, the actuation chamber (3) surrounds the valve portion (14) and the boundary of the actuation chamber (6) is in direct contact (in contrast to FIG. 62) with the flexible membrane at the boundary of the actuation chamber (101: merging position) with the outer boundary of the valve portion. This example of a microfabricated valve is advantageous, as the fabrication process is simplified due to lateral etching possibilities located on the right side.

FIG. 71 shows an example of a portion of the microfabricated valve, which is similar to FIG. 67. The connection channel (13) is connected to the first channel (11) by at least one first opening (2) and the connection channel (13) is connected to the second channel (12) by at least one second opening (1). In this example, the number of the second openings (1) are different, comprising a first second opening (102) and the second opening (103), whereas the first first opening (104) has a different geometry/dimension than the first second (102) and the second opening (103). In a particular embodiment, one or more of the openings, however especially preferred the first opening, comprises baffel structures 105 for disturbing the fluid stream thereby increasing the mixing efficiency.

FIG. 72 shows an example of a portion of the microfabricated valve, which is similar to FIG. 64. Further indicated are the openings, wherein three openings are illustrated, comprising a first second opening (102), a second opening (103) and first opening (104), wherein the openings are not coaxial.

FIG. 73 shows an example of a portion of the microfabricated valve, which is similar to FIGS. and 71. In this example, the flexible membrane (15) comprises an inner boundary 4 forming the outer wall of the connection channel (13) or encompassing at least one section of the connection channel (13) and an outer boundary 5 forming the outer wall of the flexible membrane. The inner boundary 4 is defined by different inner boundary sections (106, 107), each encompassing a different section of the connection channel (13). In this example, the microfabricated valve comprises multiple openings (102, 104), which are located within a different section of the connection channel (13). This advantageous embodiment allows forming separated spaces within a connection channel that can prevent that two different fluids might get into contact within the valve portion. For instance, the openings can be connected to different channels and/or to the same channel.

FIG. 74 shows an example of a portion of the microfabricated valve, which is similar to FIG. with a triangular shape as in FIG. 64. The inner boundary (4) is defined by three different inner boundary sections, each encompassing a different section of the connection channel (13). The microfabricated valve comprises multiple openings (108, 109, 110), which are located within a different section of the connection channel (13).

FIG. 75 shows an example of a portion of the microfabricated valve, which is similar to FIG. with a rectangular shape as in FIG. 65. The inner boundary (4) is defined by four different inner boundary sections, each encompassing a different section of the connection channel (13). The microfabricated valve comprises multiple openings (118, n=4), which are located within a different section of the connection channel (13).

FIG. 76 shows an example of a portion of the microfabricated valve, which is similar to FIG. with a pentagonal shape as in FIG. 65. The inner boundary (4) is defined by five different inner boundary sections, each encompassing a different section of the connection channel (13). The microfabricated valve comprises multiple openings (118, n=5), which are located within a different section of the connection channel (13). In one advantageous embodiment, up to five openings are each connected to separate sections of the connection channel, enabling co-injection of up to five fluids into one or more common channel(s).

FIG. 77 shows an example of a portion of the microfabricated valve, which is similar to FIG. 70. In this example, two separate actuation chambers (111A, 111B) surround the valve portion (14) and a portion of the boundaries of the actuation chamber (6) is in direct contact with the flexible membrane at the boundary of the actuation chamber (101: merging position) with the outer boundary of the valve portion, which corresponds to a first membrane section and a second membrane section. The connection channel (13) is separated from the second actuation chamber 111B by a second section of the flexible membrane 107, wherein the second section of the flexible membrane 107 and the first section 106 of the flexible membrane 15 are different sections, wherein the valve portion (14) is adapted to be selectively transferred into an open and/or closed and/or intermediate shape upon modification of a pressure difference between the second actuation chamber 111B and the connection channel (13) by modification of the pressure inside the second actuation chamber 111B, wherein the pressure inside the first actuation chamber 111A and the pressure inside the second actuation chamber 111B can be modified independently. This example of a microfabricated valve is advantageous, as the membrane section can be actuated by an individual actuation command.

FIG. 78 shows an example of a portion of the microfabricated valve, which is similar to FIG. 62. In this example, the inner boundary and the outer boundary of the flexible membrane have a biconvex shape. At least two membrane sections deflect towards each other. Moreover, the flexible membrane comprises etching access structures (112) located at the corners of the inner boundary/boundaries. This is advantageous, as the lateral etching enables the etching of thin membranes. Furthermore, the actuation chamber can comprise support structures (113) stabilizing the master mold and narrowing (114) of the actuation chamber (narrowed section) for prevention of upside deflection of material separating actuation channel and a flow channel that might be located above/below said narrowed section.

FIG. 79 shows an example of a portion of the microfabricated valve, which is similar to FIG. 64. In this example, the inner boundary and the outer boundary of the flexible membrane have a triangular shape. Further, the flexible membrane comprises etching access structures (112, 113) located at the three corners of the inner boundary/boundaries. This is advantageous, as the lateral etching enables the etching of thin flexible membranes, wherein the flexible membrane has a thinned section which has a reduced thickness compared to at least one other section of the flexible membrane. Thus, the flexible membrane has an inhomogeneous thickness (thinnest section 121). In addition, the actuation chamber can comprise a narrowed section (114).

FIG. 80 shows an example of a portion of the microfabricated valve, which is similar to FIGS. and 74. In this example, the inner boundary and the outer boundary of the flexible membrane have a triangular shape. Further, the flexible membrane comprises etching access structures (112) and support structure (113) located at the three corners of the inner boundary/boundaries. The inner boundary (4) is defined by three different inner boundary sections, each encompassing a different section of the connection channel (13). The actuation chamber can comprise a narrowed section (114).

FIG. 81 shows an example of a portion of the microfabricated valve, which is similar to FIG. 65. In this example, the edges of the flexible membrane comprise an etching access structure (112), support structure (113) and the actuation chamber can comprise a narrowed section (114).

FIG. 82 shows an example of a portion of the microfabricated valve, which is similar to FIG. 68. In this example, the edges of the flexible membrane comprise an etching access structure (112), support structure (113) and the actuation chamber can comprise a narrowed section (114). This example is in particular advantageous for large particles.

FIG. 83 shows an example of a portion of the microfabricated valve, which is similar to FIG. 73. In this example, a first second opening 1 connects the second channel 12 with a first section (116) of the connection channel (13) and a second opening 1 connects the second channel 12 with a second section 117 of the connection channel (13).

The first second channel 12 may contain a first fluid, the second channel 12 may contain a second fluid and the (common) first channel (11) may contain a third fluid. This embodiment therefore allows injecting two different fluids in a channel which already contains a third fluid.

FIG. 84 shows an example of a microfabricated valve, which is similar to FIG. 69A. The example shows a three dimensional schematic structure, wherein two second channels comprise openings toward a biconvex valve portion, wherein fluid can selectively enter the connection channel (13) and be injected into a second channel (12).

FIG. 85 shows an example of a microfabricated valve, which is similar to FIGS. 69A and 72. The example shows a three dimensional schematic structure, wherein two second channels comprise openings (102, 103) toward a triangular valve portion, wherein fluid can selectively enter the connection channel (13) and be injected into a second channel (12) through a third opening (104). This example is highly effective in mixing due to triangular geometry and arrangement of openings in the corner of the triangle and an optional membrane deflection increases mixing efficiency.

Table 85 shows simulation results for the pressure (MPa) required for fully closing the microfabricated valve. Simulation results are shown for different basic geometries (e.g. circular, rectangular etc.), thickness of the elastomeric membrane and total deflection distance (diameter) for fully closing the valve (nominal diameter).

FIG. 86a shows a preferred embodiment in which the thickness depends on the deflection distance of the flexible membrane 15. The deflection distance is the distance of the position of a point on the inner boundary of the flexible membrane 15 while the flexible membrane is in the closed shape and the position of this point while the inner flexible membrane 15 is in the opened position. In this exemplary embodiment the flexible membrane 15 has a biconvex shape and it is shown in the opened shape. The deflection distance rises from a minimal value at the point S0 and reaches its maximum at in the middle, where the distance between two opposite points on the inner boundary of the flexible membrane is maximal. At this point (as shown by FIG. 86b), the thickness of the membrane dM,min is minimal (thinnest section). By moving further to the point S3 along the S-axis, it is shown that the deflection distance decreases steadily (dD). Hence, the distance between two opposite points on the inner boundary of the flexible membrane decreased. At that point the thickness of the membrane dM increased in comparison to dM,min. Moving further to point S3, the deflection distance reaches a further minimum, however the thickness of the membrane dM,max is maximal.

Thus, the embodiment of FIG. 86 shows a flexible membrane comprising a thinned section, wherein the thinned section is at the position of the maximal deflection distance.

FIG. 7 shows an electrostatic actuation of elastomer valve 10.

FIG. 8 shows microscopy images of microfabricated elastomer valve actuation and corresponding experimental data. A) Microfabricated elastomer valve with geometry: d=50 μm, r=125 μm, a=120 μm. B) Microfabricated elastomer valve with geometry: d=50 r=250 μm, a=400 μm.

FIG. 9 is an illustration of generation of droplets (A) and encapsulation of cells or particles (B) using the described microfabricated elastomer valve 10. FIG. 9A) The second upper channel is filled with fluid 1 (e.g. an aqueous phase) and the first bottom channel 11 is filled with fluid (e.g. oil) whereas both fluids are immiscible. Applying a pressure within the upper channel 21 and subsequent opening of the elastomer valve portion 14 for a defined time results in the generation of droplets 31. Droplet size can be tuned by changing the applied pressure and the opening time. FIG. 9B) Encapsulation of single or multiple cells (in the following commonly referred to as “particles 20) within droplet 31. The upper channel is filled with a cell/particle suspension 36. Droplet generation results in particles 20 that are located within droplets 31. The particles are Poisson distributed.

FIG. 10 is an illustration of a particle trap 17 for encapsulation of a single particle. The trap 17 is located above the microfabricated elastomer valve portion 14. FIG. 10A: The top microfabricated layer 23 is first perfused with a particle suspension 36. Single particles 20 are trapped and immobilized in the hydrodynamic trap 17 located above a microfabricated valve portion 14. Subsequent opening of the microfabricated valve portion 14 results in a fluid flow from the top layer 23/second channel 12 into the bottom layer 21/first channel 11 that is filled with an immiscible (with the respect to the fluid within the second channel) second fluid 37, in particular an oily fluid. The trapped cell 20 is thereby transferred into the formed droplet 31, wherein the fluid of the cell suspension 36 surrounds the captured particle 20. The fluid of the cell suspension 36 and the particle constitutes a droplet 31.

FIG. 10B: is an illustration of the particle trap 17 of FIG. 10A in top view. The generic single particle trap 17 is located above/adjacent to the microfabricated elastomer valve portion 14. The trap 17 comprises a bottleneck section 16, which fluid opening is smaller than the particle 20 to be trapped. A first particle arriving at the trap is captured by the trap. All further particles arriving subsequently at the trap take the way along a bypass section 18. 38 illustrates an optional impedance measuring device, 39 illustrates an optional radio frequency application device.

FIG. 10 C) is an illustration of an amended trap group for the immobilization of two particles 20, in particular cells, located in two separate neighboring traps 17n above the microfabricated valve portion 14. Opening of the valve portion 14 may result in a co-encapsulation of two trapped particles 20 into one droplet 31, because the valve portion 14 leads from both traps 17n into the same first channel 11 below both traps 17n. With the help of this embodiment, two different particles 20 can be encapsulated within one single droplet 31.

FIG. 10D shows a trap group in schematic view. Each of the neighboring traps 17n is loaded from a separate channel 12′, 12″, in which the same pressure p2 is applied to the fluid, to achieve droplets of the same size. At first the traps 17n are loaded; when all traps 17n are loaded a washing fluid can be applied to clean the trapped particles or cells. Subsequently the valve portions 14 are opened to include the particles 20 (e.g. cells) through one valve section 14 simultaneously into one droplet 31. A plurality of such trap groups having two neighboring traps 17n can be arranged in one test device.

The test device can be provided with an impedance measurement device 38. Individual droplet, cells or particles thereby be applied with a voltage or a current. Based on the measured impedance properties of the droplet, the cell or the particles can be obtained. The impedance measurement device 38 can be located at any particle trap 17 (e.g. at trap 17 in FIG. 10B) or droplet trap 33 (e.g. at trap 33 in FIG. 23), at a particle centering station (see FIG. 12), anywhere at observation chamber 32, or at any other location where a droplet, a cell and/or a particle, in particular a hydrogel particle/matrices, is held stationary, in particular for at least more than 0.1 seconds. In another particular embodiment, a hydrogel particle/matrix is held stationary for at least 0.5 ms, 1 ms, 10 ms, 50 ms, 100 ms.

The test device can be provided with a radio frequency application device 39. Individual droplet, cells or particles, in particular hydrogel particle/matrices can be applied with a radio frequency. Based on the chemical or physical or chemical properties the droplets and or hydrogel particle, the cells or the particles can be heated. Thereby the frequency has to be adapted to the properties of the droplets, the cells or the particles can be heated. The functionality may be similar to the functionality of microwave oven. The radio frequency application device 39 can be located at any trap 17, 33 (e.g. at trap 17 in FIG. 10B, e.g. at trap 33 in FIG. 23), at a particle centering station (see FIG. 12), anywhere at observation chamber 32, or at any other location where a droplet, a cell and/or a particle is held stationary, in particular for at least more than 0.1 seconds.

FIG. 11 is an illustration of droplet mixing after on demand droplet generation using microfabricated elastomer valve and subsequent hydrogel formation. A) A fluid (e.g. a cell suspension) is located in the second top channel (left channel) 12A. This fluid contains a first hydrogel precursor. A second fluid in the other second top channel (on right channel) 12B contains a cross-linker for initiating the hydrogel formation. An immiscible fluid (e.g. fluorinated oil) is located in the first bottom channel 11. B) Opening of the microfabricated valves portion 13 located below the two upper channels 12A, 12B results in the formation of two droplets 31A, 31B: one droplet 31A containing fluid 1 and a second droplet 31B containing fluid 2 that are located within the immiscible fluid 3 (C). D) Applying a flow to the first channel results in the coalescence of the two droplets 31A, 31B. E) As one droplet contains a hydrogel precursor and a second droplet contains a cross-linker, a combined hydrogel is formed within the mixed droplet 31AB.

FIG. 12A shows an embodiment of a particle centering station. Here a particle 20 centering within droplets 31 by inducing droplet rotation. A droplet 31 containing a particle 20, in particular cell, is immobilized within a microfabricated geometry that results in an increased hydrodynamic pressure located below the droplet 31. This pressure results in a rotation of the trapped droplet 31 and thus in a centripetal force acting on the encapsulated particle 20 which results in a centering of the particle 20 in the center of the droplet 31. Subsequent hydrogel formation results in a spherical hydrogel matrix containing a particle 20 in its center. 38 illustrates an optional impedance measuring device, 39 illustrates an optional radio frequency application device.

FIG. 12b shows another embodiment of a particle centering station 70. As described in the former embodiment here also the droplet containing a particle is brought into rotation, resulting in a centering effect of the particle within the droplet. The particle centering station 70 may comprise a droplet trap 33 in particular having a bottleneck section 16.

The droplet centering station 70 has a plurality of channels allowing a fluid flowing along different paths 71, 72, 73 of fluid. A set valves V1, V2, V3, V4, V5 may be provided to control the flow of fluid along the different paths 71, 72, 73 of fluid. In another embodiment, the flow of fluid may be controlled by using fixed hydrodynamic resistances and varying pressure sources. The valves can be designed in manner as described within other areas of the present description. The centering station 70 can be located within a feeding channel 41 as described with other areas of the description. In this particular example initially a first and a second valve V1, V2 is open, the remaining valves V3, V4, V5 are closed.

In a first step (FIG. 12b, first and second image) the droplet 31 is supplied along a first path of fluid 71, in particular from the feeding channel 41, guiding the droplet 31 into a droplet trap 33. In this embodiment the trap 33 comprises a bottleneck section 16 having a smaller diameter than the diameter of the droplet as described within other areas of the description.

In a second step (FIG. 12b, third image) the droplet 31 is trapped within the trap 33. Now a fluid is supplied within a second path of fluid 72. The second path of fluid 72 is adapted to contact the trap 33 in a manner that the fluid flowing along the second path brings the droplet into rotation, thereby preventing that the droplet leaves the trap 33. The fluid touches the droplet 31 in a tangential direction. In this particular example a fifth and a fourth valve V5, V4 is open, the remaining valves V1, V2, V3 are closed.

In a third step (FIG. 12b, fourth image) the droplet 31 is released from the trap 33. Now a fluid is supplied within a third path of fluid 73. The third path of fluid 73 flows through the trap in opposite direction compared first path thereby urging the droplet 31 out of the trap 33. In particular the droplet is brought back to the feeding channel 41. In this particular example a third and a fourth valve V3, V4 is open, the remaining valve V1, V2, V5 are closed.

In particular the second path 72 of fluid has a minimum diameter than the first and/or second path and/or feeding channel 41 through which the droplet (31) is delivered. This may result in higher flow velocity during the second step.

In particular during the second step the fluid urging the droplet in a direction C away from a bottleneck section 16 (see arrow C in FIG. 12b, third image). Here the risk is reduced that the droplet 31 may be accidentally pushed through the bottleneck section 16.

In particular the second path of fluid 72 is not passing the bottleneck section 16.

FIG. 12 c shows an illustration of an incident flow/propulsive jet 72 that causes a stationary held droplet 31 located in a centering station 70 to rotate as well as the critical parameter for calculating the rotational speed of a droplet as a function of the volume flow of the incident flow/propulsive jet 72. They droplet 31 may contain at least one particle 20. The propulsive jet has a volume flow dV/dt and a flow velocity v₀. The channel that provides the propulsive jet is assumed to be round for simplified calculations with an inner radius of r. The droplet has a radius R and the rotation speed ωR.

FIG. 13 shows a microfabricated geometry for droplet trapping and rotation. A) Front view of trapping structure illustrating two channels for the volume flow which leads to a droplet rotation. B) Single droplet trapping. C) Trapping of two droplets with a gap between the droplets preventing droplet contact during rotation.

FIG. 14 shows hydrogels composed of hydrophilic poly-(2-oxazoline) polymers for long-term 3D cell cultivation. Physiochemical properties can be changed by varying the polymer content, molecular weight and functionalization sites.

FIG. 15 shows the functionalization of poly-(2-oxazoline) polymers with unsaturated imides during cationic ring opening polymerization. The underlying mechanism is a copolymerization of unsaturated imides as electrophilic monomers and 2-oxazoline as a nucleophilic monomer.

FIG. 16 shows hydrogels composed of hydrophilic poly-(2-oxazoline) and unsaturated imides or alkenyl groups. Because mechanical and physiochemical properties can be changed by varying the polymer content, molecular weight and functionalization sites, this copolymer is perfectly suitable for long-term 3D cell cultivation and analysis. 2-methyl-oxazoline is shown as an example for an oxazoline substituted in position 2.

FIG. 17 shows stable three-dimensional hydrogel formation via hydrogen bonds between LNAs and/or PNAs.

FIG. 18 is an illustration of a demulsification method using the microfabricated valve 10. A) A droplet is trapped using a microfabricated geometry as a trap 33 located below a microfabricated elastomer valve. The droplet contains a spherical hydrogel matrix with an encapsulated particle/cell 20. Due to the density difference between the immiscible oil (higher density) and the droplet 31 (lower density), a buoyant force is acting on the droplet pushing the droplet towards the microfabricated elastomer valve. Subsequent opening of the microfabricated elastomer valve results in a coalescence between the droplet 31 containing a spherical hydrogel matrix and the aqueous phase located in the upper channel. B) Illustration of the particle centering principle before droplet demulsification. A droplet containing a particle 20 is first trapped using a microfabricated geometry located below a microfabricated valve. Afterwards the particle is centered by rotation and a spherical hydrogel matrix 31 is formed. Subsequent opening of the microfabricated elastomer valve results in demulsification of the droplet content.

FIG. 19 is an illustration of selective droplet demulsification by using a DEP based quadrupole trap 33 located below a microfabricated valve 10. A generated droplet 31 is trapped by using a DEP force acting on that droplet 31. If the droplet 31 contains a single cell/particle 20 a hydrogel is formed and the droplet 31 is subsequently demulsified by using the technique described previously. The quadrupole having four poles 45 constitutes a DEP force generator 44, which in this case is a part of the trap 33. An example is 3D electrodes made of conducting SUB.

FIG. 20 shows if a droplet 31 is trapped using a DEP field. In addition to the DEP based trapping shown in FIG. 19 the droplet 31 is positioned in front of a microfabricated droplet geometry 46 that causes the droplet 31 to rotate. A particle 20 within the droplet 31 is subsequently centered within the droplet 31. Afterwards the droplet 31 content can be demulsified by opening a microfabricated valve 10 located above the trap 33.

FIGS. 19A and 20A show the respective trap 33 before the DEP force is applied. FIGS. 19B and 20B show the respective trap 33, when the DEP force is applied, consequently the droplet 31 is retained in the trap 33.

FIG. 21 is an illustration of hydrodynamic resistances R0, R1, R2, R3, R4 within one observation chamber 32, here at the example of observation chamber 32m2n2 in position of column m2 and row n2. R0 indicates the hydrodynamic resistances at a droplet trap 33, R1-R4 indicate the hydrodynamic resistances of different paths within the observation chamber 32, with R1, R4>R2, R3. P1 indicates an entrance of a main fluid flowing through the observation chamber 32 to an exit indicated by P2. The main feeding channel 41 optional here.

A) During normal operation the main fluid stream moves from top to down (first direction of flow S1 along first path of flow 51 or optional along main feeding channel 41), since the stream takes the “easier way” through smaller resistances R2, R3. Merely a negligible part of the fluid flows through path of resistances R1, R4. Here all triggering commands Cm2, Cn2 are set to zero.

B) By triggering a valve Vm2 by command Cm2=1 in the path of R2, resistance R2 of this path will significantly increase. The main fluid now moves from P1 to P2 via paths of resistances R4 and R3 along third path of flow 53. The flow at R0 is now stopped, but not reversed.

C) By triggering a valve Vn2 in the path of R3 command Cn2=1, resistance R3 of this path will significantly increase. The main fluid now moves from P1 to P2 via paths of resistances R2 and R1 along fourth path of flow 54. The flow at R0 is now stopped, but not reversed.

D) Only when both the resistances in paths of R2 and R3 is increased, by triggering the valves Vm2 and Vn2 by commands Cm2 and Cn2 set to 1, the flow at position R0 within the droplet trap is reversed. The main fluid now moves from P1 to P2 via paths of resistances R4, R0 and R1 along fourth path of flow 54. A droplet 31 that is located within the droplet trap 33 at R0 is subsequently removed from the trap position. The group of the both valves Vm2, Vn2 is here called at the valve arrangement 40m2n2 of the observation chamber 32m2n2 exemplary.

FIGS. 22a to 22c show the observation chamber 32 of figure at position m2 n2 within the microfabricated test device 30 in different embodiments. Here in sum sixteen observation chambers 32 similar to the one described above with reference to FIG. 21 are arranged in matrix with four columns (m1-m4) and four lines (n1-n4). In the column m2, all valves Vm2 in the path of respective resistance R2 are connected to the same triggering control line (not shown) which is in particular an air pressure line or an hydraulic pressure line. So with triggering the command Cm2, all valves Vm2 off all chambers 32 in the column m2 are closed. Same applies also for all other columns m1, m3, m4.

Same concept is also realized in the lines n1-n4. In the column n2, all valves Vn2 in the path of respective resistance R3 are connected to the same triggering control line which is in particular an air pressure line or an hydraulic pressure line. So with triggering the command Cn2, all valves Vn2 off all chambers 32 in the line n2 are closed. Same applies also for all other lines n1, n3, n4.

In FIG. 22a the situations D of FIG. 21D can be seen. Only in chamber 32 at position m2, n2 both valves Vm2, Vn2 are triggered; here the flow of fluid through droplet trap 33 is reversed (situation D of FIG. 21). In the other chambers of positions in column m2 or line n2 (not m2 and n2), merely a stop of fluid in the droplet trap is achieved, but no reverse flow (situation B or C of FIG. 21). In all other chambers (chamber is outsides of columns m2 and outside of line n2) fluid flows in first direction S1. So with merely 8 (n+m) triggering lines (pressure air line) selectively in each of the sixteen (n×m) chambers can be reversed.

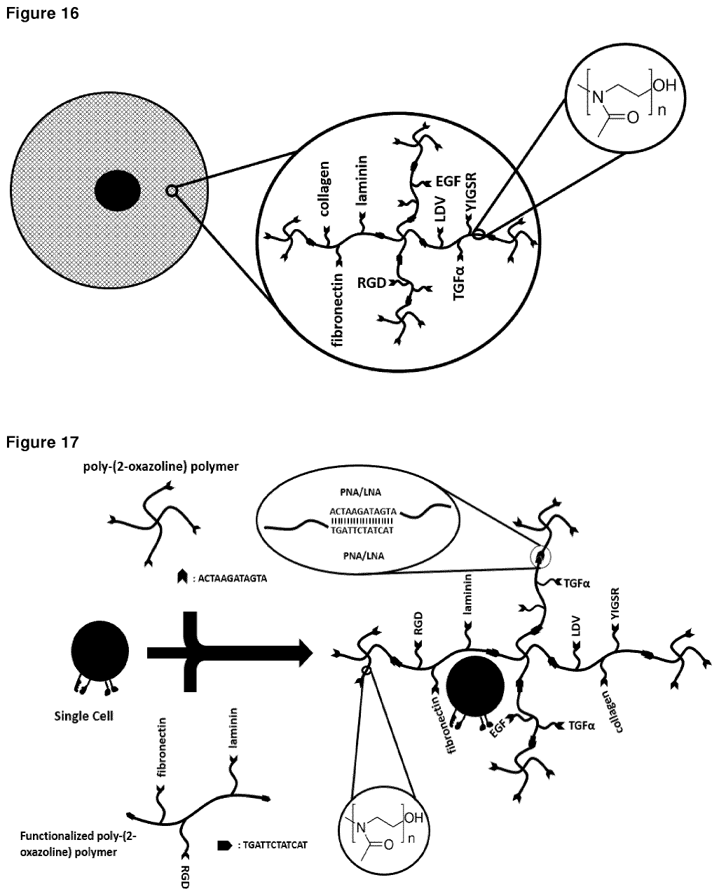

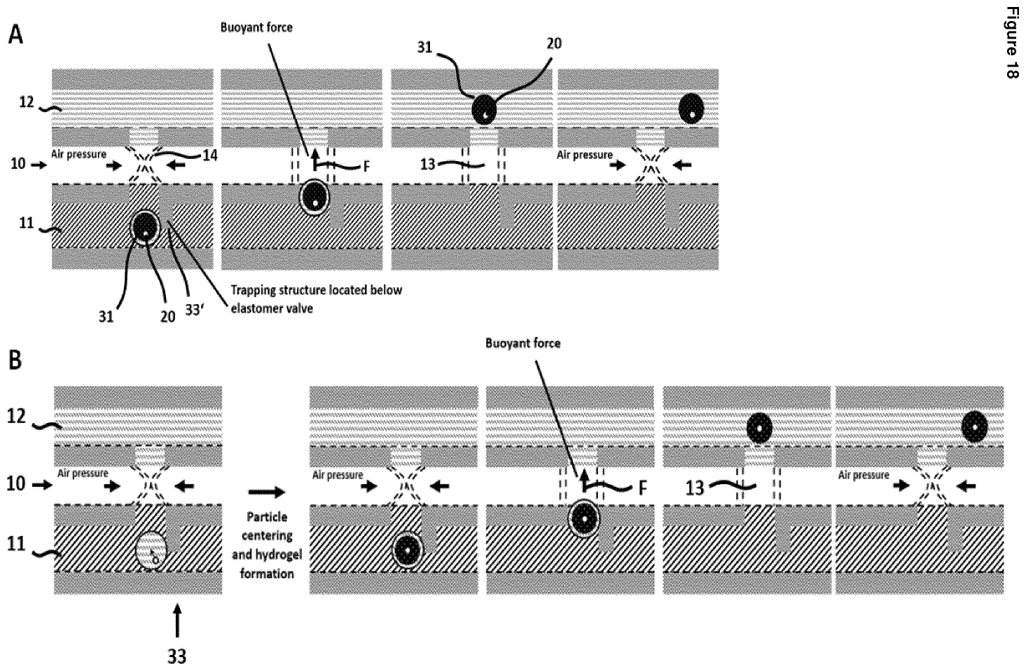

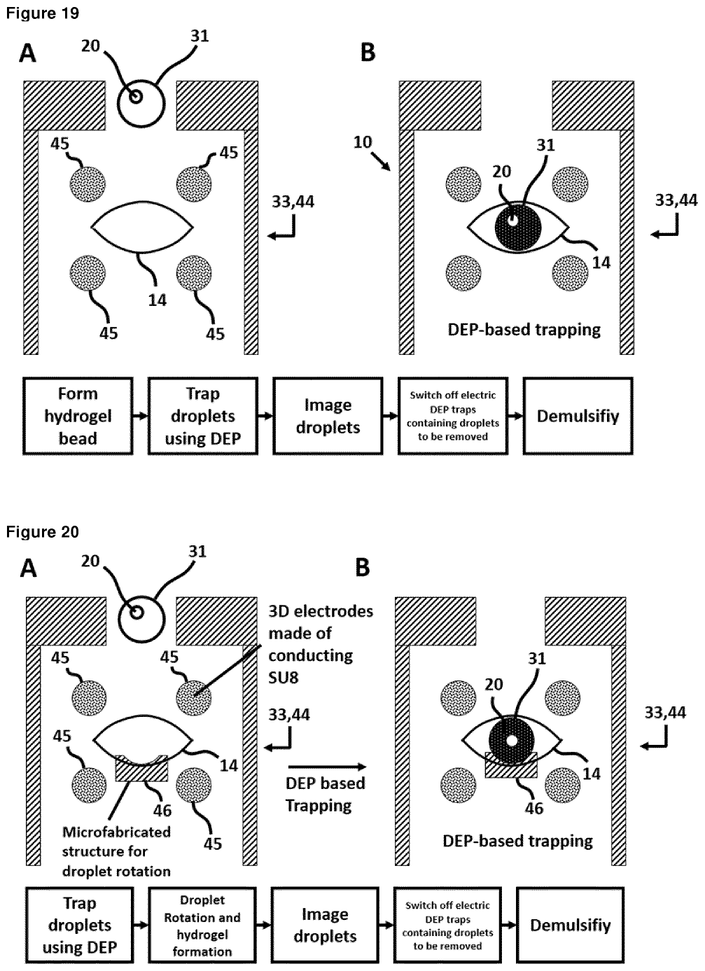

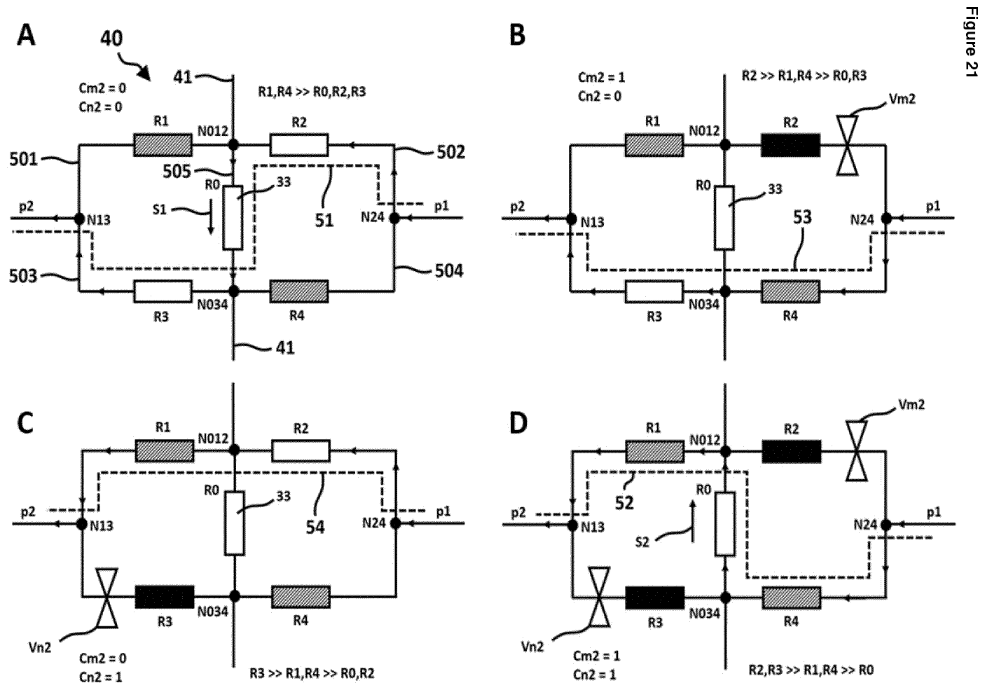

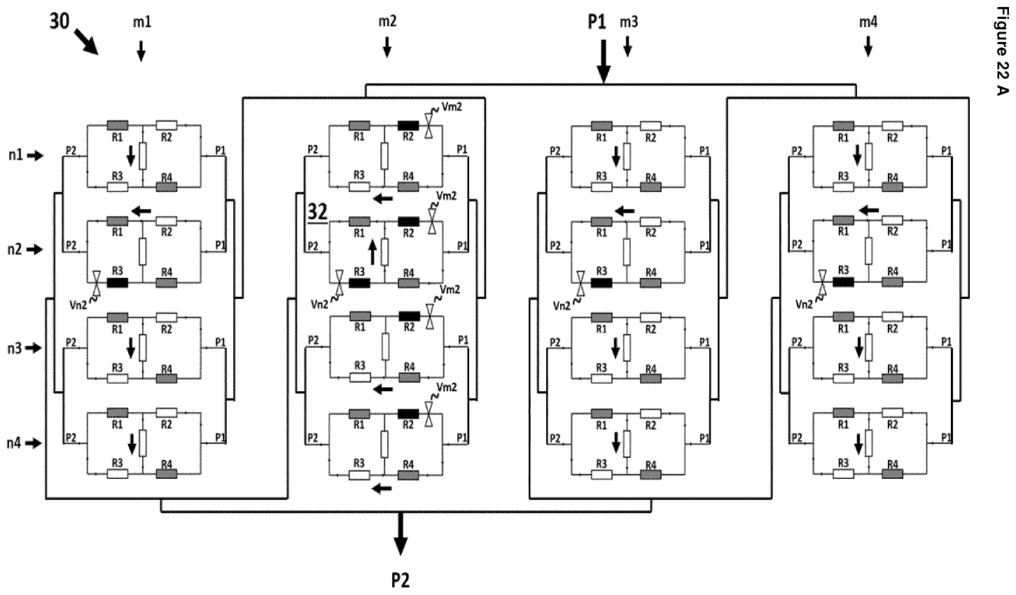

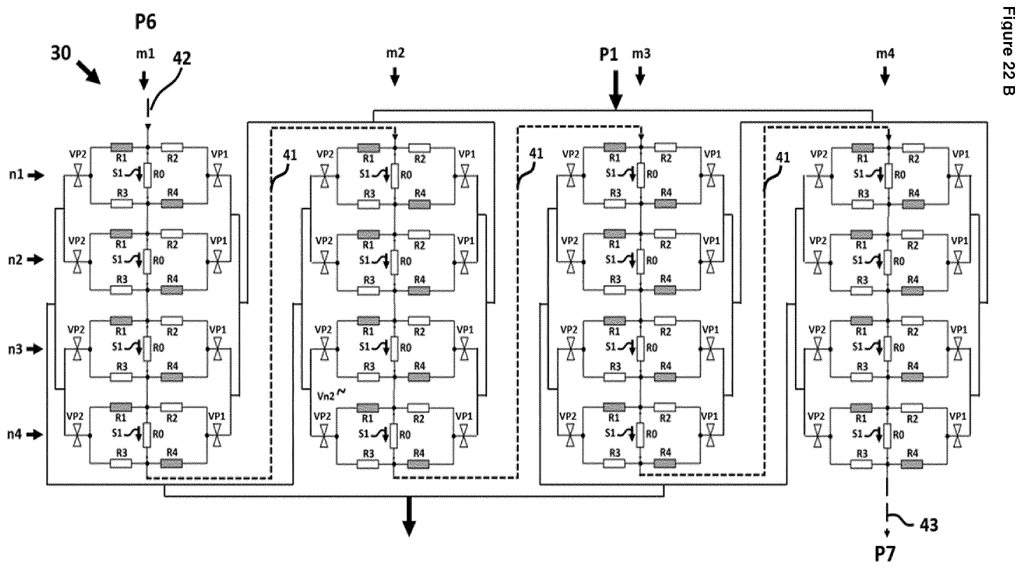

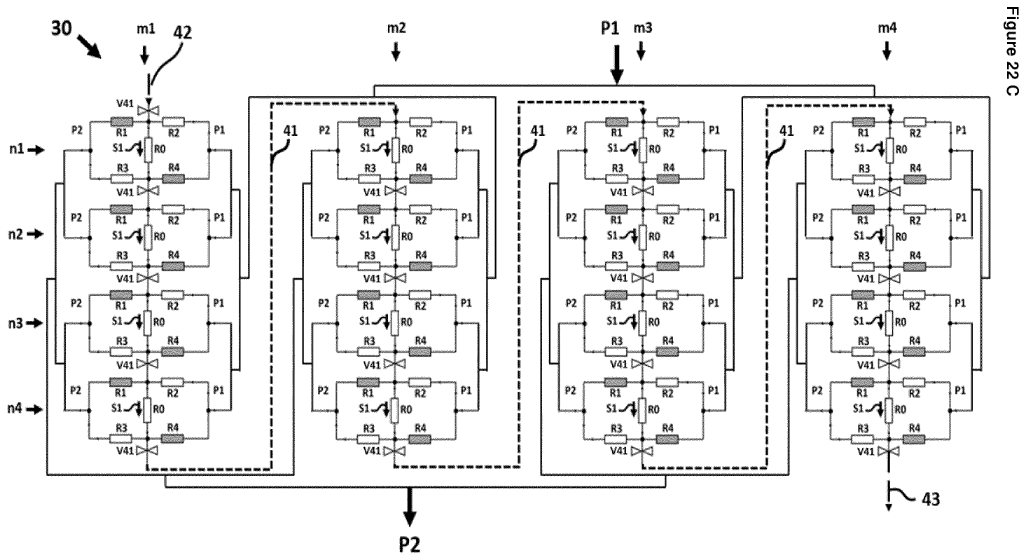

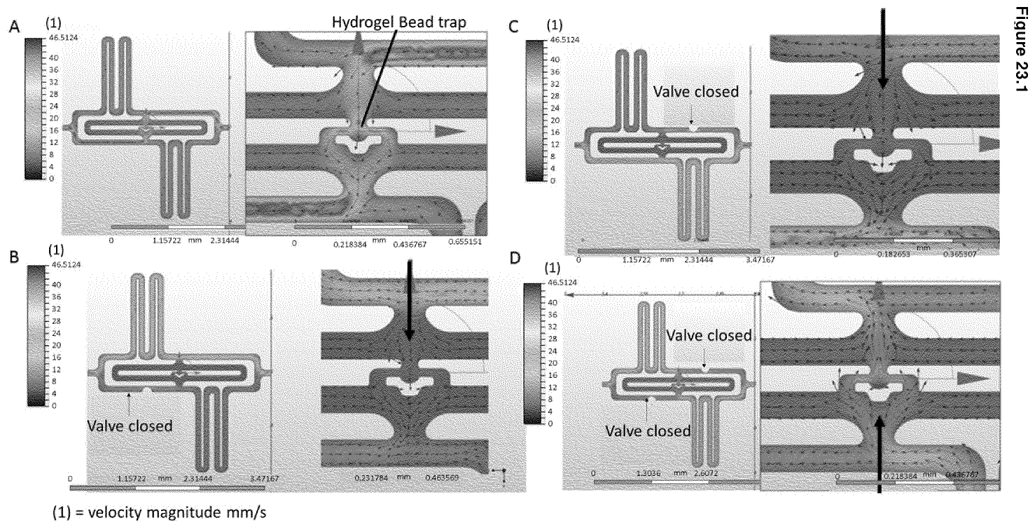

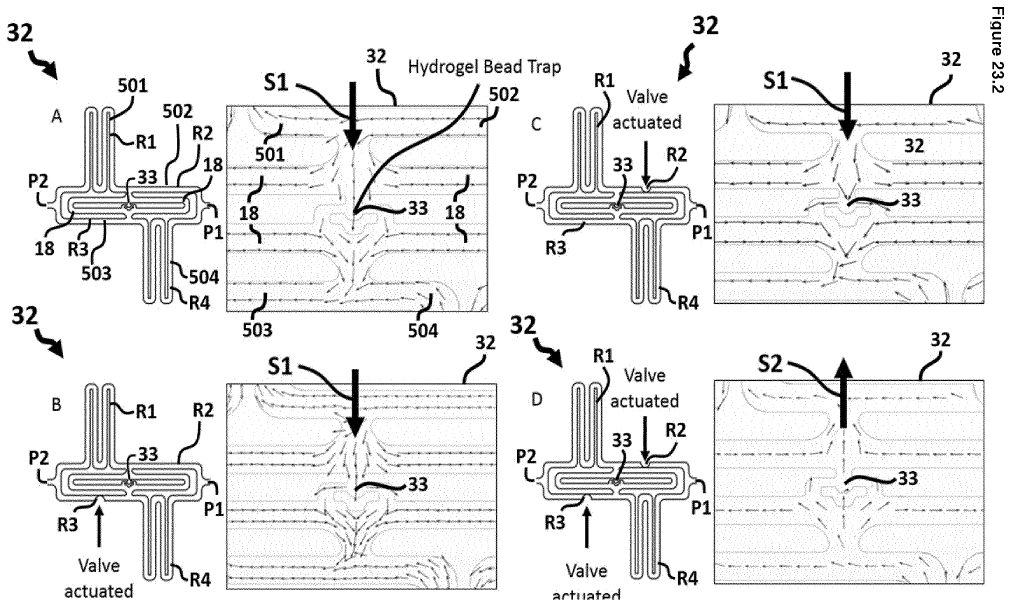

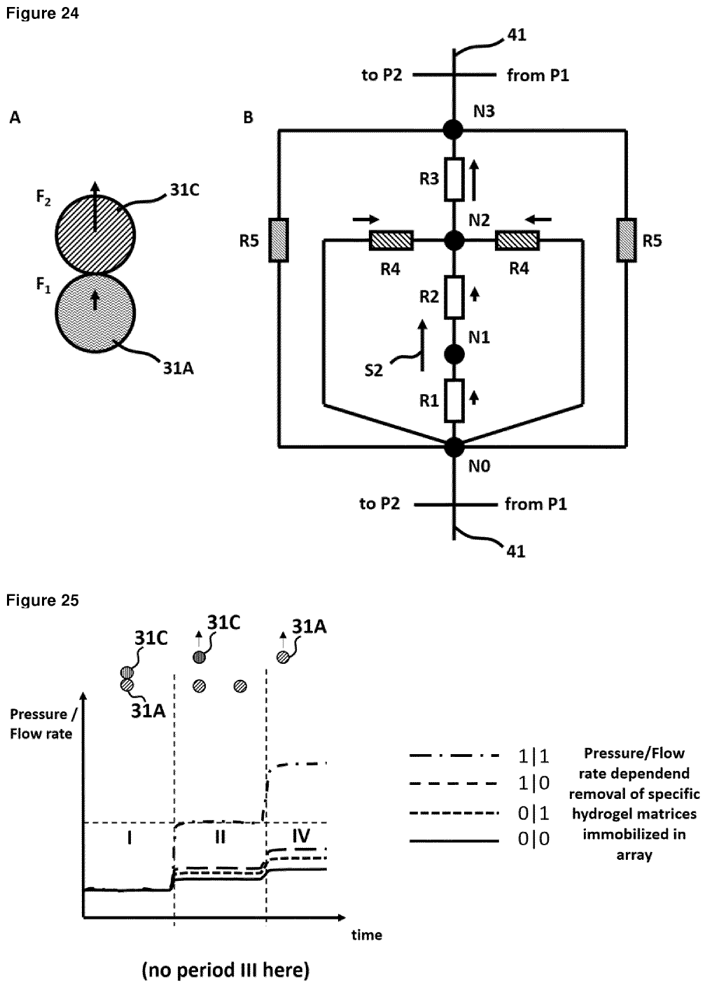

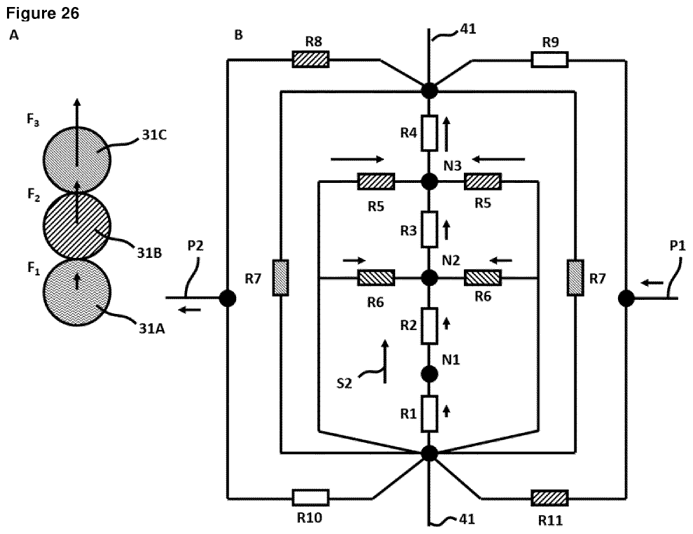

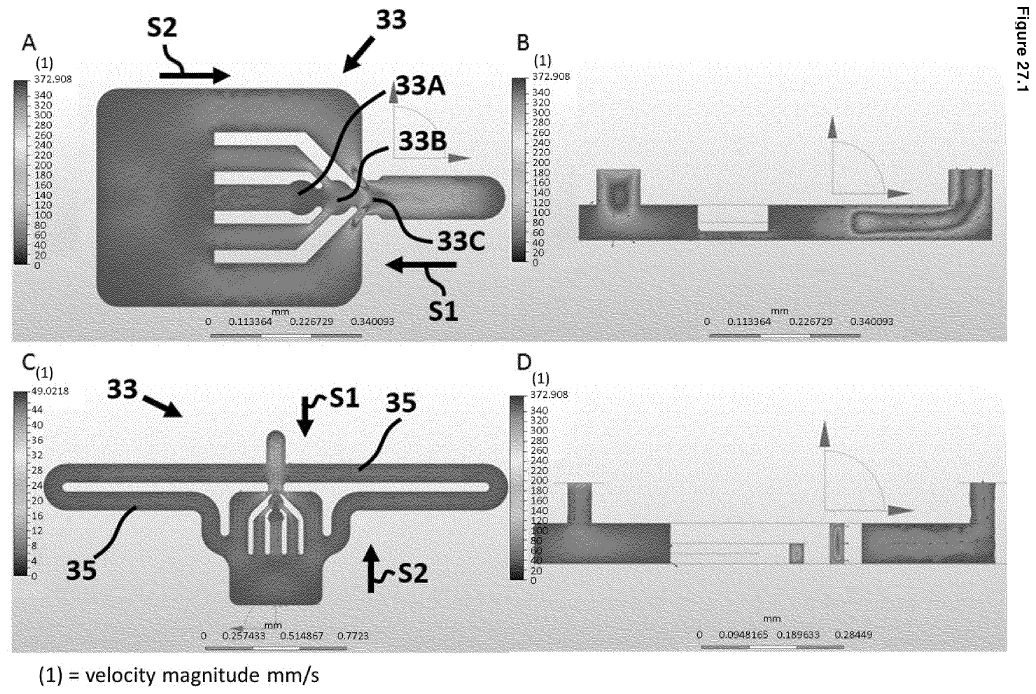

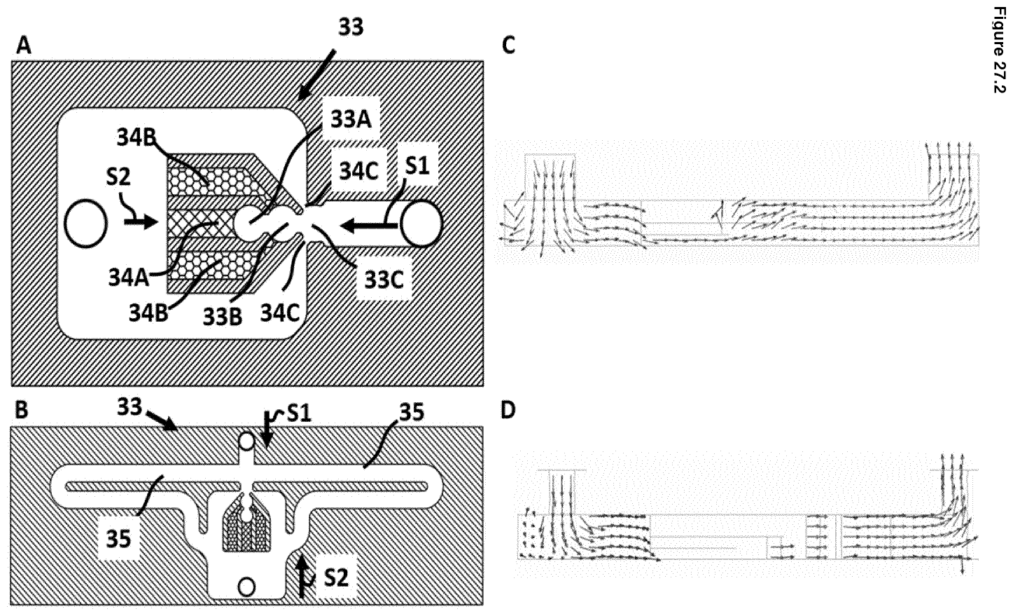

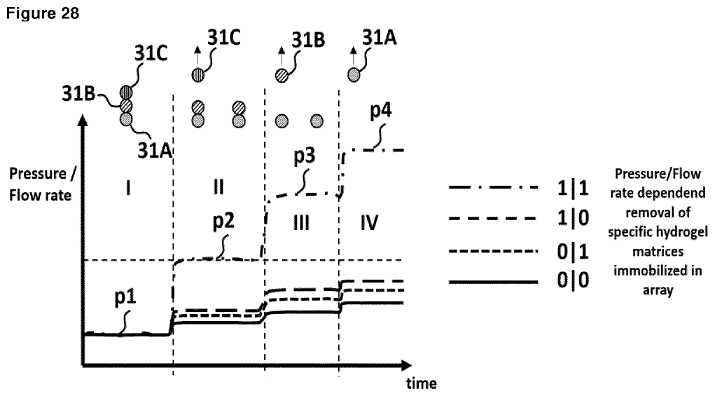

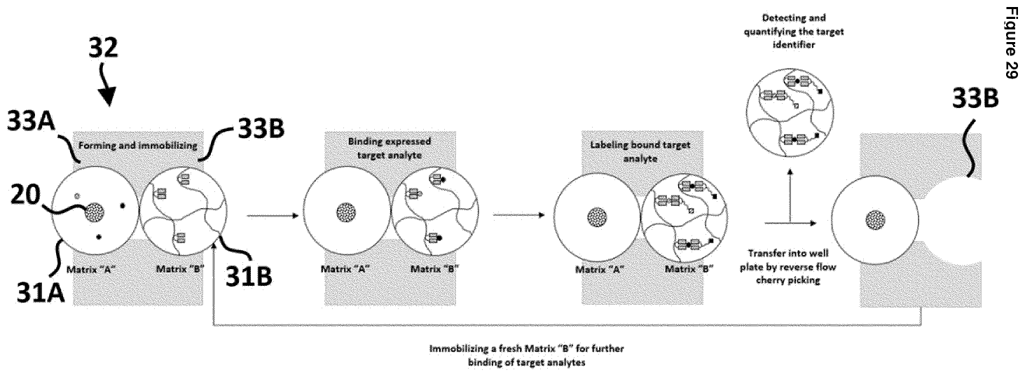

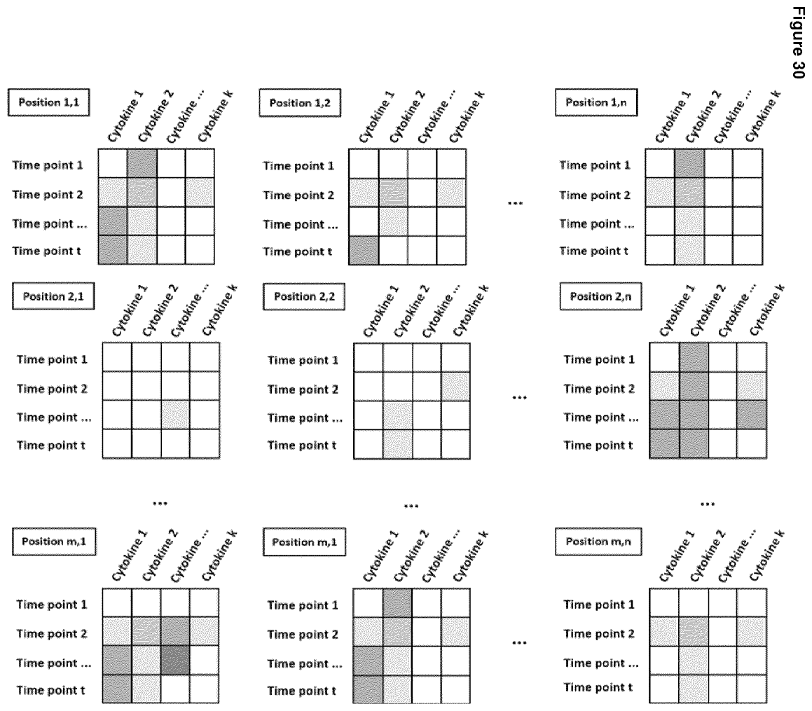

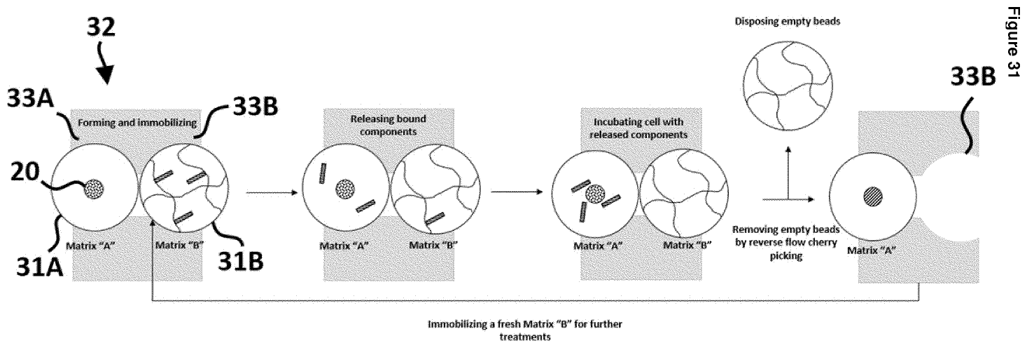

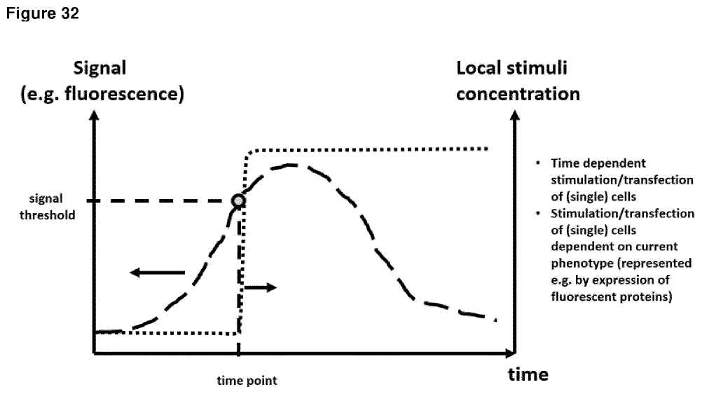

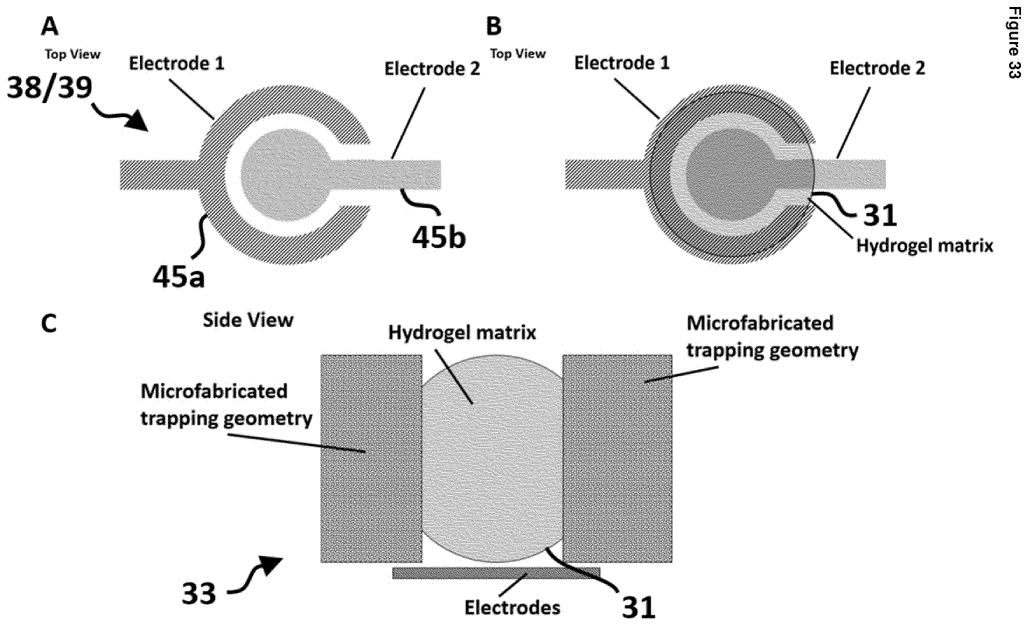

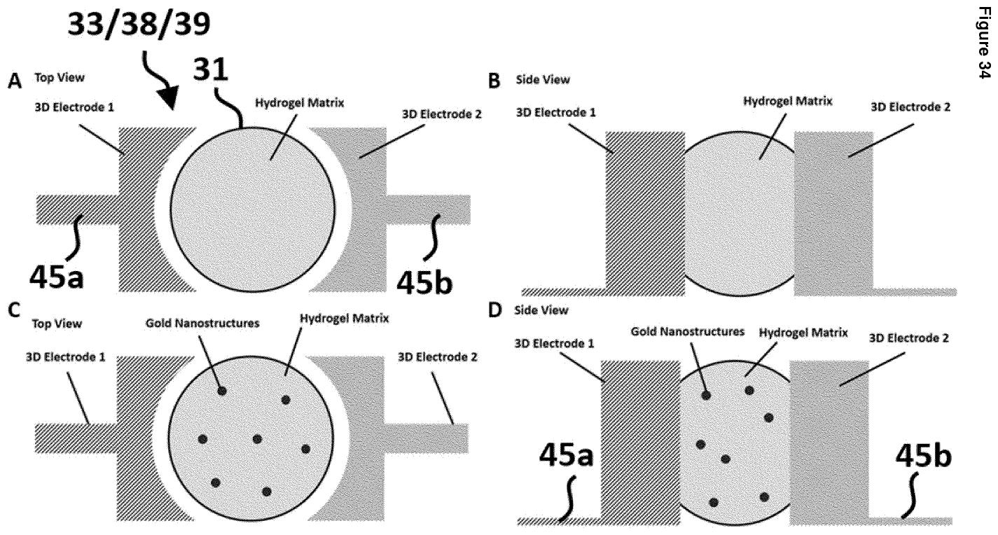

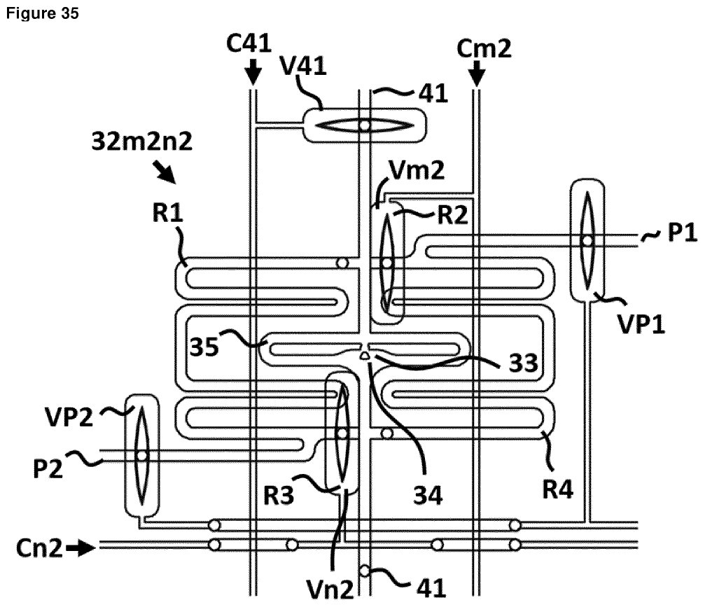

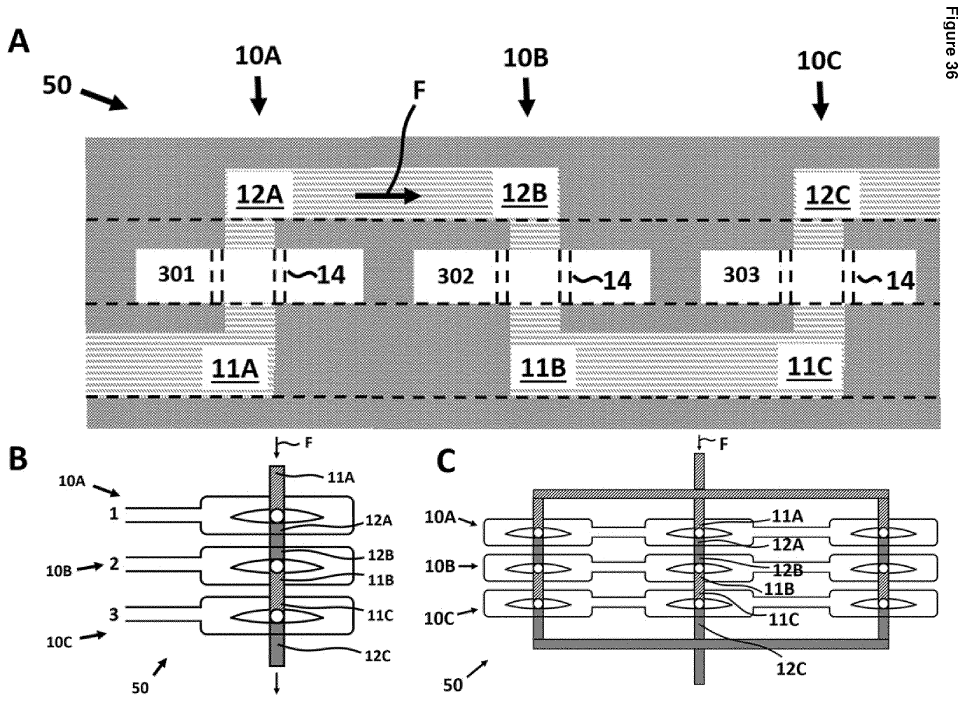

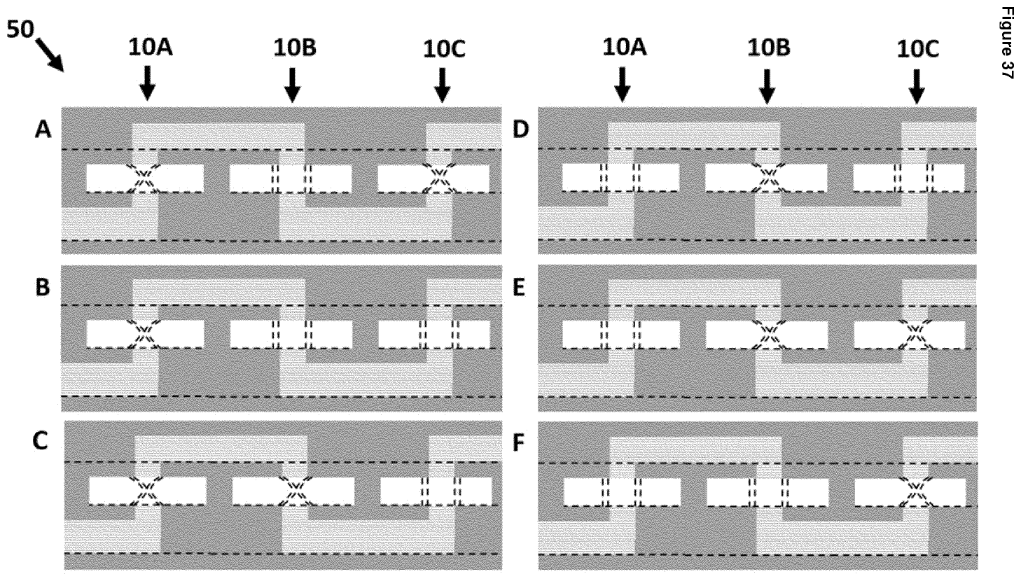

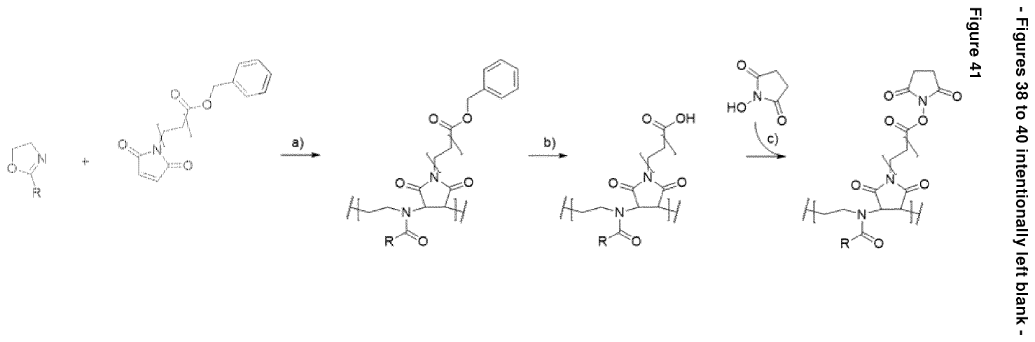

In the device according to FIG. 22a initial filling of the locations 32 is performed through inlet portion P1. In the embodiment shown in FIGS. 22b and 22c initial filling of the locations 32 is performed through a common feeding line 41 (see also FIG. 2) connecting the locations in series from starting from a common feeding inlet P1 for loading to a common feeding exit 43. The principle of operating the valve structures 40 within a location 32 are the same in the embodiment shown of FIG. 22 and the embodiment shown in FIGS. 22b and c.

FIG. 22b shows the device 30 during filling. The inlet and exit portion P1 and P2 are closed by a valve; initial filling of the locations 32 from feeding inlet 42 to feeding exit is possible via feeding line 41.

FIG. 22c shows the device 30 during perfusion and reverse flow generation. The feeding channel 41 is closed by valves, so each of the locations 32 are isolated from each other. Now the valves within each location 32 can be controlled individually to enable change of fluid directions as further described in detail with reference to FIGS. 21 and 23.

FIG. 23 shows CFD Simulations with generic microfabricated geometry for trapping spherical hydrogel matrices in a specific location 32, which is also described in more with reference to the circuit diagram of FIG. 21. FIGS. 21A and 23A) Normal operation. No microfabricated valves are closed, consequently resistances R2 and R3 in fluid lines 502 and 503 are much smaller than resistances R1 and R4 in fluid lines 501 and 504. The fluid flow perfuses the trap geometry 33 from top to bottom in direction S1. Thus, a particle is immobilized within the trapping structure 33. FIGS. 21B and 23B) The bottom left microfabricated valve represented by resistance R3 is closed. The main fluid stream goes through the upper channel. FIGS. 21C and 23C) The main fluid stream goes through the bottom channel. A particle is pushed into the trap. FIGS. 21D and 23D) Only when both microfabricated valves represented by resistances R2 and R3 are closed the reverse fluid flow in direction S2 removes that particle from the trapping structure 33.

FIG. 24 shows sequential removal of two hydrogel matrices (31C, 31A) by RFCP. A) Two hydrogel matrices might be located within close proximity. A reverse flow results in a force F2 acting on hydrogel matrix 2 (31C) and in a force F1 acting on hydrogel matrix 1 (31A) with F2 being larger than F1. Thus, at a certain flow rate only hydrogel matrix 2 is removed. B) Corresponding hydrodynamic resistances for generating two different forces acting on said hydrogel matrices.

FIG. 25 shows the removal of hydrogel matrices (31C, 31A) located within a RFCP geometry by using different reverse flow rates. An increase of the reverse flow rate might result in a removal of a first hydrogel matrix while all hydrogel matrices located within different microfabricated chambers might remain within their position. A further increase of the flow rate might result in a removal of a second hydrogel matrix from the same microfabricated chamber without removing hydrogel matrices located within other microfabricated chambers.

FIG. 26 shows the sequential removal of three hydrogel matrices (droplets 31-A-C) by RFCP. A), which are trapped in one single droplet trap. Also two hydrogel matrices might be located within close proximity. A reverse flow results in a force F3 acting on a hydrogel matrix 31C, in a force F2 acting on hydrogel matrix (droplet 31C) and in a force F1 acting on hydrogel matrix (droplet) 31A with F3 being larger than F2 being larger than F1. Thus, at a certain flow rate only hydrogel matrix (droplet 31C) is removed. Increasing the reverse flow rate leads to the sequential removal of the other hydrogel matrices. B) Corresponding hydrodynamic resistances for generating three different forces acting on said hydrogel matrices. An exemplary embodiment is also shown in FIG. 27.

FIG. 27C shows a generic location 32, details of which are shown in FIG. 27A. The location comprises two bypass sections 35 circumventing a group of positioner 33. Here three bottleneck sections 34A, 34B, 34C are provided in sequence each defining a positioner 33A, 33B, 33C. During loading of the location a first droplet 31A arriving at the positioners 33 will move up to the first positioner 33A and will be retained in the first positioner 33A. A second droplet 34B arriving subsequently will move up to the second positioner 33B upstream of the first positioner 33A and will be retained in the second positioner 33B. A third droplet 34C arriving subsequently will move up to a third positioner 33C upstream of the second positioner 33B and will be retained in the third positioner 33C. It is possible to provide any number of bottleneck sections 34/positioners 33 to enable a row of droplets 31 of a predetermined number. When all the positioners are occupied further droplets will follow the bypass section 35 and approach the locations at a downstream position along first fluid direction S1.

When the fluid is reversed to untrap the droplets at first droplet upstream (when viewed in first fluid direction S1) in third bottleneck section 34C will be untrapped. Due to the hydraulic design in the droplet trap the droplets retained in the upstream positioner 33C will be subject of an increased hydraulic pressure compared to the droplets retained in the downstream positioner 33A, 33B. So upon reversal of the fluid direction into the second fluid direction S2 at first the droplet in the most upstream positioner 33C will be untrapped and can be delivered to an exit section e.g. at P2 (see FIG. 21). at second the fluid pressure between P1 and P2 will be increased, so that subsequently also the droplets retained in the more downstream positioner 33A, 33B will be untrapped and will also be delivered to exit at P2. A suitable hydraulic design can be obtained by CFD simulations.

FIG. 28 shows a sequential removal of three hydrogel matrices in a trap having 3 bottleneck sections each by a first (downstream) droplet 31A, second droplet 31B and third (upstream) droplet 31C, without affecting hydrogel matrices located within other microfabricated chambers. During a first untrapping period I low pressure or flow rate p1 is applied through fluid, so that all droplets remain trapped. During a second period II an increased pressure or flow rate p2 is applied through the fluid, which is strong enough to remove merely upstream droplet 31C; the other droplet 31B, 31A remain trapped. During a third period III a further increased pressure or flow rate p3 is applied through the fluid, which is strong enough to remove second droplet 31B; the downstream droplet 31A remains trapped. During a fourth period IV a further increased pressure or flow rate p4 is applied through the fluid, which is strong enough to remove third upstream droplet 31A. The pressure can be applied through input P1 (see FIG. 21). The pressure can be regulated by an external fluid pump (not shown) in particular by a pump 50 described below.

FIG. 24 and FIG. 25 show the same concept as described with reference to FIGS. 26 to 28, but merely for the use of two droplet 31A, 31C to be retained within one droplet trap, having two bottleneck sections 34A, 34C.

FIG. 29 is an illustration of workflow for generating time-lapse cytokine profiles. To this end, at least two droplets/hydrogel matrices (31A, 31B) are positioned in a first step within a trap (33A, 33B) located within a location (32). This may be a trap for selective removal of trapped droplets/hydrogel matrices as a exemplary embodiment is also shown in FIGS. 24 and 25. A first droplet/hydrogel matrix (31A) may contain at least one cell (20). In addition, said droplet/hydrogel matrix (31A) may be held stationary for a defined period. A second droplet/hydrogel matrix (31B) may be positioned next to the first droplet/hydrogel matrix (31A). The second droplet/hydrogel matrix may contain capture molecules (e.g. antibodies, antibody-DNA conjugates, aptamers) for capturing of molecules secreted by at least one adjacent cell (20). In a particular embodiment, the fluid surrounding the trapped droplets/hydrogel matrices might be replaced by an oily fluid in a next step. Thus, the reaction volume is decreased to approximately the volume of both droplets/hydrogel matrices (31A,31B). This has the advantage, that the reaction volume is fixed to a defined volume and the concentration of secreted molecules is increased thereby increasing the measurement sensitivity of a potential detection mechanism. In a next step, both droplets/hydrogel matrices (31A, 31B) may be held stationary for a defined period in which secreted molecules might bind to capture molecules located within droplet/hydrogel matrix 31B. Afterwards, the fluid surrounding said droplets/hydrogel matrices might be exchanged again enabling washing of trapped droplets/hydrogel matrices and adding a second capture molecule that is labeled (e.g. with a DNA-barcode or with a fluorescent molecule). The second droplet/hydrogel matrix (31B) is then removed by applying a reverse flow as disclosed and collected in another format while the first droplet/hydrogel matrix 31A is held stationary. Afterwards, a new second droplet/hydrogel matrix (31B) is positioned again in 33B and the process is repeated. This method has the advantage, that secreted molecules can be captured in a time-lapse manner and analyzed either within the location (32) or after collection of said hydrogel matrices (31B). Secreted molecules may be cytokines.

FIG. 30 is an illustration of data that might be generated using the described time-lapse cytokine profiling technique.

FIG. 31 shows a workflow for the on-demand multi step stimulation of immobilized cells. To this end, at least two droplets/hydrogel matrices (31A, 31B) are positioned in a first step within a trap (33A, 33B) located within a location (32). This may be a trap for selective removal of trapped droplets/hydrogel matrices as a exemplary embodiment is also shown in FIGS. 24 and 25. A first droplet/hydrogel matrix (31A) may contain at least one cell (20). In addition, said droplet/hydrogel matrix (31A) may be held stationary for a defined period. A second droplet/hydrogel matrix (31B) may be positioned next to the first droplet/hydrogel matrix (31A). The second droplet/hydrogel matrix may contain molecules (e.g. growth factors) that can be released upon application of a stimulus (e.g. exposure to UV-light). In a particular embodiment, molecules may be bound to a hydrogel matrix by a photocleavable spacer and the stimulus for releasing bound molecules might be light, in particular UV-light. In a particular embodiment, the fluid surrounding the trapped droplets/hydrogel matrices might be replaced by an oily fluid in a next step. Thus, the reaction volume is decreased to approximately the volume of both droplets/hydrogel matrices (31A,31B). This has the advantage, that the reaction volume is fixed to a defined and known volume enabling to calculate the concentration of bound molecules. In a next step, both droplets/hydrogel matrices (31A, 31B) may be held stationary for a defined period in which bound molecules might be released to diffuse to droplet/hydrogel matrix 31B. Afterwards, the fluid surrounding said droplets/hydrogel matrices might be exchanged again enabling washing of trapped droplets/hydrogel matrices. The second droplet/hydrogel matrix (31B) is then removed by applying a reverse flow as disclosed while the first droplet/hydrogel matrix 31A is held stationary. Afterwards, a new second droplet/hydrogel matrix (31B) with the same bound molecule type or a different one is positioned again in 33B and the process is repeated. This method has the advantage, that molecules can be provided to at least one cell located within a location (32) in a time-lapse manner. Bound molecules may be growth-factors, in particular growth-factors of the following families: FGF, TFG, Hedgehog, Wingless, Delta and Serrate, Ehprine. In another embodiment, bound/released molecules may be CRISPR/Cas complexes, in particular for transfection adjacent cells.

FIG. 32 is an illustration of event-triggered cell stimulation.

FIG. 33 shows a two dimensional electrode arrangement having two electrodes 45A, 45B for the impedance measurement 38 as well as for the radiofrequency 39 excitation of hydrogel beads 31. A) Top view of an electrode arrangement. B) Hydrogel bead 31 positioned on top of the electrode arrangement. C) Hydrogel 31 on top of the electrode arrangement positioned in a microfabricated trapping geometry 33.

FIG. 34 shows a three dimensional electrode arrangement having two electrodes 45A, 45B for the impedance measurement 38 as well as for the radiofrequency 39 excitation of hydrogel beads 31 as well as hydrogel beads containing gold nanostructures. In a particular embodiment, the three dimensional electrode arrangement is a trap 33. A/B) Hydrogel Bead located within 3D electrodes. C/D) Electrode arrangement with immobilized Hydrogel Bead containing gold nanostructures such as gold nanocrystals.

FIG. 35 shows schematically an embodiment of an observation chamber 32, as already described with reference to FIG. 21, herein with the exemplary labeling of the observation chamber at position m2 n2. By the feeding channel 41 the droplets are delivered into the chamber 32. The feeding channel 41 may be directly connected to one of the first channels 11 in a microfabricated valve 10 in particular of FIG. 5, 9, 10, 11, which generates the droplets in the fluid. The first droplet 31 approaching the chamber 32 will be trapped by the droplet trap 33. All further droplets will take the bypass section 35 and will be supplied to the next chamber via the line 41. Valve V41 controls the flow via the channel 41. All Valves V41 of all chambers are connected to a common control line, which supplies a common command C41.

Valves VP1, VP2 controls the pressure and/or fluid rate between inlet P1 and exit P2. Valves VP1 and VP2 are connected to a common control line, which supplies a common command VP1 to all Valves VP1 and VP2 of all observation chambers 32.

Valve Vm2 represents the variable resistance R2; valve n2 represents the variable resistance R3 (see FIG. 21). All observation chambers 32 in columns m2 comprises a valve Vm2, which are commonly connected to the common command line providing the command Cm2 to all chambers in columns m2. All observation chambers 32 in line n2 comprises a valve Vn2, which are commonly connected to the common command line providing the command Cn2 to all chambers in line n2. Only when Cm2 and Cn2 are set to 1, the valve arrangement of Vm2 and Vn2 can reverse the direction of the fluid in the droplet trap 33.

The control lines are adapted to provide a command via a fluid such as pressured air or silicone oil.

FIGS. 36 and 37 shows a peristaltic pump comprising a plurality of valves.

FIGS. 52a and 52b shows a valve arrangement for isolated droplet generation using a pressure damping device 65.

FIGS. 53a and 53b shows an embodiment for extraction of cells located within immobilized hydrogel matrices and subsequent transfer into another format.

FIGS. 54a 5b shows an embodiment of generation of defined array compositions using the RFCP-based sorting mechanism that is described in the present disclosure.

FIG. 41. To avoid crosslinking of these groups within the main chain, protective groups are used during SZWIP. After polymerization the protecting group is cleaved from the polymer backbone.

FIG. 42. Copolymerization between heterobifunctional compounds as ME and cyclic imino ether as MN.

FIG. 43 shows a hydrogel with an encapsulated particle, which can be a cell. The magnification shows a hydrogel network structure comprising polymers with a multi-arm or star-shaped structure and a linear structure. In order to crosslink the polymer, different crosslinking mechanisms can be employed. In one example, crosslinking can be achieved by hybridization with nucleic acids or modifications thereof, such as PNA, wherein the crosslinking by hybridization can take place by hybridization between two complementary, hybridizing PNA sequences of the polymer or by adding a nucleic acid (e.g. DNA, PNA) comprising sequences complementary to the PNA sequences of the polymers. In another example, crosslinking can be achieved by moieties, which chemically react with each other, such as the reaction between a thiol-group and a maleimid-group. Between the crosslinking group and the polymer backbone, an enzyme degradable target site, preferably a matrix metalloprotease (MMP) target site can be present. The polymers can comprise a biologically active molecule. In a specific example, the biologically active molecule can be a protein derived from the extracellular matrix, such as collagen, fibronectin, and/or laminin, or a peptide sequence derived from extracellular matrix proteins, e.g. RGD-, LDV-, or YIGSR-sequence, or a cytokine, such as TFG-α. The coupling of a biologically active molecule to the polymer can take place between an amine-group of the bioactive molecule and an N-hydroxysuccinimide ester group of the polymer, whereby a covalent bond is established. The polymer can comprise a poly-(2-oxazoline)-based backbone, wherein the chemical moiety of the backbone (“R”) can be a hydrogen atom, methyl, ethyl, n-propyl, iso-propyl, n-butyl, iso-butyl, sec-butyl, tert-butyl, pentyl, iso-pentyl, neopentyl, sec-pentyl, hexyl, heptyl, octyl, nonyl, or decyl, more preferably methyl or ethyl. Importantly, the hydrogel network can comprise a large variety of polymer building blocks, including poly-(2-oxazoline) or copolymers thereof, as well as other hydrophilic polymers, which may comprise a PNA-sequence.

FIG. 44 shows an exemplary structure of the hydrogel network. The hydrogel network comprises a combination of linear and multi-arm or star-shaped polymers, wherein the polymers comprise functional groups, which allow direct or indirect crosslinking to each other. In case of direct crosslinking, for instance, the functional groups of a linear polymer are selected to react with the functional groups of a multi-arm or star-shaped polymer and/or vice versa. In one example, these functional groups can crosslink by hybridization between complementary nucleic acids, or modifications thereof, such as PNA. In another example the functional groups can crosslink chemically, as for instance by crosslinking thiol groups and maleimid groups. In case of indirect crosslinking, a crosslinking agent can be added, which couples to the functional groups of the polymers. For instance, a hybridizing nucleic acid sequence can be added to PNA-functionalized polymers or a crosslinking agent, which comprises functional groups that chemically react with functional groups of the polymer can be added, e.g. functional group comprising polymers, such as carboxy-, thiol-, or amine-functionalized polyethylene glycol (PEG), such as poly(ethylene glycol) bis(amine) or poly(ethylene glycol) dithiol or di(N-succinimidyl) functionalized components with dithiol moieties, such as dithiodipropionic acid di(N-hydroxysuccinimide ester or carboxy-functionalized disulfides, such as 2-Carboxyethyl disulfide. Furthermore, the polymer can comprise a biologically active molecule, which can couple to functional groups of the polymer and/or the functional groups are terminal functional groups that react with the biologically active molecule. In another embodiment, the functional groups are located along the polymer strands, which can allow coupling of a high degree of biologically active molecules.

FIG. 45 shows an exemplary structure of the hydrogel network. The hydrogel network comprises a combination of linear and multi-arm or star-shaped polymers, wherein the polymers comprise functional groups, which allow direct or indirect crosslinking to each other. The hydrogel network can comprise a number of different linear and multi-arm or star-shaped polymers, wherein the polymers represent building blocks for the hydrogel network. In this exemplary structure, various polymers and/or copolymers were combined including:
A linear copolymer “Polymer A”, which is based on polyoxazoline and “Y”, which can comprise functional moieties, such as N-hydroxysuccinimide esters, capable of coupling to biologically active molecules comprising primary amine groups. Furthermore, “Polymer A” comprises terminal moieties comprising PNA sequences, which can form crosslinks by hybridization with complementary PNA sequences or, alternatively, crosslink chemically when additionally comprising a thiol-group at the PNA-sequence, which can couple, for instance, to a maleimide compound.
A star-shaped copolymer “Polymer A+”, which can have the same properties as “Polymer A” but comprises a multi-arm or star-shaped polymer structure.
A linear copolymer “Polymer C”, which is based on polyoxazoline and “Y”, which can comprise functional moieties, such as N-hydroxysuccinimide esters, capable of coupling to biologically active molecules comprising primary amine groups. Furthermore, “Polymer C” comprises terminal moieties with chemically crosslinkable groups, such as maleimide or alken, which can form crosslinks with other polymers, for instance by coupling to terminal thiol groups.
A star-shaped copolymer “Polymer C+”, which can have the same properties as “Polymer C” but comprises a multi-arm or star-shaped polymer structure.
A linear “Polymer B”, which is based on polyoxazoline and can comprise functional moieties in the backbone, for instance, at the 2-substituent position (e.g. R4 group), such as N-hydroxysuccinimide esters, capable of coupling to biologically active molecules comprising primary amine groups. Furthermore, “Polymer B” comprises terminal moieties comprising PNA sequences, which can form crosslinks by hybridization with complementary PNA sequences or, alternatively, crosslink chemically when additionally comprising a thiol-group at the PNA-sequence, which can couple, for instance, to a maleimide compound.
A star-shaped copolymer “Polymer B+”, which can have the same properties as “Polymer B” but comprises a multi-arm or star-shaped polymer structure.
A linear “Polymer E”, which is based on polyoxazoline and can comprise functional moieties in the backbone, for instance, at the 2-substituent position (e.g. R4 group), such as N-hydroxysuccinimide esters, capable of coupling to biologically active molecules comprising primary amine groups. Preferably, “Polymer E” comprises at least two different groups at the 2-substituent. Furthermore, “Polymer E” comprises terminal moieties with chemically crosslinkable groups, such as maleimide or alken, which can form crosslinks with other polymers, for instance by coupling to terminal thiol groups.
A star-shaped copolymer “Polymer E+”, which can have the same properties as “Polymer E” but comprises a multi-arm or star-shaped polymer structure.
A linear “Polymer D”, which is based hydrophilic polymeric residue, preferably independently derived from monomers independently selected from oxazoline, ethylene glycol, propylene glycol, acetal lactic acid, glycolic acid, vinyl alcohol, and can comprise functional moieties in the backbone, capable of coupling to biologically active molecules. Importantly, “Polymer D” comprises terminal moieties comprising PNA sequences, which can form crosslinks by hybridization with complementary PNA sequences or, alternatively, crosslink chemically when additionally comprising a thiol-group at the PNA-sequence, which can couple, for instance, to a maleimide compound.
A star-shaped copolymer “Polymer D+”, which can have the same properties as “Polymer D” but comprises a multi-arm or star-shaped polymer structure.

The resulting hydrogel network can be independently tuned regarding a multitude of characteristics, including density and number of biologically activated molecules, hydrophilicity and hydrophobicity of the matrix, pore size and network/crosslinking density, hydrogel mechanics (e.g. stiffness, elasticity, ductility, viscoelasticity, etc.), and degradability.

FIG. 46 shows an example of a preferred structure of the hydrogel network. The hydrogel network comprises a combination of linear and multi-arm or star-shaped polymers, wherein the polymers comprise functional groups, which allow crosslinking to each other, either directly via hybridization of PNA-sequences located at the terminal ends of the polymers, or indirect by a crosslinking agent (e.g. a complementary oligomer comprising DNA or PNA). The hydrogel network can comprise a number of differently functionalized linear and multi-arm or star-shaped polymers, wherein the functionalization takes place between functional groups of the polymer and one or more biologically active molecules. The linear and multi-arm or star-shaped polymers are copolymer (above named “Polymer A/A+”), which is based on 2-substituted oxazoline and the comonomer “Y”, which can comprise functional moieties, such as N-hydroxysuccinimide esters, which may be attached to the copolymer through a linker, which can be degradable. The functional moieties, such as N-hydroxysuccinimide esters are capable of coupling to biologically active molecules comprising primary amine groups. A library of biologically active molecules may be attached to the copolymer, which will be recognized by the skilled person. These inter alia include antibodies, the RGD peptide sequence, and/or epidermal growth factor (EGF). The copolymer backbone comprises an inert 2-substituent of the oxazoline moiety which can be a hydrogen atom or a hydrocarbon compound, preferably a methyl or ethyl group. Furthermore, the copolymer comprises terminal moieties comprising PNA sequences, which can form crosslinks by hybridization with complementary PNA sequences or, alternatively, crosslink chemically when additionally comprising a thiol-group at the PNA-sequence, which can couple, for instance, to a maleimide compound. The copolymer can optionally or partially comprise enzyme degradable target sites, such as a matrix metalloprotease sensitive target site, which are preferentially located between the PNA moiety and the copolymer.

Subsequently, independently selectable, but preferred features of the hydrogel structure crosslinked by PNA sequences are listed, the features are preferably used in combination: Polymeric composition: Linear and multi-armed. The linear polymers comprise functional groups for coupling to bioactive molecules. The multi-armed polymers comprise saturated NHS esters and are biologically inert for cells. They can be added to cell suspension prior to hydrogel formation.

Cross-Linking of polymeric precursors: Crosslinking of linear and multiarmed precursors. The hydrogel is formed by alternating linear and multi-arm precursors resulting in uniform hydrogels. The pore size is adjusted by the length of the polymer. Gelation after mixing of two different precursor polymers.

2-substituent for physiochemical properties of the gel: Alkane based substituents from Methyl to Dodecyl or hydrogen. The length of the hydrocarbon defines the physiochemical character of the hydrogel.

Functional site of the 2-substituent: No functional sites as 2-substituent.

Functional site of co-polymerized pendent moiety Y: NHS-ester

Direct coupling/release of bio-active com-pounds: Direct linkage of bioactive compounds to NHS-ester of the linear polymer. On-demand release via degradation of the linker (k).

Cross-linking: Crosslinking through complementary PNA-sequences. Crosslinking has no effect on cells (phenotype and viability).

An according hydrogel is degradable via denaturation of PNA sequence hybridization by on-demand addition of complement PNA Sequences in molar excess. In addition the gel is degradable by cell secreted enzymes such as MMPs. The stiffness is fine-tunable and completely independent from the degree of functionalization with bioactive compounds.

The mesh size/gel shell is fine tunable by the length of the polymers.

FIG. 47 shows the termination of a cationic ring opening polymerization reaction by an amino or carboxy-group of PNA. A positively charged oxazolinium species can react with the amino-, thiol-, acrylic acid or carboxy-group of another molecule, such as PNA or a biologically active molecule and terminate the reaction. As a result, polymers can be produced that have terminal moieties comprising a desired molecule, such as a PNA-sequence, biologically active molecule, and/or a crosslinking functional group.

FIG. 48 shows some of the PNA crosslinking strategies to form a hydrogel. Crosslinking by hybridization of PNA can be achieved directly by mixing two polymers that comprise PNA-sequences, which are complementary to each other (1.). Furthermore, crosslinking can be achieved indirectly by supplying a PNA (2.) or DNA sequence (3.) that is complementary to the PNA sequences of the polymers with the proviso that the PNA sequences of the polymers are not complementary to each other. In addition, a crosslinking compound can be added comprising two terminal PNA sequences complementary to the PNA sequences of the polymers and further comprising an enzyme degradable target site, preferably a matrix metalloprotease (MMP) target site between the two terminal PNA sequences, allowing enzymatic degradation (4.). Alternatively, the compound between the PNA sequences is not degradable. In other embodiments the PNA crosslinking strategies 1., 2., and 3. comprise an enzyme degradable target site, preferably a matrix metalloprotease (MMP) target site between the terminal PNA sequence and the polymer (1a., 2a., and 3a.). In another alternative, the applied nucleic acids or modifications thereof are less than 100% complementary to each other in order to adjust the strength of the hybridization crosslinks. In yet another alternative, the hybridizing nucleic acids or modifications thereof comprise mismatched base pairs (5.). Alternatively, in the PNA-crosslinking strategies other nucleic acids or modifications thereof than PNA can be applied, such as DNA, RNA, LNA or HNA (6.). Alternatively, DNA/PNA hybridization can take place via Watson-Crick and/or Hoogsteen base pairing (under triple helix formation, e.g. 3II.).

FIGS. 49 and 50 shows some of the applicable chemistries to form a gel-shell around a hydrogel matrix (gel-shell bead). Residual functional groups of the hydrogel bead or polymer network can be utilized to catalyze crosslinking at the surface or at close proximity to the surface, resulting in gel-shell formation. For example, residual N-hydroxysuccinimide ester moieties can couple to an amine-group comprising polymeric compound, which can be selected from poly(allylamine), (branched) amino-polyethyleglycol (PEG), (branched) polyethylenimine (PEI), polylysine, poly amidoamine (PAMAM) dendrimer, poly(β-amino ester), chitosan, amino-PaOX, and, optionally, 2-amino-1,3-propanediol, 3-amino-1,2-propanediol, which can be applied to modify the thickness of the gel shell (FIG. 49). In another example, amine-functionalized small polymers or diamines, such as 1,3-diamino-2-propanol are present in the hydrogel bead/polymer network and N-hydroxysuccinimide ester moieties of added polymers, such as PaOX-NHS-ester or PEG-NHS-ester, are catalyzed to react at the location of the amine-functionalized small polymers or diamines (FIG. 50). As the larger added polymers cannot pass the hydrogel network, polymerization is only catalyzed at the surface resulting in gel-shell formation. Additionally, the diamine compound may comprise an enzyme degradable target site, such as a matrix metalloprotease sensitive target site. In this case, the shell is not directly crosslinked to the hydrogel. In another embodiment, the diamine can be replaced by a dithiol comprising compound, such as 2,2′-(ethylenedioxy)diethanethiol or short dithiol functionalized polymers with enzyme degradable target site, such as a matrix metalloprotease sensitive target site, which then reacts with maleimide comprising compounds, which are moieties of added polymeric compounds (FIG. 50). In yet another example, polymers comprising one or more PNA sequences can be added to the hydrogel bead/polymer network, which can hybridize to residual PNA-sequences of compounds which freely diffuse through the hydrogel bead/polymer network to form a gel-shell. Preferably, the added polymer comprising PNA sequences is linear and the compound which freely diffuses is a multi-arm or star-shaped polymer comprising hybridizing PNA sequences. Hence, the shell forming compounds comprise different PNA sequences than those, which may form the hydrogel matrix crosslinks. Therefore, the PNA sequences of the crosslinking polymer are different from the PNA sequences of the core-polymer and are only complementary to the residual PNA sequences (FIG. 50). In general, the gel-shell can be adjusted in the thickness and density inter alia by choice of the molecular weight of the added compounds, the structural properties of the added compounds (e.g. linear, multi-arm, etc.) and the availability and amount of residual and added functional groups.

FIG. 51 A. Termination of the copolymerization between heterobifunctional compounds as ME and cyclic imino ether as MN. The Copolymerization is terminated by acrylic acid resulting in α-acrylic and ω-acid end-groups, respectively; m is the lengths of a linker.

FIG. 51 B. Termination of the copolymerization between heterobifunctional compounds as ME and cyclic imino ether as MN. The copolymerization is terminated by addition of a nucleophile, an electrophile or a combination of a nucleophile and an electrophile. During termination the reactive cyclic oxazolinium is ring-opened by the nucleophile and the electrophile reacts with the carbene from ME; m is the lengths of a linker.

### DETAILED DESCRIPTION OF THE INVENTION

The present invention describes a novel microfabricated and programmable array of spherical hydrogel matrices or cell-laden spherical hydrogel matrices, microfabricated structures and chemical compounds for producing said array and methods for the cultivation of cells and analysis of cells and cell components (e.g. mRNAs, miRNAs, DNA, secreted molecules) located in said array. In detail, the present invention includes microfabricated structures, chemical compounds and methods for the generation of spherical hydrogel matrices with defined compositions, the encapsulation of a defined number of cells in said matrices, the immobilization of biological molecules in said matrices, the controlled positioning of said matrices in a microfabricated array as well as their controlled removal at any time point from any position and transfer into established formats. The present structures, chemical compounds and methods are ideally suited for producing, positioning and handling (cell-laden) spherical hydrogel matrices, but are not so limited.

The novel microfabricated array containing spherical hydrogel matrices provides several advantages over currently existing arrays:

Programmable particle positioning. The first advantage is that spherical hydrogel matrices with defined characteristics (such as size, composition (e.g. immobilization of compounds or cells)) can be positioned on said array in a programmable manner. For example, if said array has n×m microfabricated individual chambers (n representing the number of rows and m representing the number of columns), a defined number of spherical hydrogel matrices with defined characteristics can be positioned in each of the n×m microfabricated individual chambers. Thus, one microfabricated chamber might contain one or more spherical hydrogel matrices that might contain single or multiple cells of the same or of different type or that might contain immobilized biological or bioactive compounds such as proteins (antibodies, growth factors), nucleic acids or small molecules. For example, a first spherical hydrogel matrix that contains one single cell of cell type 1 might be positioned next to a second hydrogel matrix that contains one single cell of cell type 2 in one microfabricated chamber.

Programmable particle removal and transfer. A second advantage in comparison to other arrays is that said immobilized spherical hydrogel matrices can be removed in a defined way from said array at any time-point and from any position and said removed hydrogel matrices can subsequently be transferred into another format such as a well plate or similar format. In addition, removal of said hydrogel matrices does not affect hydrogel integrity and thus results in a higher cell viability as well as in a maintenance of any information (such as bound barcoded antibodies) that might be coupled to said hydrogel matrices. For example, if a first spherical hydrogel matrix is located within a microfabricated chamber at position (n, m) and a second hydrogel matrix is located within close proximity to the first hydrogel matrix or is in direct contact with the first hydrogel matrix, the second hydrogel matrix might be removed first while the first hydrogel matrix stays within the microfabricated chamber. Afterwards, the first hydrogel matrix might be removed in a second step. This can also be done for more than two hydrogel matrices.

Reduction in actuator number. A third advantage of said array is that for specifically removing hydrogel matrices from a position (n,m) only n+m actuators are necessary (instead of n×m actuators−one actuator for one microfabricated chamber). This dramatically decreases the number of required actuators from n×m to n+m thus by (n+m)/(n*m)-fold. For example, if said array has 30×30=900 microfabricated chambers, only 30+30=60 actuators (instead of actuators) are needed for removing hydrogel matrices from any position (n,m).

Simultaneous removal of hydrogel matrices. A further advantage of said array is that hydrogel matrices located within different microfabricated chambers can be removed simultaneously. For example, a first hydrogel matrix located within a microfabricated chamber (n₁, m₁) might be removed at the same time at which a second hydrogel matrix located within a microfabricated chamber (n₂, m₂) is removed. This can also be done for more than two hydrogel matrices located at more than two different positions. Thus, the advantage is a dramatic reduction of time needed for removing said hydrogel matrices and transferring them into another format suitable for a corresponding downstream analysis.

Individual perfusion of chambers. A further advantage of said array is that microfabricated chambers can be individually perfused with a fluid. For example, cells located in said spherical hydrogel matrices positioned in said array can be continuously or step wise perfused with fresh cultivation medium resulting in a removal of cellular waste products and supply with fresh nutrients. Thus, cells can be cultivated within n×m microfabricated chambers for an extended period as new nutrients can be supplied continuously whereas all microfabricated chambers might have the same culture conditions.

Sequential perfusion. A further advantage of said array is that microfabricated chambers can be sequentially perfused with fluids of different compositions of the same or of different type. For example, microfabricated chambers with immobilized hydrogel matrices containing cells might be first perfused with a solution containing a first fluorescent antibody against specific cell surface proteins. Afterwards, said array might be perfused with a solution that removes the fluorescent signal from the first antibody. Afterwards, said array might be perfused with a second fluorescent antibody with another specificity resulting in the staining of a second cell surface marker. This process might be repeated many times resulting in a cell surface profile of cells located within n×m microfabricated chambers.

Alternating biphasic compartment generation. A further advantage of said array is that immobilized hydrogel matrices located within microfabricated chambers can be repeatedly transferred into a reduced volume compartment without changing the position of said hydrogel matrices thereby reducing the reaction volume and thus increasing the local concentration of analytes (e.g. mRNAs, PCR-Products) which increases the sensitivity of potential detection mechanisms. For example, a microfabricated chamber at position (n, m) containing a hydrogel matrix might be first perfused with an aqueous phase. In a second step, said microfabricated chamber might be perfused with an immiscible phase (e.g. fluorinated oil) resulting in a water-oil interface located around an immobilized hydrogel matrix. Thus, the volume reduces from the volume of one isolated microfabricated chamber to the volume of one hydrogel matrix. In a third step, said microfabricated chamber (containing fluorinated oil) might again be perfused with an aqueous phase. Thus, an immobilized hydrogel matrix would be located again within an aqueous phase and detected analytes can be washed away after their detection. The process might be repeated for detecting multiple analytes in sequential order.

Radiofrequency heating of hydrogel matrices. A further advantage of said array is that immobilized hydrogel matrices might be heated to a desired temperature in a very fast manner by using a radio frequency and a microfabricated radio antenna located within said microfabricated chambers. This fast heating mechanism results in a dramatic time reduction of processes where a sequential heating to different temperatures is required (e.g. PCR). For example, immobilized hydrogel matrices might contain immobilized gold nanostructures that react to an applied radio frequency field. Said radio frequency field might be generated by an electrode located within said microfabricated chambers acting as radio frequency antenna.

Impedance measurements of hydrogel matrices. Another advantage of said array is the fast determination of colony sizes and growth rates of cell colonies using impedance measurements and thus a reduction of system complexity. For example, cell-laden hydrogel matrices might be positioned in microfabricated chambers that contain a microfabricated electrode structure surrounding said hydrogel matrix. By applying an alternating electric field and measuring the response current, the colony size and growth rate of cell colonies might be determined.

Coupling of time-lapse phenotypic data with genomic data. Another advantage of said array is that cells can be cultivated and imaged over an extended period at n×m positions and cells can be removed from positions n×m at any timepoint and as soon as a defined requirement is fulfilled. Afterwards, removed cells might be analyzed with conventional methods such as qRT-PCR or sequencing. Thus, a further advantage is the coupling of time-lapse data such as microscopy data and data from other techniques such as qRT-PCR or sequencing. For example, a single cell located within a hydrogel matrix at position (n, m) might express a fluorescent protein that is coupled to a specific promotor. The single cell might start to proliferate resulting in a small cell colony. As soon as the fluorescent signal of said colony reaches a certain value the hydrogel matrix located at position (n, m) containing said colony might be removed and analyzed with qRT-PCR or NGS. Thus, a further advantage would be the coupling of an observed, time-lapse phenotype with genotypic data.

On-demand cross-talk. Another advantage is that said microfabricated chambers can be isolated from each other on-demand preventing any cross-communication between said microfabricated chambers. For example, if cells are cultivated in a first microfabricated chamber and other cells are cultivated in a second microfabricated chamber that is located next to the first microfabricated chamber and if said cells secret molecules such as cytokines that affect cell behavior, the cross-communication between said microfabricated chambers can be controlled. Thus, cross-communication between two microfabricated cultivation chambers might be prevented if it is not desired.

Particle centering. A further advantage of said array is that encapsulated cells located within spherical hydrogel matrices positioned on said array are located within the center of said spherical hydrogel matrices. For example, a single cell located within the center of a hydrogel matrix might start to proliferate resulting in an increased colony size. A proliferating cell located at the edge of the hydrogel matrix might escape from the hydrogel matrix. Thus, the advantage is that a growing cell does not leave the spherical matrix due to the prior centering of said cell within the center of said hydrogel matrix.

Programmable hydrogel composition. Another advantage of said array is that the composition of the hydrogel matrix can be programmed. Thus, at each position within said array a hydrogel matrix with a different composition might be positioned. For example, stem cell differentiation is affected by immobilized growth factors immobilized in said hydrogel matrices. Thus, different growth factors might be immobilized within hydrogel matrices located at different positions within said array. This would have the advantage that said array might be used for screening for hydrogel matrices affecting cell behavior such as stem cell differentiation.

Synthetic hydrogel character enables defined structures. Another advantage of said array is that the used hydrogel matrices located within said array persist of completely defined and fine-tunable structures. Said novel hydrogel matrices are composed of a copolymer constructed of heterocyclic chemical compounds like 2-oxazoline and unsaturated imides like 3-(maleimido)-propionic acid N-hydroxysuccinimide esters that possess different properties such as different chemical, mechanical, and biomechanical properties regarding the building blocks used for copolymerization. These properties are of importance as they influence the cell phenotype, cell fate and cell secretion patterns such as proliferation, self-renewal, colony- and tumor sphere formation, differentiation, migration and polarization. The fine-tunable structure makes it possible to investigate responses from precise alterations of mechanical and biochemical properties in systematic ways to independently control key parameters responsible for cell behavior and cell characteristics. Depending on the 2-substitution on one hand the water-solubility can be adjusted from highly hydrophilic (2-methyl-) or slightly amphiphilic being comparable to polyethylene glycol (PEG), to highly hydrophobic (e.g. 2-nonyl-), on the other hand reactive side-chains can be easily introduced by using functional monomers.

Stealth characteristics of the hydrogel matrix backbone. Another advantage of the novel hydrogel matrices located within said array are their so called stealth characteristics that render the hydrogel backbone completely undetectable for living cells. This has the advantage that no unwanted pathways within the cell are activated by the hydrogel backbone. Usually hydrogels from natural origin like agarose, alginate or gelatin lack this stealth characteristic leading to unwanted activation of enzymatic cascades and altered cell responses.

Absence of toxins and undefined molecules within hydrogel matrices. In contrast to hydrogels raised from natural sources the novel synthetic hydrogel matrices located in said lack toxins and other undefined molecules. These unknown molecules might significantly interact with cells of interest and thus alter the cell response making the investigation of precise responses upon defined stimuli impossible. Thus, the absence of toxins and undefined molecules is important regarding the use of the hydrogel matrices for cell cultivation and cell analysis. In addition, the absence of said unknown molecules is an inalienable premise for clinical and diagnostical applications.

No batch to batch variability between hydrogel matrices. Another advantage of the novel hydrogel matrices is the dramatic decrease in batch to batch variability. To date the most popular natural hydrogel is the Matrigel manufactured by Corning. The Matrigel is of tumorigenic source and thus exhibits a high batch to batch variability regarding mechanical and bio-chemical properties making it impossible to compare results from different batches or even to compare results from different researchers. Because of the high batch to batch variability the Matrigel is inapplicable for clinic research or diagnostic applications. In contrast, said novel hydrogel matrices located in said array show no variability between different batches.

High degree of hydrogel matrix functionalization. Another advantage of said array is that immobilized hydrogel matrices enable a high degree of functionalization due to the presence of a highly-increased number of functionalization sites in comparison to established matrices. With the use of these sites the novel hydrogel can be engineered to present different adhesive ligands, bioactive compounds and functional biomolecules such as adhesive compounds of the extra cellular matrix (ECM), growth factors, antibodies, CRISPR-Cas and nucleic acids. Commonly used synthetic hydrogel compositions such as Polyethylene glycol (PEG) hydrogels lack this high degree of functionalization. They are restricted to end-functionalization of the polymers limiting the number of incorporated compounds in highly-crosslinked hydrogels. In addition, said increased degree of functionalization results in a higher dynamic range in terms of analytes that might be detected using probes immobilized in said hydrogel matrices.

Fast hydrogel gelation process by cell compatible cross-linking. Another advantage of said array is that the formation of said hydrogel matrices occurs in a highly cell-compatible manner as hydrogel precursor molecules can be crosslinked by all cell-compatible crosslinking reactions. These reactions comprise reactions based on (i) covalent bond formation, chosen from the group consisting of a) enzymatically catalyzed reactions, and b) not-enzymatically catalyzed and/or uncatalyzed reactions, and/or ii) non-covalent bond formation such as of hydrophobic interactions, H-bonds, van-der-Waals or electrostatic interactions. In addition, said cell-compatible crosslinking reaction might include hydrogen bond formation between two peptide nucleic acid (PNA) molecules with different base sequences or two locked nucleic acid (LNA) molecules with different base sequences or a combination of one PNA molecule and one LNA molecule.

Degradation sites for on demand degradation of the hydrogel matrices. A further advantage of said array is that cells immobilized within said hydrogel matrices can enzymatically modify the surrounded matrix for cell migration and motility. The enzymatic modification of surrounded matrices represents a critical aspect of a cells' natural environment and thus is critical for a correct cell function and response. Thus, hydrogel matrices possess multiple degradation targets for secreted enzymes such as MMP target sites to enable matrix remodeling by incorporated cells. A further advantage of said hydrogel matrices is that said hydrogel matrices can be degraded by increasing the temperature. Thus, for a fast analysis of cell characteristics and cell behavior the hydrogel matrices can additionally be degraded by the user by heating up said hydrogel matrices.

Tunable stiffness of the hydrogel matrices. Another advantage of said array is that the mechanical properties of said hydrogel matrices can be adjusted by changing the concentrations of the used hydrogel precursor molecules. The mechanical properties of the three-dimensional hydrogel matrices are influenced by the concentration of precursor molecules and the molecular weight of the precursor molecules. Both parameters can independently be adjusted and combined. In contrast to other synthetic hydrogels such as PEG-based hydrogels the stiffness of the hydrogel matrices is completely independent from the number of functional sites, because these sites do not compete with the sites for crosslinking reactions. Thus, e.g. the stiffness of the matrix, represented by Young's moduli (E), can vary between 300 to 5400 Pa with the same number of functional sites.

Tunable mesh size of the hydrogel matrices. A further advantage of said array is that the mesh size of immobilized three-dimensional hydrogel matrices is also influenced by the concentration of precursor molecules and the molecular weight of the precursor molecules which can be independently adjusted and combined. In contrast to other synthetic hydrogels such as PEG-based hydrogels the mesh size of the hydrogel matrices is completely independent from the number of functional sites, because these sites do not compete with the sites for crosslinking reactions. The tunable mesh size makes the hydrogel matrices perfectly suitable for diffusion of different adhesive ligands, bioactive compounds and functional biomolecules such as antibodies and nucleic acids for ELISA, immunostaining, PCR, flow cytometry and sequencing.

Low auto fluorescence of the hydrogel matrices. Another advantage of said array is that the backbone of said hydrogel matrices exhibits only a very low fluorescence. Thus, the hydrogel matrices formed by the precursor molecules are suitable for all fluorescence-based detection mechanisms such as fluorescence microscopy and FACS as well as for spectrophotometry.

### Polymers

Polymers function as building-blocks of the inventive hydrogel matrices. During the gelation process said polymers are crosslinked via functional groups for crosslinking. Said polymers also may comprise functional groups for binding biologically active compounds to the polymer backbone. Said biologically active compounds may be linked to said functional group prior to gelation or after gelation or during gelation.

It is particularly preferred, that a polymer as a building block for hydrogel matrices is not self-crosslinking. Preferably at least two different polymers, preferably at least one linear polymer of this invention and at least one star-shaped polymer of this invention, crosslink in order to form said hydrogel.

Preferred functional groups for crosslinking (i.e. crosslinking of polymers) are independently selected from amine, N-hydroxysuccinimide, sulfo-N-hydroxysuccinimide, isothiocyanate, maleimide, thiol, azide, alkyne, alkene, hydrazide, aminoxy, aldehyde, carboxyl, carboxylate, hydroxyl, acrylate, vinyl ether, epoxide (preferably from amine, maleimide, alkyne, alkene, azide, carboxyl, carboxylate, methacrylate, acrylate, thiol).

Preferred functional groups for binding a biologically active compounds are independently selected from amine, N-hydroxysuccinimide, sulfo-N-hydroxysuccinimide, alkyne, alkene, hydrazide, epoxide, glycidyl, carboxyphenyl, methoxycarbonyl, carboxyl, carboxylate, isothiocyanate, maleimide, aminoxy, hydroxyl, vinyl ether (preferably from amine, N-hydroxysuccinimide, sulfo-N-hydroxysuccinimide, hydrazide, epoxide, glycidyl, phenyl acrylate, methoxycarbonyl, carboxyl, carboxylate).

It is particularly preferred, if said polymer comprises at least one functional group independently selected from arene, amine, alkyne, azide, anhydride, acid anhydride, ketone, haloalkane, imidoester, diol, hemiacetal, acrylate, alkene, thiol, ether, ester, isocyanate, isothiocyanate, succinimide, N-hydroxysuccinimide, sulfo-N-hydroxysuccinimide, amide, maleimide, N-heterocyclic carbene, acyl halide, N-heterocyclic phosphine, hydrazide, nitrile, aminoxy, imidazolide, imine, aldehyde, azo compound, imide, carbodiimide, haloacetyl, pyridyl disulfide, carboxamide, vinyl ether, carboxyl, carboxylate, phenyl, phenol, indol, methylthiol, pyridyldithiol, hydroxyl, epoxide, carbonyl, methoxycarbonyl, glycidyl or carboxyphenyl (particularly preferred independently selected from selected from the group consisting of protected N-hydroxysuccinimide-esters, unprotected N-hydroxysuccinimide-esters, sulfo-N-hydroxysuccinimide esters, vinyl sulfone, sulfonyl chloride, aldehyde, epoxides, thiol, maleimide or carbonate).

The polymers of the present invention are preferably selected from homopolymers of hetecocyclic chemical compounds or copolymers of heterocyclic chemical compounds or copolymers of heterocyclic chemical compounds with a monomer different from said heterocyclic chemical compound. The polymers of the invention are selected from linear polymers, random copolymers, block copolymers, graft polymers, multi-arm polymers, crosslinked polymers (crosspolymers), polymers with dendritic structure, star-shaped polymers.

The backbone of the polymers according to this invention is preferably formed by hydrophilic peptide-like polymers such as poly-2-oxazoline based polymers (POx), especially poly-2-methyl-2-oxazoline (PMOx)-based polymers, most preferably linear and multiarm POx-based polymers that are functionalized and able to be crosslinked by cell-compatible crosslinking reactions (Table 1). These polymers are pseudo-peptides with a high biocompatibility and show structural similarities to naturally occurring polypeptides.

All inventive polymers are prepared by preparation methods and preparation strategies which are well known to the skilled artisan and are described as follows.

The polymer according to this invention is formed by living cationic ring-opening polymerization (LCROP) and/or spontaneous zwitterionic copolymerization (SZWIP), preferably of cyclic imino ethers (CIE), most preferably of oxazolines or oxazines or oxazepines, substituted at position 2, respectively.

In an advantageous embodiment of this invention the spontaneous zwitterionic copolymerization (SZWIP) is used to produce copolymers of poly-2-oxazoline and heterobifunctional reagents most preferably for cell culture microenvironments.

The SZWIP of diverse compatible nucleophilic and electrophilic monomers can be used for the preparation of different polymer classes with various functionalities. The SZWIP, which was initially discovered in the 1970s by Saegusa and coworkers, takes place by the reaction of nucleophilic and electrophilic monomers by forming a propagating species which exhibits both a cationic and an anionic end group. Due to intramolecular and intermolecular reactions of the propagating species the propagation can either occur from one site in a cationic or anionic mechanism or by cation-anion reactions between zwitterions. The preparation of polymers from heterocyclic chemical compounds like oxazolines is described in the art (see: Kempe, Macromol. Chem. Phys., 2017, 218, 1700021 (DOI: 10.1002/macp.201700021)).

SZWIP requires no initiator or catalyst. Instead of an initiator, a nucleophilic monomer (MN) spontaneously reacts through a dipole-dipole interaction with an electrophilic monomer (ME) under the formation of a zwitterion+MN-ME-. In this genetic zwitterion the cationic MN and anionic ME are in general covalently bound. Alternatively, the two monomers can form a charged complex resulting in an equilibrium between the genetic zwitterion, neutral monomers and the charged complex. The zwitterion itself serves as an initiator and propagating species. Following the standard mechanism for step-growth polymerizations two of these genetic zwitterions react with each other through their charged end-groups to form a dimeric zwitterion. Further propagation steps lead to growing oligomeric or polymeric macro-zwitterions +MN-[MEMN]n-ME-. This macro-zwitterion in turn reacts with further zwitterions and macro-zwitterions of different lengths. Due to the cation-anion coupling mechanism predominantly alternating copolymers are formed during SZWIP.

Propagation can continue as long as there are other zwitterionic species to react with. Alternatively, the reaction is terminated by the reaction of the charged ends with their charged counterpart. To prevent premature termination these reactions are mainly performed in anhydrous polar aprotic solvents such as acetonitrile or N,N-dimethylformamide (DMF) and optionally in the presence of radical inhibitors. Under these conditions, the number-average molecular weights obtained by SZWIP of these monomers are typically in the order of 500-5000 g mol⁻¹.

Solvent, temperature and polymerization-times have been extensively studied resulting in the following preferable conditions for SZWIP: acetonitrile or N,N-dimethylformamide as solvent, 40-130° C. and 12-48 h. The use of dipolar aprotic solvents not only enhance an alternating copolymerization, but also increase the yields of the polymers after purification.

To date, numerous monomer combinations have been reported to react in a SZWIP-like fashion. The largest class of SZWIP copolymers constitutes those synthesized from CIEs. In the context of SZWIP, 2-oxazolidines (Ox) and 2-oxazines (Oz) have been extensively studied. These CIEs have been shown to be nucleophiles MN reacting with electrophiles ME including acrylic acid, acrylamide, propiolactone, anhydrides and sulfolactones. In case of (meth)acrylic acid and derivatives thereof, after Michael addition of Ox to (meth)acrylic acid (derivatives), a carbanion intermediate is formed, which then rearranged via a proton transfer into a more stable genetic zwitterion. Radical photopolymerization of (meth)acrylic acid can be prevented by addition of a small amount of a radical inhibitor (e.g. p-methoxyphenol (MEHQ)).

One preferred termination mechanism for the SZWIP of CIEs and (meth)acrylic acid (derivative) consists of the introduction of an α-(meth)acrylic end group. The introduction of two possible w end groups, carboxylic acids or amides, has also previously been identified. These termination mechanisms enable the preparation of heterotelechelic materials.

In an advantageous embodiment of this invention the spontaneous zwitterionic copolymerization (SZWIP) between CIEs and heterobifunctional monomer systems is preferably used to produce functional side groups within the resulting polymers. CIEs are used as powerful nucleophiles MN reacting with heterobifunctional reagents as electrophiles ME.

The heterobifunctional reagents comprise two different functional groups separated by an optional spacer which is degradable or inert. The first functional group is an electrophilic group and copolymerizes with a CIE during SZWIP.

The spacer is for example an inert hydrocarbon, polyethylene glycol or an aliphatic water-soluble molecule. Optionally, a degradable moiety (for examples vide infra) is incorporated within the spacer.

In one embodiment, said spacer is particularly preferred a degradable spacer, most preferred degradable by change of the pH-value (e.g. spacer comprises a hydrazone moiety for acidic degradation), by action of an enzyme (spacer comprises a peptide as a target site for enzymatically degradation (e.g. hydrolysis)), by action of reducing agents (e.g. spacer comprises a disulfide-moiety for degradation by glutathione and DTT), by action of oxidizing agents (e.g. spacer comprises vicinal Diols for degradation by periodate oxidation), by action of miscellaneous chemical agents (e.g. spacer comprises a thioether moiety for proteolytic degradation), by action of electromagnetic waves (preferably UV) (spacer comprises photocleavable moieties (e.g. nitrobenzyl) for UV degradation).

The second functional group is used for biochemical incorporation of bioactive substances. The first functional group is chosen from: methacrylic acid derivates, diacrylamide derivates, electrophilic monomers without a labile proton from a carboxylic acid group, acrylic acid derivates, methacrylic acid, acrylamide, β-propiolactone and maleimide derivatives, ethylensulfonamide, succinic anhydride and phtalic anhydride, phenyl acrylate. The second functional group is chosen from: esters of protected N-hydroxysuccinimide, esters of unprotected N-hydroxysuccinimide, carboxylic acid hydrazide, sulfo-N-hydroxysuccinimide ester, anhydride of carboxylic acid, vinyl sulfone, sulfonyl chloride, aldehyde, epoxide, thiol, maleimide and carbonate.

Said heterobifunctional reagents are preferably represented by compounds of formula

R₁-k-R₂

wherein
R₁ is a first functional group for the copolymerization with said heterocyclic chemical compound, preferable said CIE,
R₂ is a moiety, comprising at least one second functional group, independently selected from a functional group
   for crosslinking and/or
   for binding biologically active compounds, and
k is a direct bond or preferably a spacer moiety (most preferably a degradable spacer moiety).

In order to prevent participation of the functional group for crosslinking and/or for binding biologically compounds, it may be useful to protect said second functional group by introducing a protecting group for this second functional group into said heterobifunctional reagent. After polymerization the protecting group is cleaved from the polymer backbone. The introduction and cleavage of protecting groups as a strategy to prevent functional groups of a compound from reaction during organic synthesis is a method well known to the skilled artisan.

Following reactive functional groups commonly interfere during SZWIP:: Aldehydes, Alcohols, Ketones, Amines and Carboxylic acids and Thiols. These reactive functional groups can be incorporated by using common protecting groups (see below FIG. 41).

The reactive functional groups are protected during SZWIP by common protecting groups. For alcohols, aldehydes and ketones ether-protecting groups are used which can be divided into subcategories: Silyl ether protecting groups, acetal protecting groups, ketal protecting groups and alkyl ether protecting groups. Examples are: trimethylsilyl, triethylsilyl, tert-butyldimethylsilyl (TBDMS), tert-butyldiphenylsilyl (TBDPS), benzyl ether, phenylether.

For carboxylic acids common protecting groups are used especially esther-protecting groups such as ethyl, methyl, t-butyl, benzyl and phenyl-protecting groups.

For amines common protecting groups are used especially carbamate protecting groups such as Di-tert-butyloxycarbonyl (Boc), Fluorenylmethyl carbonyl (Fmoc) and Benzyloxycarbonyl protecting groups (CBZ).

For thiols common protecting groups are used such as chloromethyl methyl ether (MOM-C1) or acid-catalyzed reaction with dimethoxymethane.

In an advantages embodiment of this disclosure, N-Hydroxysuccinimid-ester (NHS-ester), epoxides and hydrazides are directly used as a second functional group during SZWIP (see FIG. 42). These functional reactive groups can be robust against attacks of carbenes and anionic maleimides, respectively.)

In an advantageous embodiment of this disclosure the spontaneous zwitterionic copolymerization (SZWIP) between CIEs and heterobifunctional monomer systems is initiated by macromolecules comprising at least two CIEs, preferably more than two marginal 2-oxazoline moieties. The SZWIP takes simultaneously place on each arm.

In an advantageous embodiment of this disclosure the spontaneous zwitterionic copolymerization (SZWIP) between CIEs and heterobifunctional monomer systems is terminated by an excess of (meth)acrylic acid. The molar excess is achieved either by purification of the zwitterionic macromolecules and subsequently addition of (meth)acrylic acid or by direct addition of (meth)acrylic acid at the end of the SZWIP. The termination by (meth)acrylic acid leads to α-acrylate and ω-carboxylic acid end groups resulting in heterotelechelic copolymers. In general nucleophiles and electrophiles can be used to terminate the SZWIP (FIG. 51).

The LCROP is usually initiated by an initiator and oxazoline monomers by heating to 75° C. in acetonitrile or by microwave technology. The living polymer is terminated by addition of a terminator. One advantages of the CROP of 2-oxazolines in terms of synthesis are the high degree of polymerization control, the resulting well-defined polymeric structures and the large variety of end- and side-group functionalities, which can be introduced using appropriate initiators/terminating agents and substituted monomers, respectively. The modularity of this polymer class enables the synthesis of highly functional materials with tailormade properties. Scheme 14 illustrates the mechanism of the polymerization and the incorporation of functional molecules for cell culture and cell analysis. In total four classes of molecules are needed for the CROP: Initiators for initiation of the reaction preferably with an electrophilic character, heterocyclic chemical compounds as monomers for the polymer backbone, unsaturated imides and/or alkenyl groups for functionalization of the polymer backbone and terminating agents for terminating the living polymer.

The initiators used for the CROP to produce polymers for the fabrication of said array consist of an organic moiety with an attached leaving group, which acts as the counter ion for the oxazolinium species during polymerization. The initiators used are chosen from a group of different tosylates, triflates or alkyl halides of small aliphatic molecules or small PEGs. Most preferably bifunctional initiators such as triethylene glycol di(p)-toluenesulfonate are used for the synthesis of linear polymers. In this case both sides of the living polymer can be terminated by the same species of terminating molecules leading to homo-bifunctional linear polymers. Alternatively, the nature of the initiator can be altered to synthesize hetero-bifunctional linear polymers with a functional group F1 incorporated by the initiator and a functional group F2 incorporated by the terminating molecule. The terminating molecules are chosen from a group of nucleophiles, amines, azides or acids especially carboxylic acids. The functional groups F1 and F2 are suitable for cell-compatible crosslinking reactions (Table 1). Combining these different synthesis strategies for linear polymers lead to a variety of possible structures. For the multiarm polymers initiators used are chosen from a group of different multi-tosylates, -triflates or -alkyl halides of small aliphatic molecules or small PEGs. Most preferably multifunctional initiators such as pentaerythritol tetrabromide, pentaerythritol tetrakis(benzenesulfonate) or p-toluenesulfonyl chloride modified N,N,N′,N′-Tetrakis(2-hydroxyethyl)ethylenediamine are used for the synthesis of multiarm polymers.

In an advantageous embodiment of this invention the heterotelechelic copolymers can be coupled to multi-arm substances with compatible functional end groups leading to end-functionalized multi-arm copolymers. These multi-arm copolymers can be used to form hydrogels in combination with linear copolymers.

In an advantageous embodiment of this invention PNA sequences can be coupled to the ends of the linear and multi-arm copolymer. The coupling can be done by direct incorporation of PNAs or by coupling of PNA molecules to the heterotelechelic copolymers. For the direct incorporation of PNA molecules, PNA molecules with attached nucleophilic moieties are added to the SZWIP or CROP, respectively. The nucleophile of the PNA molecule leads to a termination of both polymerization types and ω-PNA end groups. In an alternative embodiment zwitterionic macromolecules and living polymers are purified in a first step and terminated by addition of different PNA molecules with either electrophilic moieties or nucleophilic moieties or a combination of these PNA molecules. For the incorporation of PNA into heterotelechelic copolymers, the α- and ω-end groups of the heterotelechelic copolymers react with corresponding marginal functional groups of PNA molecules. In a preferred embodiment the marginal functional group of the PNA is a primary amine or thiol.

In an advantageous embodiment of this disclosure the heterotelechelic copolymers are used as precursor components for further polymerization reactions especially for living free radical polymerizations. During living free radical polymerizations α-acrylate groups of the copolymer are further polymerized resulting in a (meth)acrylate polymer chain which is functionalized by the copolymer.

A preferred polymer, especially a polymer as building-block for hydrogel formation, comprises at least one moiety of formula (I) and at least one moiety of formula (II)

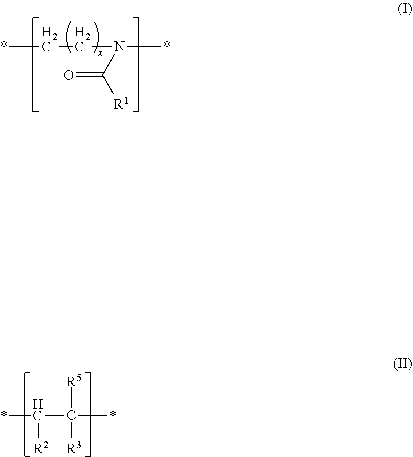

wherein
R¹ is a hydrogen atom, a hydrocarbon with 1-18 carbonatoms (preferably CH₃, —C₂H₅), a C₁-C₂₅-hydrocarbon with at least one hydroxy group, a C₁-C₂₅-hydrocarbon with at least one carboxy group, (C₂-C₆)alkylthiol, (C₂-C₆)alkylamine, protected (C₂-C₆)alkylamine (preferably-(CH₂)₂₋₆—NH—CO—R (with R=benzylhydryloxy, 9-fluorenylmethoxy)), (C₂-C₆)alkylazide, polyethylene glycol, a crosslink to R¹ of another moiety of formula (I), polylactic acid, polyglycolic acid or polyoxazoline, or wherein R₁ is a residue R⁴,
R² and R³ R² and R³ are linked to form a cyclic moiety of formula (II) comprising at least one residue R⁴
   or R² and R³ are independently selected from hydrogen, —COOH, methyl or a residue R⁴, wherein optionally, at least one of R2 and R3 is a residue R4,
R⁴ is a moiety, comprising at least one functional group, independently selected from a functional group
   for crosslinking and/or
   for binding biologically active compounds, and
   optionally comprising a (preferably degradable) spacer moiety connecting said functional group with the binding site of the respective moiety of formula (I) or formula (II), and
R⁵ denotes a hydrogen atom, a carboxymethyl group or a methyl group,
x is 1, 2 or 3, and
* denotes a chemical bond of the polymer backbone or to a terminating moiety,
with the proviso, that at least one moiety of formula (I) or formula (II) comprises a residue R⁴, wherein preferably only the moieties of formula (I) or only the moieties of formula (II) comprise at least one moiety R⁴.

If according to this invention more than one moiety or residue is defined as explicitly being “independently” selected or chosen from members of the same list, said moieties or residues are selected independently from one of another.

Additionally, if one and the same moiety or residue appears more than once in a structure and is defined as explicitly being “independently” selected or chosen from members of a list, said moieties or residues are selected independently for each position within the structure.

Additionally, if a structure is a repeating unit of a polymer and a moiety or residue of said structure is defined as being selected or chosen from members of a list, said moiety or residue is selected independently, i.e. for every single repeated structure.

A “terminating moiety” is defined as being a monovalent terminus-unit of a polymer, which functions as an “end-cap” of a polymeric backbone or a polyvalent group (preferably with 2 to 10 valence sites), which may function as a linker for at least two polymer chains (preferably as a core or branching point of the polymer (e.g. of a dendritic polymer or a star-shaped polymer).

Preferred above mentioned polymers, especially polymers as building-block for hydrogel formation, comprise moiety of formula (I), wherein at least one R¹ is a hydrogen atom or a C₁-C₁₈-alkyl group, preferably a hydrogen atom, methyl, ethyl, n-propyl, iso-propyl, n-butyl, iso-butyl, sec-butyl, tert-butyl, pentyl, iso-pentyl, neopentyl, sec-pentyl, hexyl, heptyl, octyl, nonyl or decyl, more preferably methyl or ethyl. This embodiment is particularly preferred for said polymers, comprising at least one moiety of formula R⁴ within a structure according to formula (II).

It is a general teaching of this invention, that the hydrophilicity of the polymers according to this invention, is tunable by using a combination of different residues R¹ within the polymer structure. For this reason it is particularly preferred, if said polymer comprises at least two different moieties of formula (I) having different groups R¹.

According to one embodiment, R₁ is a hydrogen or 1-18 carbonatoms (preferably CH3, —C2H5), if formula (II) comprises a residue R₄. According to one embodiment, R₁ is a residue R₄, if formula (II) does not comprises a residue R₄.

The residue R¹ also may denote a crosslink to R¹ of another moiety of formula (I). This crosslink results from polymerization of bifunctionalized CIE-compounds, preferably an α, ω-bis(1,3-oxazolidine-2-yl)-C2-C8-alkane.

In one embodiment, a polymer, especially polymer as building-block for hydrogel formation, is characterized in that R1 is a hydrogen atom or a hydrocarbon with 1-18 carbon atoms, preferably for adjusting chemical characteristics of the polymer. Furthermore, R2 and R3 are linked to form a cyclic moiety of formula (II) comprising at least one N-hydroxysuccinimide ester for binding biologically active compounds or R2 and R3 are independently selected from hydrogen, —COOH, methyl or at least N-hydroxsuccinimide bearing molecule for binding biologically active compounds. R5 denotes a hydrogen atom, a carboxymethyl group or a methyl group and x is 1. Moreover, * denotes a chemical bond of the polymer backbone or to a terminating moiety wherein the terminating moiety comprises a PNA sequence.

The moiety of formula (I) results from the polymerization of a corresponding oxazoline-derivative, oxazine-derivative or azepine-derivative respectively. It was found, that preferred polymers, especially polymers as building-block for hydrogel formation, are, characterized in, that according to formula (I) x is 1 or 2, preferably x is 1.

The moiety of formula (II) results from the polymerization of a corresponding unsaturated moiety. Preferred polymers, especially polymers as building-block for hydrogel formation, are characterized in, that the moiety of the formula (II) is derived from at least one monomer selected from an unsaturated imide (preferably derived from maleimide), an alkene, an acrylic acid, an itaconic acid, a lactone (preferably β-propiolactone, α-methyl-β-propiolactone, α,α-dimethyl β-propiolactone, β-butyrolactone), an acrylamide, a sulfonamide (preferably ethylensulfonamide), an anhydride, a methacrylic acid, an acrylamide, a methacrylamide, a N,N-diacrylamide (preferably N-methyldiacrylamide), a 1-propanesulfonic acid sultone,

with the proviso, that said monomer comprise said residue R⁴ respectively.

Particularly preferred polymers, especially polymers as building-block for hydrogel formation, comprise at least one moiety of formula (II), selected from a moiety of formula (II-a)

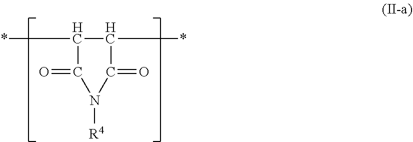

wherein
R⁴ is a moiety comprising at least one functional group
   for crosslinking and/or
   for binding biologically active compounds,
   and optionally comprising a (preferably degradable) spacer moiety connecting said functional group with the binding site of R⁴ according to formula (II-a),
and * denotes a chemical bond of the polymer backbone or to a terminating moiety.

It is preferred, when said polymer, especially polymer as building-block for hydrogel formation, comprises at least one moiety of formula (II), selected from a moiety of formula (II-b)

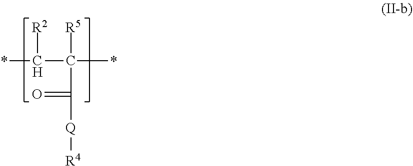

wherein
R⁵ and R⁴ is defined according to any of the preceding claims,
R² is a hydrogen atom or a carboxyl group,
Q denotes an oxygen atom or an imino group NH,
and * denotes a chemical bond of the polymer backbone or to a terminating moiety.

For the formation of a hydrogel according to this invention, it is crucial, that a polymer used as a building-block for said hydrogel comprises at least one residue R⁴ comprising a functional group, independently selected from a functional group
for crosslinking (preferred members vide supra) and/or
for binding biologically active compounds (preferred members vide supra).

Preferred polymers are characterized in, that R⁴ according to formula (I) and (II) is independently a moiety, comprising at least one functional group independently selected from arene, amine, alkyne, azide, anhydride, acid anhydride, ketone, haloalkane, imidoester, diol, hemiacetal, acrylate, alkene, thiol, ether, ester, isocyanate, isothiocyanate, succinimide, N-hydroxysuccinimide, sulfo-N-hydroxysuccinimide, amide, maleimide, N-heterocyclic carbene, acyl halide, N-heterocyclic phosphine, hydrazide, nitrile, aminoxy, imidazolide, imine, aldehyde, azo compound, imide, carbodiimide, haloacetyl, pyridyl disulfide, carboxamide, vinyl ether, carboxyl, carboxylate, phenyl, phenol, indol, methylthiol, pyridyldithiol, hydroxyl, epoxide, carbonyl, methoxycarbonyl, glycidyl, carboxyphenyl (particularly preferred selected from polymer, especially polymer as building-block for hydrogel formation, characterized in, that said functional group of residue R⁴ is independently selected from the group consisting of protected N-hydroxysuccinimide-esters, unprotected N-hydroxysuccinimide-esters, sulfo-N-hydroxysuccinimide esters, vinyl sulfone, sulfonyl chloride, aldehyde, epoxides, thiol, maleimide and carbonate, wherein preferably, the moiety of formula (II) comprises such residue R⁴).

The moiety R⁴ preferably comprises a spacer moiety, connecting said functional group with the binding site of R⁴ to the respective structural unit of said polymer, especially of said moieties according to formula (I) and formula (II).

Said spacer is particularly preferred a degradable spacer, most preferred degradable by change of the pH-value (e.g. spacer comprises a hydrazone moiety for acidic degradation), by action of an enzyme (spacer comprises a peptide as a target site for enzymatically degradation (e.g. hydrolysis)), by action of reducing agents (e.g. spacer comprises a disulfide-moiety for degradation by glutathione and DTT), by action of oxidizing agents (e.g. spacer comprises vicinal Diols for degradation by periodate oxidation), by action of miscellaneous chemical agents (e.g. spacer comprises a thioether moiety for proteolytic degradation), by action of electromagnetic waves (preferably UV) (spacer comprises photocleavable moieties (e.g. Nitrobenzyl) for UV degradation).

Preferred degradable spacer comprise an enzyme degradable target site, most preferably selected from ester linkages (esterases or lipase (hydrolysis of esters)), polyhxydroxyalkanoat-moieties (PHA depolymerases (hydrolysis of polyhydroxyalkanoate)) or peptides (proteases (hydrolysis of peptides e.g. MMP)).

It is particularly preferred that the moiety of the formula (II) is derived from monomers selected from 3-(maleimido)-propionic acid N-hydroxysuccinimide ester, 6-maleimidohexanoic acid N-hydroxysuccinimide ester, N-(Methacryloxy)-succinimideisopropenyl, BMPH (N-(β-maleimidopropionic acid)-hydrazide, EMCH (N-ε-maleimidocaproic acid hydrazide), PDPH (3-(2-pyridyldithio)propionyl hydrazide), methacrylic acid N-hydroxysuccinimide ester, N-methoxycarbonyl maleimide, acrylic acid N-hydroxysuccinimide ester, a PNA-amide of acrylic acid, a PNA-amide of methacrylic acid, a PNA-amide of acrylamide, a PNA-amide of methacrylamide, a monomer of formula

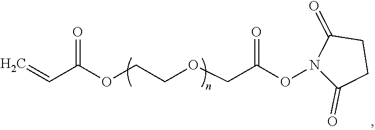

wherein n is an integer of at least 1,
a monomer of formula

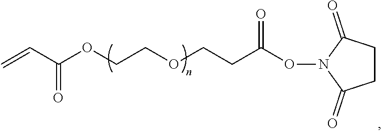

wherein n is an integer of at least 1,
a monomer of formula,

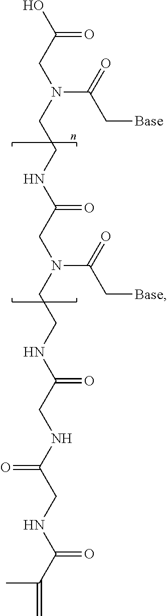

wherein n is an integer greater than 1 and Base is independently
a moiety comprising at least one nucleobase,
   or mixtures thereof.

PNA-functionalized derivatives of (meth)acrylic acid or (meth)acrylamide can be used in SZWIP-polymerization of as terminating agents according to the following general procedure:

In a dried Schlenk flask equipped with a magnetic stirrer bar, MEHQ (1 mg, 8.06×10−6 mol) was dissolved in MeCN. The CIE was subsequently added under nitrogen, followed by the addition of PNA-functionalizes (meth)acrylic acid. The mixture was placed in an oil bath (70° C.) for 24 h. Subsequently, the polymer solution was cooled down to room temperature, precipitated in Et₂O and isolated by centrifugation. The purification method was repeated two more times. To remove the Et₂O, the polymer was placed under vacuum.

Said PNA-derivatives of (meth)acrylic acid can be prepared according to the literature procedure of Chu T W et al, J Control Release., 2015, Dec. 28; 220 (Pt B), pages 608-16 (doi: 10.1016/j.jconrel.2015.09.035) incorporated herein by reference.

At least one polymer, especially polymer as building-block for hydrogel formation, comprising at least one (m is an integer of at least 1) unit having the structure of formula (III)

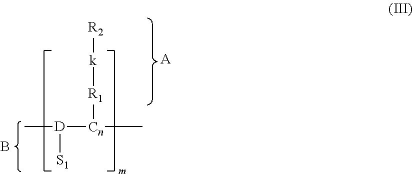

R₂ is independently a residue R⁴, comprising at least one functional group
   for crosslinking and/or
   for binding biologically active compounds,
S₁ is independently defined according to R¹ of above mentioned formula (I),
fragment D-Cn is part of the polymer backbone,
wherein said structure results from polymerization of a heterocyclic molecule B in presence of a first component A, was identified as a preferred polymer according to this invention.

The unit of formula (III) comprises a moiety D, which comprises a covalent substitution. Therefor said unit of formula (III) is a covalently functionalized D-substituted alkylamine.

The fragment D-Cn of formula (III) results from coupling of a heterocyclic molecule B with a first component A via e.g. a polymerization reaction (see FIG. 15).

Preferred moieties S₁ of formula (III) are the previously mentioned preferred embodiments of R¹ of formula (I).

Preferred moieties R⁴ or formula (III) are the previously mentioned preferred embodiments.

A preferred polymer comprising said unit of formula (III), is characterized in, that said first component A is a compound of formula (IV)

R₁-k-R₂ (IV)

wherein

R₁ is a first functional group for the copolymerization with said heterocyclic molecule B,

R₂ is said moiety R⁴,

k is a direct bond or a spacer moiety.

Particularly preferred moieties k of formulas (III) and (IV) are independently selected from a direct bond, alkylidene groups with 2 to 8 carbon atoms, hydrocarbons, and/or a degradable spacer (preferably selected from previously defined preferred spacer moieties, most preferable from peptides, PNA, polyethylene glycol).

According to a preferred embodiment of the polymer, said first component A of formula (IV) is selected from the monomers as defined to derive a structure of formula (II).

A preferred polymer comprising said unit of formula (III), is characterized in, that said heterocyclic molecule B is a 2-substituted heterocyclic compound of formula (V)

D-S₁ (V)

wherein

D is an oxazoline-moiety, oxazine-moiety or oxyazepine-moiety and

S₁ is a substituent in 2-position as defined as R¹ according to formula (I).

Particularly preferred polymers, especially for use as building-block for hydrogel formation, is a polymer of formula (P1)

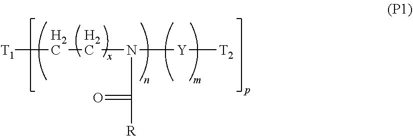

wherein
R is independently selected from a hydrogen atom, a hydrocarbon with 1-18 carbonatoms (preferably CH₃, —C₂H₅), a C₁-C₂₅-hydrocarbon with at least one hydroxy group, a C₁-C₂₅-hydrocarbon with at least one carboxy group, (C₂-C₆)alkylthiol, (C₂-C₆)alkylamine, protected (C₂-C₆)alkylamine (preferably-(CH₂)₂₋₆—NH—CO—R (with R=tert-Butyl, perfluoroalkyl)), (C₂-C₆)alkylazide, polyethylene glycol, polylactic acid, polyglycolic acid, polyoxazoline, or wherein R is a residue R⁴
Y is a moiety containing at least one graft, comprising at least one residue R⁴,
T₁ is a terminating moiety, which may contain a residue R⁴,
T₂ is a terminating moiety, which contains a residue R⁴,
p is an integer from 1 to 10,
n is an integer greater than 1 and preferably, below 500,
m is zero or an integer of at least, preferably greater than 1, and preferably, below 500, the sum n+m is greater than 10,
x is independently 1, 2 or 3, preferably x is independently 1 or 2, most preferably x is 1,
R⁴ independently comprise at least one functional group
   for crosslinking and/or
   for binding biologically active compounds, and
   optionally comprising a (preferably degradable) spacer moiety connecting said functional group with the binding site to the respective moiety of the structure of formula (P1),
   wherein the entirety of all m-fold and n-fold repeating units are distributed in any order within the polymer chain and wherein optionally, the polymer is a random copolymer or a block copolymer.

The entirety of the m-fold and n-fold repeating units of formula (P1) represent a polymer chain.

The distribution of said repeating units within said polymer chain occurs in any possible arrangement of said repeating units within said polymer chain. If at least two distinguishable repeating units are present within said polymer chain (for example the polymer comprises units with different substituents R or m is different from zero), the polymer may be a random copolymer or a block copolymer.

In the event that m is an integer greater than 1, an alternating order of repeating units is particularly preferred, wherein one repeating unit, chosen from the portion of n-fold repeating units is directly connected to a unit, chosen from the portion of m-fold repeating units. Said alternating arrangement leads to a particularly preferred embodiment of the polymer of formula (P1) according to formula (P1-1)

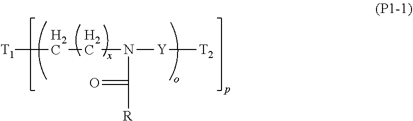

wherein o is an integer of greater than 1 and

T¹, T², x, R and Y is defined according to formula (P1).

The polymer according to formula (P1) and (P1-1) comprises an amount of p (one to ten) of said polymer chains. According to the structure of formula (P1) or (P1-1), T¹ is clearly defined as a terminating moiety, which functions dependent on the value of p either as a terminus-unit (end-cap) for p=1, or a core or branching point moiety (p=2 to 10), connecting an amount of p polymer chains. According to formula (P1), T² is clearly defined as a terminus residue (end-cap).

In a preferred embodiment of the polymer according to formula (P1) and (P1-1), Y is a moiety of formula (II) as defined above (vide supra).

Preferred polymers of formula (P1) are characterized in, that R is a hydrogen atom or a C₁-C₁₈-alkyl group, (preferably a hydrogen atom, methyl, ethyl, n-propyl, iso-propyl, n-butyl, iso-butyl, sec-butyl, tert-butyl, pentyl, iso-pentyl, neopentyl, sec-pentyl, hexyl, heptyl, octyl, nonyl, decyl) and m is an integer greater than 1.

In another preferred embodiment, a polymer, especially polymer as building-block for hydrogel formation is, characterized in, that R is a hydrogen atom, a hydrocarbon with 1-18 carbonatoms (preferably CH3, —C2H5); Y is a moiety containing at least one graft, comprising at least one degradable spacer moiety connecting at least one N-hydroxysuccinimide ester for binding biologically active compounds to the respective moiety of the structure of formula (P1); T1 is a terminating moiety, optionally comprising a peptide nucleic acid (PNA) sequence; T2 is a terminating moiety, optionally comprising a peptide nucleic acid (PNA) sequence; n is an integer greater than 1; m is an integer greater than 1; the sum n+m is greater than 10 and less than 500; and x is 1; wherein the entirety of all m-fold and n-fold repeating units are distributed in any order within the polymer chain and wherein optionally, the polymer is a random copolymer or a block copolymer.

A particularly preferred first embodiment of polymers according to formula (P1) and (P1-1) are characterized in, that
T₁ is a terminating moiety, comprising a first XNA-residue (XNA1) and optionally an EDTS-moiety,
T₂ is a terminating moiety, comprising a second XNA-residue (XNA2) and optionally an EDTS-moiety,
p equals 1 or 2, preferably equals 1,
EDTS is an enzyme degradable target site, preferably a matrix metalloprotease (MMP) target site, for site directed degradation of the polymer,
XNA is a nucleic acid or nucleic acid analog, preferably a peptide nucleic acid (PNA) sequence.

The rest of the parameters according to formula (P1) or (P1-1) are defined as mentioned above (vide supra). The polymer of this first embodiment is a linear polymer.

The preparation of said polymer is possible via spontaneous zwitterionic copolymerization of a corresponding CIE with a heterobifunctional reagent according to the general method as described above. Said preparation method may comprise the steps of
1. Copolymerisation of CIE and heterobifunctional reagent
2. workup of the resulting zwitterionic copolymer,
3. reaction of said zwitterionic copolymer of step 2 with (meth)acrylic acid,
4. coupling of a compound comprising a PNA1 sequence via its terminal amino group with the terminal carboxyl group of said zwitterionic copolymer
5. coupling of a second compound comprising the same or a different PNA2 sequence with the unsaturated moiety of the terminal ester of (meth)acrylic acid of the polymer resulting from step 3 via a primary amino group or a thiol group of the PNA moiety.

Said preparation is illustrated in the following structure:

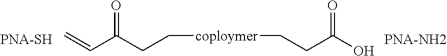

A preferred polymer of said first embodiment, is characterized in, that m is zero and no moiety Y is comprised in the polymer.

A particularly preferred second embodiment of polymers according to formula (P1) and (P1-1) are characterized in, that
T₁ is a terminating moiety, comprising no residue R⁴,
T₂ is a terminating moiety, comprising a XNA-residue, optionally linked to an EDTS-moiety,
p is an integer of 3 to 10, preferably 3 to 10, preferably 3 to 8, most preferred 3 to 6,
EDTS is an enzyme degradable target site, preferably a matrix metalloprotease (MMP) target site, for site directed degradation of the polymer,
XNA is a nucleic acid or nucleic acid analog, preferably a peptide nucleic acid (PNA) sequence.

The rest of the parameters according to formula (P1) or (P1-1) are defined as mentioned above (vide supra). The polymer of this second embodiment is a star-shaped polymer.

A preferred polymer of said second embodiment is characterized in, that m is zero and no moiety Y is comprised in the polymer.

A particularly preferred third embodiment of polymers according to formula (P1) and (P1-1) are characterized in, that,
T₁ is a terminating moiety, comprising a residue R⁴ different from a XNA-residue, wherein R⁴ is optionally linked to a EDTS-moiety,
T₂ is a terminating moiety, comprising a residue R⁴ different from a XNA-residue, wherein R⁴ is optionally linked to an EDTS-moiety,
p equals 1 or 2, preferably equals 1,
EDTS is an enzyme degradable target site, preferably a matrix metalloprotease (MMP) target site, for site directed degradation of the polymer,
XNA is a nucleic acid or nucleic acid analog, preferably a peptide nucleic acid (PNA) sequence.

The rest of the parameters according to formula (P1) or (P1-1) are defined as mentioned above (vide supra). The polymer of this third embodiment is a linear polymer.

A preferred polymer of said third embodiment is characterized in, that m is zero and no moiety Y is comprised in the polymer.

A particularly preferred fourth embodiment of polymers according to formula (P1) and (P1-1) are characterized in, that
T₁ is a terminating moiety, comprising no residue R⁴,
T₂ is a terminating moiety, comprising a residue R⁴ different from a XNA-residue, wherein R⁴ is optionally linked to an EDTS-moiety,
p is an integer of 3 to 10, preferably 3 to 10, preferably 3 to 8, most preferred 3 to 6,
EDTS is an enzyme degradable target site, preferably a matrix metalloprotease (MMP) target site, for site directed degradation of the polymer,
XNA is a nucleic acid or nucleic acid analog, preferably a peptide nucleic acid (PNA) sequence.

The rest of the parameters according to formula (P1) or (P1-1) are defined as mentioned above (vide supra). The polymer of this second embodiment is a star-shaped polymer.

A preferred polymer of said fourth embodiment is characterized in, that m is zero and no moiety Y is comprised in the polymer.

A preferred polymer according to formula (P1), (P1-1) and their four preferred embodiments are characterized in, that it is a polymer which comprises an EDTS-moiety, preferably a MMP-moiety.

A preferred polymer according to formula (P1), (P1-1) and according to their four preferred embodiments is characterized in, that it comprises at least two different moieties R.

A preferred polymer according to formula (P1), (P1-1) and according to their four preferred embodiments is characterized in, that p is an integer of 3 to 10, preferably 3 to 10, preferably 3 to 8, most preferred 3 to 6.

Another preferred polymer of this invention, especially polymer as a building-block for hydrogel formation, is a polymer of formula (P2)

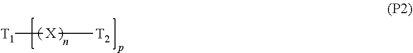

wherein
T₁ is a terminating moiety, which contains a residue -XDTS-XNA1,
T₂ is a terminating moiety, which contains a residue -XDTS-XNA2,
XDTS is independently selected from a direct bond or an EDTS-moiety, wherein EDTS is an enzyme degradable target site, preferably a matrix metalloprotease (MMP) target site, for site directed degradation of the polymer,
XNA1 is a nucleic acid or nucleic acid analog, preferably a peptide nucleic acid (PNA) sequence,
XNA2 is the same or a different nucleic acid or nucleic acid analog compared to XNA1, preferably a peptide nucleic acid (PNA) sequence,
p is 1 or 2, preferably 1,
X is a hydrophilic polymeric residue, preferably independently derived from monomers independently selected from oxazoline, ethylene glycol, propylene glycol, acetal lactic acid, glycolic acid, vinyl alcohol,
n is an integer greater than 1, preferably from 1 to 10000,
according to one embodiment at least one X is different from oxazoline.

A preferred embodiment of the polymer according to formula (P2) is characterized in that
T₁ is a terminating moiety, comprising no XNA-residue,
T₂ is a terminating moiety, comprising a XNA-residue and optionally a EDTS-moiety,
p is an integer of 3 to 10, preferably 3 to 8, most preferred 3 to 6,
X hydrophilic polymeric residue, preferably independently derived from monomers independently selected from oxazoline, ethylene glycol, propylene glycol, acetal lactic acid, glycolic acid, vinyl alcohol,
EDTS is an enzyme degradable target site, preferably a matrix metalloprotease (MMP) target site, for site directed degradation of the polymer,
XNA is a nucleic acid or nucleic acid analog, preferably a peptide nucleic acid (PNA) sequence,
n is an integer greater than 1, preferably from 1 to 10000,

A preferred embodiment of all mentioned polymer according to this invention, especially polymer as building-block for hydrogel formation, are characterized in, that the polymer is functionalized by at least one biologically active compound, preferably, at least two different biologically active compounds, preferably by reaction of an amino group of the biologically active compound with a functional group of residue R⁴.

A preferred embodiment of all mentioned polymers according to this invention, is characterized in, that the biologically active compound selected from the group consisting of peptides, proteins, CRISPR-Cas enzyme complex, apoptosis-inducing active substances, adhesion-promoting active substances, anti-inflammatory active substances, receptor agonists and receptor antagonists, growth-inhibiting active substances (and in particular from proteins of the extracellular matrix, cell surface proteins, antibodies, growth factors, sugars, lectins, carbohydrates, cytokines, DNA, RNA, siRNA), aptamers, and fragments thereof, or mixtures thereof.

A preferred embodiment of all mentioned polymers according to this invention, is characterized in, that the polymer comprises at least one biologically active compound selected from the group consisting of peptides, proteins, CRISPR-Cas enzyme complex, apoptosis-inducing active substances, adhesion-promoting active substances, anti-inflammatory active substances, receptor agonists and receptor antagonists, growth-inhibiting active substances (and in particular from proteins of the extracellular matrix, cell surface proteins, antibodies, growth factors, sugars, lectins, carbohydrates, cytokines, DNA, RNA, PNA, LNA, siRNA), aptamers, and fragments thereof, or mixtures thereof.

A preferred embodiment of all mentioned polymers according to this invention, is characterized in, that the polymer comprises at least one biologically active compound selected from a Peptide nucleic acid (PNA) and/or a locked nucleic acid (LNA), preferably wherein the PNA-moiety independently comprise a structure of formula (VI)

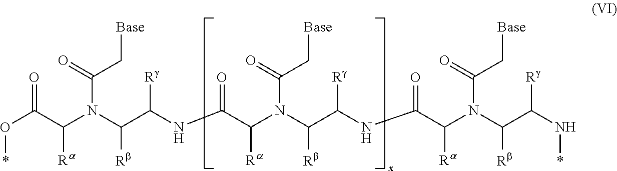

wherein

x is an integer greater than 1,

Base is independently a moiety comprising at least one nucleobase (preferably selected from adenin, cytosin, guanine, thymine, 2,6-diaminopurine, analogs of thymine and cytosine, hypoxanthine, derivatives thereof functionalized with a fluorescent dye (preferably thiazole orange)),

Rα and Rβ are independently selected from hydrogen atom, any residue bound to the alpha-carbon atom of any of the proteinogenic amino acid,

Rγ is a hydrogen atom, a moiety with at least one ionic residue.

The synthesis of PNA-molecules is well known in the art using the well known Bhoc strategy. PNAs are highly tolerant to modifications at their α-, β- or γ-positions. In particular modifications at the γ-position improve functionality of PNAs and the properties of the PNA, especially in terms of hydrophilicity.

Modification of PNAs from iterative Ugi couplings allow modular modifications at the α, β and γ position of the PNA backbone as described in Bioorganic & Medicinal Chemistry, 2017, Volume 25, Issue 19, pages 5171-5177, being fully incorporated by reference.

Preferred polymers of this invention are characterized in, that they comprises at least one biologically active compound, selected from a Peptide nucleic acid (PNA) comprising a matrix metalloprotease target site for the site directed degradation (MMP). It is further preferred, that said polymer, is characterized in, that is comprises at least one additional biologically active compound, selected from the group consisting of peptides, proteins, CRISPR-Cas enzyme complex, apoptosis-inducing active substances, adhesion-promoting active substances, anti-inflammatory active substances, receptor agonists and receptor antagonists, growth-inhibiting active substances (and in particular from proteins of the extracellular matrix, cell surface proteins, antibodies, growth factors, sugars, lectins, carbohydrates, cytokines, DNA, RNA, siRNA), aptamers, and fragments thereof, or mixtures thereof.

Preferred polymers of this invention are characterized in, that the polymer has a linear structure (preferably a graft polymer, grafted with at least one residue R⁴) or a dendritic structure (preferably a linear structure or a star shaped structure).

Preferred polymers of this invention are characterized in, that the polymer is a random polymer, a block-copolymer or a dendrimer. It is furthermore particularly preferred, that the polymer according to this invention has a star-shaped structure comprising at least three arms.

The polymers according to this invention, especially the preferred polymers as mentioned and defined above, are prepared by at least one polymerization step, selected from living cationic ring-opening polymerization (CROP), spontaneous zwitterionic copolymerization (SZWIP) or a combination of both. A detailed description for carrying out said polymerization reaction was already given (vide supra).

Preferred polymers of this invention are characterized in, that the polymerization, preferably the living cationic ring-opening polymerization, is initiated by an initiator with an electrophilic character. Preferably polymers according to formula (P1) or (P1-1) wherein m equals zero are prepared by using living cationic ring-opening polymerization.

Preferably polymers according to formula (P1) or (P1-1) wherein p is an integer from 2 to 10 are prepared by using living cationic ring-opening polymerization, initiated by initiators with more than one site for initiation of the reaction. Preferred polymers of this invention, are characterized in, that the initiator for polymerization, especially for living cationic ring-opening polymerization, is selected from triethylene glycol di (p)-toluenesulfonate, pentaerythritol tetrabromide, pentaerythritol tetrakis(benzenesulfonate) or p-toluenesulfonyl chloride modified N,N,N′,N′-Tetrakis(2-hydroxyethyl)ethylenediamine.

Preferred polymers of this invention are characterized in, that the polymerization, preferably the living cationic ring-opening polymerization, is terminated by addition of a terminating molecule selected from nucleophiles, amines, azides or acids (preferably carboxylic acids). Polymer, especially polymer as building-block for hydrogel formation, according to any of any of claims 45 to 48, characterized in, that the polymerization, preferably the living cationic ring-opening polymerization, is terminated by addition of a terminating molecule selected from peptide nucleic acid (PNA), preferably peptide nucleic acid (PNA) with unprotected carboxylic acid group at the C-terminus and protected amino group at the N-terminus or peptide nucleic acid (PNA) with unprotected amino group at the N-terminus and protected carboxylic acid group at the C-terminus).

Suitable protective groups for amino groups or for carboxyl groups of PNA are already mentioned above for SWIP-polymerization (vide supra) and are particularly selected from benzylhydryloxycarbonyl (Bhoc), 9-fluorenylmethoxycarbonyl for the protection of amino groups. For the protection of carboxylic acid groups, the protective groups are selected from tert-butoxy, methoxy, ethoxy, n-butoxy, allyloxy, benzyloxy, forming carboxylic acid esters.

Preferred polymers of the invention, especially the preferred polymers as mentioned and defined above, are characterized in, that the polymerization, preferably the spontaneous zwitterionic copolymerization, is terminated by addition of a terminating molecule selected from electrophiles, preferably selected from α,β-unsaturated carboxylic acids, α,β-unsaturated carboxylic acidamides, mixtures thereof, most preferred from acrylic acid, methacrylic acid, acryl amide, methacryl amide, functionalized with at least one residue R⁴ as defined in any of the preceding claims respectively (most preferred functionalized with -MMP-PNA respectively).

Preferred polymers of the invention, especially the preferred polymers as mentioned and defined above, are characterized in, that said initiator and/or said terminating molecule incorporates a moiety R⁴ as defined according to formula (I) and formula (II) (vide supra).

Preferred polymers of the invention, especially the preferred polymers as mentioned and defined above, are characterized in, that the polymerization, preferably the spontaneous zwitterionic copolymerization, is terminated by addition of a terminating molecule selected from selected from α,β-unsaturated carboxylic acids, α,β-unsaturated carboxylic acid amides, mixtures thereof (most preferred from acrylic acid, methacrylic acid, acryl amide, methacryl amide) followed after optional workup by a coupling of a residue comprising PNA and a thiol functionality.

Preferred polymers of the invention, especially the preferred polymers as mentioned and defined above, are characterized in, that a residue comprising PNA and a thiol functionality is coupled to a maleimide as a functional group of residue R⁴. R⁴ is defined according to formula (I) and (II) (vide supra).

Cryopreservation and cell expansion after cryopreservation. A further advantage of said array is that cells located in hydrogel matrices positioned in microfabricated chambers can be cryopreserved and afterwards thawed with a dramatic increase in cell viability and reduction of compounds used for cell expansion. For example, cells might be incorporated into hydrogel matrices acting as cryoprotectant. Afterwards, said cell-laden hydrogel matrices might be positioned in microfabricated chambers and perfused with an aqueous phase containing a soluble cryoprotectant such as glycerol or DMSO. Subsequent freezing of said array would result in an increased cell viability due to the surrounding hydrogel matrix which reduces the formation of ice crystals ensuring cell membrane integrity. Thawing of said array at a later time-point has the advantage, that cells located in said hydrogel matrices might first be expanded and only cells that show viability and proliferation might be removed from the chip for further cell expansion. The small culture volume of the microfabricated chambers subsequently results in a dramatic decrease of the number of compounds (e.g. media) needed for cell expansion.

Time-lapse cytokine profiling of single cells. In another aspect, the present disclosure relates to a method for measuring the number of specific molecules that are secreted by single or multiple cells located in hydrogel matrices immobilized in microfabricated chambers of said array in a time-lapse manner. For example, a hydrogel matrix containing an immune cell that secretes specific cytokines (e.g. TNF-a, IL-10) might be located within a microfabricated chamber at position (n, m). In addition, a second hydrogel matrix containing primary antibodies against specific cytokines is positioned in close proximity to said cell-laden hydrogel matrix at position (n, m). As the microfabricated chamber represent a closed compartment, secreted molecules subsequently diffuse to the adjacent second hydrogel matrix containing primary antibodies.

Thus, analytes are bound by said primary antibodies and collected for a defined period dt. After this period, the immobilized hydrogel matrices are washed by perfusion with an aqueous phase. Afterwards, a mix of barcoded secondary antibodies with different specificities is added to the perfusion phase. The secondary antibodies are labeled with an oligonucleotide (which represents the barcode) that enables the identification of the antibody specificity. The secondary antibodies subsequently bind to second epitope of the analytes that are bound to the primary antibodies located in said hydrogel matrix. After a further washing step, the second hydrogel matrix containing now primary antibodies, bound analytes and barcoded secondary antibodies is remove from position (n, m) and transferred into a well plate or similar format. Afterwards, a new hydrogel matrix containing only primary antibodies is loaded again to position (n, m) and secreted molecules are collected again for a period dt. This process is repeated several times. The removed hydrogel matrix can now be analyzed using qRT-PCR and/or sequencing. This aspect of the present disclosure offers several advantages:
Firstly, it enables to perform a time-lapse analysis of secreted molecules derived from single cells located at n×m positions which is not possible with existing methods
Secondly, it enables to perform a time-lapse analysis of molecules which are secreted upon cell-cell interactions
Thirdly, the described method is highly compatible with established techniques and simplifies its integration into existing workflows

On-demand multi step stimulation. In a further aspect, the current disclosure relates to a method for stimulating cells located in said array at position (n, m) on-demand in a time-lapse manner. This has the advantage that single cells or multiple cells can be stimulated dependent on their current phenotype. As each cell population shows a certain degree of cell heterogeneity, single cells of such population might be respond in a different way to a given stimulus. For example, cells might be in a different cell cycle phase and a given stimulus might result in a different outcome depending on the current cell phenotype (e.g. cell cycle phase). Another advantage of said array is that cells located at a specific position can be stimulated multiple times with the same or a different stimulus. For example, stem cells usually pass through a differentiation cascade composed of various differentiation states and each state might require a different stimulus for directing the desired cell differentiation.

The processes and methods according to the present disclosure may be used among others for the analysis of cells, cell-cell and cell-matrix interactions.

### Methods for Producing Said Array

In one aspect, the present disclosure pertains to novel microfabricated structures and methods for producing said array having n×m microfabricated chambers containing immobilized hydrogel matrices for cell cultivation, stimulation, analysis and recovery/harvesting.

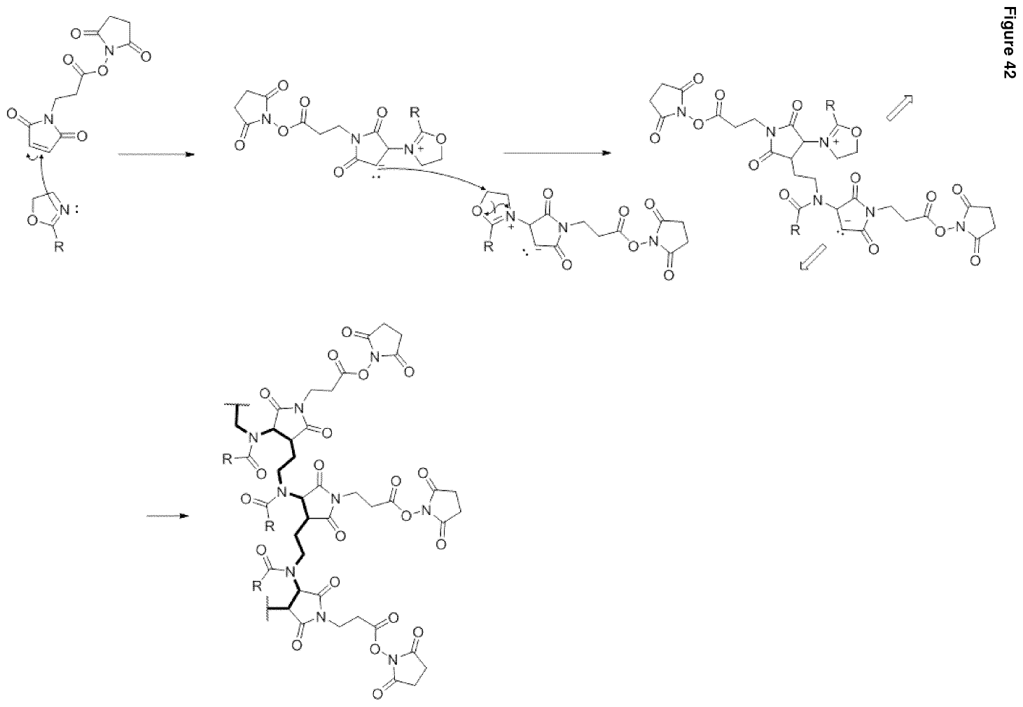

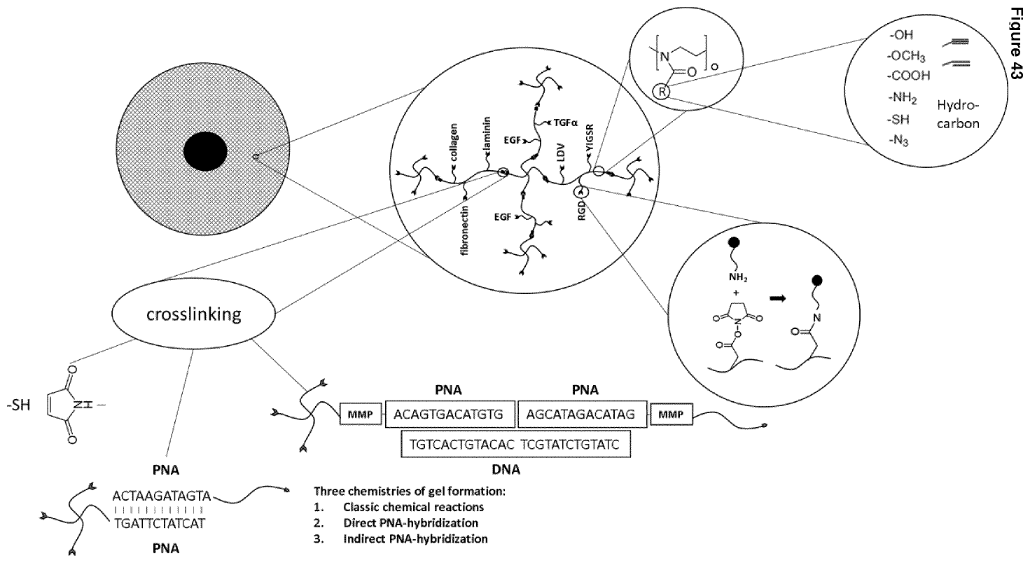

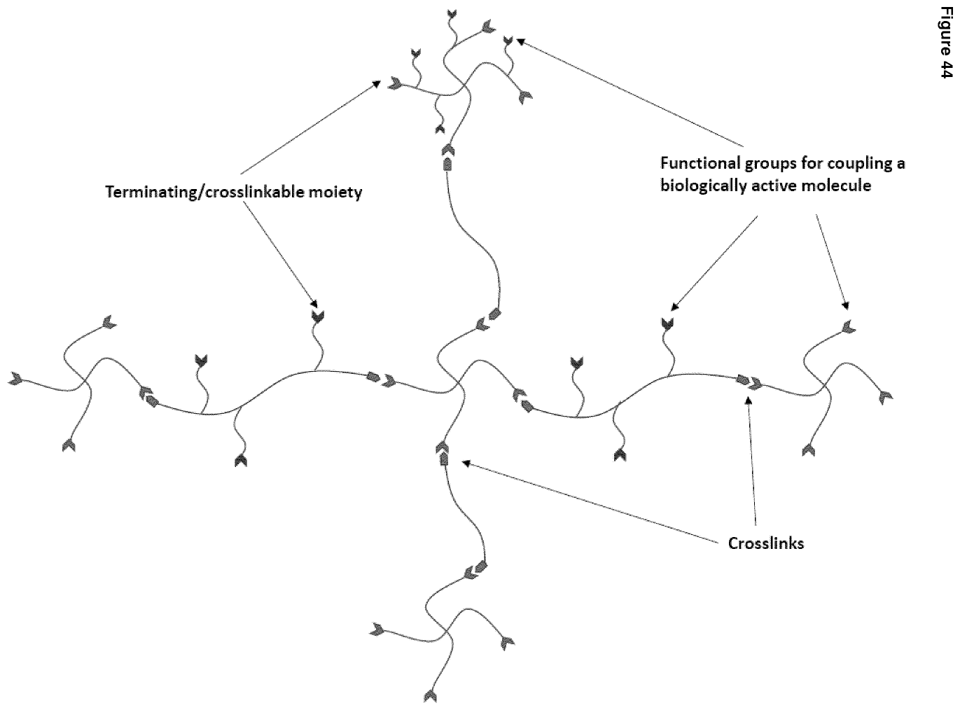

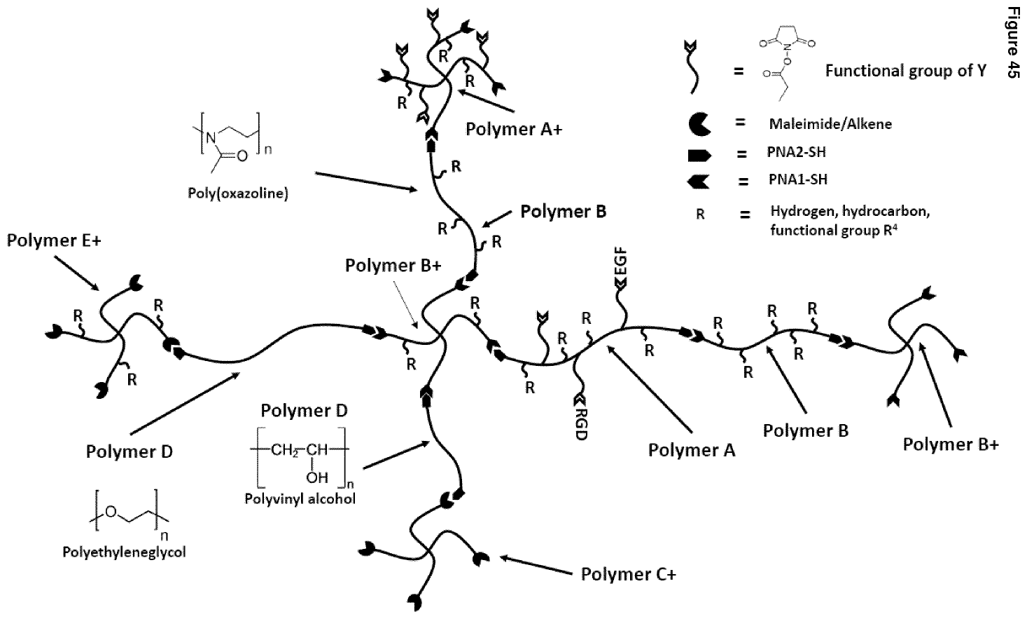

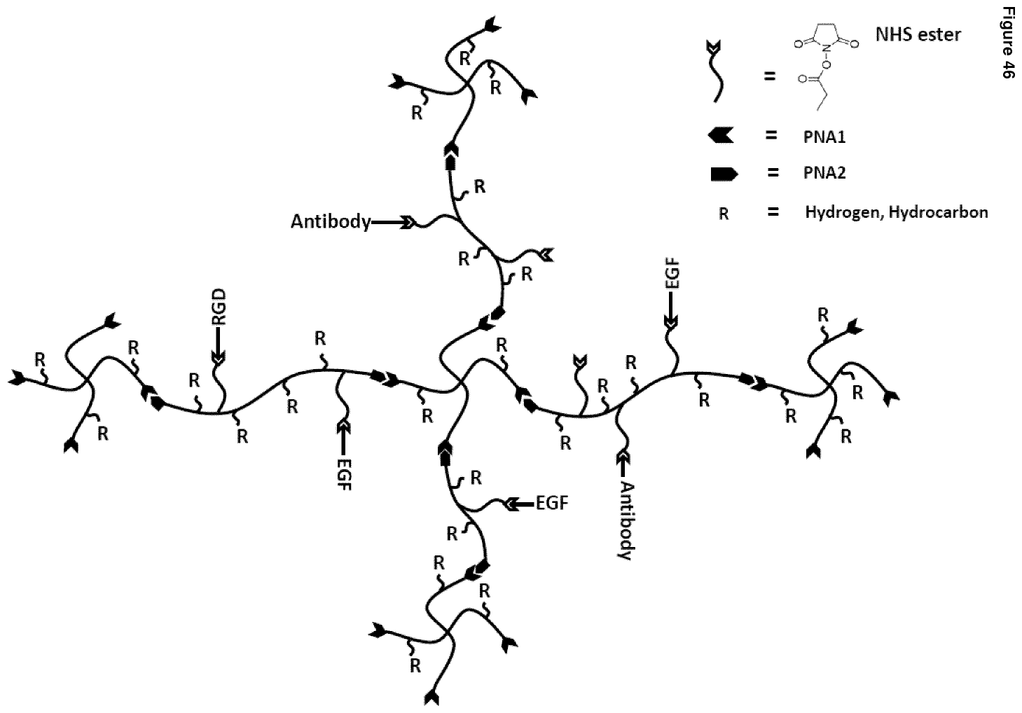

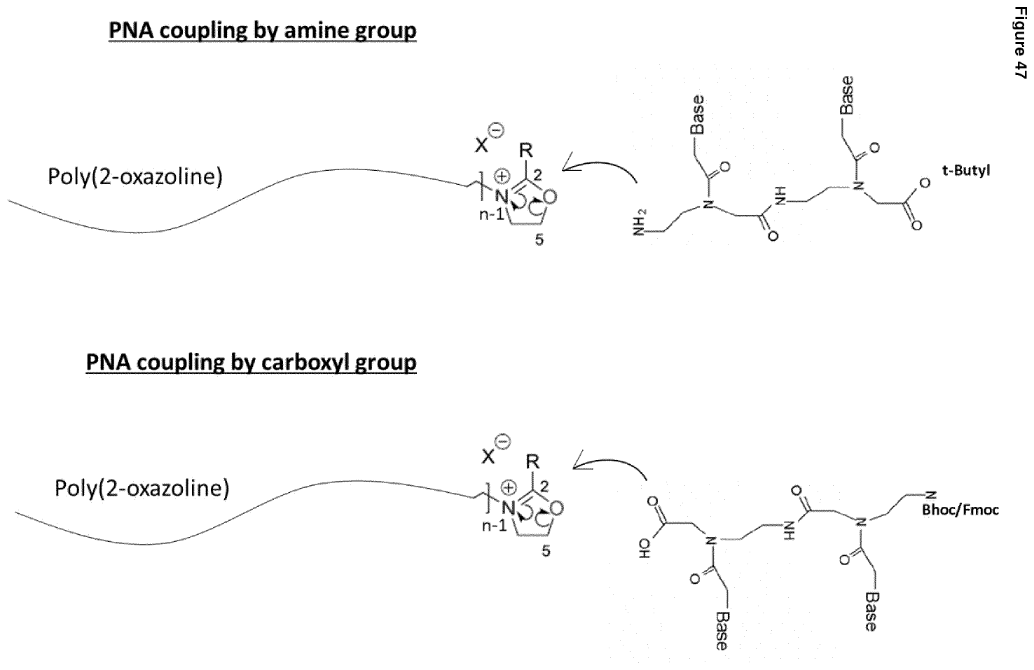

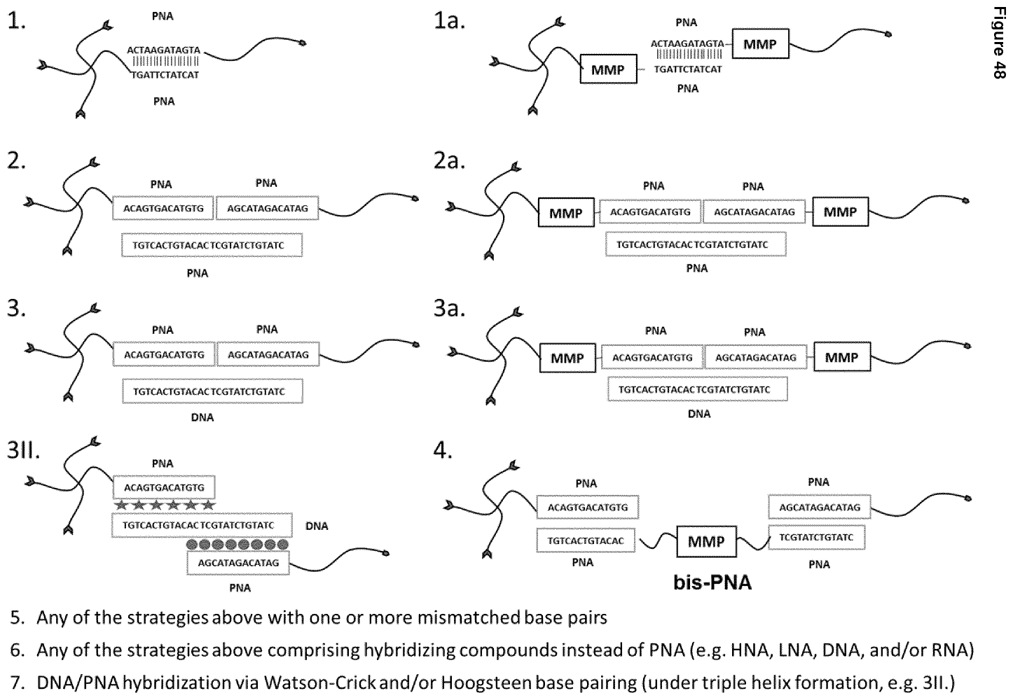

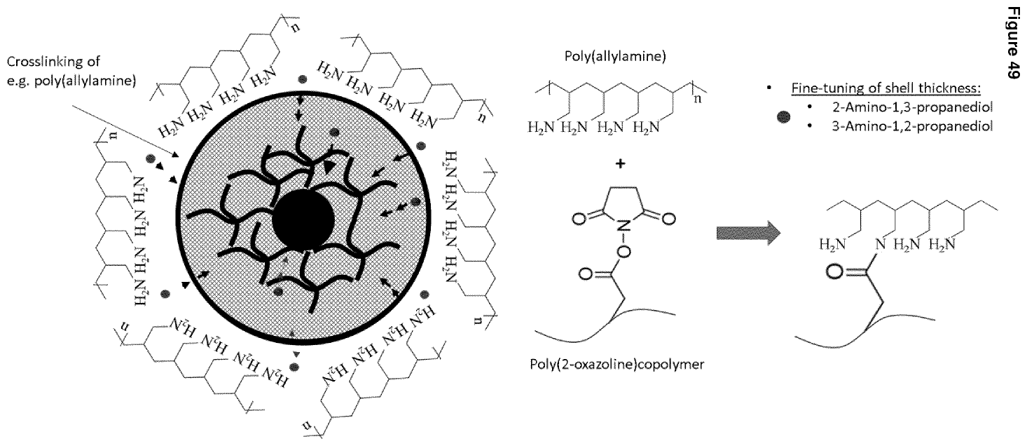

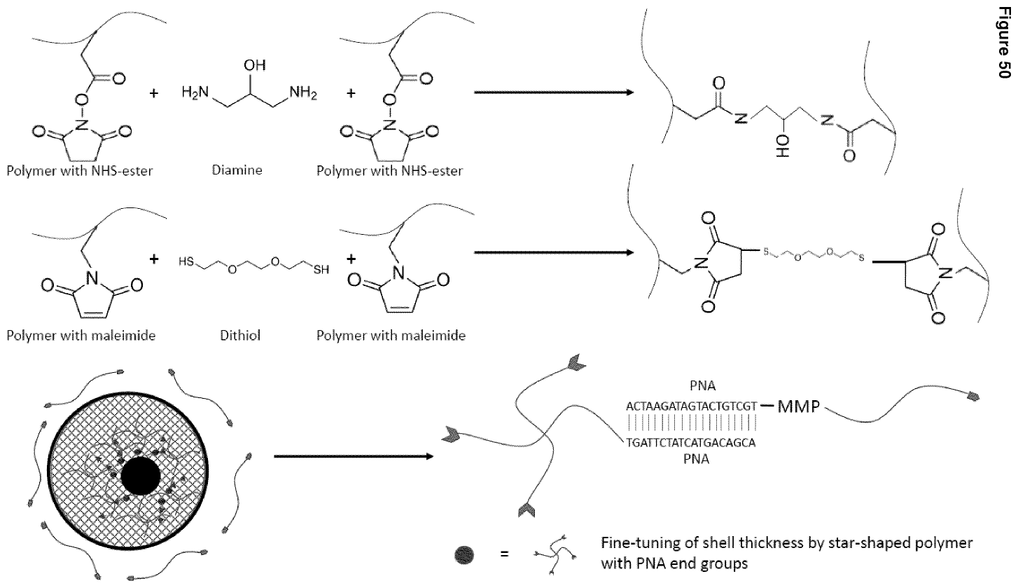

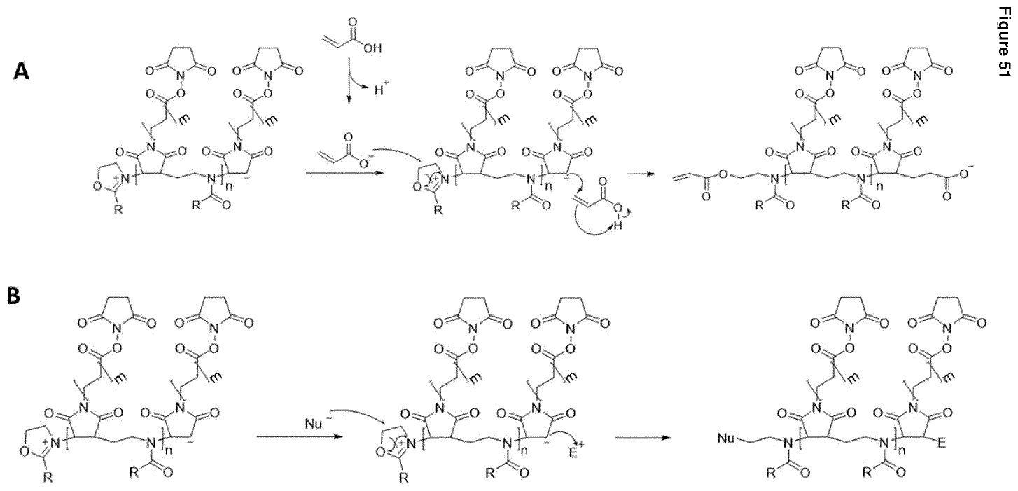

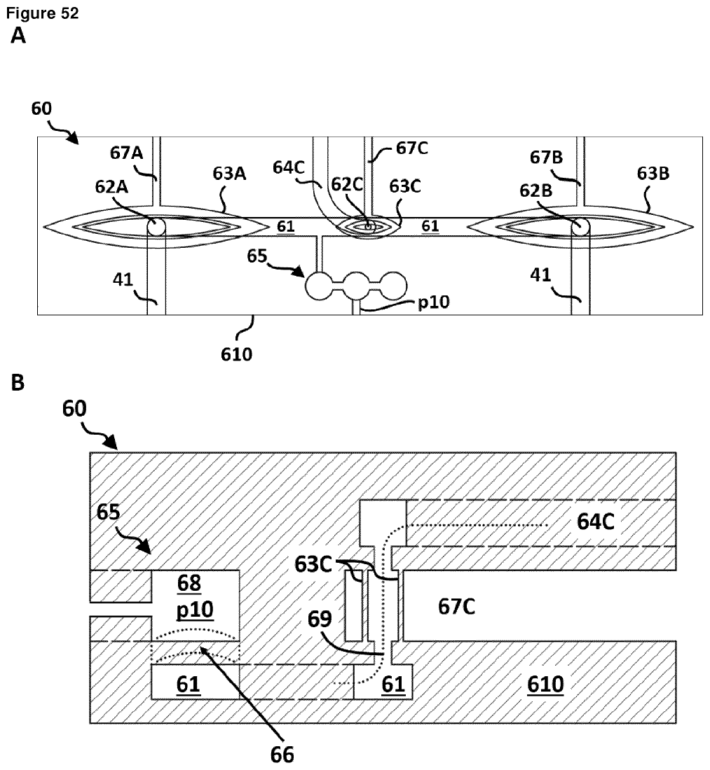

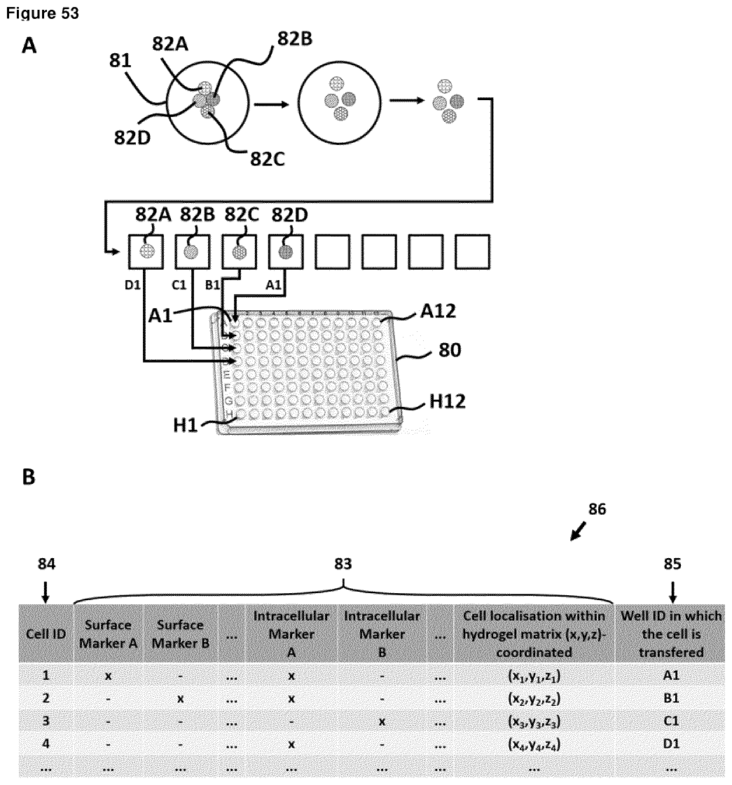

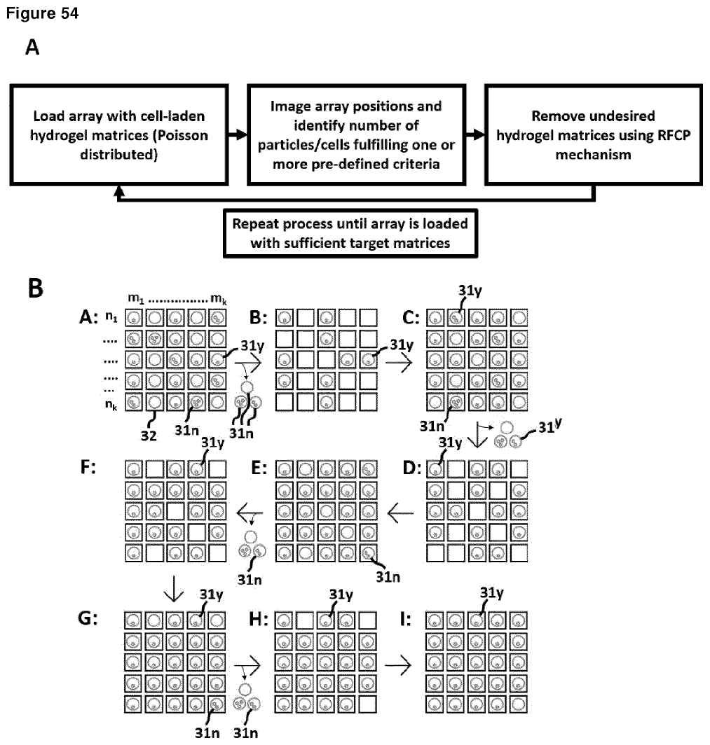

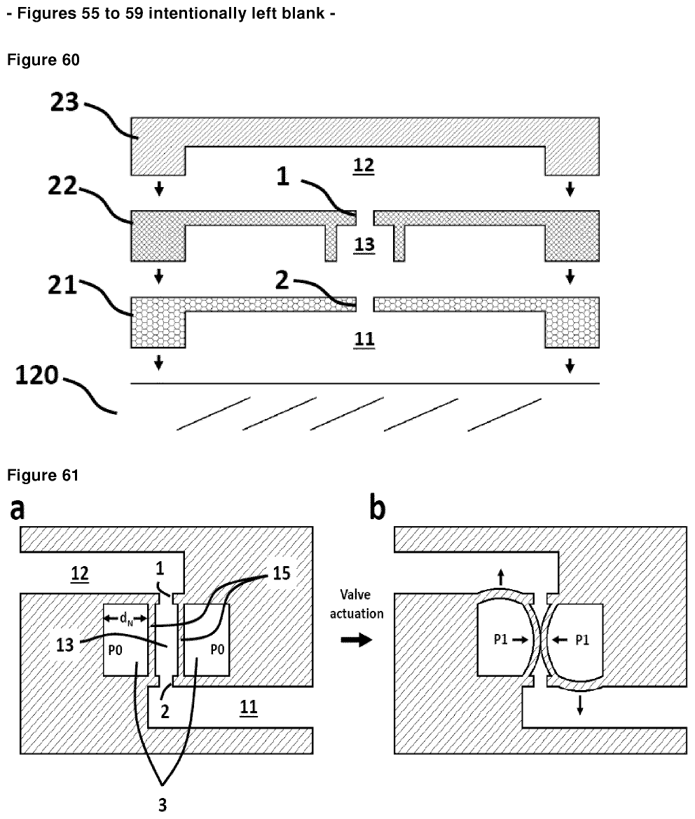

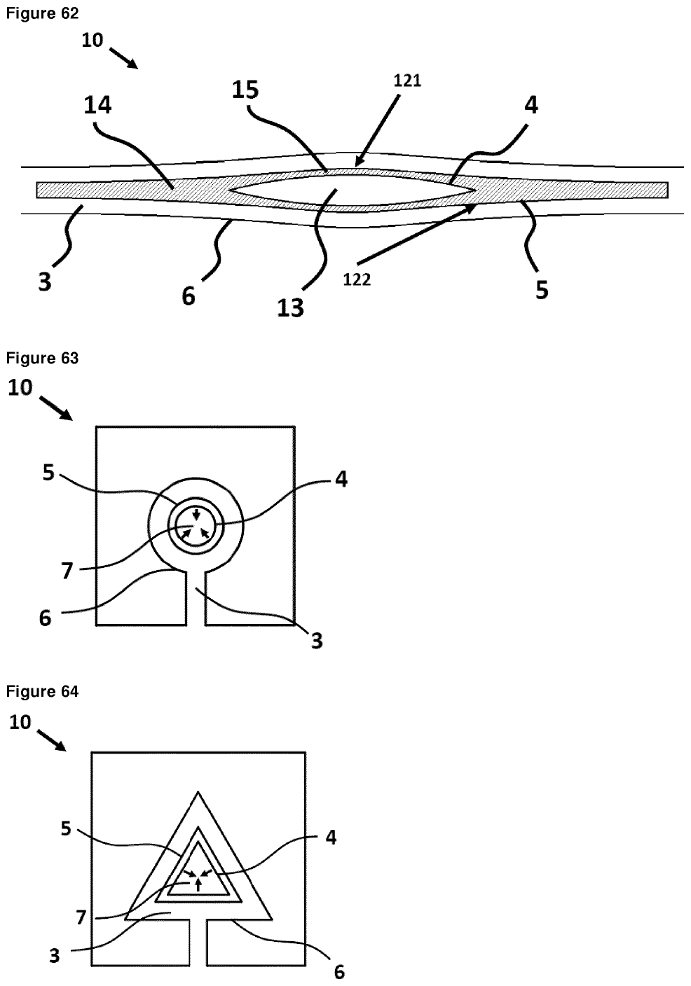

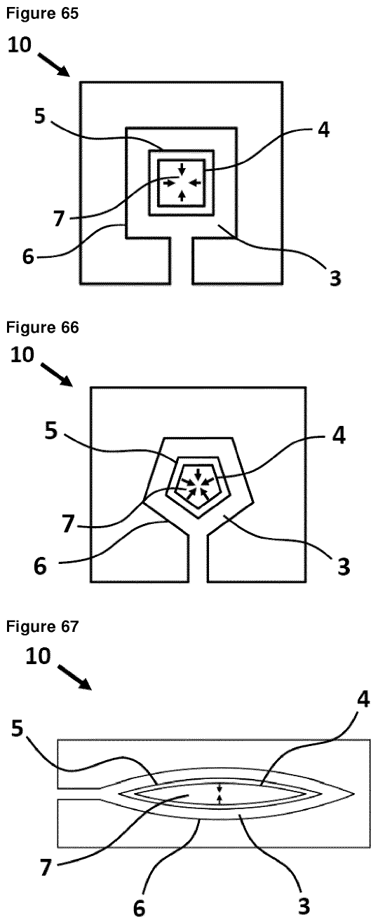

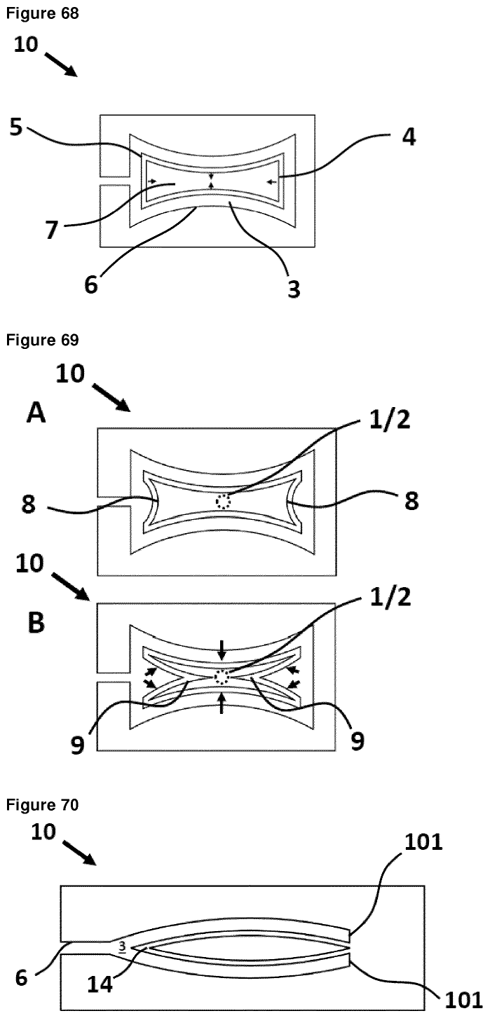

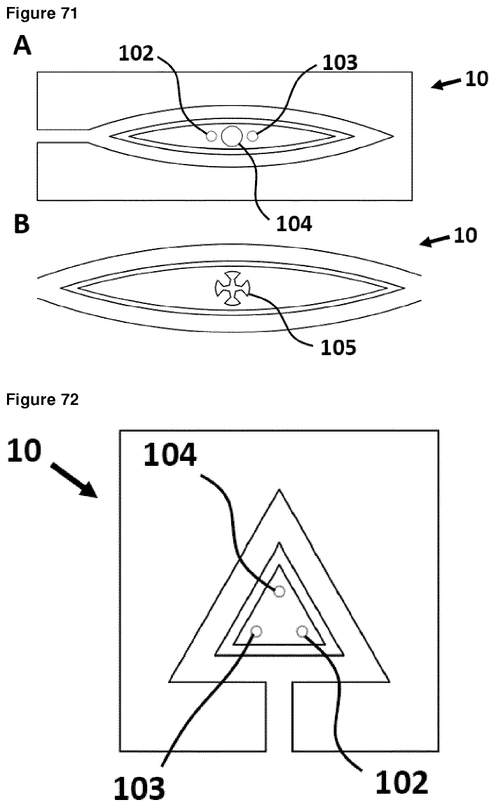

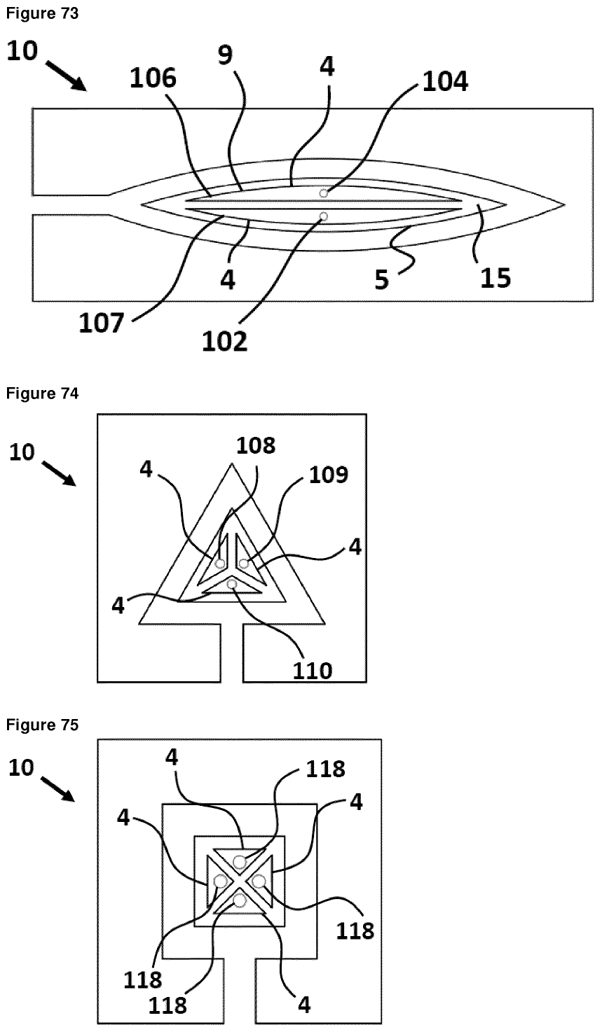

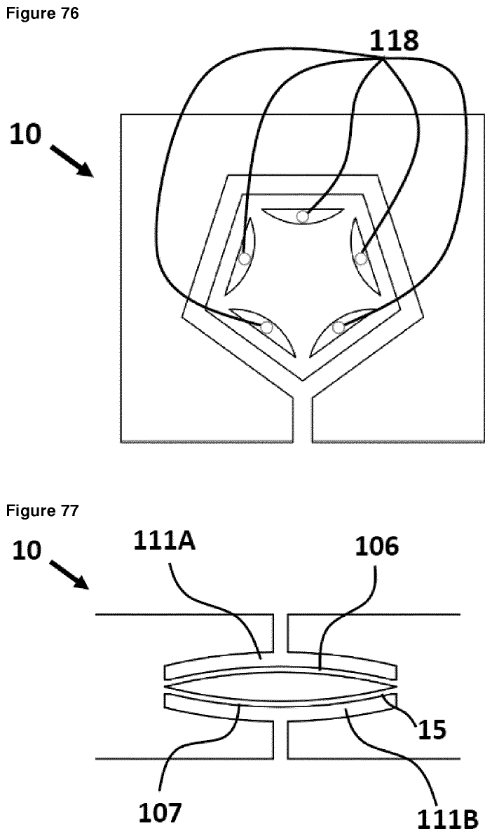

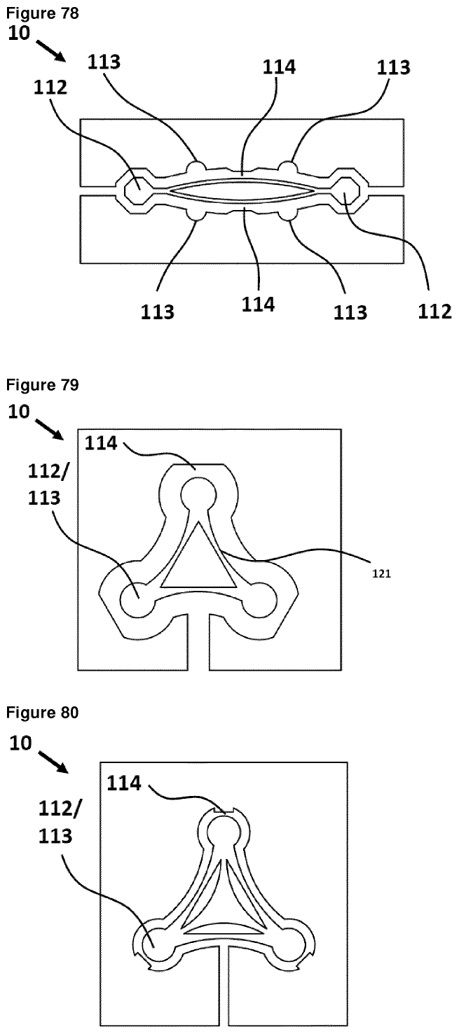

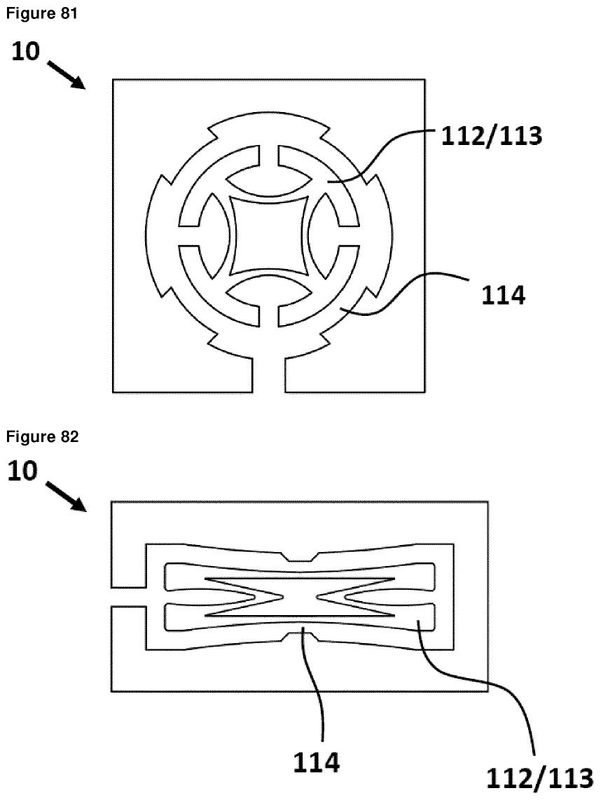

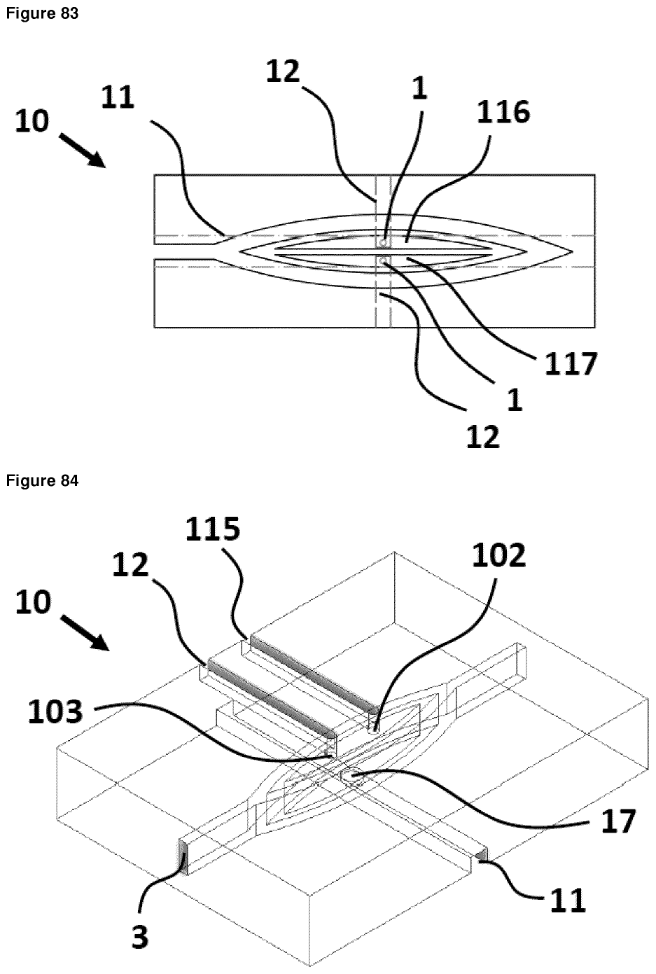

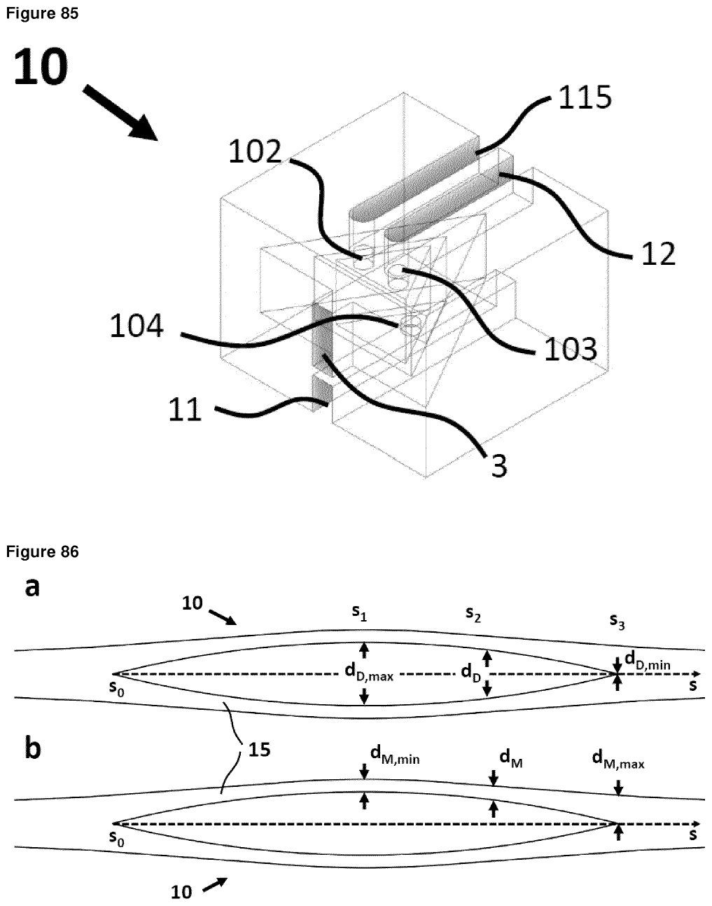

Description of elastomer valve. In a first aspect, the present disclosure relates to microfabricated structures and methods for the control of fluid flows within said array using a novel microfabricated elastomer valve. One of the main advantages of said microfabricated elastomer valve is that it can be used for performing and improving the most critical and important processes used in microfluidic devices as well as in the field of microdroplet microfluidics and in particular, for the generation of the disclosed array. In particular, these processes include control of fluid flows, fluid pumping and fluid mixing in microfluidic devices as well as the formation of droplets, formation of encapsulation, in particular single-cell encapsulations, co-encapsulation, droplet mixing, the formation of hydrogel matrices and droplet de-mulsification in terms of microdroplet-based microfluidics. The main advantage of said microfabricated elastomer valve is the low actuation pressure (<100 mbar) that is needed for its actuation as well as the nominal diameter that is suitable for the transport of larger hydrogel matrices. Another advantage of the elastomer valve is that it can be fabricated in a cost-effective and simple manner using standard multilayer lithography methods. In a first embodiment said microfabricated structure for flow control consists of a first microfabricated layer with recesses comprising a first microfabricated channel which is defined as “first flow channel” and a second microfabricated layer that has a recess which connects the first microfabricated channel with the space above the second microfabricated channel (FIG. 4). This recess is defined as “connection channel”. The connection channel is separated by a second recess of the second microfabricated layer by a thin elastomeric membrane with a thickness between 1 μm and 80 μm. The first flow channel might contain a first fluid and the space above the second microfabricated layer might contain a second fluid of the same or of different type. The recess within the second microfabricated layer that is separated by an elastomeric membrane from the connection channel is here defined as “actuation channel”.

The term object within fluid may in particular comprise droplets and/or particles.

A droplet may comprise hydrogel particles, a hydrogel matrix, hydrogel beads, hardened and/or gelled and/or polymerized hydrogels or any other accumulated particles in particular are bonded to each other in a chemical or physical way (e.g. by surface tension), that keeps the particles together and delimits the accumulated particles from the environment, in particular a fluid surrounding the particles. In particular the droplet may also be selected from one of: a water in oil droplet, an oil in water droplet, double emulsion, triple emulsions, multiple emulsion. The droplet may have a spherical shape but the shape can deviate from the spherical shape; in particular the droplet may be a plug or may be plug shaped. A particle may comprise biological cell or cells, microstructures, in particular microfrabricated electrodes, nanostructures, gold nanocrystals, biological compound, wherein the term biological compound comprises DNA, RNA proteins, in particular antibodies, LNA, PNA, small molecules, photocleavable linker. In particular one of more particles, such as one or more cells may be contained within a droplet.

The droplet may contain any kind of particles, in particular biological or chemical particles which may be subject of an observation. A particle may be a cell. The droplet may contain a hydrogel surrounding the particle. In particular, the droplet comprises a hydrogel/hydrogel matrix composed of an organic monomer, organic building block and/or an organic polymer according to the present disclosure.

The term “channel” requires at least any cavity which is adapted to accommodate a fluid. In an embodiment the channel may constitute a part of a conduct for conducting a stream of fluid. A channel may be a formed by a fluid conduct; a channel may be formed by a reservoir. Such a reservoir may be closed or may be open with a connection to the atmosphere. In an embodiment the channel may be a reservoir. For example, this reservoir may be closed except for the opening which connects it to another channel. Alternatively the reservoir may be open, for instance it may have an open upper end. In a one embodiment the second channel is a reservoir, in particular an open reservoir.

Opening and closing of valve. In one embodiment, this actuation channel contains a fluid such as air or fluorinated oil (e.g. HFE-7500 (Novec)). Upon increasing the pressure in said actuation channel, a pressure difference between the connection channel and the actuation channel is generated. Thus, an actuation force is acting on the elastomeric membrane separating the connection channel and the actuation channel. This actuation force results in a bending of the membrane and a closing of the connection channel thereby separating the first flow channel from the space above the second microfabricated layer. After removing said pressure, the connection channel opens again due to the elastomeric characteristics of the used membrane. In a particular embodiment, the deflection distance of the membrane might be in the range of 1 μm to 100 μm. In another embodiment, the connection channel is not fully closed and thus the hydrodynamic resistance of the connection channel can be controlled in a defined manner by changing the applied pressure and thus the actuation force acting on the membrane. In one embodiment, the pressure might be varied between 0 mbar and 4000 mbar (absolute pressure) in steps of 1 mbar to adjust the hydrodynamic resistance of the connection channel. In particular embodiments the actuation force might be applied by using fluids (hereinafter also referred to as control fluid or actuating fluid) of the following type:
Gases such as air, nitrogen and argon
Liquids such as water, silicon oils, fluorinated oils and other oils
Solutions containing salts and/or polymers such as polyethylene glycol or glycerol
Ferromagnetic fluids
Hydrogels that are capable of swelling and shrinking upon application of a stimulus. For example said stimulus might be one of the following types: temperature, ionic strength, electric field strength, magnetic field strength, pH value

In addition to applying an actuation force via a pressure-based actuation system, valve actuation might be performed by other actuation systems that might be of the following types: electrostatic, magnetic, electrolytic or electrokinetic.

Valves can be actuated by injecting gases (e.g., air, nitrogen, and argon), liquids (e.g., water, silicon oils and other oils), solutions containing salts and/or polymers (including but not limited to polyethylene glycol, glycerol and carbohydrates) and the like into the control channel, a process preferred to as “pressurizing” the control channel. In addition to elastomeric valves actuated by pressure-based actuation systems, monolithic valves with an elastomeric component and electrostatic, magnetic, electrolytic and electrokinetic actuation systems may be used. See, e.g., US 20020109114; US 20020127736, and U.S. Pat. No. 6,767,706.

In particular embodiments valves (including valves with dimensions as described above) do not completely block the flow channel lumen with the membrane is fully actuated by a control channel pressure of 30, 32, 34, 35, 38 or 40 psi.

Biconvex shape. In another advantageous embodiment, the elastomeric membrane separating the connection channel and the actuation channel has a biconvex shape with one circle having a radius r₁, the second circle having the radius r₂, a distance between the centers of both circles of s and with a separating elastomeric membrane having a thickness d (FIG. 6 a-b). In a particular embodiment, the radii r1 and r2 are equal. One of the main advantages of a biconvex shape is the low actuation pressure that is needed for completely closing the connection channel. A second advantage of using a biconvex shape is that the nominal diameter of said valve is suitable for the transport of larger hydrogel matrices.

Triangular shape. In another advantageous embodiment, the elastomeric membrane separating the connection channel and the actuation channel has a triangular shape with a separating elastomeric membrane having a thickness d, one side of a triangle having the length a, a second side of a triangle having a length b and a third side of a triangle having a length c (FIGS. 6 c-d). The advantage of a triangular shape is the reduced footprint of the valve. Thus, the number of valves per mm² can increased. Numerous geometries are also illustrated in the figures.

Fluid injection. In another advantageous embodiment, the space above the second microfabricated layer is composed of a recess within a third microfabricated layer that is defined as “second flow channel” (FIG. 5). The second flow channel might contain a fluid of type 2 and the first flow channel might contain a fluid of type 1 with fluid of type 2 and fluid of type 1 being miscible. A defined amount of the fluid of type 2 might be injected into the fluid of type 1 by applying a hydrodynamic pressure within the second flow channel that is larger than the hydrodynamic pressure in the first flow layer and by opening said elastomer valve for a defined time (e.g. 0.1 ms to 500 ms). The main advantage of using said microfabricated elastomer valve for injection of a fluid is the short opening and closing time that is needed due to the low actuation pressure resulting in a very fast valve operation. The opening time may be for example be 1, 2, 3, 4, 5 ms, s. or min.

Electric actuation. In another advantageous embodiment, said microfabricated elastomer valve having in particular a biconvex shape is actuated using a modification of a voltage applied to the valve portion, in particular an actuation force generated by an electric field. This has the advantage, that no external valves such as solenoid valves are necessary for valve actuation. To this end, the two sides of a biconvex elastomer valve that have direct contact to the actuation channel may be coated with a, in particular thin, electrostatic chargeable polymer (for example, the actuation channel may be coated with conducting nanoparticles, in particular gold nanoparticles or carbon black nanoparticles using conventional surface chemistry) layer that enables to charge one side of the elastomer valve positively and the other side negatively (FIG. 7). Thus, an electric field is generated between the two sides of said microfabricated elastomer valve. Applying a voltage to said polymer layers results in an electrical actuation force acting on the membrane separating the connection channel and the actuation channel which closes the connection channel. The main advantage of using an electrical actuation force is the decreased time needed for applying an actuation force which results in a much faster valve operation. If the valve portion is adapted to be selectively opened and closed upon modification of a voltage applied to the valve portion, the valve portion, in particular the membrane, may be a piezoelectric element.

Parallel actuation. In another advantageous embodiment, multiple microfabricated elastomeric valves might be actuated simultaneously which increases the process speed by parallelization. To this end, multiple microfabricated valves are located within the same actuation channel. If an actuation force is applied in said actuation channel, all microfabricated valves are closed at the same time. Each microfabricated valve might have a first and a second flow channel as described above which are separated from the first and second flow channels of the other microfabricated valves. Thus, different fluids located in the second flow channels might be injected simultaneously into different fluids located in the first flow channel. In another embodiment, all microfabricated valves are connected to the same second flow channel.

Description of peristaltic pump using arrangements of said elastomeric valves. In another advantageous embodiment, multiple elastomeric valves 10A 10B 10C are arranged in form of a peristaltic pump 50 to perfuse a fluid with a defined flow rate through said array (FIGS. 36 and 37). To this end, a first elastomeric valve 10A connects a first flow channel 11A at a first position with a second flow channel 12A. Said second flow channel 12A is connected at a different position to a first flow channel 11B by using a second elastomeric valve 10B. This second position is in turn connected to a second flow channel 12C at a third position using a third elastomer valve 10C. All three elastomer valves 10A, 10B, 10C can be operated individually by either using a pneumatic or hydraulic pressure or by using an electric field via a control fluid line 301, 302, 303. A fluid located within the first and second flow channels 11,12 can then be pumped through the flow channels by operating said elastomer valves 10A, 10B, 10C in a defined manner along a direction of fluid F.

Said elastomer valves 10A, 10B, 10C might be operated in the following cycle with “0” presenting a closed valve and “1” presenting an open valve (see FIG. 37: 1|0|1 (FIG. 37A (first elastomer valve 10A|second elastomer valve 10B|third elastomer valve 10C), 1|0|0 (FIG. 37B), 1|1|0 (FIG. 37C), 0|1|0 (FIG. 37D), 0|1|1 (FIG. 37E), 0|0|1 (FIG. 37F)). Said cycle might be repeated to pump multiple fluid volumes. The main advantage of using said microfabricated elastomer valves 10 as a peristaltic pump is that fluid located within the flow channels can be pumped with very precise flow rates in the range of several nL/min. The flow rate is based inter alia on the valve geometry.

Adjustment of peristaltic pump flow rates. In another advantageous embodiment, the maximum flow rate of said peristaltic pump might be adjusted by either adjusting the elastomer valve geometry or by using multiple elastomer valves in a parallel manner for peristaltic pump operation (see FIG. 36C). To this end, more than one valve 10 is used at one stage of the peristaltic pump. Thus there are three first valves 10A arranged in parallel, three second valves 10B arranged, and three third valves 10C arranged in parallel in one peristaltic pump as described previously are connected to the same inlet and to the same outlet. The first, the second and the third elastomer valve of said multiple peristaltic pumps are operated simultaneously and or in a coordinated manner (sequentially). The main advantage of using said peristaltic pump in a parallel manner is that the maximum volume flow rate and or the maximum pressure can be precisely adjusted.

In another aspect, the present disclosure relates to methods for the on-demand formation of droplets with defined sizes and at very high frequencies.

According to the invention, the valve portion is arranged within the connection channel. In this context, the term “within” in particular means that the valve portion is at least part of the connection channel. In a preferred embodiment, the valve portion constitutes the outer wall of the connection channel. In the case where the valve portion comprises the flexible membrane, it is advantageous that the flexible membrane forms at least part of the outer wall of the connection channel, especially preferred the flexible membrane forms the entire outer wall of the connection channel.

In a preferred embodiment, the valve portion comprises at least one flexible membrane which is adapted to be selectively transferred between an open and a closed shape. In particular, it is additionally adapted to be transferred into an intermediate shape. Thereby, it is possible to adjust the flow resistance in the valve. As a result, this embodiment enables for example to regulate the flow rate. Especially preferred the flexible membrane may be hold for a predetermined time into the intermediate shape, whereby it is possible to control the fluid flow rate. In an advantageous embodiment, the valve portion may consist of or substantially consist of the flexible membrane.

In an advantageous embodiment, the longitudinal axis of the connection channel is not parallel to the longitudinal axis of the first channel and/or to the longitudinal axis of the second channel, in particular the longitudinal axis of the connection channel is substantially orthogonal to the first channel and/or to the second channel.

For the purposes of the invention, the term “orthogonal” in particular means an angle of 90°, taking into account the usual manufacturing tolerances in the present technical field. (This applies accordingly to other definitions using the word “substantially”.) In alternative embodiments, the longitudinal axis of the connection channel is at another angle, for example of substantially 15°, 30°, 45°, 60° or 75°, to the first channel and/or to the second channel.

In the sense of the invention, the term “longitudinal axis” usually refers to an axis which runs in the direction of the longest extension of the channel. In most cases, the longest extension of the channel corresponds to the direction of the fluid flow, at least of the main fluid flow.

In an advantageous embodiment, the longitudinal axis of the connection channel is substantially parallel or at an angle between 0° and 90°, in particular between 0° and 45°, to the normal vector of the surface of the first channel facing the connection channel and/or the longitudinal axis of the connection channel is substantially parallel or at an angle between 0° and 90°, in particular between 0° and 45°, to the normal vector of the surface of the second channel facing the connection channel.

Especially in the case where a longitudinal axis of the first/second channel cannot be identified due to its specific geometry or where the first/second channel is a reservoir comprising an axis (for example a rotation axis) which is not substantially orthogonal to the axis of the longitudinal axis of the connection channel, the arrangement of the channels may be defined by means of the normal vector of the surface of the first/second channel facing the connection channel.

In an alternative embodiment, the longitudinal axis of the connection channel is substantially parallel or at an angle between 0° and 90°, in particular between 0° and 45°, to the normal vector of the surface of the first channel being opposite the connection channel and/or the longitudinal axis of the connection channel is substantially parallel or at an angle between 0° and 90°, in particular between 0° and 45°, to the normal vector of the surface of the second channel being opposite the connection channel.

In a preferred embodiment, the valve portion comprises at least one flexible membrane, the flexible membrane is adapted to be selectively transferred between an open shape and a closed shape, and in particular between an intermediate shape. In particular, in the open shape a transfer of fluid between the first channel and the second channel and/or vice versa is enabled and in the closed shape a transfer of fluid between the first channel and the second channel and/or vice versa is disabled. In particular the membrane is adapted to be selectively transferred into an intermediate shape, wherein in the intermediate shape a flow resistance in the valve is increased compared to the open shape.

In the sense of the invention the term “open shape” refers to the shape of the valve with the widest possible opening. In this shape the flow resistance is minimal. This shape is obtained in particular when there is a vacuum in the actuation chamber. Thus, in the intermediate shape, the opening of the flexible membrane is less wide than in the open shape and therefore the flow resistance is larger.

In a preferred embodiment the flexible membrane extends along the entire length of the connection channel. The flexible membrane forms at least part of the outer wall of the connection channel. Advantageously, the flexible membrane is an elastomeric membrane.

In a preferred embodiment, the flexible membrane may be transferred into at least one intermediate shape. Especially preferred, the flexible membrane may be hold into the intermediate shape for a predetermined time. This enables to vary the flow resistance in the valve as required for a specific application.

In a preferred embodiment, the connection channel is connected to the first channel by at least one first opening and the connection channel is connected to the second channel by at least one second opening. Especially preferred, the first opening is located within the first channel and/or the second opening is located within the second channel. Thereby, the first opening and/or the second opening enable fluid flow between the first channel and the connection channel and/or between the second channel and the connection channel. Preferably, the first opening is provided in the first channel in such a way that its axis is substantially perpendicular to the longitudinal axis of the channel. Preferably, the second opening is provided in the second channel in such a way that its axis is substantially perpendicular to the longitudinal axis of the channel.

In a preferred embodiment, the first opening is adjacent to a first end of the connection channel and/or the second opening is adjacent to a second end of the channel (13). Especially preferred, the first opening is directly adjacent to a first end of the connection channel and/or the second opening is directly adjacent to a second end of the channel (13). Thereby, a fluid flowing through the first opening directly enters the connection channel through the opening of the connection channel at its end surface or vice versa. The same applies to the second opening: a fluid flowing through the first opening directly enters the connection channel through the opening of the connection channel at its end surface or vice versa.

In a preferred embodiment, the first end of the connection channel is a first end_face of the connection channel and/or the second end of the connection channel is a second end face of the connection channel.

Usually, the first end face and the second end face serve as inlet and outlet of the connection channel. That means, that the channel is configured to enable a fluid flow from the first face end to the second face end or vice versa. In particular, the first end face and the second end face are open. Alternatively, one of the end faces are open, the other is closed except for the first or the second opening. Especially preferred, the outer (especially circumferential) border of the end face is the outer wall of the connection channel, in particular the outer wall of the flexible membrane.

In a preferred embodiment, the shape of the first opening differs from the shape of the cross section of the connection channel, in particular from the shape of the first end of the connection channel, and/or the shape of the second opening differs from the shape of the cross section of the connection channel, in particular form the shape of the second end of the connection channel.

In the sense of the invention, the “cross section of the connection channel” is limited by the outer wall of the connection channel. The outer wall is the outer boundary of the connection channel which limits the connection channel to the outside. In the case where the flexible membrane forms the connection channel, the shape of the first opening and/or the shape of the second opening differ from the shape of the cross section of the connection channel (i.e. the flexible membrane), regardless weather the flexible membrane is in a deformed or non-deformed state.

In an especially preferred embodiment, the cross section of the connection channel is larger than the first opening and/or the second opening. In particular, this applies to the end face of the connection channel. It is a basic advantage of the invention that the shape and size of the first opening and/or the second opening which form the inlet and/or outlet of the connection channel are not dependent on the shape and size of the cross section of the connection channel. These openings may differ in size and shape and thereby open a plurality of new applications of the microfabricated valves.

In a preferred embodiment, the shape of the first opening and the shape of the second opening are identical or different.

The first and/or second opening may have for example a round or polygonal shape. The polygon may have 3, 4, 5, 6, 7, 8, 9, 10 etc. corners. The corners may be pointed or rounded or a combination of both. The shape may for example comprise at least one edge being curved, in particular convex or concave, for example plano-convex or plano-concave. In an preferred embodiment, the shape may be polygon with curved edge and straight edges. It is also possible, that the shape may be a combination of convex and/or concave and/or biconvex/biconcave and/or polygonal with curved and/or straight edges.

One of the main advantages of a variable shape of the first as well as of the second opening is that by adjusting the opening geometry the flow profile within the connection channel can be controlled/influenced. The desired flow profile within the connection channel may depend on the application.

In a preferred embodiment, the first opening and second opening have the same shape. In a preferred embodiment, the shape of the first opening and the shape of the second opening are round. This preferred shape serves for transport of small particles such as cells through the first/second opening. The round shape is preferred because the velocity profile through the round opening is usually parabolic. The parabolic velocity profile results in a positioning of the particle on or near the central axis of the round opening. Thus, the first and the second opening preferably have the same shape (preferably round) as this generates a flow profile which positions a passing particle on or near the axis of the valve portion thereby preventing a potential accumulation of particles within the connection channel.

In an alternative advantageous embodiment, the first opening and second opening have a different shape. This is advantageous for mixing of at least two fluids: For this purpose, at least two second openings are provided. The first second opening may for example connect the first second channel containing a first fluid and the connection channel, whereas the second opening may connect a second channel containing a second fluid and the connection channel. These two fluids that have to be mixed enter the connection channel through the according second opening. Preferably, the first and/or second opening may provide a small hydrodynamic resistance. For instance, this can be realized by a round shape with a large radius. A small resistance means that a higher flow rate can pass through the channel. In contrast, a high flow resistance means that the flow rate is lower. If the first and second fluids enter the connection channel through the according second openings, in a preferred embodiment these fluids come into contact with each other so that they can be mixed. The fluids come into contact with each other so that they are mixed. In order to retard the flow of the fluids out of the connection channel in order to mix the fluids as thoroughly as possible, a common first opening can be provided, which has an increased flow resistance. An increased flow resistance can, for example, be achieved by a small cross-section of the first opening. Especially, the common first opening has a shape which facilitates the generation of turbulences for effective mixing. In an exemplary embodiment, this can be achieved by providing baffles within the first opening. Thus, the different shapes of the first and second openings might be especially used for generating flow profiles suitable for mixing of at least two fluids. Other useful applications are conceivable.

In a preferred embodiment, the first opening and the second opening are substantially coaxial. This enables a direct fluid flow from the first channel through the connection channel to the second channel, especially when the first channel and the second channel are located vertical above each other. In an alternative embodiment, the first opening and the second opening are not coaxial. This embodiment is advantageous for applications in which the fluid must stay within the connection channel for a while, for example if fluids are to be mixed within the connection channel. By arranging the openings non-coaxial the fluid after entering the connection channel through the first opening is impelled to reach the second opening through a diversion. The fluid thereby stays for a longer time within the connection channel.

In a preferred embodiment the number of the first openings and the number of the second openings are different. This embodiment is particularly advantageous, if at least two fluids from different channels are to be mixed or injected into at least one common channel. Various other applications are possible.

In a preferred embodiment, the valve portion is adapted to be selectively opened and closed, in particular transferred into an intermediate shape, upon modification of a pressure, in particular fluid pressure of a control fluid, in particular compressed air or silicone oil, acting onto the membrane. In particular the flexible membrane is transferred into the open shape and/or transferred into the closed shape and/or into the intermediate shape upon decreasing/increasing the fluid pressure. Thereby, the control fluid stream produces a force which acts on the flexible membrane transferring it in the shape as desired.

In a preferred embodiment, the microfabricated valve comprises at least one actuation chamber, wherein the connection channel is separated from the actuation chamber by at least one section of the flexible membrane. In context of the expression “the connection channel is separated from the actuation chamber by at least one section of the flexible membrane”, the term “connection channel” is to be understood as the recess through which the fluid may flow. The section of the flexible membrane separating the connection channel from the actuation chamber is in this context not part of the channel. This is also applicable to the expression “the connection channel is separated from the second actuation chamber by at least one section of the flexible membrane”. It is preferred, that the section of the flexible membrane extends over the entire circumference of the connection channel.

In the sense of the invention, the term “actuation chamber” in particular refers to a cavity that may contain a fluid. In a preferred embodiment, the chamber is a closed cavity. In a preferred embodiment, the chamber can have an inlet through which a fluid can flow into the chamber and/or an outlet through which the fluid can flow out of the chamber. In another preferred embodiment, the chamber may be or comprise a channel. In particular, fluid pressure of the control fluid acts onto the membrane within the chamber.

In an especially preferred embodiment, the valve portion is adapted to be selectively opened and closed, and in particular transferred into an intermediate shape, upon modification of a pressure difference between the actuation chamber and the connection channel by modification of the pressure inside the actuation chamber, wherein the pressure inside the actuation chamber is adjusted.

One way to adjust the pressure inside the actuation chamber is realized by an actuation_fluid which can flow into the actuation chamber to increase the pressure inside the chamber or to flow out of the chamber to decrease the pressure inside the chamber, in particular to generate a vacuum inside the actuation chamber. The actuating fluid can be of the same type as the control fluid. However, adjusting the pressure inside the chamber is not limited to such solution. For example, the pressure can be increased by increasing the temperature within the chamber or it can be decreased by decreasing the temperature within the chamber. For instance, that may have the effect, that a fluid which is located inside the chamber increases (decreases) its volume due to the increased (decreased) temperature. In this example, the temperature serves as stimulus to increase the pressure inside the chamber. Another example is a stimulus in order to swell a hydrogel which is located inside the chamber. Such stimulus may be for example the modification of the pH value.

In a preferred embodiment, the microfabricated valve comprises at least a second actuation chamber, wherein the connection channel is separated from the second actuation chamber by a second section of the flexible membrane, wherein the second section of the flexible membrane and the first section of the flexible membrane are different, wherein the valve portion is adapted to be selectively transferred into an open and/or closed and/or intermediate shape upon modification of a pressure difference between the second actuation chamber and the connection channel by modification of the pressure inside the second actuation chamber, wherein the pressure inside the second actuation chamber is adjusted, in particular by a actuation fluid which can flow into the second actuation chamber to increase the pressure inside the second actuation chamber or to flow out of the second actuation chamber to decrease the pressure inside the second actuation chamber, in particular to generate a vacuum inside the second actuation chamber. However, adjusting the pressure inside the chamber is not limited to such solution and other exemplary solutions are described above in connection with the first actuation chamber.

In an advantageous embodiment, the pressure inside the first actuation chamber and the pressure inside the second actuation chamber can be modified independently. Thereby, the pressure within the first actuation chamber can be adjusted without affecting the pressure of the second actuation chamber.

In an alternative embodiment, the valve portion is adapted to be selectively opened and closed upon modification of a voltage applied to the valve portion, in particular the valve portion comprises at least one electrostatic chargeable layer, in particular polymer layer, which is adapted to change its form upon modification of the voltage.

Electric actuation. In another advantageous embodiment, said microfabricated elastomer valve having in particular a biconvex shape is actuated using a modification of a voltage applied to the valve portion, in particular an actuation force generated by an electric field. This has the advantage, that no external valves such as solenoid valves are necessary for valve actuation. To this end, the two sides of a biconvex elastomer valve that have direct contact to the actuation channel may be coated with a, in particular thin, electrostatic chargeable polymer layer that enables to charge one side of the elastomer valve positively and the other side negatively (FIG. 7). Thus, an electric field is generated between the two sides of said microfabricated elastomer valve. Applying a voltage to said polymer layers results in an electrical actuation force acting on the membrane separating the connection channel and the actuation channel which closes the connection channel. The main advantage of using an electrical actuation force is the decreased time needed for applying an actuation force which results in a much faster valve operation. If the valve portion is adapted to be selectively opened and closed upon modification of a voltage applied to the valve portion, the valve portion, in particular the membrane, may be a piezoelectric element.

In an especially preferred embodiment, the microfabricated valve comprises at least three layers, wherein the first channel is located within a first layer, the second channel is located within a third layer, the valve portion is located within a second layer and the second layer is arranged between the first and the third layer.

The use of three layers enables manufacturing of a vast number of different microfabricated valves. This increases the design variety and allows designing microfabricated valves according to different process requirements, like mixing of different fluids.

Moreover, this embodiment provides a vast number of possible valve designs and allows to configure the microfabricated valve according to the desired application.

In a preferred embodiment, the first opening is located within the first layer and/or the second opening is located within the third layer. Thereby, it is possible to design the first/second opening independently of the shape of the connection channel. This enables a plurality of different designs. For example, the first/second opening may differ from the connection channel in shape, number and size. In particular, an open end face of the connection channel can be closed by the first/third layer. The part of the layer closing the open end face may at least provide one first/second opening to enable fluid flow from the first channel to the second channel through the connection channel.

In an alternative embodiment, the first opening is located within the first layer and the second opening is located within the second layer or the second opening is located within the third layer and the first opening is located within the second layer.

In this embodiment, the connection channel has an open end face and a closed end face. The first/second opening is inserted into the closed end face of the connection channel. The open end face of the connection channel is closed (except of the section comprising the first/second opening) by the first/third layer.

In a preferred embodiment, the actuation chamber and/or the second actuation chamber is located within the second layer. Especially preferred, the actuation chamber is arranged between the first channel and the second channel. This has the advantage that the microfabricated valve can be design to save-space and a compact design can be realized. It is not necessary to arrange the actuation chamber next to the channels, but between them. Furthermore, it is possible to design a microfabricated valve comprising an actuation chamber which encompasses the connection channel. In this case, the section of the flexible membrane separating the connection channel from the actuation chamber extends over the entire circumference of the connection channel. This has the effect that the force acting onto the connection channel inside the chamber in order to transfer the channel into the closed/opened/intermediate shape is able to act on the entire circumference of the connection channel. This leads to a uniform load of the valve and causes a more reliable operation of the valve. In addition the uniform load decreases the deflection distance, thereby decreasing the required actuation pressure for fully closing the valve.

It is possible that the second layer is arranged in such a way between the first and the third layer or the layers are connected in such a way that it is not recognizable that different layers are present. However, such embodiment is also comprised by the present invention. In particular, the term “layer” at least requires that before connecting the layers or arranging the layers to each other a first, second and third layer must have been present, no matter if that is the case after the arrangement/connection of the different layers.

In an alternative embodiment, the microfabricated valve comprises one layer, wherein the first channel, the second channel, the valve portion and in particular the actuation chamber is located within the layer.

In a preferred embodiment, the flexible membrane comprises an inner boundary forming the outer wall of the connection channel or encompassing at least one section of the connection channel and an outer boundary forming the outer wall of the flexible membrane, wherein the inner boundary is adapted to be transferred between an open and closed shape, and in particular between an intermediate shape, wherein in the open shape a transfer of fluid between the first channel and the second channel through the inner boundary and/or vice versa is enabled and wherein in the closed shape a transfer of fluid between the first channel and the second channel through the inner boundary and/or vice versa is disabled, in particular the inner boundary is adapted to be selectively transferred into an intermediate shape, wherein in the intermediate shape a flow resistance in the valve is increased compared to the open shape.

Especially preferred, the inner boundary is defined by different inner boundary sections, each encompassing a different section of the connection channel, wherein the inner boundary sections are adapted to be transferred between an open and closed shape, and in particular between an intermediate shape.

In a preferred embodiment, the inner boundary sections are adapted to be transferred into an open and/or closed and/or intermediate shape independently.

In a preferred embodiment, the first section of the connection channel is separated from the actuation chamber by the at least first section of the flexible membrane, wherein the first inner boundary section is adapted to be selectively transferred between an opened and closed shape, and in particular into an intermediate shape, upon modification of a pressure difference between the actuation chamber and the first section of the connection channel by modification of the pressure inside the actuation chamber, wherein the pressure inside the actuation chamber is adjusted, in particular by the actuation fluid which can flow into the actuation chamber to increase the pressure inside the actuation chamber or to flow out of the actuation chamber to decrease the pressure inside the actuation chamber, in particular to generate a vacuum inside the actuation chamber.

Especially preferred, the second section of the connection channel is separated from the second actuation chamber by a second section of the flexible membrane, wherein the second section of the flexible membrane and the first section of the flexible membrane are different, wherein the second inner boundary is adapted to be selectively transferred between an opened and closed shape, and in particular into an intermediate shape, upon modification of a pressure difference between the second actuation chamber and the second section of the connection channel by modification of the pressure inside the second actuation chamber, wherein the pressure inside the second actuation chamber is adjusted, in particular by the actuation fluid which can flow into the second actuation chamber to increase the pressure inside the second actuation chamber or to flow out of the second actuation chamber to decrease the pressure inside the second actuation chamber, in particular to generate a vacuum inside the second actuation chamber.

In a preferred embodiment, a first opening connects the first channel with a first section of the connection channel and a second first opening connects the first channel with a second section of the connection channel and/or a first second opening connects the second channel with the first section of the connection channel and a second second opening connects the second channel with a second section of the connection channel. In particular, each of the different section of the connection channel functions in principle similar to the (main) connection channel. It is advantageous that each of these sections (or at least parts of them) connects the first channel and the second channel, in particular without interacting with each other or at least without being in fluid communication to each other. It is preferred that different sections may be actuated by different actuation chambers in order to transfer the different sections of the connection channel into an opened, closed and/or intermediate shape. That opens a vast number of different process applications. For example, different fluids from different channels may be mixed together, taking a specific mixing ratio of the different fluids into account. This is even possible in an extremely small space, like within the connection channel. Depending on the requirements of the process, the first openings may be different in shape and size for instance. It is also possible to provide first openings which are identical. The same applies to the different second openings.

It is preferred that at least a second channel is provided wherein a first second opening connects the second channel with a first section of the connection channel and a second opening connects the second channel with a second section of the connection channel and/or wherein a first opening connects the first channel with the first section of the connection channel and a second first opening connects the first channel with the second section of the connection channel. This embodiment allows mixing a first and a second fluid with a third fluid, however preventing the first and the second fluid being mixed with each other. For example a first fluid is provided within the first second channel, a second fluid is provided within the second channel and a third fluid is provided within the first channel. Now, by opening and closing the corresponding sections of the connection channel it is possible to mix the first fluid with the third fluid or alternatively the second fluid with the third fluid. This is only an exemplary embodiment and it is self-evident that the skilled person may select a different number of channels (for example four or five) and connect them in dependence on the requirements of the process in question.

In a preferred embodiment, the flexible membrane and/or at least one actuation chamber has a homogeneous thickness. Preferably, the flexible membrane or the at least one actuation chamber has an inhomogeneous thickness. In the sense of the invention, the term “thickness of the flexible membrane” means the distance between the inner boundary and the outer boundary of the flexible membrane. In particular, thickness means the shortest distance between a point on the outer boundary of the flexible membrane and a point on the inner boundary of the flexible membrane, both points are on the same plane perpendicular to the longitudinal axis of the connection channel. Accordingly, in the sense of the invention, the term “thickness of the at least one actuation chamber” means the distance between the inner boundary of the actuation chamber and the outer boundary of the flexible membrane. In particular, thickness means the shortest distance between a point on the outer boundary of the flexible membrane and a point on the inner boundary of the actuation chamber, both points are on the same plane perpendicular to the longitudinal axis of the connection channel.

Thus the invention combines the advantages of thicker and thinner structures. Thicker structures, for example, provide stability and thinner structures provide better force transmission. For example, in sections where the actuation pressure of the actuation chamber acts on the flexible membrane, the membrane may have a thinner wall. This allows the membrane to be deformed there by means of a lower force.

In a preferred embodiment, the thickness depends on the deflection distance of the flexible membrane, wherein the deflection distance is the distance of the position of a point on the inner boundary of the flexible membrane while the flexible membrane is in the closed shape and the position of this point while the inner flexible membrane is in the opened position.

Especially preferred the flexible membrane has a thinned section which has a reduced thickness compared to at least one other section of the flexible membrane, in particular this is the thinnest section, wherein the thinnest section is at the position of the maximal deflection distance. The inhomogeneous membrane thickness enables to incorporate a variable stiffness of the membrane which offers several advantages, in particular when the membrane thickness has its thinnest section at the position at which the deflection distance is maximal. Firstly, the deformability at the maximal deflection distance increases due to the decreased membrane thickness resulting in an extensive seal face (instead of a punctate seal face). The extensive seal face leads to an improved sealing. Secondly, the increased membrane thickness at the minimum deflection distance simplifies the movement of the membrane into its initial position (position of the membrane, when no actuation pressure is applied) after the membrane has been actuated. This is caused by the increased tension within the thicker membrane sections and the higher membrane stability.

In a preferred embodiment, the flexible membrane has a thinned section which has a reduced thickness compared to at least one other section of the flexible membrane, this section being the one adjacent to the first layer, and a projection of the first channel along the longitudinal axis of the connecting channel meets this thinned section and/or wherein the flexible membrane has a thinned section which has a reduced thickness compared to at least one other section of the flexible membrane, this section being the one adjacent to the third layer, and a projection of the second channel along the longitudinal axis of the connecting channel meets this thinned section.

This embodiment has the main advantage, that the first/second channel is not or less affected by the deformation of the flexible membrane. The thinned section is preferably the thinnest section.

In a preferred embodiment, the actuation chamber and/or the second actuation chamber has a thinned chamber section which has a reduced thickness compared to at least one other section of the chamber, this section being the one adjacent to the first layer, and a projection of the first channel along the longitudinal axis of the connecting channel meets this thinned chamber section and/or the actuation chamber and/or the second actuation chamber has a thinned chamber section which has a reduced thickness compared to at least one other section of the chamber, this section being the one adjacent to the third layer, and a projection of the second channel along the longitudinal axis of the connecting channel meets this thinned chamber section.

This embodiment has the main advantage, that the first/second channel is not or less affected by the deformation of the flexible membrane. The thinned chamber section is preferably the thinnest chamber section.

In a preferred embodiment the inner boundary or the inner boundary section of the flexible membrane has a biconvex, biconcave shape or a polygonal shape, in particular a triangular, rectangular, pentagonal shape or a shape where at least one edge is curved, in particular convex or concave, for example plano-convex or plano-concave. In an preferred embodiment, the shape may be polygon with curved edge and straight edges. It is also possible, that the shape may be a combination of convex and/or concave and/or biconvex and/or biconcave and/or polygonal with curved and/or straight edges.

In a preferred embodiment, the first channel comprises a positioning means suitable for positioning particles being contained in a fluid which flows through the first channel, wherein the positioning means is arranged within the first channel in such a way that a fluid flow can be reduced by the positioning means and/or the second channel comprises a positioning means suitable for positioning particles being contained in a fluid which flows through the second channel, wherein the positioning means is arranged within the second channel in such a way that a fluid flow can be reduced by the positioning means, in particular, the positioning means narrows the cross section of the channel.

In particular, the positioning means may be a stop that extends from the inner side wall of the channel in the direction of its longitudinal axis, thereby narrowing the cross section of the channel and also the flow rate of the fluid. A particle or a cell in the fluid reaching this stop is prevented from continuing to flow. Thus, the positioning means acts like a trap for cells and particles in the fluid.

In a preferred embodiment, the positioning means is arranged within the first channel in such a way that a projection of the first opening along its axis meets at least a part of the positioning means of the first channel and/or wherein the positioning means is arranged within the second channel in such a way that a projection of the second opening along its axis meets at least a part of the positioning means of the second channel.

This embodiment is particularly advantageous for processes where it is necessary to convey particles or cells through the valve. The particle can thus be trapped near the opening and the valve section only needs to be opened if there is a particle in the positioning means.

The invention also refers to a method for manufacturing a microfabricated valve according to present invention. The method comprises: inserting the first channel into the first layer, inserting the second channel into the third layer, inserting the connection channel with the valve portion into the second layer, and then arranging the second layer between the first layer and the third layer. It is conceivable that the layers can be arranged next to each other in such a way that it is not recognizable that they are different layers, but they appear to be one or two layers.

Preferably, the method may further comprise the step: inserting the actuation chamber and/or the second actuation chamber into the second layer before arranging the second layer between the first layer and the third layer.

On-demand droplet formation. In one embodiment, droplets with defined sizes are generated using said microfabricated elastomer valve. To this end, a first fluid of type 1 is located within the first flow channel and a second fluid of type 2 is located within the second flow channel with the first and the second fluid being immiscible. The generation of droplets with defined sizes comprises the following steps:
Closing said microfabricated elastomer valve by applying an actuation force
Filling the first flow channel with fluid of type 1
Filling the second flow channel with fluid of type 2
Generating a pressure difference between the second flow channel and the first flow channel at the location of the microfabricated elastomer valve with the hydrodynamic pressure within the second flow channel being larger than the hydrodynamic pressure within the first flow channel
Removing the applied actuation force so the connection channel is open
Applying an actuation force again after a period dt which leads to a closing of the connection channel
Repeating this process

For example, fluid of type 1 might be a fluorinated oil (e.g. HFE-7500 (Novec)) or FC40 and fluid of type 2 might be an aqueous phase. Due to the pressure difference between the second flow channel and the first flow channel, the fluid of type 2 enters the first flow channel upon opening of the connection channel and an interface between fluid of type 1 (fluorinated oil) and fluid of type 2 (aqueous phase) is formed as both fluids are immiscible. Afterwards, an actuation force is applied again within the actuation channel and a droplet is pinched off due to the closing of the connection channel. Said droplet (now located within the first flow channel) might be transferred to another position by applying a fluid flow within the first flow channel. The main advantage of this method in comparison to other droplet generators is that no surfactant is needed for droplet generation which reduces costs and decreases the risk of affecting cell viability when cells are handled. In addition, the droplet size can be precisely controlled by adjusting the pressure difference between the first flow channel and the second flow channel and/or by adjusting the opening time of the described microfabricated elastomer valve.

Droplet generation with highly controlled hydrodynamic resistance. In another advantageous embodiment shown in FIGS. 52a and 52b, at least one elastomer valve arrangement 60 might be used for generating a droplet as described previously whereas the droplet is generated within a droplet collection channel 61 having a first opening 62A at a first end, a second opening 62B at a second end and a third opening 62C in the first and second opening. The first opening 62A might be closed by actuating a first elastomer valve 63A, the second opening might be closed by actuating a second elastomer valve 63B and the third opening might be closed by actuating a third elastomer valve 63C. Each valve can be closed and opened by changing a pressure within the corresponding activating channels 67A, 67B, 67C. The first and the second opening 62A, 62B might be connected with a first and second channel 64A, 64B containing for example an oil phase such as a fluorinated oil (e.g. HFE-7500, FC-40). Initially, the droplet collection channel 61 contains the same oil phase. The third opening 62C is connected through a passage 69 to a third channel 64C containing for example an aqueous phase or a cell/particle suspension. A water-in-oil droplet is generated by opening the third elastomer valve 63C for a defined period as described previously. During the droplet formation, the first and the second openings 62A, 62B are closed by actuating the first and second elastomer valve 64A, 64B. The collection channel 61 has now a high hydrodynamic resistance that solely depends on the elastomer and its mechanical characteristics (e.g. elasticity) which has been used for fabricating said microfabricated geometry. A volume flow of fluid from the third channel towards the droplet collection channel depends now solely on the applied pressure and the capability of the used channel material to deform. This has the advantage that the droplet formation process is decoupled from any changes of the hydrodynamic resistance downstream of the droplet generation process. For example, these changes might occur when the number of already formed droplets is changed downstream of the droplet formation process as a droplet itself might change the hydrodynamic resistance of a channel due to any adhesive forces acting between the droplet and the channel wall. Thus, the spatial isolation of the droplet generation process results in much more monodisperse droplets when all droplets are formed with the same pressure and opening time. The droplet collection channel 61 may be a part of the feeding channel 41 as described in another area of the description.

Droplet generation with highly controlled hydrodynamic resistance—Membrane structure for controlling droplet volume. In another advantageous embodiment, the droplet collection channel of the arrangement as described above is connected to a damping device (FIG. 52b), having one or more membrane structures 65 including one or more membrane 66, which can deflect upon applying a pressure within said droplet collection channel 61. Said membrane structure might be fabricated from the same elastomer that is used for fabricating said elastomer valves (e.g. PDMS (Sylgard 184)) and/or on one piece with the hosing 610 of the valve arrangement. The membrane 66 might have a thickness between 1 μm and 120 μm. On the other side of the membrane 66 a compensation chamber 68 is provided. In the compensation chamber 66 a compensation pressure p10 may be applied to the membrane which can be the atmospheric pressure.

During generating a droplet an amount of liquid forming the droplet is pressed from the liquid third channel (the liquid supply channel) 64C through the third valve 63C into the droplet collection channel 61 increasing the volume in the droplet collection channel 61. By allowing the membrane 66 to deflect the difference in volume can be equalized without significantly increasing the pressure within the droplet collection channel 61. One of more of the valves can be designed in a manner as described within other areas of the description. In general a pressure difference between the liquid supply channel and the droplet generation channel may be max 1 bar, in particular max 0.5 bar.

In another embodiment a valve as described with in other areas of the description, in particular the first or second valve, as can be selectively opened to allow an amount of fluid flowing out of the droplet collection channel, for equalizing the amount of fluid flowing from the liquid supply channel into the droplet generation channel.

In another advantageous embodiment, which may be combined with the embodiment as previously described, the droplet collection channel/chamber 61 exhibits at least one area/section which is separated from at least one fluid reservoir 68 (pressure damping chamber) by at least one membrane 66 that can deflect upon applying a pressure difference between the droplet collection channel/chamber 61 and said fluid reservoir 68 (FIG. 52b). In a particular embodiment, the fluid reservoir contains a fluid that can be pressurized. In one embodiment, the pressure applied to said fluid is atmospheric pressure. As soon as a pressure difference between the droplet collection channel/chamber and the fluid reservoir is present, the separating membrane starts to deform and an elastic force is generated that has an opposite direction towards the force generated by the pressure difference. The magnitude of the elastic force is proportional to the deflection distance. Said elastic force depends on the material properties of the material used for the membrane section (e.g. the elastic modulus) as well as on the membrane geometry (e.g. membrane thickness, membrane shape) and the number of separating membrane sections. Said membrane deflects until the force generated by the pressure difference and the elastic force with opposite direction are in equilibrium. This has the advantage that the deflection distance can be precisely regulated by either changing said pressure difference or by changing the material properties and/or membrane geometry or by changing the fluid characteristics of the fluid located within the pressure damping chamber.

In a particular embodiment, said pressure damping chamber is used for the generation of highly monodisperse droplets with a defined droplet volume. To this end, droplets are formed as described with at least one of more selected from the following:
1. The first, the second and the third opening are closed by actuating the first and second elastomer valve.
2. The droplet collection channel contains a first fluid (e.g. fluorinated oil) that is immiscible with a second fluid (e.g. an aqueous fluid) located within the third channel.
3. The droplet collection channel exhibits at least one area that is separated from a pressure damping chamber by a membrane with defined characteristics.
4. A first pressure p1 is present within the third channel containing the second fluid.
5. A second pressure p2 is present within the droplet collection channel.
6. A third pressure p3 is present within the pressure damping chamber.
7. In a particular embodiment, the pressure p2 and pressure p3 are equal with p3 being atmospheric pressure and p1>p3.
8. The third elastomer valve is opened.
9. The second fluid is now entering the droplet collection channel and the deflectable membrane separating the droplet collection channel from the pressure damping chamber starts to deflect towards the pressure damping chamber.
10. The second fluid enters the droplet collection channel until a force equilibrium between the force generated by the pressure difference p1-p3 and the elastic force caused by the membrane deflection is reached. At this point the flow of the second fluid towards the droplet collection channel stops and a defined volume of the second fluid has entered the droplet collection channel. Said defined volume of the second fluid solely depends on the pressure difference p1-p3 and the properties of the deflectable membrane separating the droplet collection channel and the pressure damping chamber.
11. The third elastomer valve is closed.
12. A droplet having a defined volume is generated as the first fluid and the second fluid are immiscible
13. The first and the second elastomer valves are opened.
14. The droplet is removed from the droplet collection channel by applying a flow of the first fluid.
15. Repeat the process several times starting with step 1.

The use of the described pressure damping chamber for generating droplets has the advantage, that the droplet volume can be highly controlled by the pressure difference p1-p3 as well as by the properties of the deflectable membrane separating the droplet collection channel and the pressure damping chamber. In addition, the pressure p1 can be significantly reduced in comparison to a droplet collection channel/chamber without a pressure damping chamber.

In another advantageous embodiment, the first fluid and the second fluid are miscible. Thus, the pressure damping chamber can be used to inject a defined fluid volume which might be done in a highly repeatable manner.

In one advantageous embodiment of the present disclosure, the membrane section separating the droplet collection channel and the pressure damping channel has a round shape with a diameter of 50 μm, 100 μm, 150 μm. In a particular embodiment, the membrane section has a thickness of 10 μm, 20 μm, 30 μm.

In another advantageous embodiment, one pressure damping chamber 65 has multiple membrane sections 66. In a particular embodiment, said membrane sections have a round shape. Thus, each membrane section deflects independently from the other membrane sections. For a given injection volume, the deflection distance of said membrane sections decreases if the number of membrane sections per pressure damping chamber increases. The total deflection and thus the total injection volume is then a function of the deflection of all membrane section. The use of multiple membrane sections has the advantage, that the deflection distance of single membrane sections can be decreased for a given injection volume thereby also decreasing the time needed until a force equilibrium is reached. Thus, the use of multiple membrane sections increases the injection speed. This is critical in terms of the generation of droplets as a high droplet generation frequency is desirable. Thus, the advantage of using multiple membrane sections is an increase of the droplet generation frequency.

Parallel droplet generation. In another advantageous embodiment, droplets are generated with a very high frequency in a parallel manner. To this end, multiple microfabricated elastomer valves are actuated by the same actuation channel with the connection channel connecting the first flow channel with the space above the second microfabricated layer. The first flow channel contains fluid of type 1 and the space above the second microfabricated layer contains a fluid of type 2. A pressure is applied to the fluid of type 2. Droplet formation is done as described previously. The described footprint of the microfabricated elastomer valve might be below 0.02 mm². An area of 100 mm² might thus contain 5000 microfabricated valves that can be actuated simultaneously. If the actuation time is in the range of 20 ms, the droplet generation frequency is in the range of 250 kHz. With an area of 400 mm² even a droplet generation frequency of 1 Mhz might be achieved. Thus, one main advantage of the present disclosure is the high degree of parallelization and the corresponding number of droplets that can be generated in a very short period.

Droplet mixing. In another advantageous embodiment, the disclosure is used for the mixing of two droplets. To this end, a first microfabricated elastomer valve and a second microfabricated elastomer valve share a common first flow channel. In addition, the first microfabricated elastomer valve connects the common flow channel with a second flow channel containing a fluid of type 1. The second microfabricated elastomer valve connects the common flow channel with a different second flow channel containing a fluid of type 2. In the next step, two droplets are formed as described previously. The first common flow channel contains now two droplets, one having as droplet content the fluid of type 1 and a second having as droplet content the fluid of type 2. Due to the localization of the microfabricated elastomer valve, the two droplets are arranged in sequence. After applying a flow within the first common flow channel, the droplets might get into contact due to an increase of the width of the flow channel and droplet coalescence occurs as no surfactant is used for droplet formation which might prevent droplet fusion. This has the advantage, that two droplets with different droplet content can be mixed in a controlled and programmable manner with an adjustment of the compound concentration which depends on the droplet size of the two individual droplets and the droplet that is generated after mixing both droplets. This process can also be done for generating and mixing more than two droplets.

Hydrogel formation. In another advantages embodiment, the present disclosure is used for the generation of spherical or plug-like hydrogel matrices that are produced using the previously described method for droplet generation and the mixing of two droplets. To this end, one droplet is generated as described previously containing a compound A that might be a hydrogel precursor. A second droplet is generated containing compound B that might initiate the cross-linking of compound A. After generating and mixing said droplets containing compound A and compound B, a spherical hydrogel matrix is formed within the mixed droplet. This has the advantages, that hydrogel matrices with different compositions and characteristics can be produced in a programmable manner. For example, the mechanical strength of said hydrogel matrices might be varied by changing the droplet size of the droplet containing compound B and thus by changing the final molar ratio between compound B and compound A present in the fused droplet. In another embodiment, three droplets might be mixed, one containing compound A, one containing compound B and a third droplet that contains a certain compound C (e.g. proteins such as antibodies, growth factors or ECM proteins; nucleic acids such as DNA primers) which is immobilized within the hydrogel matrix.

Hydrogel properties. In one advantageous embodiment, the hydrogel might be composed of a mix of at least two different polymers and/or copolymers with different structures. At least one of these polymers has a linear structure and at least one polymer has a multiarm or star-shaped structure. Combining these different structures in varying concentrations and/or molecular weights enables defined control over hydrogel matrix size, composition and matrix stiffness. The formed hydrogel matrices have preferably a spherical form in the micrometer or sub-micrometer scale and are considered as discrete, crosslinked hydrogel matrices made of polymers and copolymers exhibiting different structures. The polymers are composed of heterocyclic chemical compounds preferably 2-oxazolines substituted only at position 2 and unsaturated imides preferably 3-(maleimido)-propionic acid N-hydroxysuccinimide ester or alkenyl groups such as isopropenyl groups. A scheme of the architecture of the hydrogel matrices is shown in FIG. 14. The hydrogel matrices are formed by cross-linking hydrogel precursor molecules of the same type or of different types. The backbone of the polymers is formed by preferably hydrophilic peptide-like polymers such as Poly-2-methyl-2-oxazoline (PMOx)-based polymers, most preferably linear and multiarm Pox-based polymers that are crosslinked by cell-compatible crosslinking reactions (Table 1). These polymers are pseudo-peptides with a high biocompatibility and show structural similarities to naturally occurring polypeptides. The polymer is formed by living cationic ring-opening polymerization (LCROP) of oxazolines substituted at position 2. In an advantageous embodiment of this disclosure unsaturated imides preferably 3-(maleimido)-propionic acid N-hydroxysuccinimide ester and/or alkenyl group preferably isopropenyl-group carrying molecules are incorporated during the CROP to form copolymers. In one example, the LCROP might be initiated by an initiator and oxazoline monomers by heating to 75° C. in acetonitrile or by microwave technology. The living polymer is terminated by addition of a terminator. One advantages of the CROP of 2-oxazolines in terms of synthesis are the high degree of polymerization control, the resulting well-defined polymeric structures and the large variety of end- and side-group functionalities, which can be introduced using appropriate initiators/terminating agents and substituted monomers, respectively. The modularity of this polymer class enables the synthesis of highly functional materials with tailormade properties. Scheme 14 illustrates the mechanism of the polymerization and the incorporation of functional molecules for cell culture and cell analysis. In total four classes of molecules are needed for the CROP: Initiators for initiation of the reaction preferably with an electrophilic character, heterocyclic chemical compounds as monomers for the polymer backbone, unsaturated imides and/or alkenyl groups for functionalization of the polymer backbone and terminating agents for terminating the living polymer.

The initiators used for the CROP to produce polymers for the fabrication of said array consist of an organic moiety with an attached leaving group, which acts as the counter ion for the oxazolinium species during polymerization. The initiators used are chosen from a group of different tosylates, triflates or alkyl halides of small aliphatic molecules or small PEGs. Most preferably bifunctional initiators such as triethylene glycol di(p)-toluenesulfonate are used for the synthesis of linear polymers. In this case both sides of the living polymer can be terminated by the same species of terminating molecules leading to homo-bifunctional linear polymers. Alternatively, the nature of the initiator can be altered to synthesize hetero-bifunctional linear polymers with a functional group F1 incorporated by the initiator and a functional group F2 incorporated by the terminating molecule. The terminating molecules are chosen from a group of nucleophiles, amines, azides or acids especially carboxylic acids. The functional groups F1 and F2 are suitable for cell-compatible crosslinking reactions (Table 1). Combining these different synthesis strategies for linear polymers lead to a variety of possible structures. For the multiarm polymers initiators used are chosen from a group of different multi-tosylates, -triflates or -alkyl halides of small aliphatic molecules or small PEGs. Most preferably multifunctional initiators such as pentaerythritol tetrabromide, pentaerythritol tetrakis(benzenesulfonate) or p-toluenesulfonyl chloride modified N,N,N′,N′-Tetrakis(2-hydroxyethyl)ethylenediamine are used for the synthesis of multiarm polymers.

Another advantage of the disclosure is the variation of the monomer substitution in the 2-position of the heterocyclic molecule for both linear and multiarm polymer synthesis. This group does not directly influence the polymerization reaction given that the nucleophilicity of the used molecule is low enough. Substitution in the 2-position are chosen from a group of alkynes, alkenes or protected amine groups. The substitution in the 2-position modulates the relevant chemical and physical properties of the whole polymer. With aromatic or long carbohydrates as side-groups, the polymer becomes hydrophobic, whereas short aliphatic chains lead to a hydrophilic character of the polymer. In addition to the solubility the critical temperature can be fine-tuned. The substitution in the 2-position is restricted to biochemical inert molecules because incorporation of functional groups at the 2-position interferes with the polymerization reaction resulting in premature termination reactions of the CROP. To overcome this circumstance the copolymerization of unsaturated imides such as 3-(maleimido)-propionic acid N-hydroxysuccinimide ester or alkenyl groups such as isopropenyl with heterocyclic chemical compounds like 2-oxazolines is part of this disclosure. The incorporation of functional groups such as maleimido-derivates or alkenyl-derivates offers more freedom in the generation of functional polymers for cell culture and cell analysis by creating statistical or random copolymers. The combination of several (functional) monomers during a well-defined CROP together with different types of initiator and terminator molecules lead to an enormous diversity in size, structure and physicochemical properties of possible structures. A second advantage of incorporating functional groups by copolymerization is that the amount of functional groups per polymer chain is only limited by the degree of polymerization. This leads to a highly modular and tunable system that allows defined control over hydrogel matrix size, composition and matrix stiffness to modulate relevant bio-chemical mechanical and physical properties. The high modular system is advantageous over existing hydrogel materials such as PEG regarding cell cultivation of single cells and small colonies and analysis of single cells and small colonies encapsulated within these hydrogel matrices. In addition, the hydrogel matrices might be used as carriers/vehicles for positioning of cells within microfabricated structures on the disclosed microfluidic array. The hydrogel matrices ensure the precise transport of cells within a hydrodynamic flow on a microfluidic array enabling cell immobilization, cell pairing and cell recovery from the microfluidic array. In addition the hydrogel matrices can be used as drug delivery devices for cell based drugs such as genetically-modified immune cells for novel therapies. In summary, the hydrogel matrices overcome the drawbacks of other hydrogel materials regarding to biocompatibility, adjustability or versatility. Thus, they are perfectly suited for biomedical research and cell based drug-development.

Hydrogel cross-linking. The said hydrogel, wherein the hydrogel matrices are built up from precursor molecules that are cross-linkable by cell-compatible reaction or by combination of multiple cell-compatible reaction(s), based on:

### Table 1:

covalent bond formation chosen from the group consisting of:
   enzymatically catalyzed reactions
      transglutaminase factor XIIIa
   not-enzymatically catalyzed
      click chemistry
      photo-catalyzed
   uncatalyzed reactions
      Copper-free highly selective click chemistry
      Michael-type addition
      Diels-Alder conjugation
non-covalent bond formation:
   Hydrogen bonds formed by:
      Nucleic acids
   hydrophobic interactions
   Van-der-Waals
   Electrostatic interactions

In another embodiment, the hydrogel matrices are built up from precursor molecules that are cross-linkable by hydrogen bonds formed by peptide nucleic acids.

Peptide nucleic acids (PNAs) are artificially synthetic homologs of nucleic acids in which the phosphate-sugar polynucleotide backbone is replaced by a pseudo-peptide polymer to which the nucleobases are linked. This structure leads to uncharged polymer backbone in contrast to the negatively charged phosphate-sugar polynucleotide backbone of natural nucleic acids. The uncharged polymer backbone has stealth characteristics which is very import in terms of cell culture and cell analysis. Hydrogels built up by polymers with stealth characteristics ensure that only effects of the functionalization are measured during investigation. Compared to natural nucleic acids PNAs have the ability to hybridize with high affinity and specificity to complementary sequences nucleic oligomers. The hybridization energy of PNA/DNA or PNA/RNA hybrids are higher than the hybridization energy between two natural nucleic acids with the same nucleobases resulting in a higher biding strength. Because a mismatch in a hybridized duplex is more destabilizing, the PNA oligomers also have a greater specificity in binding to complementary oligomers. In addition, PNAs are more stable than natural nucleic acids because they are resistant to degradation by DNAses, proteinases and pH shifts.

The formation of hydrogel matrices by PNA hybridization has several advantages compared to other established crosslinking reactions. One first advantage is the avoidance of any catalysts in form of copper, sodium ascorbate, triethanolamine and UV exposure which might influence cells incorporated into the hydrogels. These unknown influences often lead to artificial and misleading results. A second advantage is the fast gelation procedure. The PNA oligomers located at the ends of the polymers can hybridize within several minutes. A third advantage is the orthogonal mechanism of the gelation process. Different polymer precursor molecules i.e. linear polymers and multiarm polymers possess complementary PNA oligomers. Thus, only different polymer precursor molecules can hybridize leading to a perfectly defined alternating structure of linear polymers and multiarm polymers (FIG. 16). This ensures that the mechanical and biochemical properties are exactly the same at any given location within the hydrogel matrices. Another advantage is that the cross-linking reaction does not compete with reactions for the incorporation of bioactive molecules. Thus, the hydrogel formation is independent on the concentration of incorporated bioactive molecules. In addition, this procedure allows the incorporation of bioactive molecules after hydrogel formation by adding bioactive molecules to a liquid which flows through the formed hydrogel. A further advantage is the conjugation of PNAs to peptides. The synthesis of PNAs oligomers is compatible with peptide synthesis. Thus, peptides can be easily added to the growing PNA oligomer at the C′-terminus during synthesis. Alternatively, further PNA monomers can be added to the C′-terminus of a peptide resulting in a C′-terminus modification of the PNA oligomer. The used molecules are block copolymers composed of a peptide bearing a proteinase target site and a PNA oligomers.

Matrix remodeling with MMPs. A further advantage is the simple degradation of the formed hydrogel for the release of cells and analytes by applying moderate heat to the hydrogel matrices. Alternatively, the addition of PNA oligomers in excess can be used for degradation of the hydrogel matrices. Therefore, the initial PNA oligomers used for the hydrogel formation possess mismatches which lead to a decreased complementarity and thus to a decreased hybridization energy compared to the PNA oligomers used for hydrogel degradation. Alternatively, the incorporated proteinase target site can be used to degrade the hydrogel matrices. In summary, these strategies ensure a fast and cell-compatible degradation of the hydrogel matrices. Because the cells are not affected by this procedure, further molecular analysis of the native state of the cells are possible.

Incorporation of capture molecules into hydrogel matrices. Incorporation of capture molecules into said array (oxazoline-based hydrogel matrices) is implemented by reaction(s), based on:
covalent bond formation chosen from the group consisting of:
   enzymatically catalyzed reactions
      transglutaminase factor XIIIa
   not-enzymatically catalyzed
      click chemistry
      photo-catalyzed
   uncatalyzed reactions
      Copper-free highly selective click chemistry
      Michael-type addition
      Diels-Alder conjugation
non-covalent bond formation:
   Hydrogen bonds formed by:
      Nucleic acids
   hydrophobic interactions
   Van-der-Waals
   Electrostatic interactions

Preferably, incorporation of capture molecules into said array (oxazoline-based hydrogel matrices) is implemented by peptide nucleic acids. PNA oligomers are incorporated by amide bond formation between the NHS-ester from the hydrogel precursor molecule and the primary amine of a PNA oligomer. The capture molecule is fused to a complementary PNA oligomer. The fusion product is then immobilized by hydrogen bond formation between the two PNA oligomers. The capture molecule can be removed by addition of a molar excess of complementary PNA oligomers. The complementary PNA oligomers compete with the PNA/capture fusion product. Alternatively, the capture molecule is fused to a complementary modified PNA oligomer. The modification comprises of a photo-cleavable linker between two PNA molecules. After hydrogen bond formation between the two PNA oligomers, the capture molecule can be easily removed by UV irradiation. In both cases the capture molecule comprises a small molecule, an antigen, an antibody, a protein binding domain, a nucleic acid, a polysaccharide or an aptamer. Preferably, the target molecule is identified by an identification molecule. This identification molecule is a fusion molecule between a capture molecule and a nucleic acid oligomer with a target specific sequence. The capture molecule comprises a small molecule, an antigen, an antibody, a protein binding domain, a nucleic acid, a polysaccharide or an aptamer. The binding partner (target molecule) of the capture molecule can be analyzed directly within the hydrogel matrices or after separation of the capture molecule by said strategies. This procedure enables a time-lapse cytokine profiling of single cells or of small colonies.

Hydrogel formation. In another advantages embodiment, the present disclosure is used for the generation of spherical or plug-like hydrogel matrices that are produced using the previously described method for droplet generation and the mixing of two droplets. To this end, one droplet is generated as described previously containing a compound A that might be a multiarm hydrogel precursor. A second droplet is generated containing compound B that might be a linear hydrogel precursor. Optionally, a third droplet is generated to initiate the cross-linking of compound A with compound B. After generating and mixing said droplets containing compound A, compound B and the cross-linking agent, a spherical hydrogel matrix is formed within the mixed droplet. This has the advantages, that hydrogel matrices with different compositions and characteristics can be produced in a programmable manner. For example, the mechanical strength of said hydrogel matrices might be varied by changing the droplet size of the droplet containing compound B and thus by changing the final molar ratio between compound B and compound A present in the fused droplet. In another embodiment, four droplets might be mixed, one containing compound A, one containing compound B one containing a crosslinking agent C and a fourth droplet that contains a certain compound D (e.g. proteins such as antibodies, growth factors or ECM proteins; nucleic acids such as DNA primers, peptide nucleic acids such as PNA oligomers)) which is immobilized by a stable amide bond within the hydrogel matrix.

Gel-Shell matrix formation. In another advantages embodiment, the present disclosure is used for the generation of spherical or plug-like hydrogel matrices that are surrounded by defined gel-shells. They are produced using the previously described method for droplet generation and the mixing of multiple droplets. To this end, one droplet is generated as described previously by fusion of multiple droplets. The gel shell might be formed by one of the following strategies:
1. The previously formed hydrogel matrix located within a first droplet A is fused with a second droplet B containing a polymer which comprises primary amines such as poly allylamine polymers. The fusion of droplet A with droplet B results in a larger droplet C containing said hydrogel matrix with the volume of the hydrogel matrix being smaller than the volume of the droplet C. Within droplet C, said hydrogel matrix is surrounded by said polymer from droplet B and a crosslinking of the hydrogel polymers at the edge of the hydrogel matrix occurs as said polymer from droplet B diffuses into the hydrogel matrix. The diffusion of said polymer into the hydrogel matrix might be limited by the molecular weight of said polymer. Alternatively, the droplet B might contain primary amine bearing polymer molecules such as poly allylamine and small primary amines such as 3-Amino-1,2-propanediol with the polymer molecule having a smaller diffusion coefficient than the small primary amine. Thus, the primary amine diffuses faster into said hydrogel matrices than the polymer molecule. This results in a thinner shell as the small primary amine diffuse into said hydrogel matrix thereby blocking the NHS-esters of the hydrogel matrix. The polymer molecule (such as poly allylamine) can then only react with marginal unreacted NHS-esters. Preferably the small primary amines are added with a short delay after the poly allylamine polymers.
2. The previously formed droplet A containing said hydrogel matrix is fused with a second droplet B containing the previously described copolymer with an oxazoline backbone and incorporated NHS-esters resulting in a larger droplet C. Said droplet C might be fused with a droplet D containing small diamines such as 2,2-Dimethyl-1,3-propanediamine or 1,5-Diaminopentane. This fusion leads to marginal crosslinking reaction between the two copolymers added by droplet D and the hydrogel matrix located within droplet C. Alternatively, are previously mixed together with a molar excess of primary amine groups.
3. As an alternative, the previously formed hydrogel matrix located within droplet A might be trapped within a microfabricated trapping geometry while being surrounded by an oil phase. Afterwards, the hydrogel matrix might be perfused with a hydrophilic phase for demulsification. Primary amine containing polymers such as poly allylamine or poly oxazoline are added to the hydrophilic phase. This leads to a marginal crosslinking reaction between primary amines and NHS-esters within the hydrogel matrix.
4. As an alternative, the previously formed hydrogel matrix located within droplet A is placed below the previously described elastomer-valve. Primary amine containing polymers such as poly allylamine or poly oxazoline are added to the hydrophilic phase above the closed elastomer valve. As soon as the elastomer valve is opened said hydrogel matrix moves towards the hydrophilic phase driven by the density gradient between the oil phase and the hydrophilic phase. Within the hydrophilic phase a marginal crosslinking reaction between primary amines and NHS-esters within the hydrogel matrix takes place.
5. In another embodiment, a hydrogel matrix might be generated, demulsified and trapped as described previously. Afterwards, trapped hydrogel matrices might be perfused with a defined amount of fluid containing primary amine bearing polymer molecules such as poly allylamine and for a defined period. Thus, only a limited amount of said primary amine bearing polymer molecules diffuses into the hydrogel matrix and reacts with the hydrogel backbone.

Cell encapsulation into droplets. In another aspect, the present disclosure relates to methods for the encapsulation of single or multiple cells of the same or of different types in droplets or hydrogel matrices.

Poisson distributed cell encapsulation. In one embodiment, droplets are produced on-demand as described previously with a fluid of type 1 located within the first flow channel and a fluid of type 2 located within the second flow channel. Fluid of type 1 and fluid of type 2 are immiscible. Fluid of type 2 is a cell suspension with a defined concentration. The subsequent on demand-formation of droplets results in the encapsulation of cells within said droplets. Encapsulated cells are Poisson distributed within formed droplets. The main advantage is that cells can be encapsulated at a high frequency which is necessary for performing high-throughput biological experiments.

High efficiency single cell encapsulation. In another advantages embodiment, the encapsulation of single cells into droplets is performed with a very high efficiency (exactly one cell per droplet) by using a microfabricated geometry for the trapping of single cells which is located above the described microfabricated elastomer valve. The microfabricated geometry for trapping of single cells is thus located within the second flow channel. The high efficiency encapsulation of single cells into droplets comprises the following steps:
Closing the described microfabricated elastomer valve by applying an actuation force
Filling the first flow channel with fluid of type 1
Filling the second flow channel with a cell suspension located in a fluid of type 2 that is immiscible with the fluid of type 1
Immobilizing single cells within a microfabricated geometry for hydrodynamic cell trapping that is located directly above an elastomeric valve
Optionally washing away single cells that have not been trapped by perfusing the second flow channel with a fluid of type 3 that does not contain any cells
Generating a pressure difference between the second flow channel and the first flow channel at the location of the microfabricated elastomer valve with the hydrodynamic pressure within the second flow channel being larger than the hydrodynamic pressure within the first flow channel
Removing the applied actuation force so the connection channel is open
Applying an actuation force again after a period dt which leads to a closing of the connection channel
Repeating this process

This method has the main advantage that exactly one cell is encapsulated in one droplet resulting in a highly efficient encapsulation of single cells.

High efficiency co-encapsulation. In another aspect, the present disclosure relates to microfabricated structures and methods for the co-encapsulation of a first cell/particle with a second cell/particle into droplets and/or hydrogel matrices with defined compositions and with high encapsulation efficiency. To this end, a third microfabricated layer is fabricated that contains a microfabricated geometry for the spatial immobilization of two cells/particles that might be of different type in close proximity and that is located within the second flow channel. In this embodiment, the second flow channel is composed of two individual channels, a first channel for a cell/particle suspension of type 1 and a second for a cell/particle suspension of type 2. The microfabricated geometry for immobilization of two cells/particles in close proximity might be a hydrodynamic trap that is directly located above a microfabricated elastomer valve as described previously (FIG. 10). The method for the co-encapsulation of a cell/particle of type 1 with a cell/particle of type 2 comprises the following steps:
Closing the described microfabricated elastomer valve by applying an actuation force
Filling the first flow channel with fluid of type 1
Filling a first channel of the second flow channel with a cell/particle suspension of type located in a fluid of type 2 that is immiscible with the fluid of type 1
Filling a second channel of the second flow channel with a cell/particle suspension of type 2 located in a fluid of type 3 that is immiscible with the fluid of type 1
Immobilizing single cells/particles within a microfabricated geometry for the hydrodynamic trapping of two different cells/particles of different type in close proximity that is located directly above an elastomeric valve
Optionally washing away single cells/particles that have not been trapped by perfusing the second flow channel with a fluid of type 4 that does not contain any cells/particles
Generating a pressure difference between the second flow channel and the first flow channel at the location of the microfabricated elastomer valve with the hydrodynamic pressure within the second flow channel being larger than the hydrodynamic pressure within the first flow channel
Removing the applied actuation force so the connection channel is open
Applying an actuation force again after a period dt which leads to a closing of the connection channel
Repeating this process

With currently existing methods, a co-encapsulation of one cell/particle with a second cell/particle results in a double Poisson distribution. Thus, only a low percentage of droplets contain one cell/particle of type 1 and a second cell/particle of type 2. Thus, the presented microfabricated geometries and method has the main advantage that a co-encapsulation can be performed with a very high efficiency resulting in a high percentage of droplets containing exactly one cell/particle of type 1 and a second cell/particle of type 2.

Parallelization of single cell encapsulation. In another advantages embodiment, the encapsulation of a single cell/particle is performed in a parallel manner resulting in a dramatic increase of encapsulation speed. To this end, multiple microfabricated elastomer valves are located below multiple microfabricated geometries for the spatial immobilization of one cell/particle. The hydrodynamic pressure at the trapping position is the same for all microfabricated traps so cells/particles can be encapsulated into highly uniform droplets in a parallel manner. Droplet formation and parallelization is performed as described previously. Thus, the main advantage of parallelizing the single cell encapsulation is the significantly reduced time needed for encapsulation of single cells/particles into droplets and the highly-increased encapsulation frequency.

Parallelization of co-encapsulation. In another advantages embodiment, the co-encapsulation of a first cell/particle with a second cell/particle is performed in a parallel manner resulting in a dramatic increase of encapsulation speed. To this end, multiple microfabricated elastomer valves are located below multiple microfabricated geometries for the spatial immobilization of two cells/particles of the same or of different time. The hydrodynamic pressure at the trapping position is the same for all microfabricated traps so cells/particles can be encapsulated into highly uniform droplets in a parallel manner. Droplet formation and parallelization is performed as described previously. Thus, the main advantage of parallelizing the co-encapsulation is the significantly reduced time needed for co-encapsulation of cells/particles into droplets and the highly-increased encapsulation frequency.

Encapsulation of cells into hydrogel matrices. In another advantageous embodiment, cells/particles might be encapsulated into spherical or plug-like hydrogel matrices with defined characteristics. To this end, cells might be first encapsulated into a first droplet with a defined size using the methods described previously (such as Poisson encapsulation of cells/particles, encapsulation of single cells/particles or co-encapsulation of cells/particles) whereas cells might be located within a fluid of type 1 that contains a hydrogel precursor molecule a at a defined concentration. A second droplet with a defined size might be generated in parallel or sequential as described previously. This second droplet might contain a fluid of type 2 that contains a hydrogel precursor molecule b with a defined concentration. Afterwards, the formed droplets—one containing one or multiple cells/particles and hydrogel precursor a and one containing hydrogel precursor b—might be fused as described previously. The fusion of said droplets results in a larger droplet that contains now the hydrogel precursor molecules a and b. Afterwards, the hydrogel formation might occur due to the mixing of said hydrogel precursor molecules. This has the advantage, that the concentration of the hydrogel precursor molecules can be fine-tuned by changing the sizes of the first droplet and the second droplet while mainlining the final size of the hydrogel matrix.

In another advantageous embodiment, either the first or the second droplet might contain additional compounds such as biological active molecules (e.g. antibodies) that are immobilized within the hydrogel matrix before or during the hydrogel formation. This has the advantage, that biological compounds or bioactive compounds might be immobilized within said hydrogel matrices during hydrogel matrix formation.

In another advantageous embodiment, hydrogel formation might be initiated by changing the surrounding temperature or by irradiating said droplets with UV-light for hydrogel formation.

Particle centering. The spatial position of cells within hydrogel matrices is of utmost importance, as cells located close to the edge of a hydrogel matrix tend to escape from said hydrogel matrix during cell proliferation and/or migration. Escaped cells are hardly accessible any more for further analysis as the hydrogel matrix acts among other as vehicle for the cell transport. In addition, escaped cells lose the biological and physical information provided by the three dimensional microenvironment established within said hydrogel matrices. To reduce the amount of escaping cells and ideally to prevent cell escape, a positioning of cells in particular and particles in general is necessary. Especially, when biological cells have to be cultivated and analyzed for several days, as centering of cells within the center of hydrogel matrices has been reported to prolong successful cultivation periods.

Thus, in still another aspect, embodiments of this disclosure provide methods for the centering of single cells/particles within the center of droplets and hydrogel matrices. To this end, a droplet containing one or more cells/particles is positioned within a microfabricated geometry, in which the droplet is perfused with a fluid that results in droplet rotation (FIG. 12). In one particular embodiment the droplet content might have a lower density than the surrounding fluid, thus the droplet experiences a buoyant force tending upwards. The hydrodynamic pressure below said droplet might be higher than the hydrodynamic pressure above the droplet due to the used microfabricated geometry (FIG. 12a). Said process comprises the following steps:
Trapping said droplet within a microfabricated geometry in which said droplet stays at a certain position and in which the trapped droplet is perfused in a way that the droplet starts to rotate
Rotating the droplet for a defined time dt and with a defined rotation speed which results in a centering of the particle located within the droplet.
Generating a hydrogel during or after droplet rotation. Said hydrogel might be formed by a polymerization reaction. In a particular embodiment, the hydrogel formation might be initiated/controlled by adjusting one of the following parameter:
   Temperature (e.g. cooling or heating to a certain temperature)
   Exposure to light, in particular UV-light (e.g. if UV-crosslinkable hydrogel monomers are used),
   pH value (e.g. by adding additional compounds that affect the pH value),
   Electromagnetic field;
Removing said droplet from the trapping position and/or accessing the droplet content using a demulsification method as described in the following sections

The formation of a hydrogel during or after droplet rotation is an essential step as it fixes a centered particle/cell within its position. If the droplet rotation is stopped without hydrogel formation, a centered particle might leave the center position for example by sedimentation or if the droplet is moved again after rotation by a fluid stream that is generated within the droplet due to droplet movement. Thus, the formation of a hydrogel is highly advantageous as it hinders a centered particle/cell from moving away from the center position.

In another advantageous embodiment, a droplet containing at least one particle/cell is positioned within a microfabricated geometry that retains said droplet within its position and enables to apply an incident flow/propulsive jet which flow direction has a defined angle with regard to the droplet surface (FIG. 12 B). In a particular embodiment, the flow direction of said incident flow/propulsive jet is tangential to the droplet surface and thus orthogonal to the normal vector of the droplet surface. In addition said microfabricated geometry prevents the escape of the droplet from the microfabricated geometry during droplet rotation and thus application of the incident flow/propulsive jet. For example, due to the incident flow/propulsive jet a droplet might experience a force that is orthogonal (normal force) towards the flow direction which pushes the rotating droplet towards a defined direction. Said microfabricated geometry is designed in a way that the droplet experiencing a force generated by the incident flow/propulsive jet is pushed towards a closed corner of the microfabricated geometry which has no opening/openings through which said droplet might be removed from the trapping position.

In a particular embodiment, the droplet trapping and rotation might be performed using the following procedure:
Delivering the droplet containing at least one particle/cell to a trapping geometry using a first droplet supply channel
Applying an incident flow using a second channel which flow direction has a defined angle towards the droplet surface. The droplet starts to rotate due to said incident flow.
Rotating the droplet for a defined time dt and with a defined rotation speed which results in a centering of the particle located within the droplet
Generating a hydrogel during or after droplet rotation. Said hydrogel might be formed by a polymerization reaction. In a particular embodiment, the hydrogel formation might be initiated/controlled by adjusting one of the following parameter:
   Temperature (e.g. cooling or heating to a certain temperature)
   Exposure to light, in particular UV-light (e.g. if UV-crosslinkable hydrogel monomers are used)
   pH (e.g. by adding additional compounds that affect the pH)
   Electromagnetic field
Removing said droplet from the trapping position and/or accessing the droplet content using a demulsification method as described in the following sections

Due to the droplet rotation, a particle located inside said droplet experiences a force towards the center of the droplet. This centering effect as well as the calculation of typical volume flows for achieving particle centering is explained in the following section (An illustration of the critical parameter for estimating the rotational speed of a droplet is given in FIG. 12c). The droplet rotation results in a radial pressure gradient. Due to the centrifugal force, the pressure at the periphery of the droplet is larger than the pressure at the droplet center. The pressure curve is represented by a parabola. The pressure gradient results in a centripetal force acting on a test specimen such as a particle or cell resulting in a movement of said test specimen towards the droplet center. The centripetal force depends on the radius of the test specimen as well as the distance of the test specimen from the droplet center. The pressure difference Δp acting on a specimen such as a cell can be calculated using the following formula:

Δp=2*ρ_{D}*ω²*r*x
ρ_{D}: Density of the droplet content surrounding the test specimen
ω: Angular velocity of the rotating droplet
r: Radius of the test specimen
x: Distance of the test specimen from the droplet center

The pressure difference can be used for the determination of the acceleration acting on the specimen which is given by the following formula:

$$a = \frac{{ρ}_{D}}{{ρ}_{S}} * {ω}^{2} * x$$

$${ρ}_{S} \text{:} Density of the test specimen$$

$$a \text{:} Acceleration acting on the specimen$$

To achieve efficient particle centering, an acceleration of 0.1 g might be sufficient which results in a required rotational speed of 30 droplet rotations per second. The rotational speed of a rotating droplet can be calculated based on the incident flow according to the FIG. 12C. The calculation assumes an equilibrium between a decelerating friction force and an accelerating force generated by an incident flow. The decelerating friction force is generated by the friction between the rotating droplet and the wall of the microfabricated trapping geometry. In addition, it is assumed that a slip exists between the rotary speed of the droplet (ω*R) and the velocity of the incident flow (v₀) and that a minor contact surface is present between the droplet and the wall of the microfabricated geometry. The accelerating force generated by the incident flow is then given by the following equation:

$${F}_{A} = \frac{( {v}_{0} - ω * R )}{r * η * A}$$

$${F}_{a} \text{:} Accelerating force generated by an incident flow$$

$${v}_{0} \text{:} Velocity of the incident flow$$

$$R \text{:} Radius of droplet$$

$$r \text{:} Radius of channel providing incident flow$$

$$η \text{:} Viscosity$$

$$A \text{:} Contact surface$$

The decelerating friction force is given by the equation:

$${F}_{D} = \frac{ω * R}{δ * η * A}$$

$${F}_{D} \text{:} Density of the test specimen$$

$$δ \text{:} Thickness of the gap between droplet and channel wall$$

In both force calculations, it is assumed that a Couette flow is present, thus the velocity profile is linear and the velocity gradient is given in both cases by the ratio of the change of velocity and the thickness of the gap and the thickness of the incident flow, respectively.

Thus, in force equilibrium the equations can be arranged to calculate the velocity of the incident flow/propulsive jet:

$${F}_{A} = {F}_{D} \\ \frac{( {ν}_{0} - ω * R )}{r * η * A} = \frac{ω * R}{δ * η * A} \\ \frac{( {ν}_{0} - ω * R )}{r} = \frac{ω * R}{δ} \\ {v}_{0} = ω * R * ( \frac{r}{δ} + 1 )$$

The critical parameter that is required when working with microfluidic devices is the volume flow dV/dt which can be related to the droplet volume V_{Dr}:

$$\dot{V} = \frac{dV}{dt} = {v}_{0} * {A}_{C} = {v}_{0} * π * {r}^{2}$$

$$\dot{V} \text{:} Volume flow of the incident flow$$

$${A}_{C} \text{:} Area of cross \text{-} section of channel providing incident flow$$

$${V}_{DR} = \frac{4}{3} * π * {R}^{3}$$

$${V}_{DR} \text{:} Droplet volume$$

$$\frac{\dot{V}}{{V}_{DR}} = \frac{( \frac{dV}{dt} )}{{V}_{DR}} = \frac{3}{4} * {v}_{0} * \frac{{r}^{2}}{{R}^{3}}$$

$${v}_{0} = ω * R * ( \frac{r}{δ} + 1 ) = 2 * π * N * R * ( \frac{r}{δ} + 1 )$$

$$\frac{\dot{V}}{{V}_{DR}} = \frac{( \frac{dV}{dt} )}{{V}_{DR}} = \frac{3}{2} * π * N * ( \frac{r}{δ} + 1 ) * {( \frac{r}{R} )}^{2}$$

$$\frac{( \frac{dV}{dt} )}{{V}_{DR}} = \frac{3}{2} * π * N * {( \frac{r}{R} )}^{2} * ( \frac{( \frac{r}{R} )}{( \frac{δ}{R} )} + 1 )$$

$$N \text{:} Number of droplet rotation per second$$

This equation can be simplified for the estimation of the required volume flow to:

$$\frac{( \frac{d V}{d t} )}{{V}_{D r}} \approx 3000 * {( \frac{r}{R} )}^{3}$$

In an exemplary embodiment, the trapped droplet has a radius of 40 μm and the radius of the channel providing the incident flow is 15 μm. Thus, the ratio r/R is approximately ⅓. The incident flow has to have a volume flow (per second) that is 110 times larger than the droplet volume to achieve a droplet rotation speed of 30 rotations per second. A further reduction to r/R=0.2 results in a volume flow of the incident flow that is 24 times the droplet volume. For example, if an immobilized droplet has a volume of 268 pL (R=40 μm), the required volume flow for generating efficient particle centering has to be 1.77 μl/min. This volume flow is in the typical range of volume flows used in microfluidic devices.

Thus, the disclosed centering mechanism has the advantage, that it can be easily integrated into microfluidic devices thereby enabling fully automated and highly controlled cell centering within droplets/and hydrogel matrices.

Demulsification of trapped droplets. In another advantageous embodiment, the content of droplets that have been trapped within a microfabricated geometry for droplet rotation as described previously might be accessed by replacing the surrounding fluid of type 1 that is immiscible with the fluid located within said trapped droplet with a second fluid of type 2. The fluid of type 2 might be miscible with the droplet content. Thus, a cell/particle-laden droplet might be first trapped, the cell/particle might be centered and a hydrogel matrix might be formed. Afterwards, the surrounding immiscible fluid might be replaced by a fluid miscible with the droplet content resulting in a hydrogel matrix which is trapped in said microfabricated geometry used for droplet rotation and located within a fluid of type 2. The method for coupling cell/particle centering and accessing the droplet content after cell centering might comprise the following steps:
Trapping a cell/particle-laden droplet within a microfabricated geometry in which said droplet stays at a certain position and in which the trapped droplet is perfused in a way that the droplet starts to rotate. The perfusion fluid might be a fluid of type 1 that is immiscible with the droplet content.
Rotating the droplet for a defined time dt and with a defined rotation speed which results in a centering of the particle located within the droplet
Generating a hydrogel during droplet rotation
Perfusing the microfabricated geometry with a fluid of type 2 that is immiscible with the fluid of type 1 but miscible with the droplet content

This method has the advantage that cells/particles might be first centered within a hydrogel matrix to prevent cell escape upon cell proliferation and afterwards hydrogel matrices might be transferred into a fluid that is for example suitable for supplying said cells with new nutrients.

Demulsification of droplets. In another aspect, the present disclosure relates to microfabricated structures and methods for gaining access to the content 20 of formed droplets 31 (demulsification of droplets). To this end, a droplet 30 is spatially immobilized within a microfabricated geometry in form of a trap 33, that is located below a microfabricated elastomer valve 10 having a valve portion 14 as described previously (see FIG. 18A). The first flow channel 11 is filled with a fluid of type 1 that is immiscible with a fluid of type 2 located within a second flow channel 12. Thus, the trapped droplet 31 is located within the fluid of type 1 and might contain a fluid of type 3 that is miscible with fluid of type 2 but not with fluid of type 1. The droplet 31 density is lower than the density of the immiscible fluid (fluid of type 1) surrounding said droplet. For example, the droplet 31 might be composed of water with a density of 1 g/cm³ and a droplet volume of 270 pL (diameter of approximately 80 μm). The fluid surrounding said droplet 31 might be composed of fluorinated oil such as HFE-7500 with a density of 1.614 g/cm³. Due to the density difference, a buoyant force F is acting on the trapped droplet 31 when located in the fluid of type I in the first channel 11. In this exemplary embodiment, the force F acting on the droplet 31 has a value of 1.62 nN and a direction towards the microfabricated elastomer valve 10. If the microfabricated elastomer valve is closed (an actuation force is applied) the droplet remains within the first flow channel 11. As soon as the microfabricated elastomer valve 10 is opened, an interface between fluid of type 1 and fluid of type 2 is formed within the connection channel 13. This interface remains stable if there is no pressure difference between the first flow channel and the second flow channel. A droplet 31 located below said microfabricated valve 10 experiencing a buoyant force F is pushed towards the interface upon opening of said elastomer valve 10. As no surfactant is used for droplet 31 formation and as the droplet content 31 (fluid of type 3) is miscible with fluid of type 2 the droplet 31 fuses with the fluid of type 2 located within the second flow channel. Thus, the droplet 31 content is released into the second flow channel. The main advantage is that the content of droplets can be released on-demand and in an automated manner resulting in a fully controllable access to said droplet content.

In another advantages embodiment, droplets 31 containing a cell/particle 20 are first trapped within a microfabricated geometry in form of a droplet trap 33 (FIG. 18B) that initiates cell/particle centering as described previously. After that a hydrogel matrix has been formed the surrounding fluid of type 1 that might be fluorinated oil is washed away using a fluid of type 2 with fluid of type 1 and fluid of type 2 being immiscible and fluid of type 2 and the droplet content of the trapped droplet being miscible. Thus, the fluid of type 1 can be fully removed resulting in a hydrogel matrix located within fluid of type 2.

Demulsification of hydrogel matrices. In another embodiment, droplets that are trapped below an elastomer valve for demulsification as described previously might contain hydrogel matrices. Thus, as described previously the opening of said elastomer valve results in a merging of the droplet content with the fluid of type 2 located within the second flow channel as the droplet content and the fluid of type 2 are miscible. A hydrogel matrix located within said droplets is subsequently released into the second flow channel. Afterwards, the elastomer valve might be closed again and a volume flow might be generated within the second flow channel that transfers said hydrogel matrix to another position. This has the advantage, that hydrogel matrices can be generated and transferred into an aqueous environment in a fully automated manner.

Demulsification coupled droplet sorting. In another advantageous embodiment, droplets of interest that might contain cells/particles are sorted before demulsification (FIG. 19). To this end, droplets are positioned below an elastomer valve using an electric field that generates a dielectrophoretic (DEP) force acting on said droplets. This dielectrophoretic force traps said droplet so the droplet stays within its position. In a second step, said droplet is analyzed using an optical set-up such as a microscopy or a laser. If the droplet is of interest (e.g. contains a certain number of cells) the elastomer valve above said droplet is opened and electric field is turned off. Afterwards, said droplet merges with the aqueous phase located above the elastomer valve. If the droplet is not of interest (e.g. the droplet does not contain any cells) the elastomer valve is not opened, the electric field is switched off and the droplet if washed away by applying a volume flow.

Demulsification coupled droplet sorting after particle centering. In another advantageous embodiment, droplets of interest that might contain cells/particles are positioned within a microfabricated geometry that enables droplet rotation for particle centering as well as the generation of a DEP field for droplet trapping (FIG. 20). Thus, generated droplets might be first trapped within said trapping geometry. The trapping only occurs if an electric field is applied. The dielectrophoretic force traps said droplet so the droplet stays within its position. In a second step, said droplet might be perfused with a fluid and the droplets starts to rotate while staying within its position and cell/particle centering as well as hydrogel formation occurs as described previously. In a third step, said droplet is analyzed using an optical set-up such as a microscopy or a laser. If the droplet is of interest (e.g. contains a certain number of cells) the elastomer valve above said droplet is opened and the electric field as well as the perfusion flow is turned off. Afterwards, said droplet merges with the aqueous phase located above the elastomer valve. If the droplet is not of interest (e.g. the droplet does not contain any cells) the elastomer valve is not opened, the electric field is switched off and the droplet if washed away by applying a volume flow.

Microfabricated chambers for droplet/hydrogel matrix immobilization and removal. In another aspect, the present disclosure relates to microfabricated structures and methods for the controlled positioning and sequential removal of hydrogel matrices within microfabricated chambers.

In a first advantageous embodiment, microfabricated chambers located within said array might have at least one inlet and one outlet. A first microfabricated chamber at position (1,1) might be connected to a second microfabricated chamber (2,1). To this end, the outlet of microfabricated chamber (1,1) acts as an inlet for microfabricated chamber (2,1). Microfabricated chamber (2,1) might be connected to a third microfabricated chamber (3,1). Thus, all microfabricated chambers from one column n might be connected so that microfabricated chamber (n−1,1) is connected to microfabricated chamber (n,1). In addition, a microfabricated chamber positioned at (n,1) might be connected to a microfabricated chamber (1,2) which might be connected to a microfabricated chamber positioned at (2,2). This might be repeated so that all microfabricated chambers can be perfused simultaneously with the same fluid. The inlet of microfabricated chamber (1,1) might be connected to a reservoir for supply with different fluids. The outlet of the microfabricated chamber (n,m) might be connected to a collection reservoir. Thus, all connected microfabricated chambers might be perfused with the same fluid. For example, said perfusion fluid might be an aqueous phase containing nutrients or a suspension containing one or more hydrogel matrices. The inlets and outlets of said microfabricated chambers might be closed by using an elastomer valve as described within the present disclosure. Microfabricated chambers might be first loaded with a fluid and then isolated from each other by closing said valves. Thus, a fluid volume located within microfabricated chamber (1,1) cannot be mixed with a fluid volume located within another microfabricated chamber (n,m). This has the advantage that the cell-cell communication between cells located within different microfabricated chambers might be prevented which is of importance as any secreted molecules from cells located within a first microfabricated chamber might influence the cell response of cells located within a second microfabricated chamber.

Sequential positioning. In another embodiment, said connected microfabricated chambers might be perfused with a solution containing one or more hydrogel matrices. Said microfabricated chambers might contain a microfabricated geometry for the hydrodynamic trapping of hydrogel matrices. If a first microfabricated chamber does not contain any hydrogel matrices, a first hydrogel matrix entering said microfabricated chamber will be positioned within a microfabricated trapping geometry. The positioning of said first hydrogel matrix might change the hydrodynamic resistance of the microfabricated chamber so that a second hydrogel matrix that enters said microfabricated chamber moves into a bypass channel and afterwards enters a second microfabricated chamber. Said second hydrogel matrix might be immobilized within the second microfabricated chamber. A third hydrogel matrix might then bypass the first and the second microfabricated chamber, entering the third microfabricated chamber. Thus, hydrogel matrices might be positioned in connected microfabricated chambers in a sequential manner—a first incoming hydrogel matrix might be positioned within a first microfabricated chamber, a second incoming hydrogel matrix might be positioned within a second microfabricated chamber and so on.

Defined positioning of hydrogel matrices with different compositions. In another embodiment, hydrogel matrices located within microfabricated chambers of said array might have different compositions. For example, a first hydrogel matrix of type 1 might be generated by the on-demand formation and fusion of several droplets into one larger droplet and subsequent positioning of said droplet for cell/particle centering, hydrogel formation and demulsification as described previously. The demulsified hydrogel matrix might be located within a microfluidic channel that is connected to a first microfabricated chamber. Thus, a pressure might be applied so that the hydrogel matrix enters said microfabricated chamber and said hydrogel matrix of type 1 might be positioned in said first microfabricated chamber. Said process might be repeated for the generation of a hydrogel matrix of type 2 which is subsequently positioned within a second microfabricated chamber located next to said first microfabricated chamber. This process composed of hydrogel matrix generation and immobilization might be repeated until all microfabricated chambers contain one hydrogel matrix.

Positioning of two hydrogel matrices within one microfabricated chamber. In another embodiment, said microfabricated chambers might have a microfabricated geometry for the positioning of two hydrogel matrices of the same or of different type either in contact or in close proximity. To this end, a first microfabricated chamber might have a trapping geometry as well as a bypass channel. If a first hydrogel matrix enters said first microfabricated chamber, the hydrogel matrix moves into the trapping geometry as the main volume flow goes through said trapping geometry. A second hydrogel matrix entering said first microfabricated geometry might enter the same trapping geometry as the hydrodynamic resistance of the bypass channel is larger than the hydrodynamic resistance of the trapping geometry containing one hydrogel matrix. After trapping of two hydrogel matrices the hydrodynamic resistance of said microfabricated trapping geometry increases and a third hydrogel matrix moves into the bypass channel and afterwards to a second microfabricated chamber.

Positioning of three hydrogel matrices within one microfabricated chamber. In another embodiment, said microfabricated chambers might have a microfabricated geometry for the positioning of three hydrogel matrices of the same or of different type either in contact or in close proximity. To this end, a first microfabricated chamber might have a trapping geometry as well as a bypass channel. If a first hydrogel matrix enters said first microfabricated chamber, the hydrogel matrix moves into the trapping geometry as the main volume flow goes through said trapping geometry. A second hydrogel matrix entering said first microfabricated geometry might enter the same trapping geometry as the hydrodynamic resistance of the bypass channel is larger than the hydrodynamic resistance of the trapping geometry containing one hydrogel matrix. This is also true for a third hydrogel matrix entering said first microfabricated chamber. After trapping of three hydrogel matrices the hydrodynamic resistance of said microfabricated trapping geometry increases and a fourth hydrogel matrix moves into the bypass channel and afterwards to a second microfabricated chamber.

Deformation of hydrogel matrices. In another embodiment, the immobilization of hydrogel matrices into microfabricated geometries located within microfabricated chambers might require the deformation of said hydrogel matrices. Thus, the opening of a microfabricated trapping geometry might be smaller than the hydrogel diameter and a deformation and “squeezing” of a hydrogel matrix is required to position said hydrogel within the trapping geometry. Due to the required deformation, an increased force and thus an increased volume flow might be required to push a hydrogel matrix into the trapping geometry. In turn, if the volume flow is reversed, hydrogel matrices might not leave the trapping geometry as long as the reverse volume flow reaches a critical value Qcrit at which the hydrogel matrix is deformed again and removed from the trapping position. The required deformation of said hydrogel matrices for trapping has the advantage, that the positioning and removal of said hydrogel matrices is highly controllable by adjusting the volume flow rates within said microfabricated chambers.

Removal of hydrogel matrices from position (n,m). In one advantageous embodiment, hydrogel matrices might be located within a microfabricated chamber at position (n, m) within said n×m array that enables the spatial immobilization of hydrogel matrices as well as the transfer of said hydrogel matrices into another format such as a 96-well plate at a desired time-point. To this end, said microfabricated chamber might comprise a microfabricated geometry for the immobilization of droplets and/or hydrogel matrices. In addition, said microfabricated chamber might contain at least two inlets and two outlets—a first inlet and a first outlet as well as a second inlet and a second outlet. The first inlet and the first outlet might be closed by using a first microfabricated valve as described previously. In addition, the second inlet and the second outlet might be closed using a second microfabricated valve as described previously as well. For the immobilization of hydrogel matrices/droplets, said microfabricated chamber is perfused with fluid containing single or multiple hydrogel matrices/droplets from the first inlet to the first outlet while the second inlet and the second outlet are closed. Afterwards, the first inlet and the first outlet might be closed and the microfabricated chamber might be perfused with a perfusion fluid from the second inlet to the second outlet.

Embodiments of said trapping geometry will be described in a following section of this disclosure. Said trapping geometry is connected to at least four microfluidic channels with defined hydrodynamic resistances, a first and a second microfluidic channel having a hydrodynamic resistance R₂ and R₃, respectively and a third and a fourth microfluidic channel with the hydrodynamic resistances R₄ and R₁, respectively (FIG. 21). The hydrodynamic resistances of the first and the second microfluidic channel (R₂ and R₃) might be increased by using microfabricated valves such as described previously (elastomer valve) with a first microfluidic valve v₁ (Vm2) for controlling the hydrodynamic resistance R₂ and a second microfluidic valve v₂ (Vn2) for controlling the hydrodynamic resistance R₃ (An illustration of the valve arrangements is also given in FIG. 35). The microfabricated structure comprising a microfabricated geometry for the immobilization of droplets/hydrogel beads might have the resistance R₀. The first microfluidic channel might be connected on one side with the fourth microfluidic channel as well as with the microfabricated geometry for droplet/hydrogel matrix immobilization (defined here as node N₀₁₂) and on the other side with the third microfluidic channel (defined here as node N₂₄). In addition, the third microfluidic channel might be connected on the other side to the microfabricated geometry for droplet/hydrogel matrix immobilization as well as to the second microfluidic channel (defined here as node N₀₃₄). The second microfluidic channel might be connected on the other side to the fourth microfluidic channel (defined here as node N₁₃) which might be connected to the first microfluidic channel and the microfabricated geometry for droplet/hydrogel matrix immobilization (node N₀₁₂) (FIG. 21). The hydrodynamic pressure p₁ at the intersection of the first microfluidic channel and the third microfluidic channel (node N₂₄) might be higher than the hydrodynamic pressure p₂ at the intersection of the second and the fourth microfluidic channel (node N₁₃). The described hydrodynamic resistances, pressures and connections are analogous to an unbalanced Wheatstone bridge known from electronic circuits. A microfabricated geometry having said resistances and characteristics is here defined as “reverse flow cherry picking (RFCP)” geometry. A droplet/hydrogel matrix might be immobilized within said microfabricated geometry for droplet/hydrogel matrix immobilization. A volume flow of a fluid from node N₀₁₂ to node N₀₃₄ might perfuse the microfabricated geometry for droplet/hydrogel matrix immobilization and an immobilized droplet/hydrogel matrix might stay within its position. A volume flow of a fluid from node N₀₃₄ to node N₀₁₂ might result in a removal of said droplet/particle from its position as the volume flow is reversed (this condition is defined here as “reverse flow” condition). An immobilized particle might require a reverse flow with a critical flow rate of Q_{crit} to be removed. Thus, a reverse flow with a flow rate Qᵣₑᵥₑᵣₛₑ below Q_{crit} (Qᵣₑᵥₑᵣₛₑ<Q_{crit}) might not result in a removal of said droplet/particle. In contrast, a reverse flow with a flow rate Qᵣₑᵥₑᵣₛₑ larger or equal than Q_{crit} might result in a removal of said immobilized droplet/hydrogel matrix from its immobilization position. Depending on the actuation of the microfabricated valves v₁ (Vm2) and v₂ (Vn2) four different conditions might be distinguished:
1. Both valves are not actuated: In terms of this condition, the hydrodynamic resistances R₂ and R₃ are smaller than the hydrodynamic resistances R₄ and R₁. The microfabricated geometry for the immobilization of droplets/hydrogel matrices is mainly perfused from node N₀₁₂ to node N₀₃₄. Thus, an immobilized droplet/hydrogel matrix stays within its position as the volume flow is not reversed.
2. Only valve v₁ (Vm2) is actuated while v₂ (Vn2) is not actuated: In terms of this condition, the resistance R₂ is increased and the main volume flow goes from node N₂₄ to node N₀₃₄ and from node N₀₃₄ to node N₁₃. If the microfabricated valve v₁ (Vm2) is not fully closed, the volume flow at the trapping position might go from N₀₄₂ to N₀₃₄ and the volume flow is not reversed. An immobilized particle remains within its position. If the microfabricated valve v₁ (Vm2) is fully closed, a small volume flow might go from N₀₃₄ to N₀₁₂ with Qᵣₑᵥₑᵣₛₑ being smaller than Q_{crit}. Thus, an immobilized particle remains within its position.
3. Only valve v₂ (Vn2) is actuated while v₁ (Vm2) is not actuated: In terms of this condition, the resistance R₃ is increased and the main volume flow goes from node N₂₄ to node N₀₁₂ and from node N₀₁₂ to node N₁₃. If the microfabricated valve v₂ (Vn2) is not fully closed, the volume flow at the trapping position might go from N₀₄₂ to N₀₃₄ and the volume flow is not reversed. An immobilized particle remains within its position. If the microfabricated valve v₁ (Vm2) is fully closed, a small volume flow might go from N₀₃₄ to N₀₁₂ with Qᵣₑᵥₑᵣₛₑ being smaller than Q_{crit}. Thus, an immobilized particle remains within its position.
4. Both valves v₁ (Vm2) and v₂ (Vn2) are actuated: In terms of this condition, the resistance R₂ as well as the resistance R₃ are increased and the main volume flow goes from node N₂₄ to node N₀₃₄, from node N₀₃₄ to node N₀₁₂ and from node N₀₁₂ to node N₁₃. Thus, a reverse flow is generated at the trapping position that might have a flow rate of Qᵣₑᵥₑᵣₛₑ larger than Q_{crit}. Thus, an immobilized particle is removed from its position and moves via node N₀₁₂ to node N₁₃.

In another advantages embodiment, the positioner may be constituted or may comprise one of the following types/features:
A microfluidic channel
A microfluidic channel containing an area at which the channel surface is modified
   E.g. said area might be coated fluorophilic while the rest of channel is coated hydrophilic
   E.g. said area might be coated hydrophilic while the rest of the channel is coated fluorophilic/hydrophobic
A microfabricated filter structure
A trapping geometry for trapping of single cells
Multiple trapping geometries for trapping of single cells
Multiple trapping geometries (e.g. for single cells, particles, hydrogel matrices, droplets) arranged in series or in parallel
Microfabricated geometries containing electric or magnetic elements such as:
   2D, 2.5D or 3D electrodes
   Heating elements
   Electrodes for generating surface acoustic waves

In another embodiment, various types of objects may be positioned within a RFCP-geometry and retrieved as disclosed in the present disclosure. In a particular embodiment, said objects may be biological cells, such as prokaryotic and/or eukaryotic cells, in particular cells of the immune system, cells related to different types of cancer, cells of the nerve system, stem cells. In another advantageous embodiment, said objects may be cell aggregates, in particular embryonic bodies and or spheroids composed of different cell types. One of the main advantages of positioning cells within a RFCP-geometry is that cells might be first characterized when immobilized within a RFCP-geometry and subsequently sorted using the disclosed retrieval mechanism represented by a generation of a reverse flow.

In another advantageous embodiment, said objects may be hydrogel matrices, in particular having a spherical shape. Said hydrogel matrices may contain single or multiple cells that may be of the type described previously but are not so limited. In particular, said hydrogel matrices may contain paired single cells of the same or of different type. The advantage of positioning hydrogel matrices containing cells within an RFCP-geometry is that single and/or multiple cells can be cultivated and observed for an extended time period in a highly defined microenvironment that is provided by the hydrogel matrix. In another embodiment, hydrogel matrices may contain biological compounds, in particular proteins, in particular antibodies, antibody-DNA conjugates, extracellular matrix proteins, growth factors, nucleic acids, in particular DNA, RNA, PNA, LNA, lipids, cytokines, chemokines, aptamers as well as metabolic compounds, chemical compounds, in particular small molecules, in particular drugs, molecules linked via photocleable spacer/linker, nanostructures, in particular gold nanoparticles, growth promoting substance, inorganic substances, isotopes, chemical elements.

In another advantageous embodiment, said objects may be water-in-oil droplets which represent the standard format for handling of fluids, molecules and particles within the field of microdroplet microfluidics. Said water-in-oil droplets may contain single or multiple cells that may be of the type described previously but are not so limited. One of the main advantages of positioning of droplets within a RFCP-geometry is that droplets have a defined volume and a highly miniaturized batch culture can be performed.

In another advantageous embodiment, said objects may be of the following type: oil-in-water droplets, water-in-oil-in-water droplets (double emulsions), triple emulsions, multiple emulsion, particles.

The advantage of the RFCP geometry is that immobilized droplets/hydrogel matrices might be trapped and removed in a reversible manner by controlling the corresponding valve positions. In addition, as the removal process is based on a reverse flow, the removal process is cell compatible and very gentle in comparison to other methods (such as the use of a higher temperatures for generation of bubbles or for the degradation of said hydrogel matrices) which is critical for handling single cells or small cell populations. In addition, the removal process maintains the integrity of immobilized hydrogel matrices which is critical if said hydrogel matrices store any information (e.g. secreted analytes bound to probes immobilized within said hydrogel matrices) that might be accessed at later stage.

Removing a hydrogel matrix from position n,m. In another advantageous embodiment, multiple RFCP geometries might be arranged within an n×m array whereas a droplet/hydrogel matrix located at position (n,m) might be specifically removed from said array with a dramatic reduction in the number of actuators needed for removing said droplets/hydrogel matrix. To this end, the microfabricated valves v₁ from all RFCP geometries located in row n might be actuated by a first actuator Aₙ and the microfabricated valves v₂ from RFCP geometries located in column m might be actuated by a second actuator Aₘ (said actuators might be pneumatic solenoid valves). Thus, if an actuator Aₙ as well as an actuator Aₘ is actuated, only at position (n,m) both microfabricated valves v1 and v2 from the RFCP geometry are closed/actuated resulting in a removal of a droplet/hydrogel matrix immobilized at this position as described previously. Multiple microfabricated chambers having a RFCP geometry might be perfused with the same fluid by connecting said microfabricated chambers at node N₂₄ in a way that the same hydrodynamic pressure p₁ is applied to all microfabricated chambers. In addition, all nodes N₁₃ from said microfabricated chambers might be connected so that all microfabricated chamber have the same hydrodynamic pressure p₂ at node N₁₃. Thus, droplets/hydrogel matrices that are removed using said RFCP geometry might move to a common microfabricated channel which might be defined as collection channel. Said collection channel might be connected to a common outlet that enables the transfer of removed particles into another format. This has the advantage that any position (n,m) within said array having n×m positions can be addressed by using only n+m actuators instead of n×m actuators. Illustration of addressing an n×m array are given in FIG. 22.

Removing multiple hydrogel matrices simultaneously. In another advantageous embodiment, multiple positions within said n×m array might be addressed simultaneously. For example, a first actuator Aₙ₁, a second actuator Aₙ₂ and a third actuator Aₘ₁ might be actuated simultaneously. This leads to a simultaneous removal of droplets/hydrogel matrices located at the positions (n₁, m₁) and (n₂, m₁). The simultaneous removal of immobilized hydrogel matrices has the advantage that the time needed for removing said hydrogel matrices is dramatically removed.

Immobilization and removal of two hydrogel matrices. In another advantageous embodiment, two hydrogel matrices of the same or of different type that are located at a certain position (n,m) within a microfabricated chamber which is part of a RFCP geometry might be sequentially removed (FIGS. 24 and 25). To this end, two droplets/hydrogel matrices are positioned in close proximity or in contact within a microfabricated chamber. Said microfabricated chamber might have a bypass channel with the hydrodynamic resistance R_{bypass}=2×R₅ as well as a microfabricated geometry for the immobilization of two hydrogel matrices having the resistance R_{Trapping Geometry}=R₃+(R₄⁻¹+R₄⁻¹+(R₁+R₂)⁻¹)⁻¹ (FIG. 24). During the immobilization of hydrogel matrices, the main volume flow might flow from node N3 to N0 through the hydrodynamic resistance R_{Trapping Geometry} as R_{Trapping Geometry} might be smaller than the resistance of the bypass channel R_{bypass}. If a first hydrogel enters the trapping geometry, the hydrodynamic resistance R_{Trapping Geometry} increases but remains smaller than the resistance of the bypass channel. Thus, a second hydrogel matrix entering said microfabricated trapping geometry enters the trapping geometry and the hydrodynamic resistance of said trapping geometry increases so that R_{Trapping Geometry}>>R_{bypass}. A third hydrogel matrix might enter the bypass channel and move to the next microfabricated chamber. Due to the described hydrodynamic resistances, applying a reverse flow results in a force acting on the trapped hydrogel matrices with a force F₁ acting on hydrogel matrix 1 (31A) positioned at node N₁ and with a force F₂ acting on hydrogel matrix 2 (31C) positioned at node N₂ with F₁<F₂. A critical force F_{crit,1} might be needed to remove a hydrogel matrix n located at position n within a microfabricated chamber. For example, F_{crit-1} is the force necessary to remove a hydrogel matrix located at position 1 and F_{crit,2} is the force necessary to remove a hydrogel matrix located at position 2. The forces acting on said hydrogel matrices dependent on the applied pressure difference between the nodes N₃ and N₄. If all hydrogel matrices have to experience the same force F_{crit} to be removed from the microfabricated trapping geometry the reverse flow rate for removing hydrogel matrix 2 may be increased until F₂ equals F_{crit}. The force acting on the hydrogel matrices 1 and 2 is F₁ and F₂ respectively with F₁<F₂ and F₁<F_{crit}. Thus, only the hydrogel matrix 2 is removed while hydrogel matrix 1 stays within its position. A further increase of the flow rate and thus the pressure difference might result in a force F₁ acting on hydrogel matrix 1 that equals F_{crit} which leads to a removal of hydrogel matrix 1.

This has the main advantage that immobilized hydrogel matrices can be removed sequentially. For example, a hydrogel matrix located at position 2 might be removed and collected within a first well of a 96-well plate or another format. Afterwards, a hydrogel matrix located at position 1 might be removed and collected within a second well. Another advantage is that one hydrogel matrix might be paired with various second hydrogel matrices in a sequential manner. For example, hydrogel matrix of type 1 might first be positioned next to hydrogel matrix of type 2. Hydrogel matrix of type 2 might be removed after a certain period and a new hydrogel matrix might be positioned next to hydrogel matrix of type 1. This process might be repeated several times.

Immobilization and removal of three hydrogel matrices. In another advantageous embodiment, three hydrogel matrices of the same or of different type that are located at a certain position (n,m) within a microfabricated chamber which is part of a RFCP geometry might be sequentially removed. To this end, hydrogel matrices might be first immobilized as described previously (FIG. 26, FIG. 27 and FIG. 28). Applying a reverse flow results in a force acting on the trapped hydrogel matrices with a force F1 acting on hydrogel matrix 1 (31A) positioned at node N1 and with a force F2 acting on hydrogel matrix 2 (31B) positioned at node N2 with F1<F2. Applying a reverse flow results in a force acting on the trapped hydrogel matrices with a force F1 acting on hydrogel matrix 1 positioned at node N1, with a force F2 acting on hydrogel matrix positioned at node N2 and with a force F3 acting on hydrogel matrix 3 (31C) positioned at node N3 with F1<F2<F3. Applying a reverse flow results in a force acting on the trapped hydrogel matrices with a force F1 acting on hydrogel matrix 1 positioned at node N1, with a force F2 acting on hydrogel matrix 2 positioned at node N2 and with a force Fn acting on hydrogel matrix n positioned at node Nn with F1<F2< . . . <Fn. A critical force Fcrit,n is needed to remove a hydrogel matrix n located at position n within a microfabricated chamber. The forces acting on said hydrogel matrices dependent on the applied pressure difference. If all hydrogel matrices have to experience the same force Fcrit to be removed from the microfabricated trapping geometry the reverse flow rate for removing hydrogel matrix 3 may be increased until F3 equals Fcrit. The force acting on the hydrogel matrices 1 and 2 is F1 and F2 respectively with F1<F2<F3 and F1<F2<Fcrit. Thus, only the hydrogel matrix 3 is removed while hydrogel matrix 1 and hydrogel matrix 2 stay within their position. A further increase of the flow rate might result in a force F2 acting on hydrogel matrix 2 that equals Fcrit which leads to a removal of hydrogel matrix 2 while hydrogel matrix 1 stays in place. Finally, a further increase of the flow rate might result in a force F1 acting on hydrogel matrix 1 which is equal to Fcrit. Thus, the hydrogel matrix 1 is removed. This has the main advantage, that immobilized hydrogel matrices can be removed sequentially. For example, a hydrogel matrix located at position 3 might be removed and collected within a first well of a 96-well plate or another format. Afterwards, a hydrogel matrix located at position 2 might be removed and collected within a second well.

Immobilization and removal of more than three hydrogel matrices. In another advantageous embodiment, more than three hydrogel matrices of the same or of different type that are located at a certain position (n,m) within a microfabricated chamber which is part of a RFCP geometry might be sequentially removed. To this end, hydrogel matrices might be first immobilized as described previously so that multiple hydrogel matrices might be positioned in a sequence. Said hydrogel matrices might be located within a microfabricated trapping geometry in which each hydrogel matrix experiences a different force deepening on its trapping position. Applying a reverse flow results in a force acting on the trapped hydrogel matrices with a force F₁ acting on hydrogel matrix 1 positioned at node N₁, with a force F₂ acting on hydrogel matrix 2 positioned at node N₂ and with a force Fₖ acting on hydrogel matrix k positioned at node Nₖ with F1<F2< . . . <Fₖ. A critical force F_{crit,k} might be needed to remove a hydrogel matrix k located at position k within a microfabricated chamber. The forces acting on said hydrogel matrices dependent on the applied pressure difference. If all hydrogel matrices have to experience the same force F_{crit} to be removed from the microfabricated trapping geometry the reverse flow rate for removing hydrogel matrix k may be increased until Fₖ equals F_{crit}. The force acting on the hydrogel matrices 1, 2 . . . k is F₁, F₂ . . . Fₖ respectively with F₁<F₂< . . . <Fₙ and F₁<F₂< . . . <F_{crit}. Thus, only the hydrogel matrix k is removed while all hydrogel matrices 1, 2 . . . k−1 stay within their position. A further increase of the flow rate might result in a force Fₖ₋₁ acting on hydrogel matrix k−1 that equals F_{crit} which leads to a removal of hydrogel matrix k−1 while hydrogel matrix k−2 stays in place. Finally, this process might be repeated until all hydrogel matrices have been removed. This has the main advantage, that multiple immobilized hydrogel matrices can be removed sequentially and transferred into a 96-well plate or another format.

Extraction of cells located within immobilized hydrogel matrices and subsequent transfer into another format—Highly controlled cell transfer using RFCP. In another advantages embodiment as described with reference to FIGS. 53a and 53b, said array might be used to transfer single or multiple cells located within a droplet or hydrogel matrix that is positioned within said array to another format such as a 96-well plate, a 384-well plate, a 1536-well plate or a microwell plate whereas exactly one single cell might be transferred to a pre-defined well of said established formats or each similar formats. FIG. 53 shows an exemplary well plate 80. For example, a droplet/hydrogel matrix might contain initially one single cell. After cultivation for a certain time period (e.g. 3 days) said single cell might divide and proliferate and might form a spheroid 81 consisting of more than one cell 82A, 82B, 83C, 82D. The encapsulated cells 82A-D might be separated from each other and subsequently transferred into another format whereas each well of said format will only contain one single cell derived from said hydrogel matrix. For example, said extraction process might be performed in the following steps:
1. Immobilization of cell-laden hydrogel matrices. Immobilization of hydrogel matrices containing single or multiple cells 82A-D within a positioner, in particular trapping structure at which the flow can be reversed using the previously mentioned RFCP mechanism.
2. Optionally: Cell cultivation within hydrogel matrices. Cultivation of cells for an extended time period (For example cells might be cultivated for one, two or more than three days up to several weeks).
3. Event-triggered removal of immobilized hydrogel matrices. As soon as a certain event occurs, the hydrogel matrix containing said cells is removed from the trap by said RFCP mechanism and transferred to a perfusion chamber containing a filter structure that holds the hydrogel matrix in place and allows smaller particles/cells to pass through. For example, said event might be a certain fluorescence intensity of the cultivated cells (e.g. cultivated cells might express a fluorescent reporter protein), a certain cell morphology such as an increased cell size, the formation of a cell spheroid with a certain size or a certain surface profile.
4. Extraction of single cells from hydrogel matrices. The cell-laden hydrogel matrix that is hold in place at the filter structure is then perfused with a solution that enables the separation of aggregated cells that might be attached due to cell-cell or cell-matrix contacts. Said solution might contain for example a protease (e.g. trypsin) for digesting surface proteins that mediate cell-cell contacts as well as cell-cell and cell-matrix adhesion. Afterwards, the hydrogel matrix that contains now separated cells is dissolved. In particular, this might be done by perfusion with metalloproteases for hydrogels that contain degradation sites that can be cleaved by metalloproteases for hydrogel digestion.
5. Refocussing of single cells. Cells that are released from the hydrogel matrix due to hydrogel matrix removal are further separated from each other by using a re-focusing geometry or by using multiple re-focusing geometries in sequence.
6. Trapping within RFCP geometries. Re-focused cells might be trapped in a single cell trap located within a RFCP geometry. Multiple RFCP traps might be positioned in sequence connected with each other.
7. Transfer of trapped single cells into a standard format. Afterwards, single cells located with said RFCP geometries might be transferred to a standard format such as the well 80 by actuating the corresponding valves as described previously.

This has the advantage that cells derived from one single cells can be separated and further analysed with conventional methods such as RT-PCR or single-cell sequencing without losing the time-lapse information about the cultured cells that has been recorded during cell culture. For example, this time-lapse information might be among others growth data, fluorescence data or migration data.

All information referring to the particles in particular cells are registered within a database 86. The registered information contains parameters 83 of the particles which are stored together with a unique particle ID 84 and a unique position ID indicating the new location in which the isolated particles are located.

Extraction of cells located within immobilized hydrogel matrices and subsequent transfer into another format—Transfer using spatially separated single cell traps and optical detection mechanism. In another advantageous embodiment, cells that have been released from a hydrogel matrix and that have been re-focused (step 5 of the previously described process) might be trapped within single cell traps that have a defined distance from each other. All trapped cells might be released simultaneously by reversing the fluid flow. The trapping of cells thus results in a spatial separation of single cells thereby allowing a more controlled transfer of said cells into another format. After applying a reverse flow cells might be transferred towards a common outlet. Single cells within the volume flow might be detected by a conventional detection mechanism. For example the flow channel might be coupled on one side to a glass fibre that illuminates the channel with a defined wavelength such as 480 nm, 532 nm or 600 nm. Said light might be detected using a photodetector such as a photo diode. If a cell is passing the detection area, the cell disturbs the light path thereby giving a signal that can be measured. Afterwards, said cell might be transferred to another format such as a well of a 96-well plate and a subsequently arriving second cell might be transferred to another well of a 96-well plate.

Extraction of cells located within immobilized hydrogel matrices and subsequent transfer into another format—Coupling of localization information to genetic phenotype. In another advantageous embodiment, single cells might be extracted from hydrogel matrices, subsequently transferred into another format and single-cell data from downstream processes might be coupled with localization information derived from cell clusters and/or phenotypic information (such as surface profile or intracellular staining) gained during cell culture. For example, this might be performed in the following steps:
1. Immobilization of cell-laden hydrogel matrices. Immobilization of hydrogel matrices containing single or multiple cells within a trapping structure at which the flow can be reversed using the previously mentioned RFCP mechanism.
2. Optionally: Cell cultivation within hydrogel matrices. Cultivation of cells for an extended time period (For example cells might be cultivated for one, two or more than three days up to several weeks).
3. Optionally: Staining of single or multiple biological compounds located at the cell surface and/or single or multiple intracellular biological compounds such as proteins/mRNA/miRNA/DNA/lipids. After cell cultivation, cells might be stained for single or multiple intracellular or surface localized markers or any other intracellular or extracellular molecules. Said molecules might be for example proteins, mRNA, miRNA, RNA, DNA, lipids or small molecules from the cell metabolism.
4. High-resolution imaging of stained cells and localization extraction. Analysis of single or multiple cells to get spatial information about cells as well as cell characteristics. The spatial information of the cells might be received by identifying cell boundaries using a cell membrane stain such as CellTracker™ CM-DiI (ThermoFisher). By using the cell boundary information the cell centre represented by XYZ-coordinates might be calculated for example with an image analysis program such as Image). The localization information of cells might be coupled to further information gained for example via a staining mentioned in step 3.
5. (Event-triggered) removal of immobilized hydrogel matrices. As soon as a certain event occurs, the hydrogel matrix containing said cells is removed from the trap by said RFCP mechanism and transferred to a perfusion chamber containing a filter structure that holds the hydrogel matrix in place and allows smaller particles/cells to pass through. For example, said event might be a certain fluorescence intensity of the cultivated cells.
6. Extraction of single cells from hydrogel matrices. The cell-laden hydrogel matrix that is hold in place at the filter structure is then perfused with a solution that enables the separation of aggregated cells that might be attached due to cell-cell contacts. Said solution might contain for example trypsin for digesting surface proteins that mediate cell-cell contacts and cell-cell adhesion. Afterwards, the hydrogel matrix that contains now single cells is dissolved. For example, this might be done by perfusion with metalloproteases.
7. Refocussing of single cells. Cells that are released from the hydrogel matrix due to hydrogel matrix removal are further separated from each by using a re-focusing geometry or by using multiple re-focusing geometries in sequence.
8. Trapping of single cells. Re-focused cells might be trapped randomly in a single cell trap that might be located within a RFCP geometry. Multiple RFCP traps might be positioned in sequence connected with each other.
9. High-resolution imaging of cells trapped within RFCP geometries and information coupling. The trapped single cells are then imaged again to couple the trapping position of the cells with the localization information received previously in step 4. For coupling the position information of each cell with the localization information within said hydrogel matrices, it is necessary, that different cells can be identified and differentiated. This can for example be done by staining different surface markers or intracellular markers as described in step 3. Combining the trapping information with the localization information is critical as it allows afterwards the transfer of single cell into another format (step 10) without losing the localization information of the cells.
10. Transfer of trapped single cells into a standard format. Afterwards, single cells located within said RFCP geometries might be transferred to another format as described previously. Thus, the localization information of a single cell within a hydrogel matrix or spheroid might be coupled to the corresponding position of another format. Afterwards, genetic information about the cells might be received by established protocols such as single cell sequencing.

This process has the advantage that the spatial information (e.g. localization of cell within cell cluster) can be combined with downstream data, such as genetic data received by other methods (e.g. next-generation sequencing, RNA-seq, Drop-seq, Nanopore sequencing).

Impedance measurements of immobilized cell-laden hydrogel matrices. In still another aspect, embodiments of this disclosure provide methods for measuring the impedance of (cell-laden) hydrogel matrices located within said array. To this end, microfabricated electrodes might be located within close proximity of microfabricated trapping geometries for the immobilization of hydrogel matrices. Said microfabricated electrodes might be located below an immobilized hydrogel matrix.
Microfabricated electrode below trapping geometry
Microfabricated electrode surrounding hydrogel matrix (3D electrode)
Electrodes might be composed of a positive and a negative electrode
Electrodes at position (n,m) might be addressed individually

Heating by using gold nanoparticles and RF Fields. In still another aspect, embodiments of this disclosure provide methods for heating hydrogel matrices located in said array using radio frequencies. To this end, gold nanostructures are immobilized in hydrogel matrices and hydrogel matrices containing said gold nanostructures are immobilized in said array as described previously. Gold nanostructures may comprise gold nanoparticles and/or gold nanorods with sizes below 20 nm. Said nanostructures may have a silica-coating having different functional groups such as —OH, —NH₂, —SH, -MAL, —NHS. In a particular embodiment, said gold nanostructures may be immobilized within hydrogel matrices by an NHS linkage.

The incorporation/immobilization of gold nanostructures might be performed by mixing two droplets with one droplet containing a hydrogel precursor molecule A and a second droplet containing a hydrogel precursor molecule B as well as said gold nanostructures. Hydrogel formation as well as hydrogel matrix positioning on said array might be performed as described previously. Said hydrogel matrices containing nanometer-sized gold structures might be positioned within a microfabricated trapping geometry located within a microfabricated chamber. Said microfabricated trapping geometry might have a microfabricated electrode acting as a radio frequency antenna in close proximity to an immobilized hydrogel matrix containing immobilized gold nanostructures. Said electrodes might be positioned below an immobilized hydrogel matrix. Thus, applying an electric field to said microfabricated electrodes results in a electrophoretic and/or magnetic heating of immobilized nanostructures and thus in a heating of immobilized hydrogel matrices. The advantage of the described process is that hydrogel matrices might be heated in a very fast and controllable manner by applying a radio frequency.

Alternating biphasic compartment generation. In another embodiment, the present disclosure relates to a method for transferring immobilized hydrogel matrices located within said array into a reduced volume compartment without changing the position of said hydrogel matrices thereby reducing the reaction volume and thus increasing the local concentration of analytes (e.g. mRNAs, PCR-Products) which increases the sensitivity of potential detection mechanisms. To this end, said array containing immobilized hydrogel matrices is first perfused with a fluid of type 1 that is immiscible with a fluid of type 2. Immobilized hydrogel matrices might be soluble within the fluid of type 2 but insoluble within the fluid of type. In a second step, said array is perfused with a fluid of type 2. As the hydrogel matrices are fixed at defined positions within said array, the fluid of type 2 replaces the fluid of type 1 surrounding immobilized hydrogel matrices. Thus, the volume of fluid of type 1 in which said hydrogel matrices are located is reduced to the volume of said hydrogel matrices. After a certain period, said array might be perfused again with fluid of type 1. This process might be repeated several times. The described process is defined in the present disclosure as “alternating biphasic compartment generation”. For example, fluid of type 1 might be an aqueous phase containing nutrients for cultivating cells. Fluid of type 2 might be fluorinates oil such as HFE-7500 that is immiscible with fluid of type 1. After, washing with fluid of type hydrogel matrices are located within said fluorinated oil.

The first advantage of the described method is that (cell-laden) hydrogel matrices can be transferred into an isolated and reduced reaction volume. The reduction of the reaction volume results in an increased sensitivity of potential detection mechanisms as the concentration of analytes located within said hydrogel matrices is significantly increased. A second advantage of said method is that hydrogel matrices might be first transferred into an isolated and reduced reaction volume and afterwards, hydrogel matrices might be washed again with a miscible fluid. Thus, different reactions can be carried out by repeating said process. This is for example of importance, if different analytes located within said hydrogel are detected by different reaction compounds.

Structures for fluid supply and flow control. In another advantageous embodiment, the present disclosure comprises structures for supplying fluids to said array and for controlling the volume flows of fluids within said array. To this end, fluid reservoirs are positioned directly above the main inlets and outlets of said array. Said fluid reservoirs might have a first and a second opening. The first opening of said fluid reservoirs might be separated from the main inlets and outlets of said array by a filter membrane (such as a net filter) and the second opening of said fluid reservoirs might be separated from the space above the fluid reservoirs by a membrane that prevents the evaporation of fluids located within said reservoir. Said membrane might be a PTFE membrane which prevents the diffusion of vaporized water molecules through said membrane while enabling the diffusion of gases such as air. A pressure might be applied to the space above said fluid reservoir. Thus, a pressure is acting on the fluid which might be a liquid located within said reservoirs. A pressure p1 might be applied to the fluid reservoirs connected to the main inlets of said array and a pressure p2 might be applied to the fluid reservoirs connected to the main outlets of said array. The pressure p1 might be larger than p2 thus a fluid located within said reservoirs might flow from the reservoirs located above the main inlets to the reservoirs located above the main outlets.

A first advantage of the described structure is that the dead volume of said structure is zero as the reservoir is directly located above the inlet of said array. Thus, also very small sample volumes can be handled. This is a significant advantage in comparison to established system that use tubing connected to syringes as in terms of this system the handling of small sample volumes is hardly possible and syringe systems always have a certain dead volume. A second advantage of the described structure is that the evaporation of liquid fluids located within said reservoirs is prevented. This is of great importance as the fluid volume located within said reservoir might be below 500 μl. Thus, even small changes in the fluid volume due to evaporation of the liquid might result in a concentration change of dissolved compounds and thus a change of the culture conditions.

### Applications

Time-lapse microscopy and event-triggered hydrogel matrix removal. Furthermore, the present disclosure pertains to a method that enables the time-lapse monitoring of cell phenotypes and the removal of specific hydrogel matrices that fulfill a predefined requirement. To this end, cell-laden hydrogel matrices located within said array might be imaged repeatedly using an optical set-up that enables the quantification of fluorescent molecules which might be expressed by cultivated cells (such as fluorescent proteins (e.g. eGFP, RFP, YFP)) or that enables the recording of bright field images or similar microscopy data. Said optical set-up might be a conventional bright field microscope, an epifluorescence microscope, a confocal laser scanning microscope, a high-content screening system or a similar optical set-up. Said optical set-up might also consist of an excitation laser with a spot size in the range of the diameter of one hydrogel matrix (e.g. 80 μm and/or 1-500 μm) and a corresponding mechanism for the detection of a fluorescent signal (for example by using corresponding emission filters and photomultiplier tubes or photodiodes). In total, k×(n×m) hydrogel matrices might be located within a microfabricated array having n×m microfabricated chambers with k being the number of hydrogel matrices per microfabricated chamber. Said hydrogel matrices might be imaged in a sequential order and repeatedly with a period dt and the corresponding data might be saved and analyzed at the same time of the imaging procedure. In addition, multiple hydrogel matrices located at different positions might be imaged/measured simultaneously. As soon as a predefined event at a position (n,m) is detected, the removal of a hydrogel matrix located at this position might be initiated and said hydrogel matrix might be transferred into another format. Said predefined event might be a fluorescent signal that has reached a defined value. In another embodiment, event definitions might have the following type:
If the measured fluorescence signal at position (n,m) is larger/lower than S_{Threshold} (Signal threshold) a hydrogel matrix k located at position (n,m) is transferred into well (x, y) of another format such as a 96-well plate
If a first fluorescence signal at position (n,m) is larger/lower than S_{Threshold F1} and if a second fluorescence signal at position (n,m) is larger/lower than S_{Threshold F2} (Said conditions might also be used for more than two fluorescent signals) a hydrogel matrix k located at position (n,m) is transferred into well (x, y) of a 96-well plate or a similar format Said method comprises the following steps:
Go to position (n,m)
Image hydrogel matrix 1 located at position (n,m)
Calculate signal intensity
Check if predefined event is fulfilled
If predefined is fulfilled: initiate removal of hydrogel matrix k located at position (n,m) and transfer to corresponding well (x,y) of a 96-well plate or a similar format
Go to the next position and repeat process

The event-triggered removal of hydrogel matrices located at a certain position (n,m) and the subsequent transfer into another format has the following advantages in comparison to other methods:
Firstly, hydrogel matrices that have been removed and transferred into another format might be analyzed with established methods such as qRT-PCR or NGS that give information about the genotype of collected cells. As the original position of said hydrogel matrix on the microfabricated array and the position of the well in which the hydrogel matrix has been transferred is known, both information gained within the different formats can be combined. Thus, the advantage is that a time-lapse phenotype can be coupled to the corresponding genotype.
Secondly, (cell-laden) hydrogel matrices that have been removed and transferred into another format might be cultivated in a large format which enables the expansion of cells that showed a certain phenotype. Thus, the pre-selection of viable cells or cells that show a desired phenotype is possible.

Generation of defined array compositions using the previously described RFCP-based sorting mechanism. In another advantageous embodiment as shown in FIGS. 54a and 54b, the RFCP mechanism is used for the generation of an array whose locations 32 in particular comprising. trapping structures, are occupied by droplets e.g. hydrogel matrices that encapsulate a pre-defined number of cell types having certain characteristics. For example, in one embodiment all array positions shall be occupied by hydrogel matrices with each of them having exactly one cell encapsulated. To this end, an empty array is first loaded with hydrogel matrices 31 that contain Poisson-distributed cells (situation A in FIG. 54b). Afterwards, the number of cells in each hydrogel matrix at each array position is determined. This might be done by using an optical set-up such as an automated epifluorescence microscope. The number of cells within a trapped hydrogel matrix might be determined visually for all immobilized hydrogel matrices or by image acquisition and subsequent object recognition using corresponding software tools (such as Image J, Cell Profiler or matlab). In a next step, all positions that contain droplets 31n which do not conform to a predefined criterium, in particular empty hydrogel matrices or hydrogel matrices that contain more than one single cell, are listed (e.g. said list might contain the exemplary positions (n₁|m₁), (n₂|m₂), . . . , (nₖ|mₖ) with n being the row index of said array, m being the column index of said array and k being the number of positions at which a pre-defined criterium is not fulfilled (e.g. all hydrogel matrices that contain more or less than exactly one single cell)). Next, all listed hydrogel matrices are removed from the corresponding positions using the RCFP mechanism as described previously by actuating the corresponding actuators (situation B in FIG. 54b). For example, the hydrogel matrix positioned at (2,2) (row number two and column number two) might be removed by actuating the valve for row number two and the valve for column number two. The removal might be done in a sequential or simultaneous manner. Afterwards, the steps of loading, object recognition and removal are repeated (situations D-I in FIG. 54b) until the array contains the desired amount of droplets 31y which conforms to the predefined criteria, in particular hydrogel matrices that contain only one single cell (not shown in FIG. 54b). The described method for the generation of a pre-defined array composition has the advantage that it does not require any upstream sorting mechanism. In another exemplary embodiment, hydrogel matrices that fulfil one or more of the following criteria might be retained within said array while all hydrogel matrices that fulfill not one or more of the following criteria might be removed:
Hydrogel matrices containing
   exactly a pre-defined number of cells (e.g. exactly one single cell or exactly two single cells)
   cells with a pre-defined phenotype (e.g. cells that express a certain amount of a fluorescent protein such as eGFP, RFP, YFP; e.g. cells that show a certain surface profile—for example cells might have been stained with fluorescently labeled antibodies prior to loading them on said array)
   cells that have been labeled with a pre-defined probe (e.g. said probe might be a fluorescently labeled molecule that might detect intracellular or surface localized biomolecules such as RNA, DNA, proteins or lipids or small molecules of the cell metabolism)
   cells that show a pre-defined morphology (e.g. cells with different sizes or cells, that have a certain degree of cytodendrites)

In another advantageous embodiment, droplets containing single or multiple cells might be trapped instead of hydrogel matrices. The generation of said pre-defined array might be done with the same procedure as described previously.

Time-lapse and endpoint cytokine profiling—one cell-laden hydrogel matrix, one analyte. Furthermore, the present disclosure pertains to a method for the time-lapse monitoring of molecules that are secreted by single cells or cell colonies or upon the cell-cell interaction of two (single) cells or multiple cells (FIG. 29 and FIG. 30). To this end, (single) cells 20 are encapsulated into hydrogel matrices 31A and positioned on a microfluidic array as described in the present disclosure. Afterwards a second hydrogel matrix 31B that contains primary antibodies against a defined target analyte is immobilized directly next to a hydrogel matrix containing single or multiple cells. The hydrogel matrices might have the same or a different size and might be composed of the same hydrogel backbone as well as of a different backbone. In one embodiment, both hydrogel matrices might have a spherical shape with a diameter of 80 μm. Thus, one hydrogel matrix for cell cultivation and one hydrogel matrix for analyte detection are positioned in close proximity within a microfluidic chamber 32. Said microfluidic chamber can be closed as described in the present disclosure to generate a closed and isolated compartment with a defined volume. In one embodiment, the microfluidic chamber might be closed by actuating corresponding valves. The generated volume might be in the range of 10000 pl to 50000 pl. Said volume might be reduced by using the described alternating biphasic compartment generation described in the present disclosure. Thus, in one embodiment the aqueous phase surrounding the spatially immobilized hydrogel matrices might be exchanged by an immiscible fluid such as a fluorinated oil (e.g. HFE-7500) thereby reducing the volume compartment to a volume that approximately corresponds to the volume of the trapped hydrogel matrices. In one embodiment, the reduced volume of the aqueous phase containing the hydrogel matrices might be in the range of 400 to 600 pL. Upon secretion of single molecules such as specific cytokines, secreted analytes diffuse to the hydrogel matrix containing the primary antibody. After a defined period (e.g. 1 hour) the microfluidic chamber is either opened and washed (e.g. with PBS) to remove e.g. unbound analytes or medium components or if the hydrogel matrices are surrounded by for example an oil phase, the oil phase is removed by washing with an aqueous phase (e.g. with PBS). Afterwards a second antibody (secondary antibody) is added to the perfusion system. This second antibody binds to the analytes located within the hydrogel matrix (detection bead) that is already bound to the primary antibody. The second antibody binds a different epitope than the primary antibody. Afterwards, all non-bound secondary antibodies are washed away e.g. by perfusing the microfluidic chamber with a washing solution such as PBS.

In one embodiment, the secondary antibody might be labeled with a fluorescent marker such as fluorescent organic molecules (e.g. FITC) or quantum dots. The amount of analytes that are bound to the primary antibodies might be determined by measuring the fluorescence intensity of the fluorescently labeled secondary antibodies. As the fluorescence intensity of the secondary antibodies is proportional to the amount of secondary antibodies located within the hydrogel matrix which is in turn proportional to the bound analytes, an indirect quantification of bound analytes is possible. The fluorescence intensity of the hydrogel matrix (detection bead) might be analyzed using an optical set-up such as an epifluorescence microscope, a confocal laser scanning microscope, a high content screening system or a super-resolution microscope or any other optical setup. The hydrogel matrix (detection bead) containing now the primary antibody, the analyte and the secondary antibody is removed from the trap by reverse flow cherry picking while the hydrogel matrix containing the cell/s stays within the trap. Afterwards, a new hydrogel matrix is loaded again and the process is repeated. Hydrogel matrices that are removed from the trap might be collected in a well-plate or another format for further analysis and each well might corresponds to a defined position and time-point on the microfluidic chip.

The described process might be summarized in the following steps:
1. Cell encapsulation within hydrogel matrix and subsequent positioning within microfluidic array having microfabricated chambers
2. Formation/Delivery of hydrogel matrix (detection bead) that has immobilized primary antibodies having a specificity against defined target analytes (such as cytokines, chemokines, TNF or interleukins) and subsequent immobilization of the hydrogel matrix (detection bead) next to or in close proximity to the cell-laden hydrogel matrix within the same microfabricated chamber.
3. Reducing of the reaction volume by either closing corresponding valves thereby isolating the microfabricated chamber and/or by perfusing the microfabricated chamber with an immiscible fluid such as fluorinated oil (e.g. HFE-7500) (alternating biphasic compartment generation).
4. Incubation of cells for a defined time period (e.g. 1 h, 2 h or more)
5. Washing of the immobilized hydrogel matrices with washing buffer such as PBS. If an oil phase has been used for compartment generation the oil phase might be washed away.
6. Perfusing the microfabricated chamber with a solution containing a fluorescently labeled secondary antibody with a defined concentration for a defined time period.
7. Washing of the immobilized hydrogel matrices with washing buffer (e.g. PBS) to remove unbound secondary antibodies.
8. Analysis of the fluorescence intensity of the hydrogel matrix (detection bead) containing the secondary antibodies using an optical set-up.
9. Removing the hydrogel matrix (detection bead) that contains the primary antibodies, bound analytes and the secondary antibodies using the revers flow cherry picking mechanism described in the present disclosure. The hydrogel matrix containing the cell(s) stays in place. The removed hydrogel matrices (detection beads) might be collected in a controlled manner using a pipetting robot.
10. Repeating the process starting with step 2 several times.

The described process has several advantages. First, it enables the detection of molecules that have been secreted from single cells in a dynamic, time-lapse manner. Second, due to the removal of the hydrogel matrix (detection matrix) containing the bound analytes, the dynamic range of the detection system is larger. For example, if only one detection bead might be used for the whole culture time, the primary antibodies might be saturated with secreted analytes resulting in a limited dynamic range. Third, the reduction of the reaction volume increases significantly the sensitivity of the detection mechanism as the analyte concentration is significantly increased due to the volume reduction.

Time-lapse and endpoint cytokine profiling—one cell-laden hydrogel matrix, multiple analytes. In another advantageous embodiment, a (cell-laden) hydrogel matrix might be positioned next to a hydrogel matrix that contains multiple primary antibodies with specificities against different target analytes (detection bead) thereby enabling multiplexing. The reduction of the reaction volume might be performed as described previously. After a defined cultivation period, the microfabricated chamber might be perfused with a washing solution (such as PBS) and subsequently with a mix of fluorescently labeled secondary antibodies that bind to analytes located within the hydrogel matrix containing immobilized primary antibodies. The secondary antibodies might be labeled with different fluorescent molecules or quantum dots having different excitation and emission wavelength which enable the read-out of a secondary antibody with a defined specificity by using a corresponding optical-setup (multiplexing). The use of fluorescently labeled antibodies is well known from other techniques such as fluorescent-activated cell sorting (FACS). After a second washing step, the hydrogel matrix containing multiple immobilized primary antibodies, analytes of different types and the corresponding fluorescently labeled secondary antibodies is removed and transferred into another format. The use of differentially labeled secondary antibodies enables multiplexing. Thus, multiple analytes can be detected with one hydrogel matrix that contains different antibodies (e.g. screening for multiple cytokines such as TNF-alpha, IL-6, IL-10, Il-1beta).

The described process might be summarized in the following steps:
1. Cell encapsulation within hydrogel matrix and subsequent positioning within microfluidic array having microfabricated chambers
2. Formation/Delivery of hydrogel matrix (detection bead) that has immobilized multiple primary antibodies having different specificities against multiple, defined target analytes (such as cytokines, chemokines, TNF or interleukins) and subsequent immobilization of the hydrogel matrix (detection bead) next to or in close proximity to the cell-laden hydrogel matrix within the same microfabricated chamber.
3. Reducing of the reaction volume by either closing corresponding valves thereby isolating the microfabricated chamber and/or by perfusing the microfabricated chamber with an immiscible fluid such as fluorinated oil (e.g. HFE-7500) (alternating biphasic compartment generation).
4. Incubation of cells for a defined time period (e.g. 1 h, 2 h or more)
5. Washing of the immobilized hydrogel matrices with washing buffer such as PBS. If an oil phase has been used for compartment generation the oil phase might be washed away.
6. Perfusing the microfabricated chamber with a solution containing a mix of different fluorescently labeled secondary antibody with different specificities. The antibodies might have a defined concentration and might be perfused for a defined period.
7. Washing of the immobilized hydrogel matrices with washing buffer (e.g. PBS) to remove unbound secondary antibodies.
8. Analysis of the fluorescence intensity of the hydrogel matrix (detection bead) containing the secondary antibodies using an optical set-up. The fluorescence intensities might be read using corresponding excitation wavelengths and emission filters enabling multiplexing.
9. Removing the hydrogel matrix (detection bead) that contains the primary antibodies, bound analytes and the secondary antibodies using the revers flow cherry picking mechanism described in the present disclosure. The hydrogel matrix containing the cell(s) stays in place. The removed hydrogel matrices (detection beads) might be collected in a controlled manner using a pipetting robot.
10. Repeating the process starting with step 2 several times.

The described process has the advantage that multiple analytes can be detected simultaneously.

In another advantageous embodiment, a (cell-laden) hydrogel matrix might be positioned next to a hydrogel matrix that contains multiple primary antibodies with specificities against different target analytes. After a defined period, the microfabricated chamber might be perfused with a washing solution (such as PBS) and subsequently with a mix of barcoded secondary antibodies that bind to analytes located within the hydrogel matrix containing immobilized primary antibodies. After a second washing step, the hydrogel matrix containing multiple immobilized primary antibodies, analytes of different types and the corresponding barcoded secondary antibodies is removed and transferred into another format. The use of barcoded secondary antibodies enables multiplexing. Thus, multiple analytes can be detected with one hydrogel matrix that contains different antibodies (e.g. screening for multiple cytokines such as TNF-alpha, IL-6, IL-10, Il-1beta). The collected hydrogel matrices can be used for quantifying the bound analytes by detecting the barcoded oligonucleotides bound to the secondary antibody. This can for example be done by qRT-PCR or digital PCR. Another possibility might be to amplify such oligonucleotides and then sequence the amplified product (e.g. with nanopore sequencing or similar techniques).

This method might not only be used for the analysis of the secreted molecules of single cells (or cell colonies) but also for the analysis of the cell-cell interaction between two different cell types (e.g. an immune cell and a cancer cell). To this end, an immune cell and a cancer cell are co-encapsulated within one hydrogel matrix and the time-lapse secretion profile is subsequently monitored as described previously.

In another embodiment, an immune cell is encapsulated within one hydrogel matrix and a cancer cell is encapsulated within a second hydrogel matrix. Afterwards, three spherical hydrogel matrices are paired, one containing the immune cell, one containing the cancer cell and one containing the detection antibodies. The hydrogel bead with the detection antibodies can be specifically removed with the other two hydrogel beads staying on the microfluidic chip. The detection method is the same as described above. This setup has the advantage that each cell type can be located within a different hydrogel matrix having different characteristics (e.g. different mechanical strength or different immobilized ECM compounds or growth factors that influence the cell behavior). For example, a cancer cell might need different ECM compounds than an immune cell. This experimental set-up would allow the identification of communication cascades between cancer cells and immune cells which represent critical processes in terms of drug development for cancer treatment and immunotherapy.

On-demand multi step stimulation at defined positions. In addition, the present disclosure pertains to a method for the on-demand stimulation of (single) cells that are located within hydrogel matrices/vehicles (FIG. 31 and FIG. 32). To this end, a first hydrogel matrix 31A that contains (single) cells 20 is positioned directly next to a second hydrogel matrix 31B containing immobilized proteins, peptides, nucleic acids or small molecules. In a particular embodiment, these molecules are linked by a photocleavable bond thus the specific irradiation of said second hydrogel matrix with e.g. UV-light results in the cleavage of the photocleavable bond and a release of immobilized molecules. The first hydrogel matrix containing (single) cells as well as the second hydrogel matrix with immobilized molecules are located within close proximity within a microfluidic chamber that can be closed using miniaturized valves resulting in a closed compartment. Released molecules can then diffuse to the first hydrogel matrix containing (single) cells thereby stimulating these cells. After stimulation, the second hydrogel matrix is removed using the previously described RFCP geometry. Afterwards, a new third hydrogel matrix might be positioned to the first hydrogel matrix containing cells that is still located within its trapping position. The third hydrogel matrix might contain a second stimulus which might be a different one than the first stimulus from the second hydrogel matrix. In principle, this process can be repeated many times.

The advantage of this method is that cell phenotypes can be monitored (e.g. by using genetically modified cell lines that express a fluorescent protein coupled to certain transcription factors) by time-lapse microscopy (or any similar technique) and that a stimulus is given dependent on the observed phenotype. In addition, cells located within the disclosed array might be individually stimulated in a multistep and time-lapse manner.

The disclosed method might be used for the differentiation of stem cells. For example, the differentiation of hematopoietic stem cells is a sequential multi-step process in which cells pass through different differentiation states and each differentiation state needs a new individual stimulus. Thus, this system might be used for the highly-controlled differentiation of stem cell populations into desired phenotypes.

In addition, said method might be used for the on-demand transfection of (single) cells using CRISP Cas and the generation of knock-out cells. The method would be the same in terms of the on-demand stimulation of cells with prior phenotype monitoring. For example, the method might be used to knock-out certain transcription factors to investigate their influence on stem cell differentiation. Another example might be the generation of knock-out cells to identify key mutations leading to increased cell growth (growth rate can be directly quantified with our system by measuring the colony size). Another example is the transfection of single cells and subsequent screening for the right phenotype and the subsequent removal of cells that are viable and which show the desired phenotype. A third potential application might be the stimulation of cells depending on the current cell cycle status that might be monitored using conventional bright field microscopy. For examples, the testing of new drugs and their influence of cells depending on the current cell cycle status might be of great interest.

Cryopreservation. In addition, the present disclosure pertains to a method for the cryopreservation of cells located within said n×m array. To this end, cells are first encapsulated into hydrogel matrices and immobilized within said array. The hydrogel matrix might contain immobilized compounds that act as cryoprotectant such as glycerol or DMSO. Said compounds might decrease the number of ice crystals at the cell membrane resulting in a higher cell viability after thawing. Afterwards, the array might be perfused with a soluble cryoprotectant such as a solution containing glycerol or DMSO. The whole array might then be frozen, in particular to a temperature below −20° C., below −80° C. or below −190° C. After a certain storage time said frozen array is thawed again and the cell viability of encapsulated cells is monitored using an optical setup such as a microscope. The cell viability might be verified by monitoring the proliferation of cells located within said hydrogel matrices. Afterwards, proliferating cells might be transferred into another format using the described reverse flow cherry picking technique. Transferred cells might be expanded.

This method has several advantages in comparison to existing methods:
Firstly, a significantly lower number of cells is needed for cryopreservation and subsequent thawing. This is critical for the cryopreservation of rare cells such as stem cells isolated from patients (e.g. from cord blood or adult stem cells).
Secondly, due to the use of a microfabricated array the volume for cell expansion is significantly reduced which is especially important if costly compounds are needed for cell expansion
Thirdly, cells of interest (such as viable cells) can be selected and transferred into a larger format for cell expansion

The following method is for the on-demand stimulation of (single) cells that are located within spherical hydrogel matrices/vehicles. A spherical hydrogel matrix that contains (single) cells is positioned directly next to a spherical hydrogel matrix containing immobilized proteins, peptides, nucleic acids or small molecules. These molecules are linked by a photocleavable bond thus irradiation of such hydrogel matrix with e.g. UV-light results in the cleavage of the photocleavable bond and a release of immobilized molecules. The spherical hydrogel matrix containing (single) cells as well as the hydrogel matrix with immobilized molecules are located within close proximity within a microfluidic chamber that can be closed using miniaturized valves resulting in a closed compartment. Released molecules can then diffuse to the hydrogel matrix containing (single) cells thereby stimulating these cells. With the reverse flow cherry picking technique we can remove the “empty” hydrogel matrix that contained immobilized molecules and can load a new, “fresh” and loaded hydrogel matrix for a second stimulus which might be a different one. In principle, this process can be repeated many times. This set up allows, that cell phenotypes are monitored (e.g. by using genetically modified cell lines that express a fluorescent protein coupled to certain transcription factors) by time-lapse microscopy and that a stimulus is given dependent on the observed phenotype. In addition, the more than 2600 individual microfluidic chambers that can be addressed individually might be positioned on the microfluidic chip. Thus, at 2600 positions cells might be individually stimulated in a multistep and time-lapse manner.

A potential application might be the differentiation of stem cells. For example, the differentiation of hematopoietic stem cells is a sequential multi-step process in which cells pass through different differentiation states and each differentiation state need a new individual stimulus. Thus, this system might be used for the highly-controlled differentiation of stem cell populations into desired phenotypes.

A second potential application might be the on-demand transfection of (single) cells using CRISP Cas and the generation of knock-out cells. The method would be the same in terms of the on-demand stimulation of cells with prior phenotype monitoring. A key experiment might be to knock-out certain transcription factors to investigate their influence on stem cell differentiation. Another key experiment might be to generate knock-out cells to identify key mutation leading to increased cell growth (growth rate can be directly quantified with our system by measuring the colony size). The most trivial experiment might be to transfect single cells and screen for the right phenotype+subsequently pick cells that are viable and show the desired phenotype.

A third potential application might be the stimulation of cells depending on the current cell cycle status that might be monitored using conventional bright field microscopy. For examples, the testing of new drugs and their influence of cells depending on the current cell cycle status might be of great interest.

The following method is an example of the on-demand transfection of (single) cells using CRISPR Cas and the generation of knock-out cells. This method could be adapted to other applications:

The following method is for the time-lapse monitoring of molecules that are secreted by single cells or small cell colonies or upon the cell-cell interaction of two (single) cells. The aim was to develop a method that enables to generate time-lapse cytokine profiles of a larger number of single cells (above 2600). To this end, single cells are encapsulated into spherical hydrogel matrices and positioned on a microfluidic array. Afterwards a second hydrogel matrix that contains a mix of primary antibodies against defined target analytes is immobilized directly next to a hydrogel matrix containing a cell. Thus, one hydrogel matrix for cell cultivation and one hydrogel matrix for analyte detection are positioned in a closed microfluidic chamber. Upon secretion of single molecules such as specific cytokines, secreted analytes diffuse to the hydrogel matrix containing primary antibodies. After a defined time period (e.g. 1 hour) the microfluidic chamber is opened and washed (e.g. with PBS). Afterwards a second antibody that has coupled a barcoded oligonucleotide (for the identification of the antigen specificity) is added to the (washing fluid) perfusion system. This second antibody binds to the analyte located within the hydrogel matrix that is already bound to the primary antibody. The second antibody binds a different epitope than the primary antibody. Afterwards, all non-bound secondary antibodies are washed away and the hydrogel matrix containing now the primary antibody, the analyte and the secondary antibody is removed from the trap by reverse flow cherry picking while the hydrogel matrix containing the cell/s stays within the trap. Afterwards, a new hydrogel matrix is loaded again and the process is repeated. Hydrogel matrices that are removed from the trap are collected in a well-plate or another format. Thus, each well corresponds to a defined position and time-point on the microfluidic chip.

The use of barcoded secondary antibodies enables multiplexing. Thus, multiple analytes can be detected with one hydrogel matrix that contains different antibodies (e.g. screening for multiple cytokines such as TNF-alpha, IL-6, IL-10, Il-1). The collected hydrogel matrices can be used for quantifying the bound analytes by detecting the barcoded oligonucleotides bound to the secondary antibody. This can be done by qRT-PCR or digital PCR. Another possibility might be to amplify such oligonucleotides and then sequence the amplified product (e.g. with nanopore sequencing or similar techniques).

This method might not only be used for the analysis of the secreted molecules of single cells (or cell colonies) but also for the analysis of the cell-cell interaction between to different cell types (e.g. an immune cell and a cancer cell). To this end, an immune cell and a cancer cell are co-encapsulated within one hydrogel matrix and the time-lapse secretion profile is subsequently monitored as described previously.

In another embodiment, an immune cell is encapsulated within one hydrogel matrix and a cancer cell is encapsulated within a second hydrogel matrix. Afterwards, three spherical hydrogel matrices are paired, one containing the immune cell, one containing the cancer cell and one containing the detection antibodies. The hydrogel bead with the detection antibodies can be specifically removed with the other two hydrogel beads staying on the microfluidic chip. The detection method is the same as described above. This setup has the advantage that each cell type can be located within a different hydrogel matrix having different characteristics (e.g. different mechanical strength or different immobilized ECM compounds or growth factors that influence the cell behavior). For example, a cancer cell might need different ECM compounds than an immune cell. This experimental set-up would allow the identification of communication cascades between cancer cells and immune cells which represent critical processes in terms of drug development for cancer treatment and immunotherapy.

The invention has been described above and in the claims. Preferred embodiments, also referred to as items, are also disclosed in the following and with respect to the hydrogels and methods relating thereto specific advantages are again highlighted. The following items are also part of the present invention:
1. Microfabricated valve (10), comprising
   a first channel (11);
   a second channel (12);
   a connection channel (13) connecting the first channel (11) and the second channel (12);
   a valve portion (14) arranged within the connection channel (13),
   wherein the valve portion (14) is adapted to selectively open and close the connection channel (13).
2. Microfabricated valve (10) according to item 1, wherein the longitudinal axis of the connection channel (13) is not parallel to the longitudinal axis of the first channel (11) and/or to the longitudinal axis of the second channel (12), in particular the longitudinal axis of the connection channel (13) is substantially orthogonal to the first channel (11) and/or to the second channel (12).
3. Microfabricated valve (10) according to item 1 or 2, wherein the longitudinal axis of the connection channel (13) is substantially parallel or at an angle between 0° and 90°, in particular between 0° and 45°, to the normal vector of the surface of the first channel (11) facing the connection channel (13) and/or the longitudinal axis of the connection channel (13) is substantially parallel or at an angle between 0° and 90°, in particular between 0° and 90°, to the normal vector of the surface of the second channel (12) facing the connection channel (13)
4. Microfabricated valve (10) according to any of the preceding items,
   wherein the valve portion (14) comprises at least one flexible membrane (15), the flexible membrane (15) is adapted to be selectively transferred between an open shape and a closed shape, and in particular between an intermediate shape,
   in particular
   wherein in the open shape a transfer of fluid between the first channel (11) and the second channel (12) and/or vice versa is enabled and wherein in the closed shape a transfer of fluid between the first channel (11) and the second channel (12) and/or vice versa is disabled,
   in particular the membrane (15) is adapted to be selectively transferred into an intermediate shape, wherein in the intermediate shape a flow resistance in the valve (10) is increased compared to the open shape.
5. Microfabricated valve (10) according to any of the preceding items, wherein the connection channel (13) is connected to the first channel (11) by at least one first opening (2) and the connection channel (13) is connected to the second channel (12) by at least one second opening (1).
6. Microfabricated valve (10) according to the preceding item, wherein the first opening (2) is adjacent to a first end of the connection channel (13) and/or the second opening (1) is adjacent to a second end of the connection channel (13).
7. Microfabricated valve (10) according to the preceding item, wherein the first end of the connection channel (13) is a first end face of the connection channel (13) and/or the second end of the connection channel (13) is a second end face of the connection channel (13).
8. Microfabricated valve (10) according to any of the items 5 to 7, wherein the shape of the first opening (2) differs from the shape of the cross section of the connection channel (13), in particular from the shape of the first end of the connection channel (13), and/or the shape of the second opening (1) differs from the shape of the cross section of the connection channel (13), in particular from the shape of the second end of the connection channel (13).
9. Microfabricated valve (10) according to one of the items 5 to 8, wherein the cross section (7) of the connection channel (13) is larger or smaller than the first opening (2) and/or the second opening (1).
10. Microfabricated valve (10) according to any of the items 5 to 9, wherein the shape of the first opening (2) and the shape of the second opening (1) are identical or different.
11. Microfabricated valve (10) according to any of the items 5 to 10, wherein the first opening (2) and the second opening (1) are substantially coaxial or not coaxial.
12. Microfabricated valve (10) according to the any of the items 5 to 11, wherein the number of the first openings (2) and the number of the second openings (1) are different.
13. Microfabricated valve (10) according to any of the preceding items, wherein the valve portion (14) is adapted to be selectively opened and closed, in particular transferred into an intermediate shape, upon modification of a fluid pressure of a pressure, in particular of a fluid pressure of a control fluid, in particular compressed air, acting onto the membrane (15),
   in particular that the flexible membrane (15) is transferred into the open shape and/or transferred into the closed shape and/or into the intermediate shape upon decreasing/increasing the fluid pressure.
14. Microfabricated valve (10) according to item 12, comprising at least one actuation chamber (3), wherein the connection channel (13) is separated from the actuation chamber (3) by at least a section of the flexible membrane (15), wherein the fluid pressure of the control fluid acting onto the membrane (15) within the chamber (3).
15. Microfabricated valve (10) according to any of the preceding items, comprising at least one actuation chamber (3), wherein the connection channel (13) is separated from the actuation chamber (3) by at least one section of the flexible membrane (15), in particular this section extends over the entire circumference of the connection channel (13), wherein the valve portion (14) is adapted to be selectively opened and closed, and in particular transferred into an intermediate shape, upon modification of a pressure difference between the actuation chamber (3) and the connection channel (13) by modification of the pressure inside the actuation chamber (3), wherein the pressure inside the chamber (3) is adjusted, in particular by a actuation fluid which can flow into the actuation chamber to increase the pressure inside the chamber or to flow out of the chamber to decrease the pressure inside the chamber, in particular to generate a vacuum inside the actuation chamber (3).
16. Microfabricated valve (10) according to the preceding item, comprising at least a second actuation chamber (111B), wherein the connection channel (13) is separated from the second actuation chamber (111B) by a second section (107) of the flexible membrane (15), wherein the second section (107) of the flexible membrane (15) and the first section (106) of the flexible membrane (15) are different,
   wherein the valve portion (14) is adapted to be selectively transferred into an open and/or closed and/or intermediate shape upon modification of a pressure difference between the second actuation chamber (111B) and the connection channel (13) by modification of the pressure inside the second actuation chamber (111B), wherein the pressure inside the second actuation chamber (111B) is adjusted, in particular by a actuation fluid which can flow into the second actuation chamber (111B) to increase the pressure inside the second actuation chamber (111B) or to flow out of the second actuation chamber (111B) to decrease the pressure inside the second actuation chamber (111B), in particular to generate a vacuum inside the second actuation chamber (111B).
17. Microfabricated valve (10) according to the preceding item, wherein the pressure inside the first actuation chamber (111A) and the pressure inside the second actuation chamber (111B) can be modified independently.
18. Microfabricated valve (10) according to any of the preceding items, characterized in,
   that the valve portion (14) is adapted to be selectively opened and closed upon modification of a voltage applied to the valve portion, in particular the valve portion comprises at least one electrostatic chargeable layer, in particular polymer layer, which is adapted to change its form upon modification of the voltage.
19. Microfabricated valve (10) according to any of the preceding items, characterized in,
   that the microfabricated valve (10) comprises at least three layers (21, 22, 23), wherein
   the first channel (11) is located within a first layer (21);
   the second channel (12) is located within a third layer (23);
   the valve portion (14) is located within a second layer (22);
   the second layer (22) is arranged between the first (21) and the third layer (23).
20. Microfabricated valve (10) according to the preceding item, wherein the first opening (2) is located within the first layer (21) and/or the second opening (1) is located within the third layer (23).
21. Microfabricated valve (10) according to the preceding item, wherein the first opening (2) is located within the first layer (21) and the second opening (1) is located within the second layer (22) or
   wherein the second opening (1) is located within the third layer (23) and the first opening (2) is located within the second layer (22).
22. Microfabricated valve (10) according to the preceding item, wherein the actuation chamber (3) and/or the second actuation chamber (111B) is located within the second layer (22).
23. Microfabricated valve (10) according to the preceding item, wherein the actuation chamber (3) and/or the second actuation chamber (111B) is arranged at least partly between the first channel (11) and the second channel (12).
24. Microfabricated valve (10) according to any of the items 1 to 18, characterized in,
   that the microfabricated valve (10) comprises one layer, wherein
   the first channel (11), the second channel (12) the valve portion (14) and in particular the actuation chamber (3) is located within the layer.
25. Microfabricated valve (10) according to any of the items 4 to 20, wherein the flexible membrane (15) comprises
   an inner boundary forming the outer wall of the connection channel (13) or encompassing at least one section of the connection channel (13)
   and an outer boundary forming the outer wall of the flexible membrane (15), wherein the inner boundary is adapted to be transferred between an open and closed shape, and in particular between an intermediate shape,
   wherein in the opened shape a transfer of fluid between the first channel (11) and the second channel (12) passing the inner boundary and/or vice versa is enabled and wherein in the closed shape a transfer of fluid between the first channel (11) and the second channel (12) passing the inner boundary and/or vice versa is disabled,
   in particular the inner boundary is adapted to be selectively transferred into an intermediate shape, wherein in the intermediate shape a flow resistance in the valve (10) is increased compared to the open shape.
26. Microfabricated valve (10) according to the preceding item, wherein the inner boundary is defined by different inner boundary sections, each encompassing a different section of the connection channel (13),
   wherein the inner boundary sections are adapted to be transferred between an open and closed shape, and in particular between an intermediate shape.
27. Microfabricated valve (10) according to the preceding item, wherein the inner boundary sections are adapted to be transferred into an open and/or closed and/or intermediate shape independently.
28. Microfabricated valve (10) according to any of the items 25 to 27,
   wherein the first section of the connection channel (13) is separated from the actuation chamber (3) by the at least first section (106) of the flexible membrane (15),
   wherein the first inner boundary section is adapted to be selectively transferred between an opened and closed shape, and in particular into an intermediate shape, upon modification of a pressure difference between the actuation chamber (3) and the first section (106) of the connection channel (13) by modification of the pressure inside the actuation chamber (3), wherein the pressure inside the actuation chamber (3) is adjusted, in particular by the actuation fluid which can flow into the actuation chamber (3) to increase the pressure inside the actuation chamber (3) or to flow out of the actuation chamber (3) to decrease the pressure inside the actuation chamber (3), in particular to generate a vacuum inside the actuation chamber (3).
29. Microfabricated valve (10) according to the preceding item, wherein the second section (117) of the connection channel (13) is separated from the second actuation chamber (111B) by a second section (107) of the flexible membrane (15), wherein the second section (107) of the flexible membrane (15) and the first section (106) of the flexible membrane (15) are different,
   wherein the second inner boundary is adapted to be selectively transferred between an opened and closed shape, and in particular into an intermediate shape, upon modification of a pressure difference between the second actuation chamber (111B) and the second section (117) of the connection channel (13) by modification of the pressure inside the second actuation chamber (111B), wherein the pressure inside the second actuation chamber (111B) is adjusted, in particular by the actuation fluid which can flow into the second actuation chamber (111B) to increase the pressure inside the second actuation chamber (111B) or to flow out of the second actuation chamber (111B) to decrease the pressure inside the second actuation chamber (111B), in particular to generate a vacuum inside the second actuation chamber (111B).
30. Microfabricated valve (10) according to any of the items 25 to 29, wherein a first first opening (2, 104, 108) connects the first channel (11) with a first section (116) of the connection channel (13) and a second first opening (2, 109) connects the first channel (11) with a second section (117) of the connection channel (13)
   and/or
   wherein a first second opening (1, 102, 108) connects the second channel (12) with the first section (116) of the connection channel (13) and a second second opening (1, 103, 109) connects the second channel (12) with a second section (117) of the connection channel (13).
31. Microfabricated valve (10) according to item 25 to 30, comprising a second channel (115),
   wherein a first second opening (1, 102, 108) connects the second channel (12) with a first section (116) of the connection channel (13) and a second opening (1, 103, 109) connects the second channel (115) with a second section (117) of the connection channel (13)
   and/or
   wherein a first opening (2, 104, 108) connects the first channel (11) with the first section (116) of the connection channel (13) and a second first opening (2, 109) connects the first channel (11) with the second section (117) of the connection channel (13).
32. Microfabricated valve (10) according to any of the preceding items,
   wherein the flexible membrane (15) and/or the at least one actuation chamber (3, 111A, 111B) has a homogeneous or inhomogeneous thickness in particular the thickness depends on the deflection distance of the flexible membrane (15), wherein the deflection distance is the distance of the position of a point on the inner boundary of the flexible membrane while the flexible membrane (15) is in the closed shape and the position of this point while the flexible membrane is in the opened shape,
   especially preferred the flexible membrane has a thinned section which has a reduced thickness compared to at least one other section of the flexible membrane (15), in particular the thinned section is the thinnest section, wherein the thinnest section is at the position of the maximal deflection distance.
33. Microfabricated valve (10) according to the preceding item, wherein the flexible membrane (15) has a thinned section which has a reduced thickness compared to at least one other section of the flexible membrane (15), this section being the one adjacent to the first layer (21), and a projection of the first channel (11) along the longitudinal axis of the connecting channel (13) meets this thinned section and/or
   wherein the flexible membrane (15) has a thinned section which has a reduced thickness compared to at least one other section of the flexible membrane, this section being the one adjacent to the third layer (23), and a projection of the second channel (12) along the longitudinal axis of the connecting channel (13) meets this thinned section.
34. Microfabricated valve (10) according to the any preceding item, wherein the actuation chamber (3) and/or the second actuation chamber (111B) has a thinned chamber section which has a reduced thickness compared to at least one other section of the chamber, this section being the one adjacent to the first layer (21), and a projection of the first channel (11) along the longitudinal axis of the connecting channel (13) meets this thinned chamber section and/or
   wherein the actuation chamber (3) and/or the second actuation chamber (111B) has a thinned chamber section which has a reduced thickness compared to at least one other section of the chamber, this section being the one adjacent to the third layer (23), and a projection of the second channel (12) along the longitudinal axis of the connecting channel (13) meets this thinned chamber section.
35. Microfabricated valve (10) according to any of the preceding items,
   wherein the inner boundary or an inner boundary section of the flexible membrane (15) has a biconvex or biconcave shape or a polygonal shape, in particular a triangular, rectangular, pentagonal shape, or a shape where at least one edge is curved, in particular convex or concave, for example plano-convex or plano-concave.
36. Microfabricated valve (10) according to any of the preceding items, wherein the first channel (11) comprises a positioning means suitable for positioning particles (20) being contained in a fluid which flows through the first channel, wherein the positioning means is arranged within the first channel (11) in such a way that a fluid flow can be reduced by the positioning means, in particular, the positioning means narrows the cross section of the channel and/or
   wherein the second channel (12) comprises a positioning means suitable for positioning particles (20) being contained in a fluid which flows through the second channel (12), wherein the positioning means is arranged within the second channel (12) in such a way that a fluid flow can be reduced by the positioning means, in particular, the positioning means narrows the cross section of the channel.
37. Microfabricated valve (10) according to the preceding item, wherein the positioning means is arranged within the first channel (11) in such a position that a projection of the first opening (2) along its axis meets at least a part of the positioning means of the first channel (11) and/or
   wherein the positioning means is arranged within the second channel (12) in such a position that a projection of the second opening (1) along its axis meets at least a part of the positioning means of the second channel (12).
38. Method for manufacturing a microfabricated valve (10) according to any of the preceding items, comprising:
   inserting the first channel (11) into the first layer (21),
   inserting the second channel (12) into the third layer (23),
   inserting the connection channel (13) with the valve portion (14) into the second layer (22),
   and then arranging the second layer (22) between the first layer (21) and the third layer (23).
39. Method according to the preceding item, further comprising:
   inserting the actuation chamber (3) and/or the second actuation chamber (111B) into the second layer (22) before arranging the second layer (22) between the first layer (21) and the third layer (23).
40. Test device (30), in particular for biological applications, in particular comprising at least one location in particular observation chamber (32), in particular a plurality of locations (32), wherein the test device (30), in particular the observation chamber (32), is adapted to accommodate an object in a fluid, in particular the object comprising at least one droplet (31) in particular comprising a hydrogel particle and/or hydrogel matrix.
41. Test device (30) according to the preceding item, wherein the test device (30) is adapted to accommodate an object (31) selected from one or more of: droplet, in particular hydrogel particle, hydrogel bead, hydrogel droplet, fluid, in particular fluorinated oil, aqueous fluid, a water-in-oil droplet, an oil-in-water droplet, an water-in-oil-in-water droplet (double emulsion), triple emulsion, multiple emulsion, and/or at least one particle (20) or a plurality of particles (20), in particular biological cell or cells, microstructures, in particular microfabricated electrodes, nanostructures, gold nanocrystals, biological compound, wherein the term biological compound comprises DNA, RNA, proteins, in particular antibodies, LNA, PNA, small molecules, photocleavable linker,
   in particular one of more particles may be contained within a droplet.
42. Test device (30) according to any of items 40 to 41,
   characterized in
   that the test device (30) comprising at least one valve (10), in particular a plurality of valves (10), according to any of items 1 to 37.
43. Test device (30) according to any of items 40 to 42,
   characterized in,
   that the test device (30) comprises at least one in particular a plurality of positioner (33) adapted to position an object, in particular a particle (20) or droplet (31), in a predefined location (3) within the test device (30).
44. Test device (30) according to the preceding item, that the positioner (33) is a positioning means or a trap (33, 17), in particular a particle trap and/or a droplet trap, to retain a predetermined number of objects, which are provided within a stream of fluid (36) passing the positioner (33, 17), in particular in a first fluid direction (S1),
   in particular wherein the positioner (33, 17) comprising a bottleneck section (16, 34) having a smaller diameter than an object to be retained.
45. Test device (30) according to item 43 or 44,
   characterized in,
   that the positioner (33), in particular the trap (33, 17), comprising a bypass section (18, 35), in which objects can circumvent the bottleneck section (16, 34) when the positioner (33, 17) is occupied by a predetermined number, in particular one, of retained objects.
46. Test device (30) according to any of items 43 to 45,
   characterized in
   that adjacent, in particular below or above, the positioner (33, 17), a valve portion (14), in particular of a valve (10) according to any of items 1 to 37, is provided, wherein the test device (30) is adapted to selectively transfer the objects from the positioner (33, 17) through the valve portion (14) from one opening (1, 2) of the valve, to an opposite opening (1, 2) of the valve, in particular from one channel (12, 11) through a first/second opening (1, 2) into another channel (11, 12) through second/first opening (1, 2).
47. Test device (30) according to any of items 43 to 46,
   characterized in
   that the test device (30) comprises two neighbouring positioner (17n), wherein the valve portion (14) is located adjacent to, both positioner (17n), wherein the test device (30) is adapted to selectively transfer the objects from both positioner (17n) through the valve portion (14) from one second channel (12) or from two separate second channels (12′, 12″) into a separate first channel (11),
   in particular wherein in the both second channels (12′, 12″) a same second pressure (p12) is applied to the fluid.
48. Test device (30) according to any of items 40 to 47, comprising
   a collection chamber, in particular droplet collection channel (61),
   a substance supply channel, in particular a liquid supply channel (64C),
   the collection chamber (61) is adapted to be selectively opened and closed, in particular by means of a first valve (63A) located at a first end of the collection chamber (61) and a second valve (63B) located at a second end of the collection chamber (61);
   a passage (69) from the supply channel (64C) to the collection chamber (61) is adapted to be selectively opened and closed in particular by means of a third valve (63C), allowing an amount of substance, in particular liquid, to flow from the supply channel (64C) to the collection chamber (61) in particular for droplet generation,
   in particular at least one of the valves (63) is according to any of items 1 to 37.
49. Test device (30) according to the preceding item, characterized by
   a damping device (65), in particular a membrane structure, connected to the collection chamber (61),
   the damping device is adapted to increase the volume of the collection chamber (61) corresponding to the amount of substance, in particular liquid, transferred from the supply channel (64C) to the collection chamber (61).
50. Test device (30) according to the preceding item, characterized in that the damping device (65) has a membrane (66) arranged between the collection chamber (61) and a compensating pressure (p10),
   in particular the compensation pressure (p10) is provided by a liquid or a gas of, in particular known, pressure within a compensation chamber (68) or a resilient member adjacent to the membrane,
   in particular wherein compensation pressure (p10) is the atmospheric pressure and/or the compensation chamber (68) is connected to the atmosphere;
   in particular the membrane (66) is made in one piece with a housing (610) of the test device.
51. Test device (30) according to any of items 40 to 50, comprising a centering station (70), the centering station (70) is adapted to accommodate at least one droplet (31) and to bring the accommodated droplet (31) into rotation, so that a centering effect is applied to a particle (20) located within the droplet (31), in particular the centering station (70) comprising a positioner (33), in particular a droplet trap (33) in particular having a bottleneck section (16).
52. Test device (30) according to item 50 or 52,
   characterized in that
   the centering station (70) is adapted to:
      in a first step to position the droplet (31) in a predefined position, in particular with in a positioner in particular droplet trap (33), in particular by applying a flow of fluid along a first path of flow (71),
      in a second step to selectively bring the droplet (31) into rotation within the predefined position, in particular by applying a flow of fluid along a second path of flow (72);
      in a third step urge the droplet (31) out of the predefined position, in particular by applying a flow of fluid along a third path of flow (73);
   in particular the flow of fluid along one of the paths of fluid (71, 72, 73) is selectively controlled by a valve arrangement having a plurality of valves (V1-V5), which are adapted to be selectively opened and closed
   in particular the centering station constitutes the positioner (33) according to any of items 43 to 47.
53. Test device (30) according to any of item 50 to 52,
   characterized in
   that during the second step the fluid urging the droplet in a direction (C), preventing the droplet (31) to move out of the positioner (33); and/or.
   that the second path of fluid (72) and the predefined position are arranged in manner so that
      the flow of fluid flowing along the second path of fluid (72) contacting the droplet (31) in a tangential direction and and/or
      the droplet is urged by the flow of fluid along a second path of flow (72) into a condition in which it is hindered to get out of the positioner (33).
54. Test device (30) according to the any of items 43 to 53,
   characterized in,
   that the positioner (33), in particular the trap (33, 17), is adapted to selectively release a retained object, in particular adapted to selectively release a t least one retained object,
   in particular at least one of a plurality of retained objects, upon application of a fluid in a second fluid direction (S2), in particular opposite a the first fluid direction (S1).
55. Test device (30) according to any of items 40 to 54,
   characterized in,
   that test device (30) is adapted to selectively release a retained object within a selected location (32), in particular an observations chamber (32), wherein the at least one unselected location (32) is adapted to keep on retaining the at least one retained object.
56. Test device (30) according to any of the items 40 to 55,
characterized by
an exit delivery mechanism is adapted to deliver a released object to an exit portion (P2), in particular the exit portion is selected from a plurality of exit portions;
   in particular:
   the test device (30) comprises a plurality of locations (32) a plurality of exit portions (P2),
   a first group of locations (32m,32n) is connected to a first exit portion,
   a second group of locations (32m,32n) is connected to a second exit portion.
57. Test device (30) according to any of items 40 to 56,
   characterized in,
   that the positioner (33), in particular trap (33, 17), is adapted to retain a predefined sequence of objects, in particular droplets (31A, 31B, 31C) or particles, subsequently arriving at a predefined location (32), in particular observation chamber (32), at separate predefined positions,
   in particular the positioner (33, 17) comprising a plurality of bottleneck section (34A,34B,34C), in particular arranged in series defining the positions.
58. Test device (30) according to the any of items 40 to 57,
   characterized in
   that the positioner (33), in particular trap (33, 17), is designed in a way, that upon a change of the direction of fluid a specific force is applied to the objects pushing the objects out of the positioner (33), wherein the respective pushing force is different for each of the predefined subpositions (34A, 34B, 34C).
59. Test device (30) according to any of items 40 to 58,
   characterized by,
   each location (32), in particular observation chamber (32), has a valve arrangement (40) adapted to provide a fluid passing through the positioner in particular the trap (17, 33), wherein the valve arrangement (40) is adapted to selectively change the direction of fluid (S1, S2) passing the location (32), in particular wherein a fluid a first direction (S1) urging the object into the positioner (33) and a fluid in the second direction (S2) urging the object out of the positioner (33),
   and in particular fluid in the second direction (S2) delivering the object in direction of the exit section (P2).
60. Test device (30) according to any of items 40 to 59,
   characterized by,
   a dielectrophoretic (DEP) force generator (44), for generating a dielectrophoretic (DEP) force acting on an object, in particular the dielectrophoretic (DEP) force generator (44) is part of a positioner, in particular trap (33, 17), for retaining an object.
61. Test device (30) according to any of items 40 to 60,
   characterized in
   that a positioner (33), in particular a trap (33, 17), comprises a structure (46), which is adapted to stimulate the object to rotate upon application of a stream of fluid acting on the object.
62. Test device (30) according to any of the items 40 to 61,
   characterized by a camera focused on a positioner (33), in particular a trap (33, 17), adapted to take an optical image of an object, which is positioned within the positioner (33), in particular retained within the trap (33, 17).
63. Test device (30) according to any of the preceding items,
   characterized by a light source focused on a positioner (33), in particular trap (33, 17), adapted to expose an light beam onto an object, which is positioned within the positioner (33).
64. Test device (30) according to any of items 40 to 63,
   characterized in that,
   for changing the direction of flow (S1, S2) through the positioner (33) a plurality of the locations in particular observation chambers (32) each having a respective valve arrangement (40m2n2).
65. Test device (30) according to the preceding item,
   characterized in that
   each of the valve arrangements (40m2n2) are allocated
   a) to one of a first group (m2) of valves arrangements (40m2) and
   b) to one of a second group (n2) of valve arrangements (40n2),
   wherein the valve arrangements of one group can be triggered commonly by a respective common group command (Cm1, Cm2, Cm3, Cn1, Cn2, Cn3, . . . );
   in particular wherein one common group command comprises a first group commands (Cm1, Cm2, Cm3, . . . ) and a second group commands (Cn1, Cn2, Cn3, . . . ).
66. Test device (30) according to any of items 64 or 65,
   characterized in,
   that the valve arrangement (40m2, n2) is adapted to change the direction of the fluid within a positioner (33) if both group commands issue a group command (Cm2=1, Cn2=1) referring to the both groups to which the valve arrangement (40m2n2) belongs, and/or
   that the valve arrangement (40m2, n2) is adapted to release an object retained within the positioner (33) if both group commands issue a group command (Cm2=1, Cn2=1) referring to the both groups to which the valve arrangement (40m2n2) belongs.
67. Test device (30) according to any of items 64 to 66,
   characterized in,
   that the valve arrangement (40) comprising
   a first path of flow (51) directing through the positioner (33) in a first direction (S1) and
   a second path of flow (52) directing through the positioner (33) in a second direction (S2)
   in particular the first path (51) and the second path (52) connecting one common inlet (P1) with one common exit (P2),
   wherein the first path (51) comprises a hydrodynamic resistance (R0+R2+R3);
   wherein the second path (52) comprises a hydrodynamic resistance (R0+R1+R4),
   wherein the hydrodynamic resistance (R0+R1+R2) in the first path (51) can be varied upon activating a selected valve of the valve arrangement.
68. Test device (30) according to any of items 64 to 67,
   characterized in,
   that the valve arrangement (40) comprising
   at least a third path of flow (53) and/or a fourth path or flow (54) bypassing the positioner (33),
   in particular the third path (53) and the fourth path (54) connecting one common inlet (P1) with one common exit (P2),
   wherein the third path (53) comprises a hydrodynamic resistance (R1+R2);
wherein the fourth path (54) comprises a hydrodynamic resistance (R3+R4),
   wherein the hydrodynamic resistance in the third path (53) and/or in the fourth path (54) can be varied upon activating a selected valve of the valve arrangement (40).
69. Test device (30) according to any of the two preceding items,
   characterized in
   that within the valve arrangement (40) the paths of fluid (51, 52, 53, 54) comprises:
      a first fluid line (501) having a first hydrodynamic resistance (R1) located between an inlet (N012) of the positioner (33) and the common exit (P2); and/or
      a second fluid line (502) having a second hydrodynamic resistance (R2) located between the common inlet (P1) and an inlet (N012) of the positioner (33); and/or
      a third fluid line (503) having a third hydrodynamic resistance (R3) located between an outlet (N034) of the positioner (33) and the common exit (P2); and/or
      a fourth fluid line (504) having a fourth hydrodynamic resistance (R4) located between the common inlet (P1) and an outlet (N034) of the positioner (33); and/or
      a fifth fluid line (505) having a fifth hydrodynamic resistance (R0), in which the positioner (33) is arranged;
   in particular the inlet (N012) and the outlet (N034) of the positioner (33) is arranged within a feeding line (41) line of the test device (30),
   in particular fluid passing the location (33) from the inlet (N012) to the outlet (N034) in a first direction (S1) and from the outlet (N034) to the inlet (N012) in a second direction (S2).
70. Test device (30) according to items 68 or 69,
   characterized in
   that the second hydrodynamic resistance (R2) can be varied from a value smaller than the fourth hydrodynamic resistance (R4) to a value larger than the fourth hydrodynamic resistance (R4) in particular by triggering a first group command (Cm2); and/or
   that the that the third hydrodynamic resistance (R3) can be varied from a value smaller than the first hydrodynamic resistance (R1) to a value larger than the first hydrodynamic resistance (R1) in particular by triggering a second group command (Cn2).
71. Test device (30) according to any of items 40 to 70,
   characterized by
   a feeding channel (41), adapted for initially supplying objects, in particular droplets (31) or particles (20), in a fluid from an inlet into a one or a plurality of locations in particular observation chambers (32), wherein in particular the plurality of locations (32) are connected by the feeding line (41) in series.
72. Test device (30) according to any of items 40 to 71,
   characterized by
   an impedance measuring device (38) for measuring the impedance of at an object, particular droplet (31) or particle (20), in particular at a location (32), where the object is held stationary, in particular for at least 0.1 seconds,
   in particular the impedance measuring device (38) is part of a positioner (33).
73. Test device (30) according to any of items 40 to 72,
   comprising a radio frequency application device (39) for applying a radio frequency to an object, in particular droplet (31) or a particle (20), in particular at a location, where the object is held stationary, in particular for at least 0.1 seconds,
   wherein the radio frequency application device (39) is in particular adapted to the object, so that the object is heated upon application of the radio frequency,
   in particular the frequency application device (39) is part of a positioner (33).
74. Method of creating s droplet (31), in particular encapsulations, within a first fluid, comprising the following steps:
   a) providing a microfabricated valve (10) according to any items 1 to 37, wherein the first channel (11) is filled with a first fluid,
   wherein the second channel (12) is filled with a second fluid,
   in particular wherein the second fluid is unsoluble in the first fluid,
   b) applying a pressure difference (p2-p1) to the fluids, wherein the second fluid is pressurized by a second pressure (p2) and the first fluid is pressurized by a first pressure (p1), wherein the second pressure (p2) is larger than the first pressure (p1),
   c) selectively opening the valve portion (14),
   d) subsequently closing the valve portion (14) as soon as a defined quantity of the second fluid has passed the valve portion (14) in direction from the second channel (12) to the first channel (11).
75. Method according to the preceding item,
   characterized in
   that at least one particle (20) is comprised within the second fluid,
   wherein the particle (20) is retained by a positioner (33), in particular trap (33, 17) above the valve portion (14),
   wherein during selectively opening and closing the valve portion (14) at least one particle (20), in particular exactly one particle (20), passing the valve section (14) along with the defined quantity of the second fluid.
76. Method according to item 74 or 75,
   characterized in that the defined quantity is adjusted
      by varying an opening duration (t_open) of the valve portion (14), and/or
      by varying a pressure difference (p2-p1) between the second channel (12) and the first channel (11), and/or
   by varying membrane properties, in particular geometry or elasticity, of damping device (65) that is in particular connected to a collection chamber, and/or
   by varying the opening level of the valve, and/or
   by varying the hydrodynamic resistance within the channel receiving the fluid through the valve portion (14) in particular the first channel (11), and/or
   by varying the hydrodynamic resistance of the collection chamber.
77. Method according to any of items 74 to 76,
   characterized by the following steps:
   using a first valve (10A) in particular according to any of items 1 to 37 to generate a first droplet (31A) having a first ingredient;
   using a second valve (10B) in particular according to any of items 1 to 37 to generate a second droplet (31B) having at least a second ingredient;
   using a third valve in particular according to any of items 1 to 37 to generate a third droplet having at least a third ingredient;
   merging both droplets (31A, 31B), in particular the three droplets, in the first channel (11) to generate a merged droplet (31AB) comprising the first and second ingredients or in particular the three ingredients, in particular by generating a flow in the first channel (11)
   in particular the first, second and third ingredient each is selected from a fluid and/or a particle.
78. Method for performing a biological test cycle, in particular using a test device (10) according to any of items 40 to 73, comprising the steps:
   providing one or a plurality of object, in particles (20) or droplets (31), in particular the droplets (31) comprising at least one particle (20), within a stream of fluid;
   selectively positioning, in particular trapping, one individual objects or a preset number of objects within the test device (30), in particular within an location (32) in particular observation chamber (32), in particular within a trap (33, 17).
79. Method according to the preceding item,
   characterized in
   that a plurality of objects is supplied in a sequence of objects to a first location (32), a preset number, in particular one or more, of objects is retained in the first location (32), in particular according to a preset maximum numbers objects to be retained in the first location (32),
   all objects subsequently approaching the first location (32) and exceeding the preset number of objects are forwarded to a second location (32) in particular observation chamber (32), in particular via a bypass section (35) of a trap (33, 17) within the location.
80. Method according to item 78 or 79,
   characterized in
   after retaining an individual object for a given time period within the location (32) in particular observation chamber (32), selectively untrapping an individual object from the location (32) and selectively delivering the untrapped object to an exit section (P2), in particular by changing, in particular reversing, the direction of fluid within the location (32) and/or trap (33, 17).
81. Method according to any of items 78 to 80,
   characterized in,
   that in case that a plurality, in particular more than one, of objects, in particular droplets (31A-31C) or particles, are retained in a single location in particular observation chamber (32), in particular having a plurality of positioner (33A, 33B, 33C), a selected one or each of the plurality of objects is individually released from the location (32), in particular by applying different forces, in particular by different fluid pressure or fluid rates, to the location (32).
82. Method according to any of items 78 to 81,
   characterized in
   that during a first step a first object, in particular droplet (31A), is held in a first positioner (33A) and a second object, in particular droplet (31B), is held in a second positioner (33B) within one location (32),
   in particular the first object, in particular droplet (31A), and second object, in particular droplet (31B), contacting each other,
   that during a second step the first object is kept in the first positioner (33A) and the second object (31B) is removed from the second positioner (33B),
   in particular that during a third step the second positioner (33B) is again loaded with a object, wherein in the first positioner (33A) still the first object is positioned,
   in particular the first object and the new loaded object contacting each other,
   in particular that the object loaded into the second positioner is again the second object (31B) or another object.
83. Method according to the preceding item,
   characterized in
   that the first object is a first droplet (31A) comprising also at least one biological cell, in particular immune cell, cancer cell, stem cell, in particular pair of cells as mentioned before; and/or
   that the second object is a second droplet (31B) comprising also proteins in particular antibodies, antibody-DNA conjugates, RNA in particular aptamer, secreted molecules in particular cytokines, small molecules in particular hormones, photocleavable spacer, drugs.
84. Method according to any of the two preceding items,
   characterized in that
   between the first step and the second step a first fluid, in particular an aqueous fluid, surrounding the objects is removed from the positioner (33) and/or is replaced by a second fluid, in particular by a, in particular fluorinated, oil;
   in particular subsequently the both objects are held stationary within the positioner (33) for a predetermined period, in particular wherein the objects are subjected to light, in particular UV, radiation and/or wherein the objects are recorded by an image recording device, in particular a microscope,
   in particular subsequently removing the second fluid and subsequently performing the second step.
85. Method according to any of items 78 to 84,
   characterized by the following steps:
   providing a droplet (31) in a second channel (12), wherein the droplet (31) comprising one or more particles (20), in particular a particle (20);
   bringing the droplet (31) into rotation, so that a centripetal force acting on the particles (20), leading to a g effect of the particles (20) within in the droplet (31), in particular wherein the centering effect may occur before and/or during a formation, in particular polymerisation, of a hydrogel within the droplet (31).
86. Method according to any of items 78 to 85,
   characterized in the step of
   extracting an ingredient of the droplet (31) from a droplet carrier material, in particular by using a microfabricated valve (10) according to any of items 1 to 37,
   in particular the droplet carrier material is immiscible with an ingredient material, in particular the droplet carrier material is an oily or aqueous fluid and/or the ingredient is an aqueous or oily fluid.
87. Method according to any of items 78 to 86,
   characterized in the steps
   a) providing a droplet (31) within a location (32), in particular an observation chamber (32), in particular trapped within a trap (33, 17), the droplet (31) comprising an immobilized particle (20), in particular hydrogel particle or matrix, and the location is filled with a first, in particular aqueous, fluid;
   b) perfusing the location with a second, in particular oily, fluid, so that the first fluid is removed from the droplet (31).
88. Method according to the preceding item,
   characterized in the step
   c) after step b, perfusing the location with the first fluid, so that the second fluid is removed from the droplet (31)
   in particular repeating the steps b) and c) at least one time.
89. Method according to any of items 78 to 88,
   characterized in
   that the test device is filled with a cryoprotectant fluid,
   subsequently the test device (30) is frozen,
   in particular wherein during filling the cryoprotectant and freezing at least an object, in particular droplet (31) and/or particle (20), is retained in a location (32), in particular in an observation chamber (32) or in a trap (33, 17), of the test device (30).
90. Method according to any of items 78 to 89, using a test device (30), in particular a test device (30) according to any of items 40 to 73, characterized by the steps of
   i) loading a number of positions (32), in particular a plurality of positions (32) within the test device (30) with objects, in particular droplets (31y, 31n) or particles (20),
   ii) subsequently determining for one or a plurality of the loaded positions (32), whether the contained objects fulfils a predefined object criteria or not (31n),
   iii) subsequently selectively unloading those objects from the location (32), which do not fulfil the predefined criteria, in particular by using a method according to any of the previous method items,
   iv) repeating step i) to iii) until a predefined number of positions, in particular all positions, contain objects, in particular droplets (31a) or particles (20), fulfilling the predefined criteria.
91. Method for demulsification of droplet (31) comprised within a first fluid, comprising the following steps:
   a) providing a microfabricated valve (10) according to any of items 1 to 37 or a test device according to any of items 40 to 73,
   wherein the first channel (11) is filled with a first fluid,
   wherein the second channel (12) is filled with a second fluid,
   wherein in the first channel (11) a droplet (31) of a fluid different to the first fluid, in particular the second fluid, is comprised,
   in particular wherein the second fluid is insoluble in the first second fluid,
92. Method according to the preceding item, comprising the following steps:
   b) in particular applying a pressure difference (p2-p1) to the channels (11, 12), wherein the second channel (12) is pressurized by a second pressure (p2) and the first channel (11) is pressurized by a first pressure (p1), wherein the first pressure (p1) is larger than the second pressure (p2), or
   selectively opening the valve portion (14), in particular wherein the lower density of the droplet (31) is used to generate a flow from the first channel (11) through the connection channel (13) and/or valve portion (14) to the second channel (12),
   b) subsequently closing the valve portion (14) as soon as the droplet (31) has passed the valve portion (14) in direction from the first channel (11) to the second channel (12).
93. Method according to the preceding item,
   characterized in
   that the one of the channels, in particular the first channel (11) or the second channel (12) is coated hydrophilic, and/or
   that the other of the channels, in particular the second channel (12) and/or the first channel (11) is coated hydrophobic and/or fluorophilic.
94. Method according to any of items 91 to 93,
   characterized in,
   that the droplet (31) comprises an ingredient, wherein after the droplet (31) has reached the second channel (12) the ingredient is released form the droplet (31).
95. Method according any of items 74 to 94,
   characterized in,
   that the second fluid is an aqueous fluid and the first fluid is an oily fluid.
96. Method according to any of items 74 to 95,
   characterized in
   that at least one object (81), in particular hydrogel matrix, containing a plurality of particles (82A-82D) and/or cells and/or a plurality of objects (81), in particular hydrogel matrices, each containing at least one particle and/or cell (82A-82D) and/or a plurality of objects (81), in particular hydrogel matrices, each containing a plurality of particles and/or cells (82A-82D),
   wherein parameters (83) of the particles and/or cells (82A-82D) are recorded when the particles and/or cells are located within the object (81), in particular hydrogel matrix; and recorded parameters (83) are registered together with a respective unique particle ID (84) in a database (86), in particular wherein the respective unique particle ID (84) referring to the particle and/or cell from which a parameter originates;
   subsequently releasing, in particular isolating, the particles and/or cells (82A-82D) from the object (81), in particular hydrogel matrix, and positioning the released, in particular isolated, particles and/or cells (82A-82D) in a plurality of new locations (A1 . . . H12),
   wherein each of the new locations (A1 . . . H12) is identifiable by a unique position ID (85), and in particular the new locations (A1 . . . H12) comprise at maximum one particle and/or cell (82A-82D);
   wherein the unique position ID (85) is allocated in the database (86) to the respective unique particle ID (84); in particular which particle ID (84) identifies the particles and/or cells (82) contained in the allocated new location (A1 . . . H12) identified by the respective unique position ID (84).
97. Method according to the preceding item,
   characterized in
   that before positioning the released particles (82) in the new location the particles positioner in one or a plurality of positions (32) of a device (30) according to any of items 40 to 73, in particular that further observations are performed when the released particles (82) are positioned within the positions (32) of the device (30).
98. Method according to any of the two preceding items,
   characterized in
   wherein the parameters (83) are selected from at least one
      a surface marker information,
      a intracellular marker information,
      a particle location information indicating a position within the droplet, in particular indicating an absolute position and/or a relative position ion in particular referring to at least one neighbouring particle.
99. Pump (50), comprising at least two, in particular at least three, valves (10) according to any of items 1 to 37, arranged in series,
   wherein the pump (50) is adapted to pump a fluid upon, in particular a sequential, activation of the valves (10A, 10C; 10C),
   in particular wherein, considered in a direction (F) of fluid, an outlet channel (12A) of a first valve (10A) is connected to an inlet channel (12B) of a second valve (10B), and/or
   in particular wherein, considered in a direction (F) of fluid, an outlet channel (11B) of a second valve (10B) is connected to an inlet channel (11A) of a third valve (10C).
100. Pump (50) according to the preceding item,
   characterized by
   at least two first valves (10A) arranged in parallel to each other, and/or at least two second valves (10B) arranged in parallel to each other and/or at least two third valves (10C) arranged in parallel to each other,
   in particular
   wherein the inlet channels (11A) of the first valves (10A) are connected to each other and/or
   wherein the outlet channels (12A) of the first valves (10A) are connected to each other and/or
   wherein the inlet channels (12B) of the second valves (10B) are connected to each other and/or
   wherein the outlet channels (11B) of the second valves (10B) are connected to each other and/or
   wherein the inlet channels (11C) of the third valves (10C) are connected to each other and/or
   wherein the outlet channels (12C) of the third valves (10C) are connected to each other.

The present invention also provides advantageous hydrogels that are described in the following by referring to the items.
101. A hydrogel which comprises cross-linked hydrogel precursor molecules of the same type or of different types.

The hydrogel precursor molecules, which preferably are provided by polymers as described herein, serve as building blocks for the hydrogel. Suitable and preferred polymers that can be used as building-block/precursor molecules for hydrogel formation are described herein. The hydrogel is formed by cross-linking (gelation) of the polymers.
102. The hydrogel according to item 101, wherein the hydrogel is composed of at least two different polymers with different structures as hydrogel precursor molecules, wherein optionally, at least one polymer is a copolymer.
103. The hydrogel according to item 101 or 102, wherein at least one polymer has a linear structure and at least one polymer has a multiarm or star-shaped structure.
104. The hydrogel according to any one of the preceding items, comprising a polymer that was obtained by copolymerization of (i) a heterocyclic chemical compound, preferably a 2-oxazoline, and (ii) a compound comprising (aa) an unsaturated imide, preferably 3-(maleimido)-propionic acid N-hydroxysuccinimide ester or (bb) an alkenyl group such as an isopropenyl group.
105. The hydrogel according to item 104, having at least one of the following characteristics:
   (a) compound (ii) comprises a spacer and a functional group for crosslinking a biologically active molecule;
   (b) compound (ii) is a 3-(maleimido)-propionic acid N-hydroxysuccinimide ester;
   (c) the backbone of at least one polymer is functionalized with at least one biologically active molecule at the functional group of compound (ii).
106. The hydrogel according to item 104 or 105, wherein compound (i) is a hydrophilic poly-(2-oxazoline), wherein optionally, the water-solubility is adjusted by the 2-substitution of the 2-oxazoline compound.
107. The hydrogel according to any of the preceding items, wherein the backbone of the polymers is formed by hydrophilic peptide-like polymers that are crosslinked in the hydrogel by cell-compatible crosslinking reactions.
108. The hydrogel according to any of the preceding items, wherein the hydrogel comprises a 2-oxazoline-based polymer, preferably a poly-2-methyl-2-oxazoline based polymer, more preferably a copolymer.
109. The hydrogel according to any of the preceding items, wherein the hydrogel comprises (i) linear and (ii) multiarm 2-oxazoline-based polymers.
110. The hydrogel according to item 108 or 109, wherein the 2-oxazoline is substituted only at position 2 and wherein preferably, the substitution in the 2-position comprises a group selected from alkynes, alkenes, protected amine groups or short aliphatic chains such as methyl.
111. The hydrogel according to any one of items 108 to 110, having one or more of the following characteristics:
   (a) the hydrogel comprises a polymer that is formed by living cationic ring-opening polymerization of oxazolines substituted at position 2;
   (b) the hydrogel is a biomaterial for cell applications, wherein preferably, the biomaterial is composed of at least two different polymers according to any one of items 201 to 255, wherein the different polymers having different structures, wherein the first polymer has a linear structure and the second polymer has a multiarm or star-shaped structure;
   (c) the hydrogel comprises one or more biologically active molecules linked to the polymer backbone of at least one polymer/hydrogel precursor, wherein preferably, the polymer is linear and wherein more preferably, the biologically active molecule is attached via a degradable linker.
112. The hydrogel according to any one of items 101 to 111, wherein the hydrogel matrix is composed of a mixture of at least two different polymers according to any one of items 201 to 255.
113. The hydrogel according to any one of items 101 to 112, wherein the hydrogel matrix comprises at least two polymers according to:
   a) item 201 or 212, wherein the polymer further has the features of item 239, or according to item 223; and
   b) item 201 or 212, wherein the polymer further has the features of item 239 and 244, or according to item 225.
114. The hydrogel according to any one of items 101 to 112, wherein the hydrogel matrix comprises at least two polymers according to:
   a) item 201 or 212, or according to item 227; and
   b) item 201 or 212, or according to item 229.
115. The hydrogel according to any one of items 101 to 112, wherein the hydrogel matrix comprises at least two polymers according to:
   a) item 224; and
   b) item 226.
116. The hydrogel according to any one of items 101 to 112, wherein the hydrogel matrix comprises at least two polymers according to:
   a) item 204 or 222, wherein the polymer further has the features of item 242, wherein the polymer preferentially has a linear structure; and
   b) item 204 or 222, wherein the polymer further has the features of item 244 or wherein the hydrogel matrix comprises at least two different polymers, preferably at least 4 different polymers, more preferably at least 5 different polymers according to:
      a. item 223;
      b. item 225;
      c. item 227;
      d. item 229;
      e. item 224;
      f. item 226;
      g. item 228;
      h. item 230; and/or
      i. item 234.
117. The hydrogel according to any one of items 101 to 116, wherein hydrogel precursor molecules are crosslinked in the hydrogel matrix by cell-compatible crosslinking reactions.
118. The hydrogel according to any one of items 101 to 117, wherein the hydrogel precursor molecules are cross-linked in the hydrogel matrix by a reaction selected from:
   (i) a covalent bond formation, preferably selected from (aa) enzymatically catalyzed reactions, such as reactions catalyzed with transglutaminase factor XIIIa, (bb) not-enzymatically catalyzed reactions, such as click chemistry or photo-catalyzed reactions and/or (cc) uncatalyzed reactions, such as copper-free highly selective click chemistry, Michael-type addition or Diels-Alder conjugation;
   (ii) non-covalent bond formation preferably selected from (aa) hydrogen bonds, preferably formed by nucleic acids or nucleic acid analogs, (bb) hydrophobic interactions, (cc) Van-der-Waals interactions and (dd) electrostatic interactions; and
   (iii) combinations of the foregoing.
119. The hydrogel according to any one of items 101 to 118, wherein in the hydrogel, cross-links are formed via terminating moieties that are located at ends of the polymers providing the hydrogel precursor molecules, wherein optionally, cross-links are formed exclusively via terminating moieties that are located at ends of the polymers providing the hydrogel precursor molecules.

A terminating moiety may be located at ends of the hydrogel precursor molecule as is illustrated in the figures for linear as well as multimer or starshaped polymers. Accordingly, the terminating moieties of the polymers are used for gel formation and thus cross-linking. According to one embodiment, the hydrogel precursor molecules are not cross-linked in the hydrogel matrix by functional groups that are attached to the polymer backbone. Functional groups provided at the backbone of a comprised polymer are preferably used for attaching a biologically active molecule, as is described in detail herein. It is advantageous if functional groups provided at the polymer backbone and which are used for attaching one or more biologically active molecules are unable to participate in the cross-linking (gelation) reaction. Thereby, the functionalization of the comprised polymer (hydrogel precursor/building block) and hence the hydrogel is independent from and not competitive to the cross-linking reaction. This allows to provide uniform hydrogels, e.g. comprising alternating linear and multi-arm precursors. As is described herein, functionalization with a biologically active molecule may be performed before, during or after formation of the hydrogel.
120. The hydrogel according to any one of items 101 to 119, having one or more of the following characteristics:
   (a) the hydrogel is composed of at least two different polymers, preferably according to any one of items 201 to 255, wherein the different polymers are crosslinked with a carboxy-, thiol-, or amine-functionalized polymer, preferably polyethylene glycol (PEG) such as poly(ethylene glycol) bis(amine) or poly(ethylene glycol) dithiol or di(N-succinimidyl) functionalized components with dithiol moieties such as dithiodipropionic acid di(N-hydroxysuccinimide ester or carboxy-functionalized disulfides such as 2-carboxyethyl disulfide;
   (b) in the hydrogel, maleimide and thiol endfunctionalized polymer precursors are cross-linked, wherein preferably, at least one polymer precursor furthermore comprises a NHS ester as functional group attached to the polymeric backbone, preferably via a degradable linker, for functionalization with a biologically active molecule or wherein a biologically active molecule is attached thereto.
121. The hydrogel according to any one of items 117 to 120, wherein crosslinking includes hydrogen bond formation, preferably based on hybridization.
122. The hydrogel according to any one of items 117 to 121, wherein each hydrogel precursor molecule comprises a terminating moiety and wherein terminating moieties of different hydrogel precursor molecules are crosslinked by hybridization thereby forming the hydrogel.

Hence, according to one embodiment cross-linking of the polymers providing the hydrogel precursor molecules is achieved by sequence specific hybridization. Hybridization based cross-linking is advantageous, because it allows the formation of the hydrogel in the presence of living cells. In addition, the degradation of the hydrogel can be performed under conditions that preserve the viability of the cells.
123. The hydrogel according to any one of items 121 to 122, wherein hybridization based cross-links are formed between (i) a linear hydrogel precursor molecule and (ii) a multiarm or starshaped hydrogel precursor molecule.
124. The hydrogel according to any one of items 119 to 123, wherein each terminating moiety comprises bases allowing sequence specific base pairing by hydrogen bonds, in particular selected from purine and pyrimidine bases.
125. The hydrogel according to any one of items 119 to 124, having one or more of the following characteristics:
   (a) wherein each terminating moiety comprises an oligomer, preferably having ≤30, ≤25, preferably ≤20 or ≤1.5 bases,
   (b) the hydrogel precursors are crosslinked based on hybridization involving Watson-Crick base pairing and/or Hoogsteen base pairing, wherein preferably hybridization is based on Watson-Crick base pairing.
126. The hydrogel according to item 124 or 125, wherein the terminating moiety comprises a nucleic acid or nucleic acid analog, preferably selected from PNA, LNA, hexitol nucleic acid (HNA), morpholino oligomers, phosphorthioate DNA, phosphoramidate DNA and 2′O-methoxyethyl RNA, more preferably selected from PNA and LNA and most preferably is a PNA.
127. The hydrogel according to any one of items 119 to 126, wherein different hydrogel precursor molecules comprise different terminating moieties, wherein said different terminating moieties comprise moieties, preferably nucleic acids or nucleic acid analogs, that are
   (i) complementary to each other and are hybridized to each other in the hydrogel; or
   (ii) are not complementary to each other and the crosslink in the hydrogel is established by a hybridizing molecule that hybridizes to the different terminating moieties of the hydrogel precursor molecules, thereby providing a cross-link that is based on hybridization, whereby different precursor molecules are cross-linked due to hybridization, thereby providing an alternating structure of polymers, preferably linear and multimer polymers, that form the hydrogel.
128. The hydrogel according to item 127, wherein the hybridizing molecule has one or more of the following characteristics
   (i) it comprises (aa) a first hybridizing portion that hybridizes to the terminating moiety of one hydrogel precursor molecule and (bb) a second hybridizing portion that hybridizes to the terminating moiety of another hydrogel precursor molecule;
   (ii) it comprises bases allowing sequence specific base pairing by hydrogen bonds, wherein preferably, the hybridizing molecule comprises ≤60, ≤55, preferably ≤50 or ≤35 bases.

Sequence specific base pairing may be selected from Watson-Crick base pairing or Hoogsteen base pairing and preferably is based on Watson-Crick base pairing.
129. The hydrogel according to any one of items 117 to 128, wherein the hydrogel precursor molecules are cross-linked by hybrids that comprise mismatches.
130. The hydrogel according to any one of items 101 to 129, wherein the hydrogel precursor molecules comprise an enzyme degradable target site, preferably a protease target site, preferably located between the polymer backbone and the terminating moiety.

The enzyme degradable target site as disclosed herein may be e.g. degradable by esterases (hydrolysis of esters), lipases (hydrolysis of lipids, PHA depolymerases (hydrolysis of polyhydroxyalkanoate) or preferably proteases (hydrolysis of peptides). Preferably the enzyme degradable target site is a protease target site. The protease target site may be degradable by a protease that is secreted by a cell, which is in particular favorable for embodiments wherein the hydrogel comprises a cell. In a very preferred embodiment, the enzyme degradable target site is a matrix metalloprotease (MMP) target site.
131. The hydrogel according to any one of items 127 to 130, wherein the hybridizing molecule establishing the crosslink between the terminating moieties is selected from a nucleic acid or nucleic acid analog, preferably is selected from PNA and DNA,
   wherein optionally, the hybridizing molecule is a PNA molecule that comprises (aa) a first hybridizing portion that hybridizes to the terminating moiety of one hydrogel precursor molecule and (bb) a second hybridizing portion that hybridizes to the terminating moiety of another hydrogel precursor molecule, and (cc) an enzyme degradable target site, preferably a protease target site, more preferably a matrix metalloprotease target site, for site directed degradation of the polymer located between the first and second hybridizing portions.
132. The hydrogel according to any one of items 117 to 131, wherein the crosslinking reaction includes hydrogen bond formation between two peptide nucleic acid (PNA) molecules with different base sequences or two locked nucleic acid (LNA) molecules with different base sequences or a combination of one PNA molecule and one LNA molecule.
133. The hydrogel according to item 132, wherein a PNA molecule and/or the LNA molecule is located at the ends of the polymers that form the hydrogel.
134. The hydrogel according to any one of items 101 to 133, wherein the hydrogel matrix is built up from precursor molecules that are cross-linked by hydrogen bonds formed by peptide nucleic acids (PNA).

Accordingly, in a preferred embodiment the hydrogel is formed based on PNA hybridization.
135. The hydrogel according to item 134, wherein PNA oligomers are located at the ends of the polymers that form the hydrogel.
136. The hydrogel according to item 134 or 135, wherein different precursor molecules possess complementary PNA oligomers, wherein preferably, linear and multiarm polymers are used as precursors.
137. The hydrogel according to any one of items 134 to 136, wherein only different precursor molecules are cross-linked due to hybridization of the comprised PNAs, thereby providing an alternating structure of preferably linear and multimer polymers that form the hydrogel.
138. The hydrogel according to any one of items 134 to 137, wherein the complementary PNA oligomers possess mismatches.
139. The hydrogel according to any one of items 134 to 138, wherein the PNA oligomers cross-linking the hydrogel precursor molecules have one or more, preferably all, of the following characteristics
   (i) they are short oligos ≤15mers;
   (ii) they have a purine content of <50%,
   (iii) they are not self-complementarity,
   (iv) they do not comprise poly guanine sequences.
140. The hydrogel according to any one of items 101 to 139, wherein a three-dimensional hydrogel is formed via hydrogen bonds between LNAs and/or PNAs of (i) a multiarm or starshaped poly-(2-oxazoline) based polymer and (ii) a linear poly-(2-oxazoline) based polymer, wherein preferably the linear poly-(2-oxazoline) based polymer is functionalized with biologically active molecules.
141. The hydrogel according to any one of items 101 to 139, having one or more of the following characteristics:
   (a) wherein the hydrogel is formed by hybridization of complementary PNA sequences;
   (b) wherein precursor molecules comprise a peptide bearing a protease site, preferably adjacent to the PNA;
   (c) wherein the hydrogel matrix comprises multiple degradation targets for at least one enzyme that is secreted by a cell comprised in the hydrogel matrix, preferably MMP target sites;
   (d) wherein the hydrogel is degradable by increasing the temperature;
   (e) wherein the hydrogel has a spherical or plug-like structure and preferably, is spherical;
   (f) the hydrogel is three-dimensional;
   (g) the hydrogel has a size in the micrometer or sub-micrometer scale and is preferably spherical;
   (h) wherein the hydrogel provides a synthetic backbone, preferably a synthetic matrix that lacks toxins.
142. The hydrogel according to any one of the preceding items 101 to 141, wherein the hydrogel matrix comprises one or more cells and/or particles, preferably comprises at least one cell.
143. The hydrogel according to any one of the preceding items 101 to 142, wherein at least one polymer is functionalized with at least one biologically active molecule, preferably to present different adhesive ligands, bioactive compounds and functional biomolecules such as adhesive compounds of the extra cellular matrix (ECM), growth factors, antibodies, CRISPR-Cas and nucleic acids or wherein the hydrogel comprises one or more of the following:
   (i) functional molecules for cell culture and cell analysis;
   (ii) gold particles, quantum dots, growth promoting substances, cytokines, chemokines, antibody-conjugates and/or inorganic substances.
144. The hydrogel according to any one of the preceding items 101 to 143, wherein the backbone of a comprised polymer, preferably a linear polymer, is functionalized with a biologically active molecule.
145. The hydrogel according to any one of the preceding items 101 to 144, wherein the hydrogel comprises capture molecules, which may be incorporated by one or more of the cross-linking techniques as defined in item 118, wherein optionally, incorporation of the capture molecules involves peptide nucleic acids, wherein preferably, a PNA oligomer is incorporated into the hydrogel gel by amide bond formation between an NHS-ester from the hydrogel precursor molecule and the primary amine of a PNA oligomer and wherein the capture molecule is fused to a complementary PNA oligomer and wherein the capture molecule is incorporated into the hydrogel by hydrogen bond formation between the two PNA oligomers.
146. The hydrogel according to any one of the preceding items 101 to 144, wherein the hydrogel is surrounded by a gel-shell.

As is described herein, providing a gel-shell surrounded hydrogel has several advantages, in particular if at least one cell is encapsulated in the hydrogel matrix. The gel-shell reduces the pore size of the matrix compared to the hydrogel matrix that is provided in the core. The gel-shell reduces the cut-off for small molecules and may set the cut-off for small molecules down to kDa. Providing an according gel-shell allows to prevent e.g. the cross talk between different hydrogel particles that are located adjacent to each other, and thereby allows to prevent e.g. the cross talk between cells encapsulated in different hydrogel particles. Furthermore, providing an according gel-shell reduces the risk that a cell can escape from the hydrogel. This allows a longer cultivation and analysis.
147. The hydrogel according to item 146, wherein the gel-shell comprises at least one gel-shell forming compound that is optionally crosslinked to the hydrogel, wherein preferably the gel-shell forming compound is a primary amine bearing polymer molecule, e.g. a poly (allylamine).
148. The hydrogel according to item 147, wherein the gel-shell forming compound is or is derived from a compound selected from the group consisting of poly(allylamine), branched amino-polyethyleglycol (PEG), branched polyethylenimine (PEI), polylysine, poly amidoamine (PAMAM) dendrimer, poly((3-amino ester), chitosan and poly(2-amino-2-oxazoline).
149. The hydrogel according to item 147 or 148, having one or more of the following characteristics:
   (a) the gel-shell forming compound is crosslinked to a functional group of a poly(2-oxazoline)copolymer);
   (b) the gel-shell forming compound forms a gel-shell around the hydrogel matrix, wherein the gel-shell is not covalently attached to the hydrogel matrix and wherein optionally, the gel shell forming compound comprises functional groups that are cross-linked via a further compound thereby forming a gel-shell around the hydrogel matrix;
   (c) the gel-shell forming compound comprises a nucleic acid or nucleic acid analog, preferably a PNA sequence, that is hybridized to another compound comprised in the hydrogel matrix, wherein the compound is a multi-arm or star-shaped polymer, which is not crosslinked to the hydrogel matrix and wherein the gel-shell forming compound is preferably a linear polymer.
150. The hydrogel according to any one of items 101 to 149, comprising a polymer according to item 204 as hydrogel precursor that is preferably cross-linked with a multiarm polymer.
151. The hydrogel according to any one of items 147 to 150, wherein the gel-shell surrounded hydrogel was obtained by the method according to 164 to 170.
152. A method for producing a hydrogel according to any one of items 101 to 151, wherein the hydrogel matrix is formed by cross-linking hydrogel precursor molecules of the same type or of different types.

The hydrogel is formed by cross-linking (gelation) the hydrogel precursors which are as described above preferably polymers as described herein that serve as building blocks for the hydrogel matrix. The hydrogel can be formed by an orthogonal cross-linking (gelation) process.
153. The method according to item 152, comprising crosslinking hydrogel precursor molecules by a cell-compatible crosslinking reaction, preferably in the presence of a cell.
154. The method according to item 152 or item 153, wherein the crosslinking reaction is selected from:
   (i) a covalent bond formation, preferably selected from (aa) enzymatically catalyzed reactions, such as reactions catalyzed with transglutaminase factor XIIIa, (bb) not-enzymatically catalyzed reactions, such as click chemistry or photo-catalyzed reactions and/or (cc) uncatalyzed reactions, such as copper-free highly selective click chemistry, Michael-type addition or Diels-Alder conjugation;
   (ii) non-covalent bond formation preferably selected from (aa) hydrogen bonds, preferably formed by nucleic acids or nucleic acid analogs, (bb) hydrophobic interactions, (cc) Van-der-Waals interactions and (dd) electrostatic interactions; and
   (iii) combinations of the foregoing.
155. The method according to any one of items 152 to 154, wherein the crosslinking reaction includes hydrogen bond formation, preferably between
   (i) two peptide nucleic acid (PNA) molecules with different base sequences or
   (ii) two locked nucleic acid (LNA) molecules with different base sequences or
   (iii) a combination of one PNA molecule and one LNA molecule.
156. The method according to any one of items 152 to 155, wherein the hydrogel is produced using a method for droplet generation and mixing of at least two droplets, preferably the method for droplet generation as defined in any one of items 74 to 77.

The method accordingly comprises combining two droplets whereby a fused droplet is provided that comprises the hydrogel precursor molecules.
157. The method according to item 156, wherein after generating and mixing said droplets, a spherical or plug-like hydrogel matrix is formed within the mixed droplet.
158. The method according to item 156 or 157, comprising generating
   (i) a first droplet that comprises a multiarm hydrogel precursor,
   (ii) a second droplet that comprises a linear hydrogel precursor,
   (iii) optionally a third droplet for initiating the cross-linking of the multiarm and the linear hydrogel precursor, wherein after generating and mixing said droplets a spherical or plug-like hydrogel matrix is formed within the mixed droplet, wherein optionally, the first or the second droplet comprises compounds, preferably biological active molecules, wherein said compounds are immobilized within the hydrogel matrix, preferably during hydrogel formation.
159. The method according to any one of items 156 to 158, comprising generating and mixing at least four droplets, wherein the fourth droplet comprises a compound that becomes immobilized within the formed hydrogel matrix, preferably by a stable amide bond, wherein the compounds is optionally selected from proteins such as antibodies, growth factors or ECM proteins; nucleic acids such as DNA primers and peptide nucleic acids or is selected from gold particles, quantum dots, growth promoting substances, cytokines, chemokines, antibody-conjugates, inorganic substances.
160. The method according to any one of items 156 to 159, wherein the droplets are generated in parallel or sequentially.
161. The method according to any one of items 152 to 160, having one or more of the following characteristics:
   (a) the method comprises generating at least two droplets, wherein each droplet comprises a different cell type and fusing said at least two droplets to provide a first droplet that comprises at least one multiarm precursor;
   (b) the first droplet comprises at least one cell and wherein the multiarm precursor lacks functional groups that are reactive with the one or more cells under the conditions within the first droplet.
162. The method according to any one of items 152 to 161, wherein during hydrogel formation, one or more cells or particles, preferably at least one cell, becomes encapsulated in the hydrogel matrix, wherein preferably, the one or more cells or particles are combined with at least one hydrogel precursor prior to forming the gel, and wherein more preferably, the encapsulation method as defined in any of items 173 to 183 is used.

As is described herein the at least one cell is preferably contacted with at least one hydrogel precursor, e.g. in a droplet, prior to forming the hydrogel. As is described in further detail below, the composition comprising the at least one cell and the hydrogel precursor may then be contacted with a further hydrogel precursor to be cross-linked to provide the hydrogel gel. The method thereby allows to specifically encapsulate a predefined number of cells. Furthermore, it allows to encapsulate predefined cell types into the hydrogel. According to one embodiment, at least two different cell-types are selected and combined with the hydrogel precursors in a liquid composition prior to forming the hydrogel by cross-linking (gelation).
163. The method according to any one of items 152 to 162, comprising functionalizing the hydrogel with at least one biologically active molecule, wherein preferably, functionalization has one or more of the following characteristics:
   (a) functionalization occurs before, during or after encapsulating at least one cell into the hydrogel;
   (b) the biologically active molecule is cross-linked to a functional group of at least one polymer that provides a hydrogel precursor molecule, which preferably is a polymer as defined in any one of items 201 to 255, and preferably, is a linear polymer;
   (c) after hydrogel formation the method comprises adding bioactive molecules to a liquid that flows through the formed hydrogel, thereby incorporating bioactive molecules into the hydrogel matrix;
   (d) the hydrogel is functionalized with at least one biologically active molecule before a gel-shell is formed that surrounds the particle.
164. The method according to any one of items 152 to 163, for producing a gel-shell surrounded hydrogel, comprising
   (a) providing a droplet generated by fusion of multiple droplets, wherein the fused droplet A comprises the hydrogel matrix;
   (b) forming the gel-shell by fusing droplet A with a second droplet B containing a polymer which comprises primary amines, such as poly allylamine polymers, thereby providing a larger droplet C containing said hydrogel matrix with the volume of the hydrogel matrix being smaller than the volume of droplet C and wherein in droplet C, said hydrogel matrix is surrounded by said polymer from droplet B and
   (c) crosslinking of the hydrogel polymers at the edge of the hydrogel matrix.
165. The method according to item 164, wherein said polymer from droplet B diffuses into the hydrogel matrix, whereby crosslinking occurs.
166. The method according to item 164 or 165, wherein the method comprises using (i) a primary amine bearing polymer molecule, e.g. a poly allylamine and (ii) a small primary amine, e.g. 3-amino-1,2-propanediol, wherein the polymer molecule (i) having a smaller diffusion coefficient than the small primary amine (ii).

As small primary amine, e.g. an aminofunctionalyzed C3-C6-alkanediol such as 3-amino-1,2-propanediol can be used.
167. The method according to item 166, wherein the primary amine diffuses faster into said hydrogel matrix than the polymer molecule, wherein preferably the small primary amines are added with a short delay after the poly allylamine polymers.
168. The method according to any one of items 164 to 167, wherein the method further comprises fusing said droplet C with a droplet D containing a small primary amine, e.g. 3-amino-1,2-propanediol, the small primary amine having a smaller diffusion coefficient than the polymer in droplet C.
169. The method according to any one of the preceding items for producing a gel-shell surrounded hydrogel, comprising
   (a) providing a droplet generated by fusion of multiple droplets, wherein the fused droplet A comprises the hydrogel matrix;
   (b) forming the gel-shell by immobilizing and diffusing droplet A by an aqueous phase containing a small primary amine, e.g. 3-amino-1,2-propanediol and a polymer which comprises primary amines, such as poly allylamine polymers, the small primary amine having a smaller diffusion coefficient than the polymer; and
   (c) crosslinking of the hydrogel polymers at the edge of the hydrogel matrix.
170. The method according to any one of the preceding items for producing a gel-shell surrounded hydrogel, wherein the shell is formed by contacting one or more of the following compounds with the hydrogel matrix:
   i) a polymer which comprises primary amines, which is preferably selected from poly(allylamine), (branched) amino-polyethyleglycol (PEG), (branched) polyethylenimine (PEI), polylysine, poly amidoamine (PAMAM) dendrimer, poly((3-amino ester), chitosan, or amino-PaOX, and, optionally, a primary amine compound which preferably is a small primary amine compound such as an aminofunctionalyzed C3-C6-alkanediol, e.g. 2-amino-1,3-propanediol or 3-amino-1,2-propanediol,
wherein amine groups react with a residual functional group of the hydrogel matrix, e.g. a N-hydroxysuccinimide ester;
   ii) a polymer comprising a N-hydroxysuccinimide ester, preferably selected from PEG-NHS-ester or polyoxazoline-NHS-ester, and a diamine compound, e.g. a c3-c6-alkanol diamine such as 1,3-diamino-2-propanol, wherein the diamine compound is present in the hydrogel matrix prior to adding the N-hydroxysuccinimide ester comprising compound;
   iii) a polymer comprising a maleimide, and furthermore a dithiol compound, e.g. 2,2′-(ethylenedioxy)diethanethiol or short dithiol functionalized polymers with an enzyme degradable target site, such as a matrix metalloprotease sensitive target site, wherein the dithiol compound is present in the hydrogel matrix prior to adding the maleimide comprising compound; or
   iv) the gel-shell forming compound comprises a nucleic acid or nucleic acid analog, preferably a PNA sequence, that is hybridized to another compound comprised in the hydrogel matrix, wherein the compound is a multi-arm or star-shaped polymer, which is not crosslinked to the hydrogel matrix and wherein the gel-shell forming compound is preferably a linear polymer.
171. The method according to any one of the preceding items 152 to 170, comprising providing at least two polymers, preferably selected from the polymers as defined in any one of items 201 to 251, as hydrogel precursors and cross-linking the at least two polymers to provide the hydrogel.

Suitable cross-linking strategies are described above as well as compatible terminating moieties allowing cross-linking of the hydrogel precursors. It is referred to the above description which also applies here.
172. A hydrogel obtained by the method according to any one of items 152 to 171.

Also provided is a method for cell encapsulation as will be described in the following.

173. A method for encapsulating one or more cells and/or particles into a hydrogel, preferably a hydrogel as defined in any one of items 101 to 151, wherein the one or more cells and/or particles are combined with at least one hydrogel precursor prior to gel formation and are encapsulated into the hydrogel matrix during hydrogel formation.

As is described herein, the present disclosure allows to encapsulate cells during hydrogel formation, i.e. the one or more cells are in contact with hydrogel precursors before the final gel matrix is formed and therefore are present during gel formation. In contrast to prior art methods the cells are not just added after the hydrogel has already been formed. The one or more cells are preferably combined with at least one precursor polymer prior to hydrogel formation. E.g. the one or more cells may be encapsulated into droplets containing one or more precursor polymers prior to hydrogel formation. Advantageously, the hydrogel matrix is formed around the one or more cells. The cells can advantageously be provided at the center of the formed hydrogel. Suitable methods are described herein.
174. The method according to item 173, wherein one or more polyoxazoline derivatives are used as hydrogel precursor.
175. The method according to item 174, wherein one or more polymers as defined in any one of items 201 to 255 are used as hydrogel precursor, wherein preferably, at least one polyoxazoline based polymer, preferably a co-polymer comprising at least one moiety of formula (I) and at least one moiety of formula (II) as defined in item 201 and items dependent thereon, is used as hydrogel precursor.
176. The method according to any one of items 173 to 175, having one or more of the following characteristics:
   (a) wherein after encapsulation, the one or more cells are in the center of the hydrogel,
   (b) the method comprises
      preparing a liquid composition comprising (i) one or more cells and/or particles, preferably one or more cells, and (ii) the hydrogel precursors, and
      cross-linking the hydrogel precursors thereby providing a hydrogel encapsulating the one or more cells and/or particles.
   (c) the method comprises
      preparing a liquid composition using a microfabricated valve according to item 30 or 31 comprising (i) one or more cells and/or particles, preferably one or more cells, and (ii) the hydrogel precursors, and
      cross-linking the hydrogel precursors thereby providing a hydrogel encapsulating the one or more cells and/or particles.

As is described herein, the present disclosure allows to encapsulate cells during hydrogel formation, i.e. the one or more cells are in contact with hydrogel precursors before the final gel matrix is formed and therefore are present during gel formation. In contrast to prior art methods the cells are not just added after the hydrogel has already been formed. The one or more cells are preferably combined with at least one precursor polymer prior to hydrogel formation. E.g. the one or more cells may be located within a fluid in particular an aqueous fluid containing one or more precursor polymers prior to hydrogel formation. Advantageously, the hydrogel matrix is formed using microfluidic valves according to item 30 and 31. The geometries of these microfluidic valves enable separation of two fluids prior to hydrogel formation:
177. The method according to item 176, wherein the method comprises combining one or more cells and/or particles, preferably one or more cells, with at least one hydrogel precursor prior to forming the hydrogel and wherein the hydrogel is formed in the presence of the one or more cells.

In embodiments, cross-linking of the hydrogel precursors and hence the hydrogel matrix to the encapsulated cells can be reduced or prevented by the encapsulating strategy. E.g. as described herein, it is preferred to use a cross-linking strategy that is based on hybridization, e.g. terminating moieties comprising nucleic acids or nucleic acid analogs. Furthermore, it can be advantageous to combine the hydrogel precursors sequentially with the cells prior to forming the cross-links (gelation) in the presence of the cells, whereby the hydrogel matrix is formed around the cell(s). As described herein, in one embodiment the one or more cells are first combined with a hydrogel precursor comprising a terminating moiety for cross-linking that is not reactive with the one or more cells (or particles) to be encapsulated. E.g. a terminating moiety comprising a nucleic acid or nucleic acid analog is not reactive with the cell and accordingly, does not lead to a cross-linking of the hydrogel precursor with the cell. The same applies e.g. if using a hydrogel precursor comprising a thiol group for cross-linking. According to one embodiment, the one or more cells are combined with a hydrogel precursor, e.g. a multiarm or starshaped polymer, that comprises terminating moieties for cross-linking but does not comprise a further functional group that is reactive under the combination conditions with the cell(s) to be encapsulated. E.g. the hydrogel precursor may entirely lack such functional groups in the polymer backbone, or functional groups present may have been saturated e.g. by attaching a biologically active molecule and/or by attaching a compound, e.g. a small primary amine, as described herein, prior to contacting the hydrogel precursor with the cell(s). Subsequently, the composition comprising the hydrogel precursor and the cell(s) is then combined with at least one further hydrogel precursor, preferably a linear polymer comprising at least one functional group for attaching a biologically active molecule, wherein optionally, a biologically active molecule has been attached to the functional group, and the hydrogel precursors are cross-linked, whereby the hydrogel matrix is formed that encapsulates the cell(s). As described herein, one or more biologically active molecules may be attached to functional groups provided in the polymer(s) prior, during or after cell encapsulation.
178. The method according to item 173 or 177, wherein the method comprises (a) providing prior to forming the hydrogel a liquid composition, e.g. in form of a droplet, wherein the composition comprises (i) one or more cells and/or particles, preferably one or more cells, and (ii) at least one hydrogel precursor, (b) combining, e.g. mixing, said composition with at least one further hydrogel precursor and (c) forming the hydrogel in the presence of the one or more cells by cross-linking (gelation) whereby the hydrogel matrix is formed around the cell.
179. The method according to any one of items 173 to 178, comprising combining the one or more cells sequentially with the hydrogel precursors prior to cross-linking the hydrogel precursors, wherein the one or more cells are combined with at least one hydrogel precursor that lacks functional groups that are reactive with the one or more cells under the combination conditions and subsequently adding at least one further hydrogel precursor, wherein optionally, the subsequently added hydrogel precursor comprises functional groups for attaching a biologically active molecule.
180. The method according to any one of items 173 to 179, wherein the hydrogel is produced by generating and mixing at least two droplets comprising different hydrogel precursors, wherein one or more cells are comprised in at least one droplet, preferably using the method for droplet generation and fusing of at least two droplets as defined in any one of preceding items or using the method according to item 156 and items dependent thereon.
181. The method according to any one of items 173 to 180, wherein the method comprises:
   encapsulating one or more cells and/or particles into a first droplet, wherein the first droplet has a defined size and comprises a hydrogel precursor molecule (a) at a defined concentration;
   generating a second droplet, wherein the second droplet has a defined size and comprises a hydrogel precursor molecule (b) at a defined concentration
   fusing said formed droplets, thereby providing a larger droplet that contains the hydrogel precursor molecules (a) and (b) and the one or more cells and/or particles, wherein preferably, hydrogel formation occurs due to the mixing of said hydrogel precursor molecules.
182. The method according to any one of items 173 to 181, wherein the first or the second droplet comprises compounds, preferably biological active molecules, wherein said compounds are immobilized within the hydrogel matrix, preferably during hydrogel formation.
183. The method according to any one of items 173 to 182, having one or more of the following characteristics
   (a) the hydrogel is as defined in any one of items 101 to 151;
   (b) wherein the type of encapsulated cells is the same or different;
   (c) wherein the method comprises preparing at least two separate hydrogels, preferably at least two hydrogel beads, wherein the type of encapsulated cells is the same or different.
184. A method for degrading a hydrogel according to any one of items 101 to 151, comprising reversing the cross-links of the hydrogel.
185. The method according to item 184, comprising reversing hybridization based, preferably PNA based, cross-links of the hydrogel.

To reverse the hybridization based cross-links of the hydrogel, the hybrids forming the crosslinks are denatured.
186. The method according to item 184 or 185, comprising one or more of the following:
   (a) heating the hydrogel to degrade the hydrogel; and/or
   (b) increasing the ionic strength;
   (c) dehybridization of complementary PNAs by applying heat, high salt concentrations or complementary nucleic acids with a higher affinity, preferably in molar excess.
187. The method according to any one of items 184 to 186, comprising adding at least one hybridizing molecule, preferably in excess, to the hydrogel wherein the hybridizing molecule disturbs the cross-linking hybrids of the hydrogel, whereby the crosslinks are reversed.
188. The method according to item 187, wherein the at least one hybridizing molecule used for degradation is complementary to
   (aa) a terminating moiety of a hydrogel precursor molecule that participates in the crosslinking hybrid, or
   (bb) the hybridizing molecule used for crosslinking according to item (ii), and binds with a higher affinity thereto.
189. The method according to item 187 or 188, wherein the crosslinking hybrid comprises mismatches and wherein the added hybridizing molecule provides a hybrid without mismatches.
190. The method according to any one of items 187 to 189, wherein the added hybridizing molecule is selected from a nucleic acid or nucleic acid analog, and preferably is a PNA.
191. The method according to item 185, comprising adding PNA oligomers in excess to the hydrogel, wherein the complementary PNAs forming the cross-link of the hydrogel have a decreased hybridization energy compared to the PNA oligomers added for hydrogel degradation.
192. The method according to any one of items 184 to 191, comprising adding at least one enzyme to degrade the hydrogel, preferably selected from proteases and nucleases.
193. The method according to item 192, wherein the added enzyme targets a protease target site comprised in the hydrogel precursor and/or the hybridizing molecule, thereby degrading the hydrogel.

According to one embodiment, the enzyme targets the enzyme degradable target site that is comprised in at least one polymer providing a building block/precursor molecule of the hydrogel. The added enzyme cleaves the enzyme degradable target site, thereby degrading the hydrogel at the incorporated target site. Suitable enzymes are described herein and it is referred to the according disclosure. According to one embodiment, a matrix metalloprotease is used to degrade the hydrogel.
194. The method according to item 192 or item 193, wherein the added enzyme is a nuclease, preferably a DNase, and wherein the DNase degrades a hybridizing DNA molecule that establishes the hybridizing hybrid thereby degrading the hydrogel.
195. The method according to any one of the preceding items 184 to 194, wherein the hydrogel to be degraded comprises at least one cell and wherein said cell is not affected by the degradation procedure.
196. The method according to item 195, wherein the hydrogel is degraded by at least one enzyme that is secreted by the at least one cell comprised in the hydrogel, wherein optionally
   (a) the secreted enzyme targets the protease target site comprised in the hydrogel precursor and/or the hybridizing molecule, thereby degrading the hydrogel;
   (b) the secreted enzyme is a nuclease, preferably a DNase, and wherein the DNase degrades the hybridizing DNA molecule that establishes the hybridizing hybrid thereby degrading the hydrogel.
197. A droplet or combination of at least two droplets comprising a hydrogel according to any one of items 101 to 152.
198. The droplet according to item 197, wherein the droplet or the combination of at least two droplets comprises one or more cells, wherein optionally, at least two different cell types are comprised, preferably in different droplets.
199. A kit for providing a hydrogel according to any one of items 101 to 151, comprising:
   (a) a first hydrogel precursor
   (b) a second hydrogel precursor
   (c) optionally a reagent for crosslinking the first and second hydrogel precursor
   (d) optionally a test device as defined in any one of items 40 to 73
200. The kit according to item 199, having one or more of the following characteristics:
   the first and second hydrogel precursor are provided by a polymer as defined in any one of items 201 to 255,
   the first and second hydrogel precursor comprise terminating moieties comprising nucleic acids or nucleic acid analogs;
   the first hydrogel precursor is a linear polymer and the second hydrogel precursor is a multiarm or starshaped polymer;
   the first and/or the second hydrogel precursor, preferably the first and second hydrogel precursor, is selected from a polymer as defined in any one of 201 to 255;
   it comprises at least one biologically active molecule, wherein preferably, said molecule is suitable to react with a functional group of at least one hydrogel precursor, preferably a linear hydrogel precursor, and wherein more preferably, the biologically active molecule is a peptide or protein and wherein at least one hydrogel precursor comprises a functional group capable of reacting with the N-terminus of the peptide or protein, wherein preferably, the functional group of the hydrogel precursor is a NHS ester;
   it comprises a reagent for providing a gel shell;
   it comprises a cell culture medium;
   the hydrogel precursor molecules are lyophilized, wherein optionally the kit comprises a reagent for reconstituting the hydrogel precursor molecules.

The polymers have been described above and also described in the following:
201. Polymer, especially polymer as building-block for hydrogel formation, comprising at least one moiety of formula (I) and at least one moiety of formula (II)

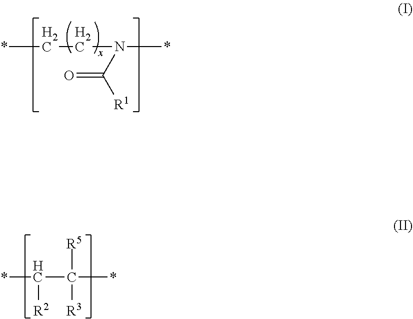

wherein
R¹ is a hydrogen atom, a hydrocarbon with 1-18 carbonatoms (preferably CH₃, —C₂H₅), a C₁-C₂₅-hydrocarbon with at least one hydroxy group, a C₁-C₂₅-hydrocarbon with at least one carboxy group, (C₂-C₆)alkylthiol, (C₂-C₆)alkylamine, protected (C₂-C₆)alkylamine (preferably-(CH₂)₂₋₆—NH—CO—R (with R=benzylhydryloxy, 9-fluorenylmethoxy)), (C₂-C₆)alkylazide, polyethylene glycol, a crosslink to R¹ of another moiety of formula (I), polylactic acid, polyglycolic acid or polyoxazoline, or wherein R1 is a residue R⁴,
R² and R³ R² and R³ are linked to form a cyclic moiety of formula (II) comprising at least one residue R⁴
   or R² and R³ are independently selected from hydrogen, —COOH, methyl or a residue R⁴, wherein optionally, at least one of R² and R³ is a residue R⁴,
R⁴ is a moiety, comprising at least one functional group, independently selected from a functional group
   for crosslinking and/or
   for binding biologically active compounds, and
   optionally comprising a (preferably degradable) spacer moiety connecting said functional group with the binding site of the respective moiety of formula (I) or formula (II), and
R⁵ denotes a hydrogen atom, a carboxymethyl group or a methyl group,
x is 1, 2 or 3, and
* denotes a chemical bond of the polymer backbone or to a terminating moiety,
with the proviso, that at least one moiety of formula (I) or formula (II) comprises a residue R⁴, wherein preferably only the moieties of formula (I) or only the moieties of formula (II) comprise at least one moiety R⁴.
202. Polymer, especially polymer as building-block for hydrogel formation, according to item 201, characterized in, that R¹ is a hydrogen atom or a C₁-C₁₈-alkyl group, preferably a hydrogen atom, methyl, ethyl, n-propyl, iso-propyl, n-butyl, iso-butyl, sec-butyl, tert-butyl, pentyl, iso-pentyl, neopentyl, sec-pentyl, hexyl, heptyl, octyl, nonyl or decyl, more preferably methyl or ethyl.
203. Polymer, especially polymer as building-block for hydrogel formation, according to item or item 202, characterized in, that it comprises at least two different moieties of formula (I) having different groups R¹.
204. Polymer, especially polymer as building-block for hydrogel formation, according to any of the preceding items, characterized in that:
   R¹ is a hydrogen atom or a hydrocarbon with 1-18 carbon atoms, preferably for adjusting chemical characteristics of the polymer;
   R² and R³ are linked to form a cyclic moiety of formula (II) comprising at least one N-hydroxysuccinimide ester for binding biologically active compounds or R² and R³ are independently selected from hydrogen, —COOH, methyl or at least N-hydroxsuccinimide bearing molecule for binding biologically active compounds;
   R⁵ denotes a hydrogen atom, a carboxymethyl group or a methyl group;
   x is 1; and
   * denotes a chemical bond of the polymer backbone or to a terminating moiety wherein the terminating moiety preferably comprises a PNA sequence.
205. Polymer, especially polymer as building-block for hydrogel formation, according to any of the preceding items, characterized in, that x is 1 or 2, preferably x is 1.
206. Polymer, especially polymer as building-block for hydrogel formation, according to any of the preceding items, characterized in, that it comprises at least one moiety of formula (II), selected from a moiety of formula (II-a)

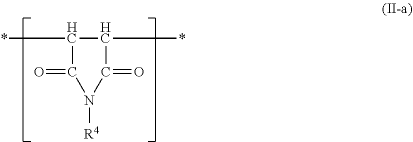

wherein
R⁴ is a moiety comprising at least one functional group
   for crosslinking and/or
   for binding biologically active compounds,
   and optionally comprising a (preferably degradable) spacer moiety connecting said functional group with the binding site of R⁴ according to formula (II-a), and
and * denotes a chemical bond of the polymer backbone or to a terminating moiety.
207. Polymer, especially polymer as building-block for hydrogel formation, according to any one of the preceding items, characterized in, that it comprises at least one moiety of formula (II), selected from a moiety of formula (II-b)

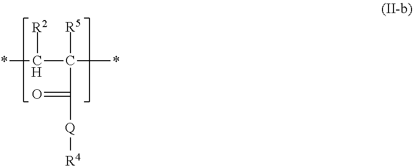

wherein
R⁵ and R⁴ is defined according to any of the preceding items,
R² is a hydrogen atom or a carboxyl group,
Q denotes an oxygen atom or an imino group NH,
and * denotes a chemical bond of the polymer backbone or to a terminating moiety.
208. Polymer, especially polymer as building-block for hydrogel formation, according to any of the preceding items, characterized in, that R⁴ is independently a moiety, comprising at least one functional group independently selected from arene, amine, alkyne, azide, anhydride, acid anhydride, ketone, haloalkane, imidoester, diol, hemiacetal, acrylate, alkene, thiol, ether, ester, isocyanate, isothiocyanate, succinimide, N-hydroxysuccinimide, sulfo-N-hydroxysuccinimide, amide, maleimide, N-heterocyclic carbene, acyl halide, N-heterocyclic phosphine, hydrazide, nitrile, aminoxy, imidazolide, imine, aldehyde, azo compound, imide, carbodiimide, haloacetyl, pyridyl disulfide, carboxamide, vinyl ether, carboxyl, carboxylate, phenyl, phenol, indol, methylthiol, pyridyldithiol, hydroxyl, epoxide, carbonyl, methoxycarbonyl, glycidyl, carboxyphenyl.
209. Polymer, especially polymer as building-block for hydrogel formation, according to any of the preceding items, characterized in, that the moiety of the formula (II) is derived from at least one monomer selected from an unsaturated imide (preferably derived from maleimide), an alkene, an acrylic acid, an itaconic acid, a lactone (preferably β-propiolactone, α-methyl-ρ-propiolactone, α,α-dimethyl β-propiolactone, β-butyrolactone), an acrylamide, a sulfonamide (preferably ethylensulfonamide), an anhydride, a methacrylic acid, an acrylamide, a methacrylamide, a N,N-diacrylamide (preferably N-methyldiacrylamide), a 1-propanesulfonic acid sultone,
   with the proviso, that said monomer comprise said residue R⁴ respectively.
210. Polymer, especially polymer as building-block for hydrogel formation, according to any of the preceding items, characterized in, that said functional group of residue R⁴ is independently selected from the group consisting of protected N-hydroxysuccinimide-esters, unprotected N-hydroxysuccinimide-esters, sulfo-N-hydroxysuccinimide esters, vinyl sulfone, sulfonyl chloride, aldehyde, epoxides, thiol, maleimide and carbonate, wherein preferably, the moiety of formula (II) comprises such residue R⁴.
211. Polymer, especially polymer as building-block for hydrogel formation, according to any of the preceding items, characterized in, that the moiety of the formula (II) is derived from monomers selected from 3-(maleimido)-propionic acid N-hydroxysuccinimide ester, 6-Maleimidohexanoic acid N-hydroxysuccinimide ester, N-(Methacryloxy)-succinimideisopropenyl, BMPH (N-(β-maleimidopropionic acid)-hydrazide, EMCH (N-ε-maleimidocaproic acid hydrazide), PDPH (3-(2-pyridyldithio)propionyl hydrazide), Methacrylic acid N-hydroxysuccinimide ester, N-methoxycarbonyl maleimide, acrylic acid N-hydroxysuccinimide ester, a PNA-amide of acrylic acid, a PNA-amide of methacrylic acid, a PNA-amide of acrylamide, a PNA-amide of methacrylamide, a monomer of formula

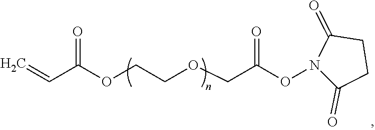

wherein n is an integer of at least 1,
a monomer of formula

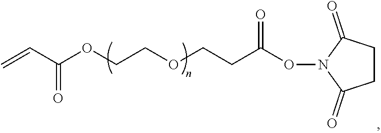

wherein n is an integer of at least 1,
a monomer of formula,

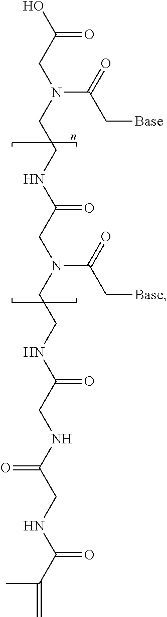

wherein n is an integer greater than 1 and Base is independently
a moiety comprising at least one nucleobase, or mixtures thereof.
212. Polymer, especially polymer as building-block for hydrogel formation, characterized in, that it comprises at least one (m is an integer of at least 1) unit having the structure of formula (III)

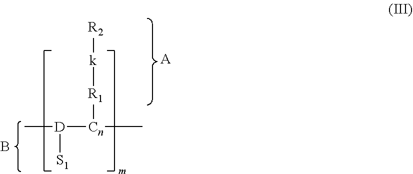

R₂ is independently a residue R⁴, comprising at least one functional group
   for crosslinking and/or
   for binding biologically active compounds,
S₁ is independently defined according to R¹ of item 201,
fragment D-Cn is part of the polymer backbone,
wherein said structure results from polymerization of a heterocyclic molecule B in presence of a first component A.
213. Polymer, especially polymer as building-block for hydrogel formation, according to item 212, characterized in, that said first component A is a compound of formula (IV)

R₁-k-R₂ (IV)
wherein
R₁ is a first functional group for the copolymerization with said heterocyclic molecule B,
R₂ is said moiety R⁴,
k is a direct bond or a spacer.
214. Polymer, especially polymer as building-block for hydrogel formation, according to item 212 or item 213, characterized in, that k is selected from a direct bond, alkylidene groups with 2 to 8 carbon atoms, hydrocarbons, and/or a degradable spacer (preferably selected from peptides, PNA, polyethylene glycol).
215. Polymer, especially polymer as building-block for hydrogel formation, according to any of the items 212 to 214, characterized in, that said first component A of formula (IV) is selected from the monomers as defined in any of the items 209 to 211.
216. Polymer, especially polymer as building-block for hydrogel formation, according to any of the items 212 to 215, characterized in, that said heterocyclic molecule B is a 2-substituted heterocyclic compound of formula (V)

D-S₁ (V)
wherein
D is an oxazoline-moiety, oxazine-moiety or oxyazepine-moiety and
S₁ is a substituent in 2-position as defined as R¹ of item 1.
217. Polymer, especially polymer as building-block for hydrogel formation, according to any of the items 212 to 216, characterized in, that said unit is a covalently functionalized D-substituted alkylamine.
218. Polymer, especially polymer as building-block for hydrogel formation, according to any of the items 212 to 217, characterized in, that it is a polymer according to any item of items 201 to 211.
219. Polymer, especially polymer as building-block for hydrogel formation, of formula (P1)

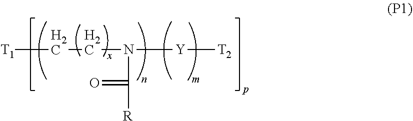

wherein
R is independently selected from a hydrogen atom, a hydrocarbon with 1-18 carbonatoms (preferably CH₃, —C₂H₅), a C₁-C₂₅-hydrocarbon with at least one hydroxy group, a C₁-C₂₅-hydrocarbon with at least one carboxy group, (C₂-C₆)alkylthiol, (C₂-C₆)alkylamine, protected (C₂-C₆)alkylamine (preferably-(CH₂)₂₋₆—NH—CO—R (with R=tert-Butyl, perfluoroalkyl)), (C₂-C₆)alkylazide, polyethylene glycol, polylactic acid, polyglycolic acid, polyoxazoline, or wherein R is a residue R⁴
Y is a moiety containing at least one graft, comprising at least one residue R⁴,
T₁ is a terminating moiety, which may contain a residue R⁴,
T₂ is a terminating moiety, which contains a residue R⁴,
p is an integer from 1 to 10,
n is an integer greater than 1 and preferably, below 500,
m is zero or an integer of at least, preferably greater than 1, and preferably, below 500, the sum n+m is greater than 10,
x is independently 1, 2 or 3, preferably x is independently 1 or 2, most preferably x is 1,
R⁴ independently comprise at least one functional group
   for crosslinking and/or
   for binding biologically active compounds, and
   optionally comprising a (preferably degradable) spacer moiety connecting said functional group with the binding site to the respective moiety of the structure of formula (P1),
wherein the entirety of all m-fold and n-fold repeating units are distributed in any order within the polymer chain and wherein optionally, the polymer is a random copolymer or a block copolymer.
220. Polymer, especially polymer as building-block for hydrogel formation, of item 219 characterized in, that Y is a moiety of formula (II) as defined in any of the items 201 to 211.
221. Polymer, especially polymer as building-block for hydrogel formation, of item 219 or item 220, characterized in, that R is a hydrogen atom or a C₁-C₁₈-alkyl group, (preferably a hydrogen atom, methyl, ethyl, n-propyl, iso-propyl, n-butyl, iso-butyl, sec-butyl, tert-butyl, pentyl, iso-pentyl, neopentyl, sec-pentyl, hexyl, heptyl, octyl, nonyl, decyl) and m is an integer greater than 1.
222. Polymer, especially polymer as building-block for hydrogel formation, of any of items 219 to 221, characterized in, that
   R is a hydrogen atom, a hydrocarbon with 1-18 carbonatoms (preferably CH3, —C2H5);
   Y is a moiety containing at least one graft, comprising at least one degradable spacer moiety connecting at least one N-hydroxysuccinimide ester for binding biologically active compounds to the respective moiety of the structure of formula (P1);
   T¹ is a terminating moiety, optionally comprising a peptide nucleic acid (PNA) sequence;
   T² is a terminating moiety, optionally comprising a peptide nucleic acid (PNA) sequence;
   n is an integer greater than 1;
   m is an integer greater than 1;
   the sum n+m is greater than 10 and less than 500; and
   x is 1;
   wherein the entirety of all m-fold and n-fold repeating units are distributed in any order within the polymer chain and wherein optionally, the polymer is a random copolymer or a block copolymer.
223. Polymer, especially polymer as building-block for hydrogel formation, according to any of the items 219 to 221, characterized in, that
   T₁ is a terminating moiety, comprising a first XNA-residue (XNA1) and optionally a EDTS-moiety,
   T₂ is a terminating moiety, comprising a second XNA-residue (XNA2) and optionally a EDTS-moiety,
   p equals 1 or 2, preferably equals 1,
   EDTS is an enzyme degradable target site, preferably a matrix metalloprotease (MMP) target site, for site directed degradation of the polymer,
   XNA is a nucleic acid or nucleic acid analog, preferably a peptide nucleic acid (PNA) sequence.
224. Polymer, especially polymer as building-block for hydrogel formation, of item 223, characterized in, that m is zero and no moiety Y is comprised in the polymer.
225. Polymer, especially polymer as building-block for hydrogel formation, according to any of the items 219 to 221, characterized in, that
   T₁ is a terminating moiety, comprising no residue R⁴,
   T₂ is a terminating moiety, comprising a XNA-residue, optionally linked to a EDTS-moiety,
   p is an integer of 3 to 10, preferably 3 to 10, preferably 3 to 8, most preferred 3 to 6,
   EDTS is an enzyme degradable target site, preferably a matrix metalloprotease (MMP) target site, for site directed degradation of the polymer,
   XNA is a nucleic acid or nucleic acid analog, preferably a peptide nucleic acid (PNA) sequence.
226. Polymer, especially polymer as building-block for hydrogel formation, of item 225, characterized in, that m is zero and no moiety Y is comprised in the polymer.
227. Polymer, especially polymer as building-block for hydrogel formation, according to any of the items 219 to 221, characterized in, that
   T₁ is a terminating moiety, comprising a residue R⁴ different from a XNA-residue, wherein R⁴ is optionally linked to a EDTS-moiety,
   T₂ is a terminating moiety, comprising a residue R⁴ different from a XNA-residue, wherein R⁴ is optionally linked to an EDTS-moiety,
   p equals 1 or 2, preferably equals 1,
   EDTS is an enzyme degradable target site, preferably a matrix metalloprotease (MMP) target site, for site directed degradation of the polymer,
   XNA is a nucleic acid or nucleic acid analog, preferably a peptide nucleic acid (PNA) sequence.
228. Polymer, especially polymer as building-block for hydrogel formation, of item 227, characterized in, that m is zero and no moiety Y is comprised in the polymer.
229. Polymer, especially polymer as building-block for hydrogel formation, according to any of the items 219 to 221, characterized in, that
   T₁ is a terminating moiety, comprising no residue R⁴,
   T₂ is a terminating moiety, comprising a residue R⁴ different from a XNA-residue, wherein R⁴ is optionally linked to an EDTS-moiety,
   p is an integer of 3 to 10, preferably 3 to 10, preferably 3 to 8, most preferred 3 to 6,
   EDTS is an enzyme degradable target site, preferably a matrix metalloprotease (MMP) target site, for site directed degradation of the polymer,
   XNA is a nucleic acid or nucleic acid analog, preferably a peptide nucleic acid (PNA) sequence.
230. Polymer, especially polymer as building-block for hydrogel formation, of item 229, characterized in, that m is zero and no moiety Y is comprised in the polymer.
231. Polymer especially polymer as building-block for hydrogel formation, according to any of the items 223 to 230, characterized in, that it is a polymer which comprises an EDTS-moiety, preferably a MMP-moiety.
232. Polymer, especially polymer as building-block for hydrogel formation, according to any of the items 219 to 231, characterized in, that it comprises at least two different moieties R.
233. Polymer, especially polymer as building-block for hydrogel formation, according to any of the items 219 to 221, characterized in, that p is an integer of 3 to 10, preferably 3 to 10, preferably 3 to 8, most preferred 3 to 6.
234. Polymer, especially polymer as building-block for hydrogel formation, of formula (P2)

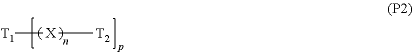

wherein
T₁ is a terminating moiety, which contains a residue -XDTS-XNA1,
T₂ is a terminating moiety, which contains a residue -XDTS-XNA2,
XDTS is independently selected from a direct bond or an EDTS-moiety, wherein EDTS is an enzyme degradable target site, preferably a matrix metalloprotease (MMP) target site, for site directed degradation of the polymer,
XNA1 is a nucleic acid or nucleic acid analog, preferably a peptide nucleic acid (PNA) sequence,
XNA2 is the same or a different nucleic acid or nucleic acid analog compared to XNA1, preferably a peptide nucleic acid (PNA) sequence,
p is 1 or 2, preferably 1,
X is a hydrophilic polymeric residue, preferably independently derived from monomers independently selected from oxazoline, ethylene glycol, propylene glycol, acetal lactic acid, glycolic acid, vinyl alcohol,
n is an integer greater than 1, preferably from 1 to 10000.

According to one embodiment, at least one X is different from oxazoline.
235. Polymer, especially polymer as building-block for hydrogel formation, according to formula (P2) of item 234, characterized in that
   T₁ is a terminating moiety, comprising no XNA-residue,
   T₂ is a terminating moiety, comprising a XNA-residue and optionally an EDTS-moiety,
   p is an integer of 3 to 10, preferably 3 to 8, most preferred 3 to 6,
   X hydrophilic polymeric residue, preferably independently derived from monomers independently selected from oxazoline, ethylene glycol, propylene glycol, acetal lactic acid, glycolic acid, vinyl alcohol,
   EDTS is an enzyme degradable target site, preferably a matrix metalloprotease (MMP) target site, for site directed degradation of the polymer,
   XNA is a nucleic acid or nucleic acid analog, preferably a peptide nucleic acid (PNA) sequence,
   n is an integer greater than 1, preferably from 1 to 10000.
236. Polymer, especially polymer as building-block for hydrogel formation, according to any of the preceding items, wherein the polymer is functionalized by at least one biologically active compound, preferably, at least two different biologically active compounds, preferably by reaction of an amino group of the biologically active compound with a functional group of residue R⁴.
237. Polymer, especially polymer as building-block for hydrogel formation, according to any of the preceding items, characterized in, that the biologically active compound selected from the group consisting of peptides, proteins, CRISPR-Cas enzyme complex, apoptosis-inducing active substances, adhesion-promoting active substances, anti-inflammatory active substances, receptor agonists and receptor antagonists, growth-inhibiting active substances (and in particular from proteins of the extracellular matrix, cell surface proteins, antibodies, growth factors, sugars, lectins, carbohydrates, cytokines, DNA, RNA, siRNA), aptamers, and fragments thereof, or mixtures thereof.
238. Polymer, especially polymer as building-block for hydrogel formation, according to any of the preceding items, characterized in, that it comprises at least one biologically active compound selected from the group consisting of peptides, proteins, CRISPR-Cas enzyme complex, apoptosis-inducing active substances, adhesion-promoting active substances, anti-inflammatory active substances, receptor agonists and receptor antagonists, growth-inhibiting active substances (and in particular from proteins of the extracellular matrix, cell surface proteins, antibodies, growth factors, sugars, lectins, carbohydrates, cytokines, DNA, RNA, siRNA), aptamers, and fragments thereof, or mixtures thereof.
239. Polymer, especially polymer as building-block for hydrogel formation, according to any of the preceding items, characterized in, that it comprises at least one biologically active compound selected from a Peptide nucleic acid (PNA) and/or a locked nucleic acid (LNA), preferably wherein the PNA-moiety independently comprise a structure of formula (VI)

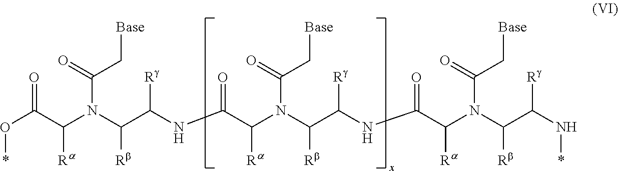

wherein
x is an integer greater than 1,
Base is independently a moiety comprising at least one nucleobase (preferably selected from adenin, cytosin, guanine, thymine, 2,6-diaminopurine, analogs of thymine and cytosine, hypoxanthine, derivatives thereof functionalized with a fluorescent dye (preferably thiazole orange)),
Rα and Rβ are independently selected from hydrogen atom, any residue bound to the alpha-carbon atom of any of the proteinogenic amino acid,
Rγ is a hydrogen atom, a moiety with at least one ionic residue.
240. Polymer, especially polymer as building-block for hydrogel formation, according to any of the preceding items, characterized in, that it comprises at least one biologically active compound, selected from a Peptide nucleic acid (PNA) comprising a matrix metalloprotease target site for the site directed degradation (MMP).
241. Polymer, especially polymer as building-block for hydrogel formation, according to item or item 240, characterized in, that is comprises at least one additional biologically active compound, selected from the group consisting of peptides, proteins, CRISPR-Cas enzyme complex, apoptosis-inducing active substances, adhesion-promoting active substances, anti-inflammatory active substances, receptor agonists and receptor antagonists, growth-inhibiting active substances (and in particular from proteins of the extracellular matrix, cell surface proteins, antibodies, growth factors, sugars, lectins, carbohydrates, cytokines, DNA, RNA, siRNA), aptamers, and fragments thereof, or mixtures thereof.
242. Polymer, especially polymer as building-block for hydrogel formation, according to any one of the preceding items, wherein the polymer has a linear structure (preferably a graft polymer, grafted with at least one residue R⁴) or a dendritic structure (preferably a linear structure or a star shaped structure).
243. Polymer, especially polymer as building-block for hydrogel formation, according to any one of the preceding items, wherein the polymer is random polymer, a block-copolymer or a dendrimer.
244. Polymer, especially polymer as building-block for hydrogel formation, according to any one of the preceding items, wherein the polymer has a star-shaped structure comprising at least three arms.
245. Polymer, especially polymer as building-block for hydrogel formation, according to any of the preceding items, wherein said functional group for crosslinking is selected from amine, N-hydroxysuccinimide, sulfo-N-hydroxysuccinimide, isothiocyanate, maleimide, thiol, azide, alkyne, alkene, hydrazide, aminoxy, aldehyde, carboxyl, carboxylate, hydroxyl, acrylate, vinyl ether, epoxide (preferably from amine, maleimide, alkyne, alkene, azide, carboxyl, carboxylate, methacrylate, acrylate, thiol).
246. Polymer, especially polymer as building-block for hydrogel formation, according to any of the preceding items, wherein said functional group for binding a biologically active compound is independently selected from amine, N-hydroxysuccinimide, sulfo-N-hydroxysuccinimide, alkyne, alkene, hydrazide, epoxide, glycidyl, carboxyphenyl, methoxycarbonyl, carboxyl, carboxylate, isothiocyanate, maleimide, aminoxy, hydroxyl, vinyl ether (preferably from amine, N-hydroxysuccinimide, sulfo-N-hydroxysuccinimide, hydrazide, epoxide, glycidyl, phenyl acrylate, methoxycarbonyl, carboxyl, carboxylate).
247. Polymer, especially polymer as building-block for hydrogel formation, according to any of the preceding items, wherein the polymer is prepared by at least one polymerization step, selected from living cationic ring-opening polymerization (CROP), spontaneous zwitterionic copolymerization (SZWIP) or a combination of both.
248. Polymer, especially polymer as building-block for hydrogel formation, according to item 247, characterized in, that the polymerization, preferably the living cationic ring-opening polymerization, is initiated by an initiator with an electrophilic character.
249. Polymer, especially polymer as building-block for hydrogel formation, according to item or item 248, characterized in, that the initiator is selected from triethylene glycol di (p)-toluenesulfonate, pentaerythritol tetrabromide, pentaerythritol tetrakis(benzenesulfonate) or p-toluenesulfonyl chloride modified N,N,N′,N′-Tetrakis(2-hydroxyethyl)ethylenediamine.
250. Polymer, especially polymer as building-block for hydrogel formation, according to any of items 247 to 249, characterized in, that the polymerization, preferably the living cationic ring-opening polymerization, is terminated by addition of a terminating molecule selected from nucleophiles, amines, azides or acids (preferably carboxylic acids).
251. Polymer, especially polymer as building-block for hydrogel formation, according to any of any of items 247 to 250, characterized in, that the polymerization, preferably the living cationic ring-opening polymerization, is terminated by addition of a terminating molecule selected from peptide nucleic acid (PNA), preferably peptide nucleic acid (PNA) with unprotected carboxylic acid group at the C-terminus and protected amino group at the N-terminus or peptide nucleic acid (PNA) with unprotected amino group at the N-terminus and protected carboxylic acid group at the C-terminus).
252. Polymer, especially polymer as building-block for hydrogel formation, according to any of any of items 247 to 251, characterized in, that the polymerization, preferably the spontaneous zwitterionic copolymerization, is terminated by addition of a terminating molecule selected from electrophiles, preferably selected from α,β-unsaturated carboxylic acids, α,β-unsaturated carboxylic acidamides, mixtures thereof, most preferred from acrylic acid, methacrylic acid, acryl amide, methacryl amide, functionalized with at least one residue R⁴ as defined in any of the preceding items respectively (most preferred functionalized with -MMP-PNA respectively).
253. Polymer, especially polymer as building-block for hydrogel formation, according to any of any of items 247 to 252, characterized in, that said initiator and/or said terminating molecule incorporates a moiety R⁴ as defined in any of items 201, 208 to 210, 245 and 246.
254. Polymer, especially polymer as building block for hydrogel formation, according to any of the items 247 to 251 and 253, characterized in, that the polymerization, preferably the spontaneous zwitterionic copolymerization, is terminated by addition of a terminating molecule selected from selected from α,β-unsaturated carboxylic acids, α,β-unsaturated carboxylic acidamides, mixtures thereof (most preferred from acrylic acid, methacrylic acid, acryl amide, methacryl amide) followed after optional workup by a coupling of a residue comprising PNA and a thiol functionality.
255. Polymer, especially polymer as building block for hydrogel formation, according to any of the items 247 to 254, characterized in, that a residue comprising PNA and a thiol functionality is coupled to a maleimide as a functional group of residue R⁴.

The present application claims priority of European patent applications 17 190 299.2 and 17 190 298.4 and U.S. provisional application 62/623,772, the content of which is herein incorporated by reference.

### LIST OF REFERENCE SIGNS

1 Second opening
2 First opening
3 Actuation chamber
4 Inner boundary of the valve portion
5 Outer boundary of the valve portion
6 Boundary of actuation chamber
7 Cross section of connection channel
8 Curved inner boundary
9 Lamella
10 Microfabricated valve
11 first channel
12 second channel
13 connection channel
14 valve portion
15 flexible membrane
16 bottleneck section
17 particle trap
18 bypass
19 recess
20 particle
21 first layer
22 second layer
23 third layer
30 microbiological test device
31 droplet/object
32 location/observation chamber
33 positioner/droplet trap
34 bottleneck section
35 bypass section
38 impedance measurement device
39 frequency application device
40 valve arrangement
41 feeding channel
42 inlet for loading
43 feeding exit
44 dielectrophoretic (DEP) force generator
45 poles
50 pump
51 first path of flow
52 second path of flow
53 third path of flow
54 fourth path of flow
501 first fluid line
502 second fluid line
503 third fluid line
504 fourth fluid line
505 fifth fluid line
51 first path of fluid
52 second path of fluid
53 third path of fluid
54 third path of fluid
60 valve arrangement
61 droplet collection channel/collection chamber
62 opening
63 valve
64 channel
65 damping device/membrane structure
66 membrane
67 actvating channel
68 air chamber/compensation chamber
69 passage
610 housing
70 centering station
71 first path of flow
72 second path of flow
93 third path of flow
V1-V5 valves
C direction
80 well
A1 . . . H12 new location within well
81 droplet
82 particle
83 parameter of particle
84 particle ID
85 position ID of new location
86 database
F1, F2, F3 forces
F1, F2 functional group
S1 first fluid direction
S1 second fluid direction
Cm1 trigger command for group m1
Cm2 trigger command for group m2
. . .
Cm5 trigger command for group m5
Cn1 trigger command for group m1
Cn2 trigger command for group m2
. . .
Cn5 trigger command for group n5
101 Merging position
102 First second opening
103 Second second opening
104 First first opening
105 Baffel structured opening
106 First section of flexible membrane
107 Second section of flexible membrane
108 First first/second opening
109 Second first/second opening
110 Third first/second opening
111A First actuation chamber
111B Second actuation chamber
112 Etching access structure
113 Support structure
114 Narrowed section
115 Third channel
116 First section of connection channel
117 Second section of connection channel
118 N^{th} first/second opening

### Further Subject-Matter

In addition, the following matters are disclosed as part of the present disclosure:
1. Microfabricated valve (10), comprising
   a first channel (11);
   a second channel (12);
   a connection channel (13) connecting the first channel (11) and the second channel (12);
   a valve portion (14) arranged within the connection channel (13),
   wherein the valve portion (14) is adapted to selectively open and close the connection channel (13).
2. Microfabricated valve (10) according to the preceding matter,
   characterized in
   that the valve portion (14) comprises at least one flexible membrane (15), the flexible membrane (15) is adapted to be selectively transferred between an open shape and a closed shape.
3. Microfabricated valve (10) according to any of the preceding matters,
   characterized in,
   that the valve portion (14) is adapted to be selectively opened and closed upon modification of a fluid pressure of a control fluid, in particular compressed air, acting onto the membrane (15),
   in particular that the flexible membrane (15) is transferred into the open shape and/or transferred into the closed shape upon decreasing/increasing the fluid pressure.
4. Microfabricated valve (10) according to any of the preceding matters,
   characterized in,
   that the valve portion (14) is adapted to be selectively opened and closed upon modification of a voltage applied to the valve portion, in particular
   the valve portion comprises at least one electrostatic chargeable layer, in particular polymer layer, which is adapted to change its form upon modification of the voltage.
5. Microfabricated valve (10) according to any of the preceding matters,
   characterized in,
   that the microfabricated valve (10) comprises at least three layers (21, 22, 23), wherein
   the first channel (11) is located within a first layer (21);
   the second channel (12) is located within a third layer (23);
   the valve portion (14) is located within a second layer (22);
   the second layer (22) is arranged between the first (21) and the third layer (23).
6. Microfabricated valve (10) according to any of the preceding matters,
   characterized in,
   that the membrane has a biconvex shape or a triangular shape.
7. Test device (30), in particular for biological applications, e.g. a microbiological test device, comprising a plurality of observation chambers (32), wherein the observation chamber (32) is adapted to accommodate at least one droplet (31), the droplet in particular comprising a hydrogel particle, provided within a fluid.
8. Test device (30) according to the preceding matter,
   characterized in
   that the test device comprising a valve (10) according to any of matters 1 to 6,
9. Test device (30) according any of matters 7 to 8,
   characterized in,
   that the test device, e.g. the observation chamber, comprises a trap (17), in particular a particle trap and/or a droplet trap, to retain a predetermined number of particles (20) and/or droplets (31), which are provided within a stream of fluid (36) passing the trap (17), in particular in a first fluid direction (S1),
   in particular wherein the trap (17) comprising a bottleneck section (16, 34) having a smaller diameter than a particle (20) or droplet (31) to be retained.
10. Test device (30) according to any of matters 7 to 8 or 9,
   characterized in,
   that the trap (17) comprising a bypass section (18, 35), in which particles (20) or droplets (31) can circumvent the bottleneck section (16, 34) when the trap (17) is occupied by a predetermined number,
   in particular one, of retained particles (20) or droplets (31).
11. Test device (30) according to any of matters 9 or 10,
   characterized in
   that below the trap (17), a valve portion (14), in particular of a valve (10) according to any of matters 1 to 6, is provided, wherein test device is adapted to selectively transfer the particles (20) or droplets (31) from the trap (17) through the valve portion (14) from one channel (12, 11) into another channel (11, 12).
12. Test device (30) according to the preceding matter,
   characterized in
   that the test device comprises two neighbouring traps (17n), wherein the valve portion (14) is located below both traps (17n), wherein the test device is adapted to selectively transfer the particles (20) or droplets (31) from both traps (17n) through the valve portion (14) from two separate second channels (12, 12″) into a separate first channel (11),
   in particular wherein in the both second channels (12, 12″) a same second pressure (p12) is applied to the fluid.
13. Test device (30) according to any of matters 9 to 12,
   characterized in,
   that the trap (17) is adapted to selectively release a retained droplet (31), in particular the trap (17) is adapted to selectively release a retained droplet or particle upon application of a fluid in second fluid direction (S2), in particular opposite to the first fluid direction (S1).
14. Test device (30) according to any of matters 9 to 13,
   characterized in,
   that test device (30) is adapted to selectively release a retained droplet (31) within a selected observations chamber (32), wherein the unselected observations chambers (32) are adapted to keep on retaining the retained droplets (31).
15. Test device (30) according to the preceding matter,
   characterized by
   an exit delivery mechanism is adapted to deliver a released droplet (31) to an exit portion (P2).
16. Test device (30) according to any of matters 9 to 15,
   characterized in,
   that the droplet trap (17) is adapted to retain a predefined sequence of droplets (31A, 31B, 31C) or particles subsequently arriving at the observation chamber (32), in particular at separate predefined positions,
   in particular the droplet trap (17) comprising a plurality of bottleneck section (34A,34B,34C), in particular arranged in series.
17. Test device according to the preceding matter,
   characterized in
   that the droplet trap (17) is designed in a way, that upon a change of the direction of fluid a specific force is applied to the droplets or particles pushing the droplets or particles out of the trap (17), wherein the respective pushing force is different for each of the predefined positions.
18. Test device (30) according to any matters 9 to 17,
   characterized by,
   each observation chamber (32) has a valve arrangement (40) adapted to provide a fluid passing the droplet trap (17), wherein the valve arrangement (40) is adapted to selectively change the direction of fluid (S1, S2) passing the observation chamber (33), in particular wherein a fluid a first direction (S1) urging the droplet (31) into the droplet trap (33) and a fluid in the second direction (S2) urging the droplet out of the droplet trap (33),
   and in particular fluid in the second direction (S2) delivering the droplet in direction of the exit section (P2).
19. Test device (30) according to any matters 7 to 18,
   characterized by,
   a dielectrophoretic (DEP) force generator (44), for generating a dielectrophoretic (DEP) force acting on a droplet (31), in particular the dielectrophoretic (DEP) force generator (44) is part of a trap (17) for retaining a droplet (31).
20. Test device (30) according the preceding matter 7 to 19,
   characterized in
   that the trap (17) comprises a structure (46), which is adapted to stimulate the droplet (31) to rotate upon application of a stream of fluid acting on the droplet (31).
21. Test device (30) according to any matters 9 to 20,
   characterized by a camera focused on a trap, adapted to take an optical image of a droplet or particle, which is retained within the trap.
22. Test device (30) according to any of matters 7 to 21,
   characterized that, a plurality of the observation chambers each having a respective valve arrangement (40m2n2), wherein each of the valve arrangements (40m2n2) are allocated
   a) to one of a first group (m2) of valves arrangements (40m2) and
   b) to one of a second group (n2) of valve arrangements (40n2),
   wherein the valve arrangements of one group can be triggered commonly by a respective common group command (Cm1, Cm2, Cm3, . . . Cn1, Cn2, Cn3, . . . ); in particular wherein one group command comprises a first group commands (Cm1, Cm2, Cm3, . . . ) and a second group commands (Cn1, Cn2, Cn3, . . . ).
23. Test device (30) according to any of matters 7 to 22,
   characterized in,
   that the valve arrangement (40m2, n2) is adapted to change the direction of the fluid if both group commands issue a group command (Cm2=1, Cn2=1) referring to the both groups to which the valve arrangement (40m2n2) belongs.
24. Test device (30) according to any of matters 7 to 23,
   characterized by
   a feeding channel (41), adapted for initially supplying droplets (31) or particles (20) in a fluid from an inlet into a plurality of observation chambers (32), wherein the plurality of observations chambers (32) are connected by the feeding line (41) in series.
25. Test device (30) according to any of matters 7 to 24,
   characterized by
   an impedance measuring device (38) for measuring the impedance of at a droplet or a particle, in particular at a location, where the droplet or particle is held stationary, in particular for at least 0.1 seconds.
26. Test device (30) according to any of matters 7 to 25,
   comprising a radio frequency application device (39) for applying a radio frequency to a droplet or a particle, in particular at a location, where the droplet or particle is held stationary, in particular for at least 0.1 seconds,
   wherein the radio frequency application device (39) is in particular adapted to the droplet and/or the particle, so that the droplet and/or the particle is heated upon application of the radio frequency.
27. Method of creating droplets, in particular encapsulations, within a first fluid, comprising the following steps:
   a) providing a microfabricated valve (10) according to any matters 1 to 6, wherein the first channel (11) is filled with a first fluid, wherein the second channel (12) is filled with a second fluid, wherein the second fluid is unsoluble in the first second fluid,
   b) applying a pressure difference (p2-p1) to the fluids, wherein the second fluid is pressurized by a second pressure (p2) and the first fluid is pressurized by a first pressure (p1), wherein the second pressure (p2) is larger than the first pressure (p1),
   c) selectively opening the valve portion (14),
   d) subsequently closing the valve portion (14) as soon as a defined quantity of the second fluid has passed the valve portion (14) in direction from the second channel (12) to the first channel (11).
28. Method according to the preceding matter,
   characterized in
   that a particle (20) is comprised within the second fluid,
   wherein the particle is retained by a trap (17) above the valve portion (14),
   wherein during selectively opening and closing the valve portion (14) at least one particle (20), in particular exactly one particle, passing the valve section (14) along with the defined quantity of the second fluid.
29. Method according to matter 27 or 28,
   characterized in that the defined quantity is adjusted
      by varying an opening duration (t_open) of the valve portion (14), and/or
      by varying a pressure difference (p2-p1) between the second channel (12) and the first channel (11).
30. Method according to any of matters 27 to 29,
   characterized by the following steps:
   using a first valve (10A) according to any of matters 1 to 6 to generate a first droplet (31A) having a first ingredient;
   using a second valve (10B) according to any of matters 1 to 6 to generate a second droplet (31B) having at least a second ingredient (19);
   merging both droplets (31A, 31B) in the first channel (11) to generate a merged droplet (31AB) comprising the first and second ingredients (19), in particular by generating a flow in the first channel (11).
31. Method for performing a biological test cycle, in particular using a test device (10) according to any of matters 7 to 26, comprising the steps providing a plurality of droplets, in particular comprising particles (20), within a stream of fluid;
   selectively trapping one individual droplet (31) or a present number of droplets within an observation chamber (32), in particular within a trap (17) of the observation chamber (32).
32. Method according to the preceding matter,
   characterized in
   that a plurality of droplets is supplied in a sequence of droplet (31) to a first observation chamber (32), a present number, in particular one or more, of droplets (31) is retained in the trap (17) of the first observation chamber (32), in particular according to a present maximum numbers of droplets to be retained in the first observation chamber (32),
   all droplets subsequently approaching the first observation chamber (31) and exceeding the present number of droplets are forwarded to a second observation chamber (32), in particular via a bypass section (35) of the trap (17).
33. Method according to matter 31 or 32,
   characterized in
   after retaining an individual droplet (31) for a given time period within the observation chamber (32), selectively untrapping an individual droplet (31) from the observation chamber and selectively delivering the untrapped droplet to an exit section (P2).
34. Method according to any of matters matter 31 to 33,
   characterized in,
   that in case that a plurality, in particular more than one, of droplets (31A-31C) are retained in a single observation chamber (32), each of the plurality of droplets (31A-31C) is individually released from the observations chamber, in particular by applying different forces, in particular by different fluid pressure or fluid rates, to the observation chamber (32).
35. Method according to any of matters 31 to 34,
   characterized by the following steps:
   providing a droplet (31) in the second channel (12), wherein the droplet (31) comprising one or more particles, in particular a particle (20);
   bringing the droplet (31) into rotation, so that a centripetal force acting on the particles (20), leading to a centering effect of the particles (20) within in the droplet (31), in particular wherein the centering effect may occur before and/or during a polymerisation of a hydrogel within the droplet.
36. Method according to any of matters 31 to 35,
   characterized in the step of extracting an ingredient (19) of the droplet (31) from a droplet carrier material, in particular by using a microfabricated valve (10) according to any of matters 1 to 6.
37. Method according to any of matters 31 to 36,
   characterized in the steps
   a) providing a droplet (31) within a location, in particular an observation chamber (32), in particular trapped within a trap (17), the droplet (31) comprising an immobilized hydrogel matrix and the location is filled with a first, in particular aqueous, fluid;
   b) perfusing the location with a second, in particular oily, fluid, so that the first fluid is removed from the droplet (17).
38. Method according to the preceding matters,
   characterized in the step
   c) after step b, perfusing the location with the first fluid, so that the second fluid is removed from the droplet (17).
39. Method according to any of matters 31 to 37,
   characterized in
   that the test device is filled with a cryoprotectant fluid,
   subsequently the test device (10) is frozen,
   in particular wherein during filling the cryoprotectant and freezing at least a droplet and/or particle is retained in an observation chamber (32) or in a trap (17) of the test device.
40. Method for demulsification of droplet comprised within a first fluid, comprising the following steps:
   a) providing a microfabricated valve (10) according to any of matters 1 to 6 or a test device according to any of matters 7 to 26,
   wherein the first channel (11) is filled with a first fluid,
   wherein the second channel (12) is filled with a second fluid,
   wherein in the first channel (11) a droplet (31) of a second fluid is comprised,
   wherein the second fluid is insoluble in the first second fluid.
41. Method according to the preceding matter, comprising the following steps:
   b) in particular applying a pressure difference (p2-p1) to the channels (11, 12), wherein the second channel (12) is pressurized by a second pressure (p2) and the first channel (11) is pressurized by a first pressure (p1), wherein the first pressure (p1) is larger than the second pressure (p2), or selectively opening the valve portion (14), in particular wherein the lower density of the droplet (31) is used to generate a flow from the first channel (11) through the valve section (13) to the second channel (12),
   c) subsequently closing the valve portion (14) as soon as the droplet (31) has passed the valve portion (14) in direction from the first channel (11) to the second channel (12).
42. Method according to any of the matters 39 to 41,
   characterized in,
   that the droplet (31) comprises an ingredient (19), wherein after the droplet (31) has reached the second channel (12) the ingredient is released form the droplet (31).
43. Method according any of matters 27 to 42,
   characterized in,
   that the second fluid is an aqueous fluid and the first fluid is an oily fluid.
44. Pump (50), comprising at least two, in particular at least three, valves (10) according to any of matters 1 to 6, arranged in series,
   wherein the pump (50) is adapted to pump a fluid upon, in particular a sequential, activation of the valves (10A, 10C; 10C),
   in particular wherein, considered in a direction (F) of fluid, an outlet channel (12A) of a first valve (10A) is connected to an inlet channel (12B) of a second valve (10B), and/or
   in particular wherein, considered in a direction (F) of fluid, an outlet channel (11B) of a second valve (10B) is connected to an inlet channel (11A) of a third valve (10C).
45. Pump (50) according to the preceding matter,
   characterized by
   at least two first valves (10A) arranged in parallel to each other, and/or at least two second valves (10B) arranged in parallel to each other and/or at least two thrid valves (10C) arranged in parallel to each other,
   in particular
   wherein the inlet channels (11A) of the first valves (10A) are connected to each other and/or
   wherein the outlet channels (12A) of the first valves (10A) are connected to each other and/or
   wherein the inlet channels (12B) of the second valves (10B) are connected to each other and/or
   wherein the outlet channels (11B) of the second valves (10B) are connected to each other and/or
   wherein the inlet channels (11C) of the third valves (10C) are connected to each other and/or wherein the outlet channels (12C) of the third valves (10C) are connected to each other.
46. An organic monomer comprising a covalently functionalized D-substituted alkylamine.
47. The organic monomer according to matter 46, wherein the covalently functionalized D-substituted alkylamine comprises
   according to variant A at least two components, wherein the first component comprises at least three different parts:
      an organyl group linking the first component to the second component,
      a functional group for crosslinking the organic monomer to a biologically active compound, and
      a spacer between the two functional groups, and
   wherein the second component is an organic amide;
   or
   according to Variant B at least two components, wherein the first component comprises at least of three different parts:
      a first functional group for the copolymerization with the second component, wherein preferably, the first functional group is an unsaturated imide or an alkene, wherein more preferably the unsaturated imide is 3-(maleimido)-propionic acid N-hydroxysuccinimide and the alkene preferably is isopropenly
      a second functional group for crosslinking to a biologically active compound, and
      a spacer between the two functional groups, and
   wherein the second component is or is derived from a heterocyclic chemical compound.
48. The organic monomer according to any one of the preceding matters per variant A, wherein the organyl group is a substituted succinimide with the formula (CH₂)₂(CO)₂NR where R represents the substituent in form of a spacer and the second functional group.
49. The organic monomer according to any one of the preceding matters per variant A, wherein the organyl group is a double-branched-chain alkane with at least five carbon atoms from which one is substituted with the spacer and the second functional group.
50. The organic monomer according to any one of the preceding matters per variant A, wherein the organyl group is a branched-chain alkane with at least two carbon atoms from which one is substituted with the spacer and the second functional group.
51. The organic monomer according to any one of the preceding matters per variant A, wherein the organyl group is a chain alkane with at least two carbon atoms from which one is substituted with the spacer and the second functional group.
52. The organic monomer according to any one of the preceding matters per variant A, wherein the first component is 3-(succinimde)-propionic acid N-hydroxysuccinimide.
53. The organic monomer according to any one of the preceding matters, wherein the second functional group is selected from the group consisting of NHS esters, anhydrides, sulfonyl chlorides, aldehydes, epoxides and carbonates.
54. The organic monomer according to any one of the preceding matters, wherein the spacer is an acyclic saturated hydrocarbon and separates the two functional groups by at least one carbon atom.
55. The organic monomer according to any one of the preceding matters, wherein the second component is 2-substituted oxazoline.
56. The organic monomer according to any one of the preceding matters, wherein the second component is 2-methyl oxazoline.
57. The organic monomer according to any one of the preceding matters, wherein the second component is substituted at position 2 with the second component.
58. The organic monomer according to any one of the preceding matters, wherein the biologically active compound is selected from the group consisting of a protein such as an antibody, a growth factor or an ECM protein like collagen, laminin, fibronectin or an artificial peptide, a nucleic acid such as a DNA primer, a CRISPR-Cas enzyme complex and a peptide nucleic acid such as a PNA oligomer.
59. The organic monomer according to any one of the preceding matters, wherein the covalently functionalized D-substituted alkylamine is composed of at least two components (A and B) and having the structure

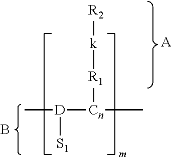

R₁ is a substituted imide,
k is a spacer like a acylic saturated hydrocarbon (C1-C5), and
R₂ a functional group for crosslinking of biological active compounds.
60. The organic monomer according to any one of the preceding matters, wherein the covalently functionalized D-substituted alkylamine is a covalently functionalized N-substituted polyethyleneimine.
61. The organic monomer according to any one of the preceding matters, wherein the covalently functionalized D-substituted alkylamine having the structure

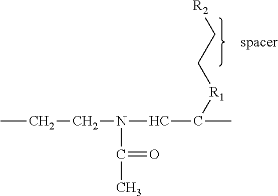

wherein,
   R₁ is a substituted imide,
   the spacer like a acylic saturated hydrocarbon (C1-C5), and
R₂ a functional group for crosslinking of biological active compounds.
62. The organic monomer according to any one of the preceding matters, wherein the organic monomer having the structure

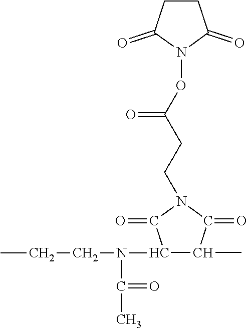 [SMILES: CCCN(C(C)=O)C1C(=O)N(CCC(=O)ON2C(=O)CCC2=O)C(=O)C1C]

63. The organic monomer according to any one of the preceding matters, wherein the organic monomer comprises further a Peptide nucleic acid (PNA) and/or a locked nucleic acid (LNA).
64. An organic polymer comprising at least two organic monomers of any one of the preceding matters, wherein.
65. The organic polymer according to matter 62, wherein organic polymer is a Poly-2-methyl-2-oxazoline (PMOx)-based polymer.
66. The organic polymer according to any one of the preceding matters, wherein the organic polymer comprises further a Peptide nucleic acid (PNA) and/or a locked nucleic acid (LNA).
67. The organic polymer according to any one of the preceding matters, wherein the Peptide nucleic acid (PNA) is located at the end of the first polymer.
68. The organic polymer according to any one of the preceding matters, wherein the polymer has a linear or multiarm/star-shaped structure.
69. A hydrogel matrix composed of a mixture of least two different organic polymers according to any one of the proceeding matters.
70. The hydrogel matrix according to matter 67, wherein the hydrogel matrix is composed of at least two different organic polymers according to any one of the proceeding matters, wherein the different polymers having different structures, wherein the first polymer has a linear structure and the second polymer has multiarm or star-shaped structure.
71. The hydrogel matrix according to any one of the preceding matters, wherein the hydrogel matrix has a spherical and/or plug-like structure.
72. The hydrogel matrix according to any one of the preceding matters, wherein the hydrogel matrix comprises a bioactive molecule.
73. The hydrogel matrix according to matter 70, wherein the bioactive molecule is selected from the group consisting of a protein such as an antibody, a growth factor or an ECM protein like collagen, laminin, fibronectin or an artificial peptide, a nucleic acid such as a DNA primer, a CRISPR-Cas enzyme complex and a peptide nucleic acid such as a PNA oligomer.
74. A microfluidic array having microfabricated structures for the generation and/or immobilization and/or recovery of a hydrogel matrix according to any one of the proceeding matters containing at least one particle and/or cell located for analysis of cell characteristics and/or behavior and methods for producing said array.
75. The microfluidic array according to matter 72, wherein the cells of at least two different cell types are encapsulated within the hydrogel matrix.
76. The microfluidic array according to any one of the preceding matters having microfabricated structures for the immobilization of at least two spherical matrices for cell cultivation of encapsulated cells of the same or of different cell types and/or analysis of cell behavior of the encapsulated cells of the same or of different cell types and/or recovery of the encapsulated cells of different cell types within the spherical matrices.
77. An organic building block comprising a substituted tertiary amide group represented by the formula:

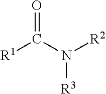

78. The organic building block according to matter 75, wherein the substitute R1 in the formula is an alkyl group.
79. The organic building block according to any one of the preceding matters, wherein the substitute R1 in the formula is a methyl group.
80. The organic building block according to any one of the preceding matters, wherein the substitute R1 in the formula is a hydrogen.
81. The organic building block according to any one of the preceding matters, wherein the substitute R2 in the formula is any one of the components according to matter 48-54 or a hydrogen.
82. The organic building block according to any one of the preceding matters, wherein the substitute R3 in the formula is any one of the components according to matter 48-54 or a hydrogen.
83. The organic building block according to any one of the preceding matters, wherein the organic monomer comprises a biologically active compound selected from the group consisting of peptides, proteins, nucleic acids, CRISPR-Cas enzyme complex, organic active substances, apoptosis-inducing active substances, adhesion-promoting active substances, anti-inflammatory active substances, receptor agonists and receptor antagonists, growth-inhibiting active substances and in particular from proteins of the extracellular matrix, cell surface proteins, antibodies, growth factors, sugars, lectins, carbohydrates, cytokines, DNA, RNA, PNA, LNA, siRNA, aptamers, and fragments thereof, or mixtures thereof.
84. A method for manufacturing an organic building block comprising a substituted tertiary amide group represented by the formula:

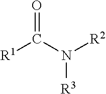

wherein the tertiary amide group results from a copolymerization of at least two components,

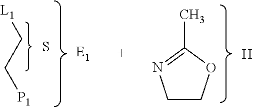

wherein the first component (E1) comprises at least of three different parts:
a first functional group (P1) for the copolymerization with the second component,
a second functional group (L1) for crosslinking to a biologically active compound, and
an optional spacer (S) between the two functional groups, and
   wherein the second component for polymerization with the first functional group is a heterocyclic chemical compound (H).
85. The method according to matter 84, wherein the first functional group (P1) is an unsaturated imide, an alkene, an acrylic acid, a lactone, an acrylamide, a sulfonamide, an anhydride, a methacrylic acid, a pyridine or an imidazole.
86. The method according to any one of the preceding matters, wherein the second functional group (L1) is selected from the group consisting of protected or unprotected NHS esters, sulfo-NHS esters, anhydrides, vinyl sulfones, sulfonyl chlorides, aldehydes, epoxides, thiols, maleimides and carbonates.
87. The method according to any one of the preceding matters, wherein the optional spacer (S) is a non-cleavable saturated hydrocarbon or polyether or a cleavable disulfide bond spacer arm and separates the two functional groups by at least one carbon atom.
88. The method according to any one of the preceding matters, wherein the first component (E1) is composed of different combination of any component according to any of matters 79 to 82.
89. The method according to any one of the preceding matters, wherein the first component (E1) is 3-(maleimido)-propionic acid N-hydroxysuccinimide ester.
90. The method according to any one of the preceding matters, wherein the first component (E1) is 6-Maleimidohexanoic acid N-hydroxysuccinimide ester.
91. The method according to any one of the preceding matters, wherein the first component (E1) is N-(Methacryloxy)succinimideisopropenyl.
92. The method according to any one of the preceding matters, wherein the first component (E1) is BMPH (N-(β-maleimidopropionic acid) hydrazide.
93. The method according to any one of the preceding matters, wherein the first component (E1) is EMCH (N-ε-maleimidocaproic acid hydrazide).
94. The method according to any one of the preceding matters, wherein the first component (E1) is PDPH (3-(2-pyridyldithio)propionyl hydrazide).
95. The method according to any one of the preceding matters, wherein the first component (E1) is Methacrylic acid N-hydroxysuccinimide ester.
96. The method according to any one of the preceding matters, wherein the second component (H) used for the copolymerization is an unsubstituted oxazoline (4,5-dihydrooxazole).
97. The method according to any one of the preceding matters, wherein the second component (H) used for the copolymerization is a 2-substituted oxazoline.
98. The method according to any one of the preceding matters, wherein the second component (H) used for the copolymerization is 2-methyl oxazoline.
99. The method according to any one of the preceding matters, wherein the substitute R1 in the formula R1-C═O—N-R2-R3 is an alkyl group.
100. The method according to any one of the preceding matters, wherein the substitute R1 in the formula R1-C═O—N-R2-R3 is a methyl group.
101. The method according to any one of the preceding matters, wherein the substitute R1 in the formula R1-C═O—N-R2-R3 is hydrogen.
102. The method according to any one of the preceding matters, wherein the disulfide bond of PDPH is cleaved on-demand with DTT, TCEP or other reducing agents.
103. The method according to any one of the preceding matters, wherein the substitute R2 in the formula R1-C═O—N-R2-R3 is any one of the components according to matter 70 or hydrogen.
104. The method according to any one of the preceding matters, wherein the substitute R3 in the formula R1-C═O—N-R2-R3 is any one of the components according to matter 70 or hydrogen.
105. The method according to any one of the preceding matters, wherein the substitute R2 and R3 in the formula R1-C═O—N-R2-R3 are any one of the components according to matter 35 or hydrogen.
106. The method according to any one of the preceding matters, wherein the biologically active compound is selected from the group consisting of peptides, proteins, nucleic acids, CRISPR-Cas enzyme complex, organic active substances, apoptosis-inducing active substances, adhesion-promoting active substances, anti-inflammatory active substances, receptor agonists and receptor antagonists, growth-inhibiting active substances and in particular from proteins of the extracellular matrix, cell surface proteins, antibodies, growth factors, sugars, lectins, carbohydrates, cytokines, DNA, RNA, PNA, LNA, siRNA, aptamers, and fragments thereof that are relevant to binding or action, or mixtures thereof.
107. The organic monomer and/or the organic building block according to any one of the preceding matters, wherein the organic monomer is used for polymerization resulting in a hydrophilic polymer comprising at least two organic monomers of any one of the preceding matters.
108. An organic polymer comprising at least two organic monomers and/or organic building blocks of any one of the preceding matters.
109. The organic polymer according to matter 103, wherein the organic polymer is functionalized by biologically active compounds.
110. The organic polymer according to any one of the preceding matters, wherein the organic polymer comprises further a peptide nucleic acid (PNA) and/or a locked nucleic acid (LNA).
111. The organic polymer according to any one of the preceding matters, wherein the Peptide nucleic acids (PNAs) or locked nucleic acid (LNA) are located at all marginal organic monomers.
112. The organic polymer according to any one of the preceding matters, wherein the polymer has a linear or multiarm/star-shaped structure.
113. A biomaterial for cell applications composed of a mixture of at least two different organic polymers according to any one of the preceding matters.
114. The biomaterial according to matter 113, wherein the biomaterial is composed of at least two different organic polymers according to any one of the preceding matters, wherein the different polymers having different structures, wherein the first polymer has a linear structure and the second polymer has multiarm or star-shaped structure.
115. The biomaterial according to any one of the preceding matters, wherein the biomaterial is composed of at least two different organic polymers according to any one of the proceeding matters, wherein the different polymers are crosslinked with carboxy-, thiol-, or amine-functionalized polyethylene glycol (PEG) such as Poly(ethylene glycol) bis(amine) or Poly(ethylene glycol) dithiol or Di(N-succinimidyl) functionalized components with dithiol moieties such as Dithiodipropionic acid di(N-hydroxysuccinimide ester or carboxy-functionalized disulfides such as 2-Carboxyethyl disulfide.
116. The biomaterial according to any one of the preceding matters, wherein the biomaterial is composed of at least two different organic polymers according to any one of the preceding matters, wherein the different polymers having complimentary PNA sequences at all marginal organic monomers with following characteristics:
   short oligos <=15mers
   Purine content of <50%
   no self-complementarity
   no poly Guanin sequences.
117. The biomaterial according to any one of the preceding matters, wherein the PNA sequences exhibits a proteolytically degradable peptide sequence which is sensitive to cell-secreted proteases such as matrix-metalloproteinases (MMPs) to allow for cell-mediated degradation of the biomaterial.
118. The biomaterial according to any one of the preceding matters, wherein the biomaterial is formed by hybridization of the complimentary PNA sequences.
119. The biomaterial according to any one of the preceding matters, wherein the biomaterial has a spherical and/or plug-like structure.
120. A method for the production of a biomaterial for cell-based applications, which method has the following consecutive steps:
   a) providing of one or more organic polymer and/or organic building blocks according to any one of the preceding matters
   b) functionalization of the polymer from step a) with at least one biologically active molecule
   c) addition of a crosslinking agent for crosslinking the polymer functionalized in step b) to generate the biomaterial.
   d) degradation of the crosslinking agent to release ingredients from the biomaterial.
121. The method as mattered in matter 120, wherein after step b), it additionally has the following step b)′:
   b)′ addition of and encapsulation of biofactors and/or a single cell of a particular cell type and/or at least two cells with at least different cell types, in particular mammalian cells.
122. The method according to any one of the preceding matters, wherein after step c), it additionally has the following step c)′:
   c)′ addition of diamines such as 1,6-Hexanediamine for crosslinking marginal functional groups such as NHS esters to form a highly crosslinked gel shell.
123. The method according to any one of the preceding matters, wherein the biofactor is selected from the group consisting of peptides, proteins, nucleic acids, organic active substances, apoptosis-inducing active substances, adhesion-promoting active substances, growth-inhibiting active substances, anti-inflammatory active substances, receptor agonists and receptor antagonists, and in particular from proteins of the extracellular matrix, cell surface proteins, antibodies, growth factors, sugars, lectins, carbohydrates, cytokines, DNA, RNA, siRNA, PNA, LNA, aptamers, and fragments thereof that are relevant to binding or action, or mixtures thereof.
124. The method according to any one of the preceding matters, wherein the biofactor is selected from the group consisting of proteins of the extracellular matrix, growth factors and fragments thereof that are relevant to binding or action, or mixtures thereof.
125. The method according to any one of the preceding matters, wherein the crosslinking agent in step c) is thiol-, or amine-functionalized polyethylene glycol (PEG) such as Poly(ethylene glycol) bis(amine) or Poly(ethylene glycol) dithiol
126. The method according to any one of the preceding matters, wherein the biomaterial is formed by hybridization of complimentary PNA sequences.
127. The method according to any one of the preceding matters, wherein the crosslinking agent is degraded on-demand with DTT, TCEP or other reducing agents.
128. The method according to any one of the preceding matters, wherein the crosslinking agent is degraded on-demand by dehybridization of the complementary PNAs by applying heat, high salt concentrations or complementary nucleic acids with a higher affinity.
129. An organic building block manufactured with a method according to any one of the preceding matters.
130. A droplet comprising a hydrogel/hydrogel matrix composed of an organic monomer, organic building block and/or an organic polymer according to any one of the preceding matters.
131. Use of an organic monomer and/or an organic building block according to any one of the preceding matters for the polymerization of a hydrophilic polymer comprising at least two organic monomers and/or organic building blocks of any one of the preceding matters.
132. A hydrogel matrix composed of a mixture of least two different organic polymers and/or organic building blocks according to any one of the proceeding matters, and/or composed of an organic polymer according to any one of the preceding matters.

1. A hydrogel which comprises cross-linked hydrogel precursor molecules of the same type or of different types.

2. The hydrogel according to claim 1, wherein the hydrogel is composed of at least two different polymers with different structures as hydrogel precursor molecules, wherein optionally, at least one polymer is a copolymer.

3. The hydrogel according to claim 1 or 2, wherein at least one polymer has a linear structure and at least one polymer has a multiarm or star-shaped structure.

4. The hydrogel according to any one of the preceding claims, comprising a polymer that was obtained by copolymerization of (i) a heterocyclic chemical compound, preferably a 2-oxazoline, and (ii) a compound comprising (aa) an unsaturated imide, preferably 3-(maleimido)-propionic acid N-hydroxysuccinimide ester or (bb) an alkenyl group such as an isopropenyl group.

5. The hydrogel according to claim 4, having at least one of the following characteristics:
(a) compound (ii) comprises a spacer and a functional group for crosslinking a biologically active molecule;
(b) compound (ii) is a 3-(maleimido)-propionic acid N-hydroxysuccinimide ester;
(c) the backbone of at least one polymer is functionalized with at least one biologically active molecule at the functional group of compound (ii).

6. The hydrogel according to claim 4 or 5, wherein compound (i) is a hydrophilic poly-(2-oxazoline), wherein optionally, the water-solubility is adjusted by the 2-substitution of the 2-oxazoline compound.

7. The hydrogel according to any of the preceding claims, wherein the backbone of the polymers is formed by hydrophilic peptide-like polymers that are crosslinked in the hydrogel by cell-compatible crosslinking reactions.

8. The hydrogel according to any of the preceding claims, wherein the hydrogel comprises a 2-oxazoline-based polymer, preferably a poly-2-methyl-2-oxazoline based polymer, more preferably a copolymer.

9. The hydrogel according to any of the preceding claims, wherein the hydrogel comprises (i) linear and (ii) multiarm 2-oxazoline-based polymers.

10. The hydrogel according to claim 8 or 9, wherein the 2-oxazoline is substituted only at position 2 and wherein preferably, the substitution in the 2-position comprises a group selected from alkynes, alkenes, protected amine groups or short aliphatic chains such as methyl.

11. The hydrogel according to any one of claims 8 to 10, having one or more of the following characteristics:
(a) the hydrogel comprises a polymer that is formed by living cationic ring-opening polymerization of oxazolines substituted at position 2;
(b) the hydrogel is a biomaterial for cell applications, wherein preferably, the biomaterial is composed of at least two different polymers according to any one of claims 101 to 155, wherein the different polymers having different structures, wherein the first polymer has a linear structure and the second polymer has a multiarm or star-shaped structure;
(c) the hydrogel comprises one or more biologically active molecules linked to the polymer backbone of at least one polymer/hydrogel precursor, wherein preferably, the polymer is linear and wherein more preferably, the biologically active molecule is attached via a degradable linker.

12. The hydrogel according to any one of claims 1 to 11, wherein the hydrogel matrix is composed of a mixture of at least two different polymers according to any one of claims 101 to 155.

13. The hydrogel according to any one of claims 1 to 12, wherein the hydrogel matrix comprises at least two polymers according to:
a) claim 101 or 112, wherein the polymer further has the features of claim 139, or according to claim 123; and
b) claim 101 or 112, wherein the polymer further has the features of claims 139 and 144, or according to claim 125.

14. The hydrogel according to any one of claims 1 to 12, wherein the hydrogel matrix comprises at least two polymers according to:
a) claim 101 or 112, or according to claim 127; and
b) claim 101 or 112, or according to claim 129.

15. The hydrogel according to any one of claims 1 to 12, wherein the hydrogel matrix comprises at least two polymers according to:
a) claim 124; and
b) claim 126.

16. The hydrogel according to any one of claims 1 to 12, wherein the hydrogel matrix comprises at least two polymers according to:
a) claim 104 or 122, wherein the polymer further has the features of claim 142, wherein the polymer preferentially has a linear structure; and
b) claim 104 or 122, wherein the polymer further has the features of claim 144
or wherein the hydrogel matrix comprises at least two different polymers, preferably at least 4 different polymers, more preferably at least 5 different polymers according to:
a. claim 123;
b. claim 125;
c. claim 127;
d. claim 129;
e. claim 124;
f. claim 126;
g. claim 128;
h. claim 130; and/or
i. claim 134.

17. A hydrogel according to any one of claims 1 to 16, wherein hydrogel precursor molecules are crosslinked in the hydrogel matrix by cell-compatible crosslinking reactions.

18. The hydrogel according to any one of claims 1 to 17, wherein the hydrogel precursor molecules are cross-linked in the hydrogel matrix by a reaction selected from:
(i) a covalent bond formation, preferably selected from (aa) enzymatically catalyzed reactions, such as reactions catalyzed with transglutaminase factor XIIIa, (bb) not-enzymatically catalyzed reactions, such as click chemistry or photo-catalyzed reactions and/or (cc) uncatalyzed reactions, such as copper-free highly selective click chemistry, Michael-type addition or Diels-Alder conjugation;
(ii) non-covalent bond formation preferably selected from (aa) hydrogen bonds, preferably formed by nucleic acids or nucleic acid analogs, (bb) hydrophobic interactions, (cc) Van-der-Waals interactions and (dd) electrostatic interactions; and
(iii) combinations of the foregoing.

19. The hydrogel according to any one of claims 1 to 18, wherein in the hydrogel, cross-links are formed via terminating moieties that are located at ends of the polymers providing the hydrogel precursor molecules, wherein optionally, cross-links are formed exclusively via terminating moieties that are located at ends of the polymers providing the hydrogel precursor molecules.

20. The hydrogel according to any one of claims 1 to 19, having one or more of the following characteristics:
(a) the hydrogel is composed of at least two different polymers, preferably according to any one of claims 101 to 155, wherein the different polymers are crosslinked with a carboxy-, thiol-, or amine-functionalized polymer, preferably polyethylene glycol (PEG) such as poly(ethylene glycol) bis(amine) or poly(ethylene glycol) dithiol or di(N-succinimidyl) functionalized components with dithiol moieties such as dithiodipropionic acid di(N-hydroxysuccinimide ester or carboxy-functionalized disulfides such as 2-carboxyethyl disulfide;
(b) in the hydrogel, maleimide and thiol end functionalized polymer precursors are cross-linked, wherein preferably, at least one polymer precursor furthermore comprises a NHS ester as functional group attached to the polymeric backbone, preferably via a degradable linker, for functionalization with a biologically active molecule or wherein a biologically active molecule is attached thereto.

21. The hydrogel according to any one of claims 17 to 20, wherein crosslinking includes hydrogen bond formation, preferably based on hybridization.

22. The hydrogel according to any one of claims 17 to 21, wherein each hydrogel precursor molecule comprises a terminating moiety and wherein terminating moieties of different hydrogel precursor molecules are crosslinked by hybridization thereby forming the hydrogel.

23. The hydrogel according to any one of claims 21 to 22, wherein hybridization based cross-links are formed between (i) a linear hydrogel precursor molecule and (ii) a multiarm or starshaped hydrogel precursor molecule.

24. The hydrogel according to any one of claims 19 to 23, wherein each terminating moiety comprises bases allowing sequence specific base pairing by hydrogen bonds, in particular selected from purine and pyrimidine bases.

25. The hydrogel according to any one of claims 19 to 24, having one or more of the following characteristics:
(a) wherein each terminating moiety comprises an oligomer, preferably having ≤30, ≤25, preferably ≤20 or ≤15 bases,
(b) the hydrogel precursors are crosslinked based on hybridization involving Watson-Crick base pairing and/or Hoogsteen base pairing, wherein preferably hybridization is based on Watson-Crick base pairing.

26. The hydrogel according to claim 24 or 25, wherein the terminating moiety comprises a nucleic acid or nucleic acid analog, preferably selected from PNA, LNA, hexitol nucleic acid (HNA), morpholino oligomers, phosphorthioate DNA, phosphoramidate DNA and 2′O-methoxyethyl RNA, more preferably selected from PNA and LNA and most preferably is a PNA.

27. The hydrogel according to any one of claims 19 to 26, wherein different hydrogel precursor molecules comprise different terminating moieties, wherein said different terminating moieties comprise moieties, preferably nucleic acids or nucleic acid analogs, that are
(i) complementary to each other and are hybridized to each other in the hydrogel; or
(ii) are not complementary to each other and the crosslink in the hydrogel is established by a hybridizing molecule that hybridizes to the different terminating moieties of the hydrogel precursor molecules, thereby providing a cross-link that is based on hybridization,
whereby different precursor molecules are cross-linked due to hybridization, thereby providing an alternating structure of polymers, preferably linear and multimer polymers, that form the hydrogel.

28. The hydrogel according to claim 27, wherein the hybridizing molecule has one or more of the following characteristics
(i) it comprises (aa) a first hybridizing portion that hybridizes to the terminating moiety of one hydrogel precursor molecule and (bb) a second hybridizing portion that hybridizes to the terminating moiety of another hydrogel precursor molecule;
(ii) it comprises bases allowing sequence specific base pairing by hydrogen bonds, wherein preferably, the hybridizing molecule comprises ≤60, ≤55, preferably ≤50 or ≤35 bases.

29. The hydrogel according to any one of claims 17 to 28, wherein the hydrogel precursor molecules are cross-linked by hybrids that comprise mismatches.

30. The hydrogel according to any one of claims 1 to 29, wherein the hydrogel precursor molecules comprise an enzyme degradable target site, preferably a protease target site, preferably located between the polymer backbone and the terminating moiety.

31. The hydrogel according to any one of claims 27 to 30, wherein the hybridizing molecule establishing the crosslink between the terminating moieties is selected from a nucleic acid or nucleic acid analog, preferably is selected from PNA and DNA,
wherein optionally, the hybridizing molecule is a PNA molecule that comprises (aa) a first hybridizing portion that hybridizes to the terminating moiety of one hydrogel precursor molecule and (bb) a second hybridizing portion that hybridizes to the terminating moiety of another hydrogel precursor molecule, and (cc) an enzyme degradable target site, preferably a protease target site, more preferably a matrix metalloprotease target site, for site directed degradation of the polymer located between the first and second hybridizing portions.

32. The hydrogel according to any one of claims 17 to 31, wherein the crosslinking reaction includes hydrogen bond formation between two peptide nucleic acid (PNA) molecules with different base sequences or two locked nucleic acid (LNA) molecules with different base sequences or a combination of one PNA molecule and one LNA molecule.

33. The hydrogel according to claim 32, wherein a PNA molecule and/or the LNA molecule is located at the ends of the polymers that form the hydrogel.

34. The hydrogel according to any one of claims 1 to 33, wherein the hydrogel matrix is built up from precursor molecules that are cross-linked by hydrogen bonds formed by peptide nucleic acids (PNA).

35. The hydrogel according to claim 34, wherein PNA oligomers are located at the ends of the polymers that form the hydrogel.

36. The hydrogel according to claim 34 or 35, wherein different precursor molecules possess complementary PNA oligomers, wherein preferably, linear and multiarm polymers are used as precursors.

37. The hydrogel according to any one of claims 34 to 36, wherein only different precursor molecules are cross-linked due to hybridization of the comprised PNAs, thereby providing an alternating structure of preferably linear and multimer polymers that form the hydrogel.

38. The hydrogel according to any one of claims 34 to 37, wherein the complementary PNA oligomers possess mismatches.

39. The hydrogel according to any one of claims 34 to 38, wherein the PNA oligomers cross-linking the hydrogel precursor molecules have one or more, preferably all, of the following characteristics
(i) they are short oligos ≤15 mers;
(ii) they have a purine content of <50%,
(iii) they are not self-complementarity,
(iv) they do not comprise poly guanine sequences.

40. The hydrogel according to any one of claims 1 to 39, wherein a three-dimensional hydrogel is formed via hydrogen bonds between LNAs and/or PNAs of (i) a multiarm or starshaped poly-(2-oxazoline) based polymer and (ii) a linear poly-(2-oxazoline) based polymer, wherein preferably the linear poly-(2-oxazoline) based polymer is functionalized with biologically active molecules.

41. The hydrogel according to any one of claims 1 to 39, having one or more of the following characteristics:
(a) wherein the hydrogel is formed by hybridization of complementary PNA sequences;
(b) wherein precursor molecules comprise a peptide bearing a protease site, preferably adjacent to the PNA;
(c) wherein the hydrogel matrix comprises multiple degradation targets for at least one enzyme that is secreted by a cell comprised in the hydrogel matrix, preferably MMP target sites;
(d) wherein the hydrogel is degradable by increasing the temperature;
(e) wherein the hydrogel has a spherical or plug-like structure and preferably, is spherical;
(f) the hydrogel is three-dimensional;
(g) the hydrogel has a size in the micrometer or sub-micrometer scale and is preferably spherical;
(h) wherein the hydrogel provides a synthetic backbone, preferably a synthetic matrix that lacks toxins.

42. The hydrogel according to any one of the preceding claims 1 to 41, wherein the hydrogel matrix comprises one or more cells and/or particles, preferably comprises at least one cell.

43. The hydrogel according to any one of the preceding claims 1 to 42, wherein at least one polymer is functionalized with at least one biologically active molecule, preferably to present different adhesive ligands, bioactive compounds and functional biomolecules such as adhesive compounds of the extra cellular matrix (ECM), growth factors, antibodies, CRISPR-Cas and nucleic acids or wherein the hydrogel comprises one or more of the following:
(i) functional molecules for cell culture and cell analysis;
(ii) gold particles, quantum dots, growth promoting substances, cytokines, chemokines, antibody-conjugates and/or inorganic substances.

44. The hydrogel according to any one of the preceding claims 1 to 43, wherein the backbone of a comprised polymer, preferably a linear polymer, is functionalized with a biologically active molecule.

45. The hydrogel according to any one of the preceding claims 1 to 44, wherein the hydrogel comprises capture molecules, which may be incorporated by one or more of the cross-linking techniques as defined in claim 18, wherein optionally, incorporation of the capture molecules involves peptide nucleic acids, wherein preferably, a PNA oligomer is incorporated into the hydrogel gel by amide bond formation between an NHS-ester from the hydrogel precursor molecule and the primary amine of a PNA oligomer and wherein the capture molecule is fused to a complementary PNA oligomer and wherein the capture molecule is incorporated into the hydrogel by hydrogen bond formation between the two PNA oligomers.

46. The hydrogel according to any one of the preceding claims 1 to 45, wherein the hydrogel is surrounded by a gel-shell.

47. The hydrogel according to claim 46, wherein the gel-shell comprises at least one gel-shell forming compound that is optionally crosslinked to the hydrogel, wherein preferably the gel-shell forming compound is a primary amine bearing polymer molecule, e.g. a poly (allylamine).

48. The hydrogel according to claim 47, wherein the gel-shell forming compound is or is derived from a compound selected from the group consisting of poly(allylamine), branched amino-polyethyleglycol (PEG), branched polyethylenimine (PEI), polylysine, poly amidoamine (PAMAM) dendrimer, poly(β-amino ester), chitosan and poly(2-amino-2-oxazoline).

49. The hydrogel according to claim 47 or 48, having one or more of the following characteristics:
(a) the gel-shell forming compound is crosslinked to a functional group of a poly(2-oxazoline)copolymer);
(b) the gel-shell forming compound forms a gel-shell around the hydrogel matrix, wherein the gel-shell is not covalently attached to the hydrogel matrix and wherein optionally, the gel shell forming compound comprises functional groups that are cross-linked via a further compound thereby forming a gel-shell around the hydrogel matrix;
(c) the gel-shell forming compound comprises a nucleic acid or nucleic acid analog, preferably a PNA sequence, that is hybridized to another compound comprised in the hydrogel matrix, wherein the compound is a multi-arm or star-shaped polymer, which is not crosslinked to the hydrogel matrix and wherein the gel-shell forming compound is preferably a linear polymer.

50. The hydrogel according to any one of claims 1 to 49, comprising a polymer according to claim 104 as hydrogel precursor that is preferably cross-linked with a multiarm polymer.

51. The hydrogel according to any one of claims 47 to 50, wherein the gel-shell surrounded hydrogel was obtained by the method according to 64 to 70.

52. A method for producing a hydrogel according to any one of claims 1 to 51, wherein the hydrogel matrix is formed by cross-linking hydrogel precursor molecules of the same type or of different types.

53. The method according to claim 52, comprising crosslinking hydrogel precursor molecules by a cell-compatible crosslinking reaction, preferably in the presence of a cell.

54. The method according to claim 52 or claim 53, wherein the crosslinking reaction is selected from:
(i) a covalent bond formation, preferably selected from (aa) enzymatically catalyzed reactions, such as reactions catalyzed with transglutaminase factor XIIIa, (bb) not-enzymatically catalyzed reactions, such as click chemistry or photo-catalyzed reactions and/or (cc) uncatalyzed reactions, such as copper-free highly selective click chemistry, Michael-type addition or Diels-Alder conjugation;
(ii) non-covalent bond formation preferably selected from (aa) hydrogen bonds, preferably formed by nucleic acids or nucleic acid analogs, (bb) hydrophobic interactions, (cc) Van-der-Waals interactions and (dd) electrostatic interactions; and
(iii) combinations of the foregoing.

55. The method according to any one of claims 52 to 54, wherein the crosslinking reaction includes hydrogen bond formation, preferably between
(i) two peptide nucleic acid (PNA) molecules with different base sequences or
(ii) two locked nucleic acid (LNA) molecules with different base sequences or
(iii) a combination of one PNA molecule and one LNA molecule.

56. The method according to any one of claims 52 to 55, wherein the hydrogel is produced using a method for droplet generation and mixing of at least two droplets, preferably the method for droplet generation as disclosed herein.

57. The method according to claim 56, wherein after generating and mixing said droplets, a spherical or plug-like hydrogel matrix is formed within the mixed droplet.

58. The method according to claim 56 or 57, comprising generating
(i) a first droplet that comprises a multiarm hydrogel precursor,
(ii) a second droplet that comprises a linear hydrogel precursor,
(iii) optionally a third droplet for initiating the cross-linking of the multiarm and the linear hydrogel precursor,
wherein after generating and mixing said droplets a spherical or plug-like hydrogel matrix is formed within the mixed droplet,
wherein optionally, the first or the second droplet comprises compounds, preferably biological active molecules, wherein said compounds are immobilized within the hydrogel matrix, preferably during hydrogel formation.

59. The method according to any one of claims 56 to 58, comprising generating and mixing at least four droplets, wherein the fourth droplet comprises a compound that becomes immobilized within the formed hydrogel matrix, preferably by a stable amide bond, wherein the compounds is optionally selected from proteins such as antibodies, growth factors or ECM proteins; nucleic acids such as DNA primers and peptide nucleic acids or is selected from gold particles, quantum dots, growth promoting substances, cytokines, chemokines, antibody-conjugates, inorganic substances.

60. The method according to any one of claims 56 to 59, wherein the droplets are generated in parallel or sequentially.

61. The method according to any one of claims 52 to 60, having one or more of the following characteristics:
(a) the method comprises generating at least two droplets, wherein each droplet comprises a different cell type and fusing said at least two droplets to provide a first droplet that comprises at least one multiarm precursor;
(b) the first droplet comprises at least one cell and wherein the multiarm precursor lacks functional groups that are reactive with the one or more cells under the conditions within the first droplet.

62. The method according to any one of claims 52 to 61, wherein during hydrogel formation, one or more cells or particles, preferably at least one cell, becomes encapsulated in the hydrogel matrix, wherein preferably, the one or more cells or particles are combined with at least one hydrogel precursor prior to forming the gel, and wherein more preferably, the encapsulation method as defined in any of claims 73 to 83 is used.

63. The method according to any one of claims 52 to 62, comprising functionalizing the hydrogel with at least one biologically active molecule, wherein preferably, functionalization has one or more of the following characteristics:
(a) functionalization occurs before, during or after encapsulating at least one cell into the hydrogel;
(b) the biologically active molecule is cross-linked to a functional group of at least one polymer that provides a hydrogel precursor molecule, which preferably is a polymer as defined in any one of claims 1 to 55, and preferably, is a linear polymer;
(c) after hydrogel formation the method comprises adding bioactive molecules to a liquid that flows through the formed hydrogel, thereby incorporating bioactive molecules into the hydrogel matrix;
(d) the hydrogel is functionalized with at least one biologically active molecule before a gel-shell is formed that surrounds the particle.

64. The method according to any one of claims 52 to 63, for producing a gel-shell surrounded hydrogel, comprising
(a) providing a droplet generated by fusion of multiple droplets, wherein the fused droplet A comprises the hydrogel matrix;
(b) forming the gel-shell by fusing droplet A with a second droplet B containing a polymer which comprises primary amines, such as poly allylamine polymers, thereby providing a larger droplet C containing said hydrogel matrix with the volume of the hydrogel matrix being smaller than the volume of droplet C and wherein in droplet C, said hydrogel matrix is surrounded by said polymer from droplet B and
(c) crosslinking of the hydrogel polymers at the edge of the hydrogel matrix.

65. The method according to claim 64, wherein said polymer from droplet B diffuses into the hydrogel matrix, whereby crosslinking occurs.

66. The method according to claim 64 or 65, wherein the method comprises using (i) a primary amine bearing polymer molecule, e.g. a poly allylamine and (ii) a small primary amine, e.g. 3-amino-1,2-propanediol, wherein the polymer molecule (i) having a smaller diffusion coefficient than the small primary amine (ii).

67. The method according to claim 66, wherein the primary amine diffuses faster into said hydrogel matrix than the polymer molecule, wherein preferably the small primary amines are added with a short delay after the poly allylamine polymers.

68. The method according to any one of claims 64 to 67, wherein the method further comprises fusing said droplet C with a droplet D containing a small primary amine, e.g. 3-amino-1,2-propanediol, the small primary amine having a smaller diffusion coefficient than the polymer in droplet C.

69. The method according to any one of the preceding claims for producing a gel-shell surrounded hydrogel, comprising
(a) providing a droplet generated by fusion of multiple droplets, wherein the fused droplet A comprises the hydrogel matrix;
(b) forming the gel-shell by immobilizing and diffusing droplet A by an aqueous phase containing a small primary amine, e.g. 3-amino-1,2-propanediol and a polymer which comprises primary amines, such as poly allylamine polymers, the small primary amine having a smaller diffusion coefficient than the polymer; and
(c) crosslinking of the hydrogel polymers at the edge of the hydrogel matrix.

70. The method according to any one of the preceding claims for producing a gel-shell surrounded hydrogel, wherein the shell is formed by contacting one or more of the following compounds with the hydrogel matrix:
i) a polymer which comprises primary amines, which is preferably selected from poly(allylamine), (branched) amino-polyethyleglycol (PEG), (branched) polyethylenimine (PEI), polylysine, poly amidoamine (PAMAM) dendrimer, poly(β-amino ester), chitosan, or amino-PaOX, and, optionally, a primary amine compound which preferably is a small primary amine compound such as an aminofunctionalyzed C3-C6-alkanediol, e.g. 2-amino-1,3-propanediol or 3-amino-1,2-propanediol,
wherein amine groups react with a residual functional group of the hydrogel matrix, e.g. a N-hydroxysuccinimide ester;
ii) a polymer comprising a N-hydroxysuccinimide ester, preferably selected from PEG-NHS-ester or polyoxazoline-NHS-ester, and a diamine compound, e.g. a c3-c6-alkanol diamine such as 1,3-diamino-2-propanol, wherein the diamine compound is present in the hydrogel matrix prior to adding the N-hydroxysuccinimide ester comprising compound;
iii) a polymer comprising a maleimide, and furthermore a dithiol compound, e.g. 2,2′-(ethylenedioxy)diethanethiol or short dithiol functionalized polymers with an enzyme degradable target site, such as a matrix metalloprotease sensitive target site, wherein the dithiol compound is present in the hydrogel matrix prior to adding the maleimide comprising compound; or
iv) the gel-shell forming compound comprises a nucleic acid or nucleic acid analog, preferably a PNA sequence, that is hybridized to another compound comprised in the hydrogel matrix, wherein the compound is a multi-arm or star-shaped polymer, which is not crosslinked to the hydrogel matrix and wherein the gel-shell forming compound is preferably a linear polymer.

71. The method according to any one of the preceding claims 52 to 70, comprising providing at least two polymers, preferably selected from the polymers as defined in any one of claims 101 to 151, as hydrogel precursors and cross-linking the at least two polymers to provide the hydrogel.

72. A hydrogel obtained by the method according to any one of claims 52 to 71.

73. A method for encapsulating one or more cells and/or particles into a hydrogel, preferably a hydrogel as defined in any one of claims 1 to 51, wherein the one or more cells and/or particles are combined with at least one hydrogel precursor prior to gel formation and are encapsulated into the hydrogel matrix during hydrogel formation.

74. The method according to claim 73, wherein one or more polyoxazoline derivatives are used as hydrogel precursor.

75. The method according to claim 74, wherein one or more polymers as defined in any one of claims 101 to 155 are used as hydrogel precursor, wherein preferably, at least one polyoxazoline based polymer, preferably a co-polymer comprising at least one moiety of formula (I) and at least one moiety of formula (II) as defined in claim 101 and claims dependent thereon, is used as hydrogel precursor.

76. The method according to any one of claims 73 to 75, having one or more of the following characteristics:
(a) wherein after encapsulation, the one or more cells are in the center of the hydrogel,
(b) the method comprises
preparing a liquid composition comprising (i) one or more cells and/or particles, preferably one or more cells, and (ii) the hydrogel precursors, and
cross-linking the hydrogel precursors thereby providing a hydrogel encapsulating the one or more cells and/or particles.
(c) the method comprises
preparing a liquid composition using a microfabricated valve as disclosed herein comprising (i) one or more cells and/or particles, preferably one or more cells, and (ii) the hydrogel precursors, and
cross-linking the hydrogel precursors thereby providing a hydrogel encapsulating the one or more cells and/or particles.

77. The method according to claim 76, wherein the method comprises combining one or more cells and/or particles, preferably one or more cells, with at least one hydrogel precursor prior to forming the hydrogel and wherein the hydrogel is formed in the presence of the one or more cells.

78. The method according to claim 73 or 77, wherein the method comprises (a) providing prior to forming the hydrogel a liquid composition, e.g. in form of a droplet, wherein the composition comprises (i) one or more cells and/or particles, preferably one or more cells, and (ii) at least one hydrogel precursor, (b) combining, e.g. mixing, said composition with at least one further hydrogel precursor and (c) forming the hydrogel in the presence of the one or more cells by cross-linking (gelation) whereby the hydrogel matrix is formed around the cell.

79. The method according to any one of claims 73 to 78, comprising combining the one or more cells sequentially with the hydrogel precursors prior to cross-linking the hydrogel precursors, wherein the one or more cells are combined with at least one hydrogel precursor that lacks functional groups that are reactive with the one or more cells under the combination conditions and subsequently adding at least one further hydrogel precursor, wherein optionally, the subsequently added hydrogel precursor comprises functional groups for attaching a biologically active molecule.

80. The method according to any one of claims 73 to 79, wherein the hydrogel is produced by generating and mixing at least two droplets comprising different hydrogel precursors, wherein one or more cells are comprised in at least one droplet, preferably using the method for droplet generation and fusing of at least two droplets as defined in any one of preceding claims or using the method according to claim 56 and claims dependent thereon.

81. The method according to any one of claims 73 to 80, wherein the method comprises:
encapsulating one or more cells and/or particles into a first droplet, wherein the first droplet has a defined size and comprises a hydrogel precursor molecule (a) at a defined concentration;
generating a second droplet, wherein the second droplet has a defined size and comprises a hydrogel precursor molecule (b) at a defined concentration
fusing said formed droplets, thereby providing a larger droplet that contains the hydrogel precursor molecules (a) and (b) and the one or more cells and/or particles, wherein preferably, hydrogel formation occurs due to the mixing of said hydrogel precursor molecules.

82. The method according to any one of claims 73 to 81, wherein the first or the second droplet comprises compounds, preferably biological active molecules, wherein said compounds are immobilized within the hydrogel matrix, preferably during hydrogel formation.

83. The method according to any one of claims 73 to 82, having one or more of the following characteristics
(a) the hydrogel is as defined in any one of claims 1 to 51;
(b) wherein the type of encapsulated cells is the same or different;
(c) wherein the method comprises preparing at least two separate hydrogels, preferably at least two hydrogel beads, wherein the type of encapsulated cells is the same or different.

84. A method for degrading a hydrogel according to any one of claims 1 to 51, comprising reversing the cross-links of the hydrogel.

85. The method according to claim 84, comprising reversing hybridization based, preferably PNA based, cross-links of the hydrogel.

86. The method according to claim 84 or 85, comprising one or more of the following:
(a) heating the hydrogel to degrade the hydrogel; and/or
(b) increasing the ionic strength;
(c) dehybridization of complementary PNAs by applying heat, high salt concentrations or complementary nucleic acids with a higher affinity, preferably in molar excess.

87. The method according to any one of claims 84 to 86, comprising adding at least one hybridizing molecule, preferably in excess, to the hydrogel wherein the hybridizing molecule disturbs the cross-linking hybrids of the hydrogel, whereby the crosslinks are reversed.

88. The method according to claim 87, wherein the at least one hybridizing molecule used for degradation is complementary to
(aa) a terminating moiety of a hydrogel precursor molecule that participates in the crosslinking hybrid, or
(bb) the hybridizing molecule used for crosslinking according to claim (ii),
and binds with a higher affinity thereto.

89. The method according to claim 87 or 88, wherein the crosslinking hybrid comprises mismatches and wherein the added hybridizing molecule provides a hybrid without mismatches.

90. The method according to any one of claims 87 to 89, wherein the added hybridizing molecule is selected from a nucleic acid or nucleic acid analog, and preferably is a PNA.

91. The method according to claim 85, comprising adding PNA oligomers in excess to the hydrogel, wherein the complementary PNAs forming the cross-link of the hydrogel have a decreased hybridization energy compared to the PNA oligomers added for hydrogel degradation.

92. The method according to any one of claims 84 to 91, comprising adding at least one enzyme to degrade the hydrogel, preferably selected from proteases and nucleases.

93. The method according to claim 92, wherein the added enzyme targets a protease target site comprised in the hydrogel precursor and/or the hybridizing molecule, thereby degrading the hydrogel.

94. The method according to claim 92 or claim 93, wherein the added enzyme is a nuclease, preferably a DNase, and wherein the DNase degrades a hybridizing DNA molecule that establishes the hybridizing hybrid thereby degrading the hydrogel.

95. The method according to any one of the preceding claims 84 to 94, wherein the hydrogel to be degraded comprises at least one cell and wherein said cell is not affected by the degradation procedure.

96. The method according to claim 95, wherein the hydrogel is degraded by at least one enzyme that is secreted by the at least one cell comprised in the hydrogel, wherein optionally
(a) the secreted enzyme targets the protease target site comprised in the hydrogel precursor and/or the hybridizing molecule, thereby degrading the hydrogel;
(b) the secreted enzyme is a nuclease, preferably a DNase, and wherein the DNase degrades the hybridizing DNA molecule that establishes the hybridizing hybrid thereby degrading the hydrogel.

97. A droplet or combination of at least two droplets comprising a hydrogel according to any one of claims 1 to 52.

98. The droplet according to claim 97, wherein the droplet or the combination of at least two droplets comprises one or more cells, wherein optionally, at least two different cell types are comprised, preferably in different droplets.

99. A kit for providing a hydrogel according to any one of claims 1 to 51, comprising:
(a) a first hydrogel precursor
(b) a second hydrogel precursor
(c) optionally a reagent for crosslinking the first and second hydrogel precursor
(d) optionally a test device as disclosed herein.

100. The kit according to claim 99, having one or more of the following characteristics:
the first and second hydrogel precursor are provided by a polymer as defined in any one of claims 101 to 155,
the first and second hydrogel precursor comprise terminating moieties comprising nucleic acids or nucleic acid analogs;
the first hydrogel precursor is a linear polymer and the second hydrogel precursor is a multiarm or starshaped polymer;
the first and/or the second hydrogel precursor, preferably the first and second hydrogel precursor, is selected from a polymer as defined in any one of 101 to 155;
it comprises at least one biologically active molecule, wherein preferably, said molecule is suitable to react with a functional group of at least one hydrogel precursor, preferably a linear hydrogel precursor, and wherein more preferably, the biologically active molecule is a peptide or protein and wherein at least one hydrogel precursor comprises a functional group capable of reacting with the N-terminus of the peptide or protein, wherein preferably, the functional group of the hydrogel precursor is a NHS ester;
it comprises a reagent for providing a gel shell;
it comprises a cell culture medium;
the hydrogel precursor molecules are lyophilized, wherein optionally the kit comprises a reagent for reconstituting the hydrogel precursor molecules.

101. Polymer, especially polymer as building-block for hydrogel formation, comprising at least one moiety of formula (I) and at least one moiety of formula (II) 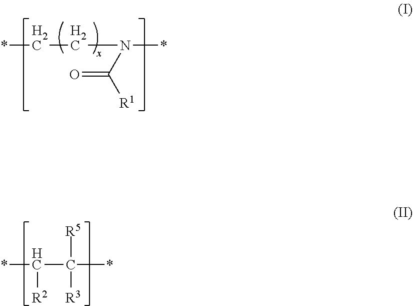
wherein
R¹ is a hydrogen atom, a hydrocarbon with 1-18 carbonatoms (preferably CH₃, —C₂H₅), a C₁-C₂₅-hydrocarbon with at least one hydroxy group, a C₁-C₂₅-hydrocarbon with at least one carboxy group, (C₂-C₆)alkylthiol, (C₂-C₆)alkylamine, protected (C₂-C₆)alkylamine (preferably-(CH₂)₂₋₆—NH—CO—R (with R=benzylhydryloxy, 9-fluorenylmethoxy)), (C₂-C₆)alkylazide, polyethylene glycol, a crosslink to R¹ of another moiety of formula (I), polylactic acid, polyglycolic acid or polyoxazoline, or wherein R¹ is a residue R⁴,
R² and R³ R² and R³ are linked to form a cyclic moiety of formula (II) comprising at least one residue R⁴
or R² and R³ are independently selected from hydrogen, —COOH, methyl or a residue R⁴, wherein optionally, at least one of R² and R³ is a residue R⁴,
R⁴ is a moiety, comprising at least one functional group, independently selected from a functional group
for crosslinking and/or
for binding biologically active compounds, and
optionally comprising a (preferably degradable) spacer moiety connecting said functional group with the binding site of the respective moiety of formula (I) or formula (II), and
R⁵ denotes a hydrogen atom, a carboxymethyl group or a methyl group,
x is 1, 2 or 3, and
* denotes a chemical bond of the polymer backbone or to a terminating moiety,
with the proviso, that at least one moiety of formula (I) or formula (II) comprises a residue R⁴, wherein preferably only the moieties of formula (I) or only the moieties of formula (II) comprise at least one moiety R⁴.

102. Polymer, especially polymer as building-block for hydrogel formation, according to claim 101, characterized in, that R¹ is a hydrogen atom or a C₁-C₁₈-alkyl group, preferably a hydrogen atom, methyl, ethyl, n-propyl, iso-propyl, n-butyl, iso-butyl, sec-butyl, tert-butyl, pentyl, iso-pentyl, neopentyl, sec-pentyl, hexyl, heptyl, octyl, nonyl or decyl, more preferably methyl or ethyl.

103. Polymer, especially polymer as building-block for hydrogel formation, according to claim 101 or claim 102, characterized in, that it comprises at least two different moieties of formula (I) having different groups R¹.

104. Polymer, especially polymer as building-block for hydrogel formation, according to any of the preceding claims, characterized in that:
R¹ is a hydrogen atom or a hydrocarbon with 1-18 carbon atoms, preferably for adjusting chemical characteristics of the polymer;
R² and R³ are linked to form a cyclic moiety of formula (II) comprising at least one N-hydroxysuccinimide ester for binding biologically active compounds or R² and R³ are independently selected from hydrogen, —COOH, methyl or at least N-hydroxsuccinimide bearing molecule for binding biologically active compounds;
R⁵ denotes a hydrogen atom, a carboxymethyl group or a methyl group;
x is 1; and
* denotes a chemical bond of the polymer backbone or to a terminating moiety wherein the terminating moiety preferably comprises a PNA sequence.

105. Polymer, especially polymer as building-block for hydrogel formation, according to any of the preceding claims, characterized in, that x is 1 or 2, preferably x is 1.

106. Polymer, especially polymer as building-block for hydrogel formation, according to any of the preceding claims, characterized in, that it comprises at least one moiety of formula (II), selected from a moiety of formula (II-a) 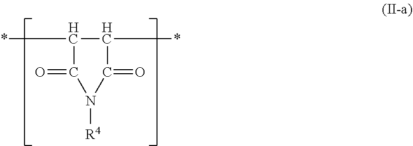
wherein
R⁴ is a moiety comprising at least one functional group
for crosslinking and/or
for binding biologically active compounds,
and optionally comprising a (preferably degradable) spacer moiety connecting said functional group with the binding site of R⁴ according to formula (II-a), and
and * denotes a chemical bond of the polymer backbone or to a terminating moiety.

107. Polymer, especially polymer as building-block for hydrogel formation, according to any one of the preceding claims, characterized in, that it comprises at least one moiety of formula (II), selected from a moiety of formula (II-b) 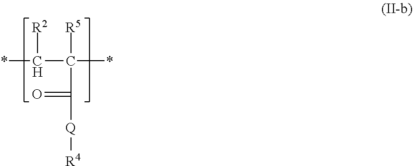
wherein
R⁵ and R⁴ is defined according to any of the preceding claims,
R² is a hydrogen atom or a carboxyl group,
Q denotes an oxygen atom or an imino group NH,
and * denotes a chemical bond of the polymer backbone or to a terminating moiety.

108. Polymer, especially polymer as building-block for hydrogel formation, according to any of the preceding claims, characterized in, that R⁴ is independently a moiety, comprising at least one functional group independently selected from arene, amine, alkyne, azide, anhydride, acid anhydride, ketone, haloalkane, imidoester, diol, hemiacetal, acrylate, alkene, thiol, ether, ester, isocyanate, isothiocyanate, succinimide, N-hydroxysuccinimide, sulfo-N-hydroxysuccinimide, amide, maleimide, N-heterocyclic carbene, acyl halide, N-heterocyclic phosphine, hydrazide, nitrile, aminoxy, imidazolide, imine, aldehyde, azo compound, imide, carbodiimide, haloacetyl, pyridyl disulfide, carboxamide, vinyl ether, carboxyl, carboxylate, phenyl, phenol, indol, methylthiol, pyridyldithiol, hydroxyl, epoxide, carbonyl, methoxycarbonyl, glycidyl, carboxyphenyl.

109. Polymer, especially polymer as building-block for hydrogel formation, according to any of the preceding claims, characterized in, that the moiety of the formula (II) is derived from at least one monomer selected from an unsaturated imide (preferably derived from maleimide), an alkene, an acrylic acid, an itaconic acid, a lactone (preferably β-propiolactone, α-methyl-β-propiolactone, α,α-dimethyl β-propiolactone, β-butyrolactone), an acrylamide, a sulfonamide (preferably ethylensulfonamide), an anhydride, a methacrylic acid, an acrylamide, a methacrylamide, a N,N-diacrylamide (preferably N-methyldiacrylamide), a 1-propanesulfonic acid sultone, with the proviso, that said monomer comprise said residue R⁴ respectively.

110. Polymer, especially polymer as building-block for hydrogel formation, according to any of the preceding claims, characterized in, that said functional group of residue R⁴ is independently selected from the group consisting of protected N-hydroxysuccinimide-esters, unprotected N-hydroxysuccinimide-esters, sulfo-N-hydroxysuccinimide esters, vinyl sulfone, sulfonyl chloride, aldehyde, epoxides, thiol, maleimide and carbonate, wherein preferably, the moiety of formula (II) comprises such residue R⁴.

111. Polymer, especially polymer as building-block for hydrogel formation, according to any of the preceding claims, characterized in, that the moiety of the formula (II) is derived from monomers selected from 3-(maleimido)-propionic acid N-hydroxysuccinimide ester, 6-Maleimidohexanoic acid N-hydroxysuccinimide ester, N-(Methacryloxy)-succinimideisopropenyl, BMPH (N-(β-maleimidopropionic acid)-hydrazide, EMCH (N-ε-maleimidocaproic acid hydrazide), PDPH (3-(2-pyridyldithio) propionyl hydrazide), Methacrylic acid N-hydroxysuccinimide ester, N-methoxycarbonyl maleimide, acrylic acid N-hydroxysuccinimide ester, a PNA-amide of acrylic acid, a PNA-amide of methacrylic acid, a PNA-amide of acrylamide, a PNA-amide of methacrylamide, a monomer of formula 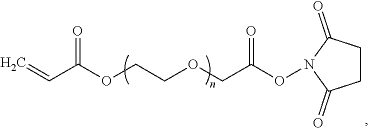
wherein n is an integer of at least 1,
a monomer of formula 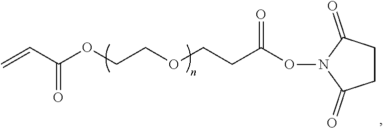
wherein n is an integer of at least 1,
a monomer of formula, 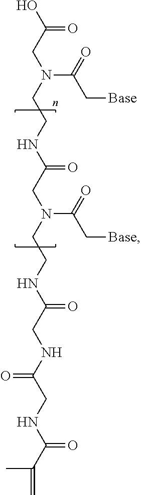
wherein n is an integer greater than 1 and Base is independently
a moiety comprising at least one nucleobase,
or mixtures thereof.

112. Polymer, especially polymer as building-block for hydrogel formation, characterized in, that it comprises at least one (m is an integer of at least 1) unit having the structure of formula (III) 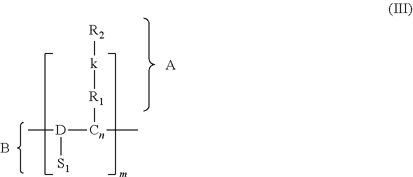
R₂ is independently a residue R⁴, comprising at least one functional group
for crosslinking and/or
for binding biologically active compounds,
S₁ is independently defined according to R¹ of claim 101,
fragment D-Cn is part of the polymer backbone,
wherein said structure results from polymerization of a heterocyclic molecule B in presence of a first component A.

113. Polymer, especially polymer as building-block for hydrogel formation, according to claim 112, characterized in, that said first component A is a compound of formula (IV)
R₁-k-R₂ (IV)
wherein
R₁ is a first functional group for the copolymerization with said heterocyclic molecule B,
R₂ is said moiety R⁴,
k is a direct bond or a spacer.

114. Polymer, especially polymer as building-block for hydrogel formation, according to claim 112 or claim 113, characterized in, that k is selected from a direct bond, alkylidene groups with 2 to 8 carbon atoms, hydrocarbons, and/or a degradable spacer (preferably selected from peptides, PNA, polyethylene glycol).

115. Polymer, especially polymer as building-block for hydrogel formation, according to any of the claims 112 to 114, characterized in, that said first component A of formula (IV) is selected from the monomers as defined in any of the claims 109 to 111.

116. Polymer, especially polymer as building-block for hydrogel formation, according to any of the claims 112 to 115, characterized in, that said heterocyclic molecule B is a 2-substituted heterocyclic compound of formula (V)
D-S₁ (V)
wherein
D is an oxazoline-moiety, oxazine-moiety or oxyazepine-moiety and
S₁ is a substituent in 2-position as defined as R¹ of claim 1.

117. Polymer, especially polymer as building-block for hydrogel formation, according to any of the claims 112 to 116, characterized in, that said unit is a covalently functionalized D-substituted alkylamine.

118. Polymer, especially polymer as building-block for hydrogel formation, according to any of the claims 112 to 117, characterized in, that it is a polymer according to any claim of claims 101 to 111.

119. Polymer, especially polymer as building-block for hydrogel formation, of formula (P1) 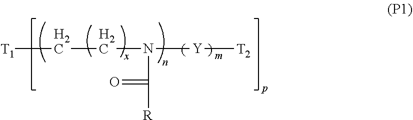
wherein
R is independently selected from a hydrogen atom, a hydrocarbon with 1-18 carbonatoms (preferably CH₃, —C₂H₅), a C₁-C₂₅-hydrocarbon with at least one hydroxy group, a C₁-C₂₅-hydrocarbon with at least one carboxy group, (C₂-C₆)alkylthiol, (C₂-C₆)alkylamine, protected (C₂-C₆)alkylamine (preferably-(CH₂)₂₋₆—NH—CO—R (with R=tert-Butyl, perfluoroalkyl)), (C₂-C₆)alkylazide, polyethylene glycol, polylactic acid, polyglycolic acid, polyoxazoline, or wherein R is a residue R⁴
Y is a moiety containing at least one graft, comprising at least one residue R⁴,
T₁ is a terminating moiety, which may contain a residue R⁴,
T₂ is a terminating moiety, which contains a residue R⁴,
p is an integer from 1 to 10,
n is an integer greater than 1 and preferably, below 500,
m is zero or an integer of at least, preferably greater than 1, and preferably, below 500,
the sum n+m is greater than 10,
x is independently 1, 2 or 3, preferably x is independently 1 or 2, most preferably x is 1,
R⁴ independently comprise at least one functional group
for crosslinking and/or
for binding biologically active compounds, and
optionally comprising a (preferably degradable) spacer moiety connecting said functional group with the binding site to the respective moiety of the structure of formula (P1),
wherein the entirety of all m-fold and n-fold repeating units are distributed in any order within the polymer chain and wherein optionally, the polymer is a random copolymer or a block copolymer.

120. Polymer, especially polymer as building-block for hydrogel formation, of claim 119 characterized in, that Y is a moiety of formula (II) as defined in any of the claims 101 to 111.

121. Polymer, especially polymer as building-block for hydrogel formation, of claim 119 or claim 120, characterized in, that R is a hydrogen atom or a C₁-C₁₈-alkyl group, (preferably a hydrogen atom, methyl, ethyl, n-propyl, iso-propyl, n-butyl, iso-butyl, sec-butyl, tert-butyl, pentyl, iso-pentyl, neopentyl, sec-pentyl, hexyl, heptyl, octyl, nonyl, decyl) and m is an integer greater than 1.

122. Polymer, especially polymer as building-block for hydrogel formation, of any of claims 119 to 121, characterized in, that
R is a hydrogen atom, a hydrocarbon with 1-18 carbonatoms (preferably CH3, —C2H5);
Y is a moiety containing at least one graft, comprising at least one degradable spacer moiety connecting at least one N-hydroxysuccinimide ester for binding biologically active compounds to the respective moiety of the structure of formula (P1);
T¹ is a terminating moiety, optionally comprising a peptide nucleic acid (PNA) sequence;
T² is a terminating moiety, optionally comprising a peptide nucleic acid (PNA) sequence;
n is an integer greater than 1;
m is an integer greater than 1;
the sum n+m is greater than 10 and less than 500; and
x is 1;
wherein the entirety of all m-fold and n-fold repeating units are distributed in any order within the polymer chain and wherein optionally, the polymer is a random copolymer or a block copolymer.

123. Polymer, especially polymer as building-block for hydrogel formation, according to any of the claims 119 to 121, characterized in, that
T₁ is a terminating moiety, comprising a first XNA-residue (XNA1) and optionally a EDTS-moiety,
T₂ is a terminating moiety, comprising a second XNA-residue (XNA2) and optionally a EDTS-moiety,
p equals 1 or 2, preferably equals 1,
EDTS is an enzyme degradable target site, preferably a matrix metalloprotease (MMP) target site, for site directed degradation of the polymer,
XNA is a nucleic acid or nucleic acid analog, preferably a peptide nucleic acid (PNA) sequence.

124. Polymer, especially polymer as building-block for hydrogel formation, of claim 123, characterized in, that m is zero and no moiety Y is comprised in the polymer.

125. Polymer, especially polymer as building-block for hydrogel formation, according to any of the claims 119 to 121, characterized in, that
T₁ is a terminating moiety, comprising no residue R⁴,
T₂ is a terminating moiety, comprising a XNA-residue, optionally linked to a EDTS-moiety,
p is an integer of 3 to 10, preferably 3 to 10, preferably 3 to 8, most preferred 3 to 6,
EDTS is an enzyme degradable target site, preferably a matrix metalloprotease (MMP) target site, for site directed degradation of the polymer,
XNA is a nucleic acid or nucleic acid analog, preferably a peptide nucleic acid (PNA) sequence.

126. Polymer, especially polymer as building-block for hydrogel formation, of claim 125, characterized in, that m is zero and no moiety Y is comprised in the polymer.

127. Polymer, especially polymer as building-block for hydrogel formation, according to any of the claims 119 to 121, characterized in, that
T₁ is a terminating moiety, comprising a residue R⁴ different from a XNA-residue, wherein R⁴ is optionally linked to a EDTS-moiety,
T₂ is a terminating moiety, comprising a residue R⁴ different from a XNA-residue, wherein R⁴ is optionally linked to an EDTS-moiety,
p equals 1 or 2, preferably equals 1,
EDTS is an enzyme degradable target site, preferably a matrix metalloprotease (MMP) target site, for site directed degradation of the polymer,
XNA is a nucleic acid or nucleic acid analog, preferably a peptide nucleic acid (PNA) sequence.

128. Polymer, especially polymer as building-block for hydrogel formation, of claim 127, characterized in, that m is zero and no moiety Y is comprised in the polymer.

129. Polymer, especially polymer as building-block for hydrogel formation, according to any of the claims 119 to 121, characterized in, that
T₁ is a terminating moiety, comprising no residue R⁴,
T₂ is a terminating moiety, comprising a residue R⁴ different from a XNA-residue, wherein R⁴ is optionally linked to an EDTS-moiety,
p is an integer of 3 to 10, preferably 3 to 10, preferably 3 to 8, most preferred 3 to 6,
EDTS is an enzyme degradable target site, preferably a matrix metalloprotease (MMP) target site, for site directed degradation of the polymer,
XNA is a nucleic acid or nucleic acid analog, preferably a peptide nucleic acid (PNA) sequence.

130. Polymer, especially polymer as building-block for hydrogel formation, of claim 129, characterized in, that m is zero and no moiety Y is comprised in the polymer.

131. Polymer especially polymer as building-block for hydrogel formation, according to any of the claims 123 to 130, characterized in, that it is a polymer which comprises an EDTS-moiety, preferably a MMP-moiety.

132. Polymer, especially polymer as building-block for hydrogel formation, according to any of the claims 119 to 131, characterized in, that it comprises at least two different moieties R.

133. Polymer, especially polymer as building-block for hydrogel formation, according to any of the claims 119 to 121, characterized in, that p is an integer of 3 to 10, preferably 3 to 10, preferably 3 to 8, most preferred 3 to 6.

134. Polymer, especially polymer as building-block for hydrogel formation, of formula (P2) 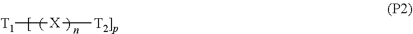
wherein
T₁ is a terminating moiety, which contains a residue -XDTS-XNA1,
T₂ is a terminating moiety, which contains a residue -XDTS-XNA2,
XDTS is independently selected from a direct bond or an EDTS-moiety, wherein EDTS is an enzyme degradable target site, preferably a matrix metalloprotease (MMP) target site, for site directed degradation of the polymer,
XNA1 is a nucleic acid or nucleic acid analog, preferably a peptide nucleic acid (PNA) sequence,
XNA2 is the same or a different nucleic acid or nucleic acid analog compared to XNA1, preferably a peptide nucleic acid (PNA) sequence,
p is 1 or 2, preferably 1,
X is a hydrophilic polymeric residue, preferably independently derived from monomers independently selected from oxazoline, ethylene glycol, propylene glycol, acetal lactic acid, glycolic acid, vinyl alcohol,
n is an integer greater than 1, preferably from 1 to 10000.
According to one embodiment, at least one X is different from oxazoline.

135. Polymer, especially polymer as building-block for hydrogel formation, according to formula (P2) of claim 134, characterized in that
T₁ is a terminating moiety, comprising no XNA-residue,
T₂ is a terminating moiety, comprising a XNA-residue and optionally an EDTS-moiety,
p is an integer of 3 to 10, preferably 3 to 8, most preferred 3 to 6,
X hydrophilic polymeric residue, preferably independently derived from monomers independently selected from oxazoline, ethylene glycol, propylene glycol, acetal lactic acid, glycolic acid, vinyl alcohol,
EDTS is an enzyme degradable target site, preferably a matrix metalloprotease (MMP) target site, for site directed degradation of the polymer,
XNA is a nucleic acid or nucleic acid analog, preferably a peptide nucleic acid (PNA) sequence,
n is an integer greater than 1, preferably from 1 to 10000.

136. Polymer, especially polymer as building-block for hydrogel formation, according to any of the preceding claims, wherein the polymer is functionalized by at least one biologically active compound, preferably, at least two different biologically active compounds, preferably by reaction of an amino group of the biologically active compound with a functional group of residue R⁴.

137. Polymer, especially polymer as building-block for hydrogel formation, according to any of the preceding claims, characterized in, that the biologically active compound selected from the group consisting of peptides, proteins, CRISPR-Cas enzyme complex, apoptosis-inducing active substances, adhesion-promoting active substances, anti-inflammatory active substances, receptor agonists and receptor antagonists, growth-inhibiting active substances (and in particular from proteins of the extracellular matrix, cell surface proteins, antibodies, growth factors, sugars, lectins, carbohydrates, cytokines, DNA, RNA, siRNA), aptamers, and fragments thereof, or mixtures thereof.

138. Polymer, especially polymer as building-block for hydrogel formation, according to any of the preceding claims, characterized in, that it comprises at least one biologically active compound selected from the group consisting of peptides, proteins, CRISPR-Cas enzyme complex, apoptosis-inducing active substances, adhesion-promoting active substances, anti-inflammatory active substances, receptor agonists and receptor antagonists, growth-inhibiting active substances (and in particular from proteins of the extracellular matrix, cell surface proteins, antibodies, growth factors, sugars, lectins, carbohydrates, cytokines, DNA, RNA, siRNA), aptamers, and fragments thereof, or mixtures thereof.

139. Polymer, especially polymer as building-block for hydrogel formation, according to any of the preceding claims, characterized in, that it comprises at least one biologically active compound selected from a Peptide nucleic acid (PNA) and/or a locked nucleic acid (LNA), preferably wherein the PNA-moiety independently comprise a structure of formula (VI) 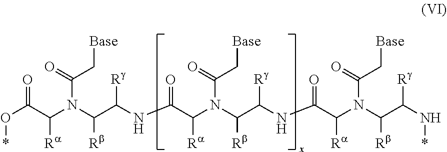
wherein
x is an integer greater than 1,
Base is independently a moiety comprising at least one nucleobase (preferably selected from adenin, cytosin, guanine, thymine, 2,6-diaminopurine, analogs of thymine and cytosine, hypoxanthine, derivatives thereof functionalized with a fluorescent dye (preferably thiazole orange)),
Rα and Rβ are independently selected from hydrogen atom, any residue bound to the alpha-carbon atom of any of the proteinogenic amino acid,
Rγ is a hydrogen atom, a moiety with at least one ionic residue.

140. Polymer, especially polymer as building-block for hydrogel formation, according to any of the preceding claims, characterized in, that it comprises at least one biologically active compound, selected from a Peptide nucleic acid (PNA) comprising a matrix metalloprotease target site for the site directed degradation (MMP).

141. Polymer, especially polymer as building-block for hydrogel formation, according to claim 139 or claim 140, characterized in, that is comprises at least one additional biologically active compound, selected from the group consisting of peptides, proteins, CRISPR-Cas enzyme complex, apoptosis-inducing active substances, adhesion-promoting active substances, anti-inflammatory active substances, receptor agonists and receptor antagonists, growth-inhibiting active substances (and in particular from proteins of the extracellular matrix, cell surface proteins, antibodies, growth factors, sugars, lectins, carbohydrates, cytokines, DNA, RNA, siRNA), aptamers, and fragments thereof, or mixtures thereof.

142. Polymer, especially polymer as building-block for hydrogel formation, according to any one of the preceding claims, wherein the polymer has a linear structure (preferably a graft polymer, grafted with at least one residue R⁴) or a dendritic structure (preferably a linear structure or a star shaped structure).

143. Polymer, especially polymer as building-block for hydrogel formation, according to any one of the preceding claims, wherein the polymer is random polymer, a block-copolymer or a dendrimer.

144. Polymer, especially polymer as building-block for hydrogel formation, according to any one of the preceding claims, wherein the polymer has a star-shaped structure comprising at least three arms.

145. Polymer, especially polymer as building-block for hydrogel formation, according to any of the preceding claims, wherein said functional group for crosslinking is selected from amine, N-hydroxysuccinimide, sulfo-N-hydroxysuccinimide, isothiocyanate, maleimide, thiol, azide, alkyne, alkene, hydrazide, aminoxy, aldehyde, carboxyl, carboxylate, hydroxyl, acrylate, vinyl ether, epoxide (preferably from amine, maleimide, alkyne, alkene, azide, carboxyl, carboxylate, methacrylate, acrylate, thiol).

146. Polymer, especially polymer as building-block for hydrogel formation, according to any of the preceding claims, wherein said functional group for binding a biologically active compound is independently selected from amine, N-hydroxysuccinimide, sulfo-N-hydroxysuccinimide, alkyne, alkene, hydrazide, epoxide, glycidyl, carboxyphenyl, methoxycarbonyl, carboxyl, carboxylate, isothiocyanate, maleimide, aminoxy, hydroxyl, vinyl ether (preferably from amine, N-hydroxysuccinimide, sulfo-N-hydroxysuccinimide, hydrazide, epoxide, glycidyl, phenyl acrylate, methoxycarbonyl, carboxyl, carboxylate).

147. Polymer, especially polymer as building-block for hydrogel formation, according to any of the preceding claims, wherein the polymer is prepared by at least one polymerization step, selected from living cationic ring-opening polymerization (CROP), spontaneous zwitterionic copolymerization (SZWIP) or a combination of both.

148. Polymer, especially polymer as building-block for hydrogel formation, according to claim 147, characterized in, that the polymerization, preferably the living cationic ring-opening polymerization, is initiated by an initiator with an electrophilic character.

149. Polymer, especially polymer as building-block for hydrogel formation, according to claim 147 or claim 148, characterized in, that the initiator is selected from triethylene glycol di (p)-toluenesulfonate, pentaerythritol tetrabromide, pentaerythritol tetrakis(benzenesulfonate) or p-toluenesulfonyl chloride modified N,N,N′,N′-Tetrakis(2-hydroxyethyl)ethylenediamine.

150. Polymer, especially polymer as building-block for hydrogel formation, according to any of claims 147 to 149, characterized in, that the polymerization, preferably the living cationic ring-opening polymerization, is terminated by addition of a terminating molecule selected from nucleophiles, amines, azides or acids (preferably carboxylic acids).

151. Polymer, especially polymer as building-block for hydrogel formation, according to any of any of claims 147 to 150, characterized in, that the polymerization, preferably the living cationic ring-opening polymerization, is terminated by addition of a terminating molecule selected from peptide nucleic acid (PNA), preferably peptide nucleic acid (PNA) with unprotected carboxylic acid group at the C-terminus and protected amino group at the N-terminus or peptide nucleic acid (PNA) with unprotected amino group at the N-terminus and protected carboxylic acid group at the C-terminus).

152. Polymer, especially polymer as building-block for hydrogel formation, according to any of any of claims 147 to 151, characterized in, that the polymerization, preferably the spontaneous zwitterionic copolymerization, is terminated by addition of a terminating molecule selected from electrophiles, preferably selected from α,β-unsaturated carboxylic acids, α,β-unsaturated carboxylic acidamides, mixtures thereof, most preferred from acrylic acid, methacrylic acid, acryl amide, methacryl amide, functionalized with at least one residue R⁴ as defined in any of the preceding claims respectively (most preferred functionalized with -MMP-PNA respectively).

153. Polymer, especially polymer as building-block for hydrogel formation, according to any of any of claims 147 to 152, characterized in, that said initiator and/or said terminating molecule incorporates a moiety R⁴ as defined in any of claims 101, 108 to 110, 145 and 146.

154. Polymer, especially polymer as building block for hydrogel formation, according to any of the claims 147 to 151 and 153, characterized in, that the polymerization, preferably the spontaneous zwitterionic copolymerization, is terminated by addition of a terminating molecule selected from selected from α,β-unsaturated carboxylic acids, α,β-unsaturated carboxylic acidamides, mixtures thereof (most preferred from acrylic acid, methacrylic acid, acryl amide, methacryl amide) followed after optional workup by a coupling of a residue comprising PNA and a thiol functionality.

155. Polymer, especially polymer as building block for hydrogel formation, according to any of the claims 147 to 154, characterized in, that a residue comprising PNA and a thiol functionality is coupled to a maleimide as a functional group of residue R⁴.
